SEQUENCE LISTING (7 sequences)

SEQ ID NO: 1-4 - skipped (4 entries)

SEQ ID NO: 5 - protein, 1008 aa, Clostridium histolyticum
IANTNSEKYDFEYLNGLSYTELTNLIKNIKWNQINGLFNYSTGSQKFFGDKNRVQAIINALQESGRTYTANDMKGIETFTEVLRAGFYLGYYNDGLSYLNDRNFQDKCIPAMIAIQKNPNFKLGTAVQDEVITSLGKLIGNASANAEVVNNCVPVLKQFRENLNQYAPDYVKGTAVNELIKGIEFDFSGAAYEKDVKTMPWYGKIDPFINELKALGLYGNITSATEWASDVGIYYLSKFGLYSTNRNDIVQSLEKAVDMYKYGKIAFVAMERITWDYDGIGSNGKKVDHDKFLDDAEKHYLPKTYTFDNGTFIIRAGEKVSEEKIKRLYWASREVKSQFHRVVGNDKALEVGNADDVLTMKIFNSPEEYKFNTNINGVSTDNGGLYIEPRGTFYTYERTPQQSIFSLEELFRHEYTHYLQARYLVDGLWGQGPFYEKNRLTWFDEGTAEFFAGSTRTSGVLPRKSILGYLAKDKVDHRYSLKKTLNSGYDDSDWMFYNYGFAVAHYLYEKDMPTFIKMNKAILNTDVKSYDEIIKKLSDDANKNTEYQNHIQELADKYQGAGIPLVSDDYLKDHGYKKASEVYSEISKAASLTNTSVTAEKSQYFNTFTLRGTYTGETSKGEFKDWDEMSKKLDGTLESLAKNSWSGYKTLTAYFTNYRVTSDNKVQYDVVFHGVLTDNADISNNKAPIAKVTGPSTGAVGRNIEFSGKDSKDEDGKIVSYDWDFGDGATSRGKNSVHAYKKTGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKATKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK

SEQ ID NO: 6 - protein, 991 aa, Clostridium histolyticum
AVDKNNATAAVQNESKRYTVSYLKTLNYYDLVDLLVKTEIENLPDLFQYSSDAKEFYGNKTRMSFIMDEIGRRAPQYTEIDHKGIPTLVEVVRAGFYLGFHNKELNEINKRSFKERVIPSILAIQKNPNFKLGTEVQDKIVSATGLLAGNETAPPEVVNNFTPIIQDCIKNMDRYALDDLKSKALFNVLAAPTYDITEYLRATKEKPENTPWYGKIDGFINELKKLALYGKINDNNSWIIDNGIYHIAPLGKLHSNNKIGIETLTEVMKIYPYLSMQHLQSADQIERHYDSKDAEGNKIPLDKFKKEGKEKYCPKTYTFDDGKVIIKAGARVEEEKVKRLYWASKEVNSQFFRVYGIDKPLEEGNPDDILTMVIYNSPEEYKLNSVLYGYDTNNGGMYIEPDGTFFTYERKAEESTYTLEELFRHEYTHYLQGRYAVPGQWGRTKLYDNDRLTWYEEGGAELFAGSTRTSGILPRKSIVSNIHNTTRNNRYKLSDTVHSKYGASFEFYNYACMFMDYMYNKDMGILNKLNDLAKNNDVDGYDNYIRDLSSNHALNDKYQDHMQERIDNYENLTVPFVADDYLVRHAYKNPNEIYSEISEVAKLKDAKSEVKKSQYFSTFTLRGSYTGGASKGKLEDQKAMNKFIDDSLKKLDTYSWSGYKTLTAYFTNYKVDSSNRVTYDVVFHGYLPNEGDSKNSLPYGKINGTYKGTEKEKIKFSSEGSFDPDGKIVSYEWDFGDGNKSNEENPEHSYDKVGTYTVKLKVTDDKGESSVSTTTAEIKDLSENKLPVIYMHVPKSGALNQKVVFYGKGTYDPDGSIAGYQWDFGDGSDFSSEQNPSHVYTKKGEYTVTLRVMDSSGQMSEKTMKIKITDPVYPIGTEKEPNNSKETASGPIVPGIPVSGTIENTSDQDYFYFDVITPGEVKIDINKLGYGGATWVVYDENNNAVSYATDDGQNLSGKFKADKPGRYYIHLYMFNGSYMPYRINIEGSVGR

SEQ ID NO: 7 - protein, 6 aa, Artificial Sequence
  Description of Artificial Sequence: Synthetic peptide
  MOD_RES (1)..(1): Carbobenzoxy-Gly
GPGGPA

(19) United States
(12) Patent Application Publication
(10) Pub. No.: US 2024/0277595 A1
(43) Pub. Date: Aug. 22, 2024

(54) COMPOSITIONS AND METHODS FOR TREATING CELLULITE

(71) Applicant: ENDO GLOBAL AESTHETICS LIMITED, Dublin (IE)

(72) Inventors: Michael MCLANE, Lansdale, PA (US); Matthew W. DAVIS, Malvern, PA (US)

(21) Appl. No.: 17/276,507

(22) PCT Filed: Sep. 4, 2019
(86) PCT No.: PCT/IB2019/000955

§ 371 Date: Mar. 16, 2021

Related U.S. Application Data
(63) Continuation of application No. PCT/IB2019/000767, filed on Jul. 11, 2019.
Application No. PCT/IB2019/000767 is a continuation of application No. PCT/IB2019/000777, filed on Jul. 12, 2019.
(60) Provisional application No. 62/733,046, filed on Sep. 18, 2018.
(60) Provisional application No. 62/788,916, filed on Jan. 6, 2019.
(60) Provisional application No. 62/812,036, filed on Feb. 28, 2019.
(60) Provisional application No. 62/823,596, filed on Mar. 25, 2019.

(30) Foreign Application Priority Data
Jul. 11, 2019 (WO) PCT/IB2019/000767
Jul. 12, 2019 (WO) PCT/IB2019/000777

(51) Int. Cl. A61K 8/66; A61Q 19/06

(52) U.S. Cl. CPC A61K 8/66; A61Q 19/06; C12Y 304/24003; A61K 2800/91

(57) ABSTRACT

The present disclosure relates to a method of treating cellulite on a thigh or buttock in a human subject by administering an effective amount of collagenase, and then assessing the reduction in the severity of cellulite by one or more scales.

### CROSS-REFERENCE TO RELATED APPLICATIONS

This application is the U.S. National Stage Application of International Patent Application No. PCT/IB2019/000955, filed Sep. 4, 2019, which claims priority to U.S. Provisional Application Ser. No. 62/733,046 filed on Sep. 18, 2018, U.S. Provisional Application Ser. No. 62/788,916 filed on Jan. 6, 2019, U.S. Provisional Application Ser. No. 62/812,036 filed on Feb. 28, 2019, U.S. Provisional Application Ser. No. 62/823,596 filed on Mar. 25, 2019, International Appl. Ser. No. PCT/IB2019/000767 filed on Jul. 11, 2019, and International Appl. Ser. No. PCT/IB2019/000777 filed on Jul. 12, 2019, the contents of each of which are incorporated herein by reference in their entirety to the full extent permitted by law.

### SEQUENCE LISTING

The instant application contains a Sequence Listing which has been submitted electronically in ASCII format and is hereby incorporated by reference in its entirety. The ASCII copy, created on Jul. 21, 2021, is named 117326.000451_SL and is 18,066 bytes in size.

### TECHNICAL FIELD

The present invention relates to the field of assessing and treating cellulite.

### BACKGROUND

Cellulite (also known as edematous fibrosclerotic panniculopathy (EFP)), is an aesthetic condition that can be understood as an imbalance between the structural characteristics and biomechanical properties (i.e., the delicate containment and extrusion forces) at the subdermal junction (Rudolph et al, “Structural Gender Dimorphism and the Biomechanics of the Gluteal Subcutaneous Tissue: Implications for the Pathophysiology of Cellulite,” Plast. Reconstr. Surg. 2019; 143(4):1077-1086). Accordingly, the goals of cellulite treatment are to strengthen the subdermal interface and/or to release the fibrous septae via various types of subcision (Rudolph et al., supra). The fibrous septae has been recognized as a contributory underlying cause of cellulite and as a target of treatment for cellulite by anatomical and image analyses studies (Hexsel et al, “Side-by-side comparison of areas with and without cellulite depressions using magnetic resonance imaging,” Dermatol Surg. 2009; 35(10):1471-1477; Hexsel et al. “Magnetic Resonance Imaging of Cellulite Depressed Lesions Successfully Treated by Subcision,” Dermatol Surg. 2016; 42(5):693-696; Mirrashed F, Sharp J C, Krause V, Morgan J, Tomanek B. “Pilot study of dermal subcutaneous fat structures by MRI in individuals who differ in gender, BMI, and cellulite grading,” Skin Res Technol. 2004; 10(3):161-168; Nurnberger and Muller, “So-called cellulite: an invented disease,” J Dermatol Surg Oncol. 1978; 4(3):221-229; Pierard et al, “Cellulite: from standing fat herniation to hypodermal stretch marks,” Am J Dermatopath. 2000; 22(1):34-37; Querleux et al, “Anatomy and physiology of subcutaneous adipose tissue by in vivo magnetic resonance imaging and spectroscopy: relationships with sex and presence of cellulite,” Skin Res Technol. 2002; 8(2):118-124). To effectively treat cellulite, a therapeutic approach is needed to lyse or otherwise disrupt the dermal septa, which are composed of collagen (FIG. 1) and cause the skin dimpling that is bothersome to many women.

There are therapies that have been utilized in an attempt to treat cellulite; however, there are no approved pharmacologic treatments. Despite multiple therapeutic modalities, there is little scientific evidence that any of the current non-pharmacologic treatments are beneficial. In fact, much of the evidence is anecdotal, subjective, or based only on patient self-assessment. Some of the historical treatments for EFP have included weight loss, topical agents, massage, liposuction, mesotherapy, radiofrequency, subcision, powered subcision, and laser therapies. Many of these treatments have undesirable side effects (Avram M M, “Cellulite: a review of its physiology and treatment,” J Cosmet Laser Ther. 2004; 6(4):181-185; Collis et al, “Cellulite treatment: a myth or reality: a prospective randomized, controlled trial of two therapies, endermologie and aminophylline cream,” Plast Reconstr Surg. 1999; 104(4):1110-1114; Khan M H, Victor F, Rao B, Sadick N S. “Treatment of cellulite: Part I. Pathophysiology.” J Am Acad Dermatol. 2010; 62(3):361-370; Hexsel D M, Mazzuco R. “Subcision: a treatment for cellulite.” Int J Dermatol. 2000; 39(7):539-544; Boyce et al, “Clinical evaluation of a device for the treatment of cellulite: Triactive.” Am J Cosmet Surg. 2005; 22:233-237; DiBernardo B E. “Treatment of cellulite using a 1440-nm pulsed laser with one-year follow-up.” Aesthet Surg J. 2011; 31(3):328-341). As such, many physicians are of the view that improvements for aesthetic conditions are not easily obtained. Thus, there remains an unmet need for safe and effective nonsurgical therapies to improve the aesthetic outcome in women with cellulite.

### SUMMARY

The present disclosure satisfies the above need and relates to methods of treating cellulite in human patients by the subcutaneous injection of a therapeutically effective amount of collagenase (as defined in the Detailed Description). Such methods relate to the pretreatment assessment of a patient's severity of cellulite using various scales and assessment techniques to establish the patient's baseline of cellulite severity. This is then followed by the treatment of the cellulite by the subcutaneous injection of collagenase. The dosing and administration of the collagenase may vary, and the collagenase may be in the form of a pharmaceutical composition comprising the collagenase and one or more pharmaceutically acceptable excipients. Such excipients may include sterile water for injection, pH adjusting agents, tonicity adjusting agents and stabilizers. Post-treatment assessments are performed to confirm the efficacy of the treatment compared to baseline. The methods of treatments of the present disclosure result in significant reductions in the appearance of cellulite.

As explained in the Detailed Description, there are four phases of treatment, although they are optional and the order is not intended to be strictly limiting.

1. In a first phase, the clinician performs a selection of cellulite dimples to be treated. Next, before injection, an assessment is performed, e.g., the clinician and/or patient independently assess the pretreatment severity of cellulite using one or more of the following scales or other assessment methods (as defined in the Detailed Description):
Hexsel Cellulite Severity Scale (Hexsel CSS)
Hexsel Depression Depth Score
Likert Scale
Dimple Analysis
Clinician Reported Photonumeric Cellulite Severity Scale (CR-PCSS)
Patient Reported Photonumeric Cellulite Severity Scale (PR-PCSS)
Investigator Global Aesthetic Improvement Scale (I-GAIS)
Subject Global Aesthetic Improvement Scale (S-GAIS)
Patient Reported Cellulite Impact Scale (PR-CIS)
PR-CIS Abbreviated
Subject Self-Rating Scale (SSRS)
Subject Satisfaction with Cellulite Treatment (SSCT)
Clinician assessment of cellulite severity (photography or other imagery)
Body-Q
Fitzpatrick scale
Thigh Cellulite Severity-Patient (TCS-P); Thigh Cellulite Severity-Clinician (TCS-C)
Any validated photonumeric or other scale used by clinicians and/or patients to assess cellulite severity, improvement, and/or patient satisfaction (e.g., Hexsel-Merz Scale (Hexsel et al., “Validated Assessment Scales for Cellulite Dimples on the Buttocks and Thighs in Female Patients,” Dermatologic Surgery: August 2019 (Volume 45) Issue p S2-S11 and poster publication at American Academy of Dermatology meeting 2019).

Further, the pretreatment assessment by clinicians and patients may be performed by analyzing a series of 1 to 15 photographs, illustrations, drawings, computer images, 3-D models, MRI images, thermograms, ultrasonograms, patient verbal feedback or the like each having a different cellulite severity rating or level.

2. In a second phase of treatment, dimples to be treated are marked by the clinician with a dot or other marking (FIG. 6). It is typically placed at the nadir of the dimple, if a nadir is present. More photographs may be taken and other assessments performed.

3. In a third phase of treatment, a therapeutically effective amount of collagenase is injected subcutaneously into the dimple(s) in a single dose or divided doses at one or more treatment areas (as defined in the Detailed Description). The doses and injection techniques vary. For example, the method may comprise an injection according to the following procedure:
A collagenase composition (e.g., CCH) is injected subcutaneously while the subject is in a prone position using a syringe with a 30-gauge 12 inch needle. As shown in FIG. 7 (hereafter “Treatment I”), each injection site receives a single skin injection of collagenase composition administered as three 0.1 mL aliquots to Positions A, B, and C, for a total injection volume of 0.3 mL. The depth of injection is 12 inch, corresponding to the length of the treatment needle from the tips of the needle to the base of the needle without downward pressure. At each injection site, the needle is positioned at 900 perpendicular to the skin surface and inserted, and a 0.1 mL aliquot of collagenase composition is injected (Position A). The needle is withdrawn slightly (but not removed from the skin) and repositioned 450 off vertical and above the long axis of the dimple, and 0.1 mL aliquot of collagenase composition is injected (Position B, in the direction of the head). The needle is again withdrawn slightly and repositioned approximately 45° off vertical and below the long axis of the dimple, and 0.1 mL aliquot of collagenase composition is injected (Position C, in the direction of the feet). After injection, the subject remains prone for 5 minutes.

In one example, Treatment I may be employed to administer 0.84 mg collagenase composition as 12 subcutaneous injections per treatment area during three treatment sessions, each occurring at least 21 days apart (+/−3 day window). For instance, a cumulative dose of 5.04 mg may be administered (i.e., 3 treatment visits×0.84 mg per treatment area×2 treatment areas). Other techniques are explained in the Detailed Description.

4. In a fourth phase of treatment, post-injection assessments are performed using the above-mentioned scales and other assessment methods (e.g., bruising analysis). Efficacy of a particular collagenase treatment may be based on a single clinician rating or patient rating, or based on a composite endpoint comprising the clinician rating and the patient rating where improvement is shown in both scales for the same subject, i.e., a pre-specified level of improvement is demonstrated in both the clinician and patient scales.

The collagenase is injected in an amount of about 0.01 mg to about 20 mg in a single dose or divided doses, and has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to 130 kDa, or about 70 to about 130 kDa, or about 80 to 120 kDa, or about 90 to 120 kDa, or about 100 to 110 kDa.
A purity by area of at least 80% as measured by reverse phase HPLC (high pressure liquid chromatography)
Potency (i.e., specific activity) of about 500 to 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden
   As used herein, the relevant kinetic parameters may be measured using the cuvette assays or microplate assays (e.g., the SRC cuvette assay, the SRC microplate assay, the GPA cuvette assay, and the GPA microplate assay) as described herein.

In some embodiments, the collagenase present in the composition comprises collagenase I and collagenase II in a ratio of approximately 1:1. Other ratios of collagenase I and collagenase II may be employed such as 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1. Each of collagenase I and collagenase II may have a purity by area of at least 80%, or 85%, or 90%, or 91%, or 92%, or 93%, or 94%, or 95%, or 96%, or 97%, or 98%, or 99%, or 100% as measured by reverse phase HPLC.

In another embodiment, the collagenase composition comprises CCH (as defined in the Detailed Description) having an AUX I and AUX II ratio of approximately 1:1. Other ratios of AUX I and AUX II may be employed such as 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1. Each of AUX I and AUX II may have a purity by area of at least 80%, or 85%, or 90%, or 91%, or 92%, or 93%, or 94%, or 95%, or 96%, or 97%, or 98%, or 99%, or 100% as measured by reverse phase HPLC.

In other examples, the collagenase composition may be a liquid or is reconstituted from a lyophilized solid form with a diluent. The dose of the mixture is measured by the amount of collagenase present without regard to diluent, and may comprise about 0.1 mg to about 20 mg in one or more injections. In another embodiment, the dose administered is about 0.06 mg, 0.48 mg, 0.84 mg, 1.68 mg, 2.52 mg, 3.36 mg, 4.2 mg, 5.04 mg, 5.88 mg, 6.72 mg, 7.56 mg, or 8.4 mg in one or more injections. For instance, about 0.06 mg, 0.48 mg, 0.84 mg, or 1.68 mg is administered in about 12 divided injections. The volume of collagenase composition injected may range from 0.01 mL to 3 mL per injection, or total about 0.2 mL to 150 mL per treatment visit (as defined in the Detailed Description). In a specific embodiment, the above doses are to a collagenase composition comprising CCH. In another embodiment, the above doses are to a collagenase composition having one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another embodiment, about 0.84 mg of CCH is injected in about 12 equally divided injections per treatment area (about 0.07 mg×12 injections=about 0.84 mg). In some cases, such treatment with 0.84 mg occurs in one treatment visit, or every 10-40 days for 2, 3, 4 or 5 treatment visits. In other cases, more than one treatment area is injected with 0.84 mg every 10-40 days for 2, 3, 4 or 5 treatment visits. Such injections may be administered in more than 5 treatment visits.

Further, as described in the Detailed Description, the collagenase injections are effective in treating cellulite. For example, significant improvements in the appearance of cellulite are demonstrated by Hexsel Depression Depth Scores, Likert scale scores and by dimple analysis.

The most common side effects of CCH injection are injection site reactions including: bruising, pain, nodule, itching, swelling, hardness, discoloration and redness. Injection site bruising generally diminishes over the treatment sessions.

Additional embodiments of the present composition, scales, methods and the like will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment or aspect. Additional aspects and embodiments are set forth in the following description and claims, particularly when considered in conjunction with the accompanying examples and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing features of embodiments will be more readily understood by reference to the following detailed description, taken with reference to the accompanying drawings, in which:

FIG. 1 is a cross-sectional illustration of skin and subdermal tissue depicting the collagen septae.

FIG. 2 is an amino acid sequence listing for AUX-I (Seq. ID No. 5).

FIG. 3 is an amino acid sequence listing for AUX-II (Seq. No. ID 6).

FIG. 4 illustrates the Hexsel cellulite severity scale (CSS) (B) depth of depressions.

FIG. 5 illustrates an example of the Thigh Cellulite Severity-Patient (TCS-P); Thigh Cellulite Severity-Clinician (TCS-C) scale.

FIG. 6 illustrates an example of subject dimple and injection site markings on the buttock.

FIG. 7 depicts the injection technique used in Treatment I.

FIG. 8 is a bar chart of the primary endpoint and key secondary endpoint of composite responders in RELEASE-1 and RELEASE-2 studies, defined as patients with greater than or equal to 2-level or greater than or equal to 1-level severity improvement from baseline in both CR-PCSS and PR-PCSS ratings for the target buttock at Day 71.

FIG. 9 is a series of photographs of composite response with CCH 0.84 mg at Day 71 compared with baseline. FIG. 9A demonstrates a 2-level improvement in both the CR-PCSS and PR-PCSS. FIG. 9B demonstrates a 1-level improvement in both the CR-PCSS and PR-PCSS.

FIG. 10 is a bar chart of the primary endpoint and key secondary endpoint of composite responders in the non-targeted buttock at Day 71 (for purposes of data analysis). Composite response was defined as patients with greater than or equal to 2-level or greater than or equal to 1-level severity improvement from baseline in both CR-PCSS and PR-PCSS ratings at Day 71.

FIG. 11 is a bar chart of the frequency of responders for PR-PCSS and S-GAIS at Day 71 in the intent-to-treat (ITT) population.

FIG. 12 is a bar chart of mean improvement from baseline in PR-CIS total score at Day 71 in the modified intent-to-treat (mITT) population. Baseline values were used for women who did not have a Day 71 PR-CIS assessment.

FIG. 13 illustrates the pre-marking image registration in a 3-D registration to grid (Day 1 Pre-Marking). The image is centered to grid in 3-D space. Using the grid as a reference, the image analysis technician (IAT) positions the Baseline image so that the approximate center of the image is placed at the grid's origin. The thigh/buttock faces forward in the +z-direction, the upper thigh/buttock points in the +y-direction and the lower thigh/buttock points in the −y-direction.

FIG. 14 is a color-by-distance map for image registration.

FIG. 15 illustrates a primary dimple of the area of interest (1). The Day 1-Post Marking image is used as a reference to locate the target dimple on the Day 1-Pre-Marking image. The technician then traces the boundary of the primary dimple on the tracked, pre-marking image.

FIG. 16 is a series of photographs transposing the primary dimple of the area of interest. The dimple tracing on the tracked, pre-marking image is transposed onto the Day 22, Day 43 and Day 71 Follow-Up images based on each Follow-Up image's unique surface tracking relationship to the Baseline.

FIG. 17 depicts the outline of the normal tissue and bruised tissue at Days 4, 8, and 15 after injection in the left buttock of a subject.

FIG. 18(A) depicts the outline of the normal tissue and bruised tissue at Days 4, 8, and 15 after injection in the left buttock and provides L*, a*, and b* color measurements in those tissues.

FIG. 18(B) depicts the outline of the normal tissue and bruised tissue at Days 4, 8, and 15 after injection in the left buttock. Average color and AEs for the normal and bruised tissues are calculated based on the L*a*b* color values.

FIGS. 19(A)-19(C) depicts an exemplary dimple analysis. FIG. 19(A) illustrates the observed and change from Day 1 pre-marking image in dimple analysis parameters. FIG. 19(B) illustrates the maximum length and maximum width of the dimple. FIG. 19(C) illustrates the surface area and volume between the dimple base and interpolated surface.

FIG. 20 is a line graph of mean PR-PCSS rating over time for the target buttock (mITT population) of Example 3. The lower line is CCH treatment vs. placebo (upper line) as described in Examples 2 and 3.

FIG. 21 is a line graph of mean PR-PCSS rating over time for the non-target buttock (mITT population) of Example 3. The lower line is CCH treatment vs. placebo (upper line) as described in Examples 2 and 3.

FIG. 22 is a line graph of mean CR-PCSS rating score over time for the target buttock (mITT population) of Example 2. The lower line is CCH treatment vs. placebo (upper line) as described in Examples 2 and 3.

FIG. 23 is a line graph of mean CR-PCSS rating score over time for the non-target buttock (mITT population) of Example 2. The lower line is CCH treatment vs. placebo (upper line) as described in Examples 2 and 3.

FIG. 24 represents two-level composite responders of the target and non-target buttocks at Day 71 (ITT Population).

FIG. 25 represents the study design of Example 5.

FIG. 26 represents mean PR-CIS Item Scores from Day 1 to Day 71 in Phase 3 (302/-303) and Day 71 to Day 180 in Example 5 (Study 304).

FIG. 27 represents the percent of SSCTA responders of CCH-treated vs. placebo-treated at Day 180 compared to Day 71 in Phase 3.

FIG. 28 represents the subject disposition during Study 202. Note a.: Until the Study 201 drug blind was broken by the sponsor, subjects underwent up to 4 observation-only visits at 3-month periods which began 90 days after Day 1 of the double-blind study (201) (i.e., within 20 days±4 days of completion of double-blind study). The Observation Phase was defined within each treated treatment area, and was defined as the time period from Screening A to the first treatment date in Study 202 or the end of Study 202 if there was no treatment received in Study 202. Note b.: The Other category included either screen failures, subjects declining to participate in the Treatment Phase, site closure on Study Day 272 of subject enrollment, or subjects not compliant with study visits. Note c.: Upon completion of treatment (Treatment Phase Day 71), the subject was followed at 3-month intervals per the Observation Assessments up to Day 360. For subjects treated with CCH in Study 201, the treatment area treated was assessed for Long-Term Durability, up to Day 720.

FIG. 29: represents mean PR-PCSS rating over time in a CCH-treated Area in Study 202.

FIG. 30 represents mean PR-PCSS rating over time for re-exposed (Buttock and Thigh-treated) subjects in Study 202.

FIG. 31 represents mean PR-PCSS rating over time for re-exposed buttock-treated subjects during the first and second treatment course in Study 202.

FIG. 32 represents mean CR-PCSS rating over time in a CCH-treated area in Study 202.

FIG. 33 represents mean CR-PCSS rating over time for re-exposed (buttock and thigh treated) subjects in Study 202.

FIG. 34 represents mean CR-PCSS rating over time for re-exposed buttock-treated subjects during the first and second treatment course in Study 202.

### DETAILED DESCRIPTION

The various aspects and embodiments will now be fully described herein. These aspects and embodiments may, however, be embodied in many different forms and should not be construed as limiting; rather, these embodiments are provided so the disclosure will be thorough and complete, and will fully convey the scope of the present subject matter to those skilled in the art. All publications, patents and patent applications cited herein, whether supra or infra, are hereby incorporated by reference in their entirety.

### A. DEFINITIONS

Unless defined otherwise, all terms and phrases used herein include the meanings that the terms and phrases have attained in the art, unless the contrary is clearly indicated or clearly apparent from the context in which the term or phrase is used. Although any methods and materials similar or equivalent to those described herein can be used in the practice or testing of the present invention, particular methods and materials are now described.

Unless otherwise stated, the use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Consequently, recitation of ranges of values herein are merely intended to serve as a shorthand method of referring individually to each separate value falling within the range, and each separate value is incorporated into the specification as if it were individually recited herein.

“Affected area” or “treatment area” as used herein means an area of cellulite on a human patient that is to be treated or has been treated with collagenase (defined below). This may include a quadrant (i.e., left buttock, right buttock, left posterolateral thigh, right posterolateral thigh). Affected area or treatment area is not limited to buttocks or thighs. Rather, any area of the body with cellulite can be treated as a treatment area.

“Adverse Events” or “AE” as used herein means any unfavorable or unintended change in body structure (signs), body function (symptoms), laboratory result (e.g., chemistry, ECG, X-ray, etc.), or worsening of a preexisting condition associated temporally with the use of the study medication whether or not considered related to the study medication.

“Body-Q” as used herein is a patient-reported outcome instrument that is commercially available under license from Memorial Sloan Kettering Cancer Center. It is based on patient perceptions of body contouring and/or weight loss. It measures 3 domains: appearance, health-related quality of life (HRQL), and patient experience of healthcare through 18 independently functioning scales. The patient-reported outcome instrument is described in BODY-Q: User's Manual BODY-Q: User's Manual, Version 1.0, July 2015, Memorial Sloan Kettering Cancer Center, McMaster University and Stefan Cano. The BODY-Q includes a scale to measure cellulite. See https://www.mskcc.org/sites/default/files/node/174457/documents/body-q-users-guide.pdf (accessed Jul. 3, 2019). For cellulite, there are 16 scaled items having response options ranging from “not at all” to “extremely bothered” over the timeframe of the past week and assuming a Flesch-Kincaid grade reading level. The patient is asked: “With your cellulite in mind, in the past week, how much have you been bothered by:” [16 questions follow where the patient ranks the response as 1-extremely bothered; 2-moderately bothered; 3-a little bothered; 4-not bothered at all]. The score ranges from 16 (extremely bothered) to 64 (not at all).

Conventionally, clinical examination of bruising comprises a visual examination of the bruised and surrounding areas in conjunction with an evaluation of the subject's medical, surgical, and concomitant medication histories. The results of this interpretation are subjective and affected by several unrelated factors, including viewing geometry, ambient lighting, color of unexposed surrounding skin, and the experience and visual acuity of the observer. “Bruising Analysis” as used herein means the detection of visible change in skin color as evaluated from the images of the collagenase-treated areas in a subject using the objective image capture and tracking methodologies disclosed in U.S. Patent Publication No. 2019/0035080 applied uniformly to all subject images. This objective analysis has the potential to aid or even replace visual and clinical examination of the bruising by the health care provider by providing the ability to quantify, differentiate, and assess the bruising both intra-subject (within the same subject at different times points) and inter-subject (between different subjects) levels. This analysis utilizes the L*a*b* color space defined by the Commission Internationale de l'Eclairage (CIE) modeled after a color-opponent theory stating that two colors cannot be red and green at the same time or yellow and blue at the same time. As shown below, L* indicates lightness/darkness, a* is the red/green coordinate, and b* is the yellow/blue coordinate. Deltas for L* (ΔL*), a* (Δa*) and b* (Δb*) may be positive (+) or negative (−). The total difference, Delta E (AE*), however, is always positive.

$$Δ {L}^{*} ( {L}^{*} sample minus {L}^{*} standard ) = difference in lightness and darkness ( += lighter , -= darker ) ; a low number ( 0 ‐ 50 ) indicates dark and a high number ( 51 ‐ 100 ) indicates light$$

$$Δ {a}^{*} ( {a}^{*} sample minus {a}^{*} standard ) = difference in red and green ( += redder , -= greener )$$

$$Δ {b}^{*} ( {b}^{*} sample minus {b}^{*} standard ) = difference in yellow and blue ( += yellower , -= bluer )$$

All three values are required to completely describe an object's color (in this case the bruising captured in the treated area image of the subject). The objective methodology for the image analysis of collagenase-treated area (pre- and post-treatment images at protocol specified time points) with data outputs in L*a*b* values allow for quick, easy, accurate, repeatable, and unbiased quantification of skin color and any change therein. This methodology rules out the inherent variability associated with the conventional subjective visual estimation of the images. A ΔE is calculated as follows:

$$Δ E ( Color difference between ‐ Bruised vs . Normal ) = SQRT [ {( {L}_{B -}^{*} {L}_{N}^{*} )}^{2} + {( {A}_{B -}^{*} {A}_{N}^{*} )}^{2} + {( {B}_{N}^{*} )}^{2} ] , where$$

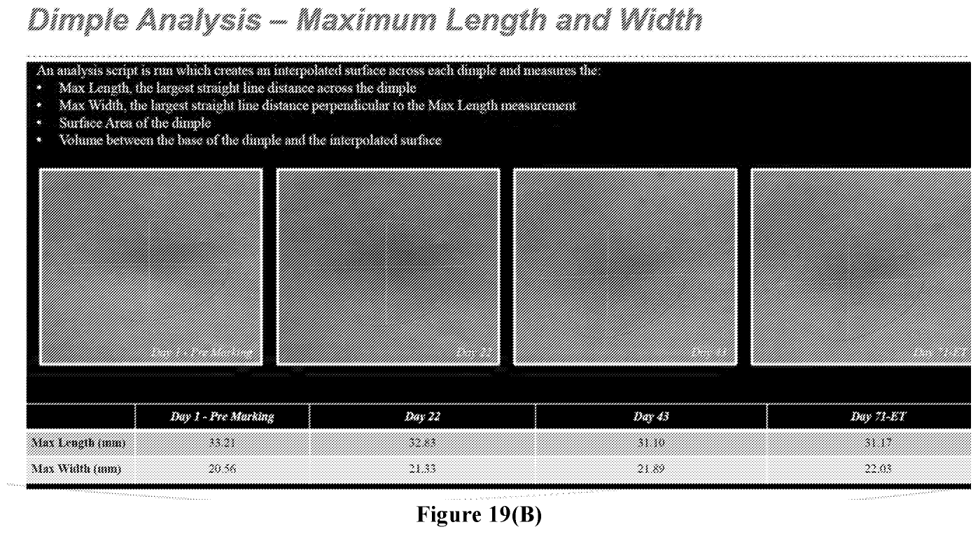

L*_{B}=Bruised Tissue L*
L*_{N}=Normal Tissue L*
A*_{B}=Bruised Tissue A*
A*_{N}=Normal Tissue A*
B*_{B}=Bruised Tissue B*
B*_{N}=Normal Tissue B*
   FIG. 18(B) illustrates a bruise analysis of a treatment area.

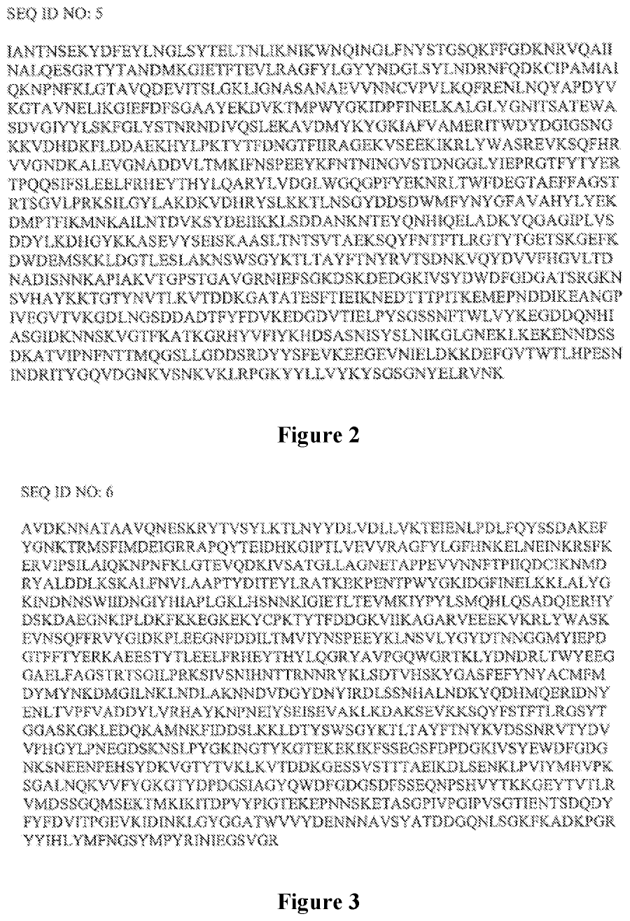

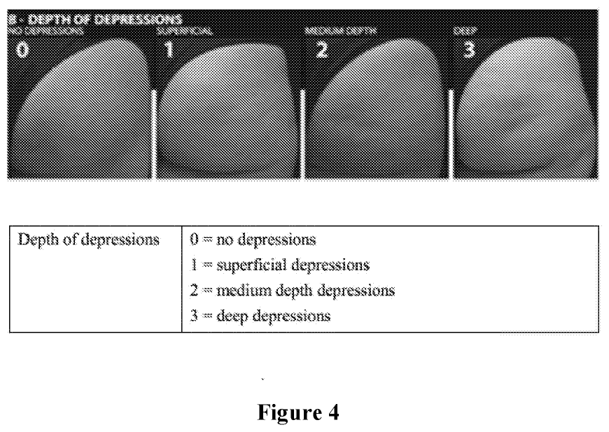

“CCH” as used herein means the AUX-I (Seq. ID No. 5 (FIG. 2)) and AUX-II (Seq. No. ID 6 (FIG. 3)) mixture of collagenases in an approximate 1:1 ratio obtained by the fermentation of Clostridium histolyticum (also known as Hathewaya histolytica). CCH is available commercially as a lyophilized powder under the trademark XIAFLEX®, which comprises the AUX-I and AUX-II mixture with particular excipients, although CCH may be used with other suitable excipients.

“Clinician Reported Photonumeric Cellulite Severity Scale (CR-PCSS)” as used herein are the photonumeric scales described in PCT Patent Application PCT/US2018/020551 (published as WO2018/160905 on Sep. 7, 2018) used by physicians/clinicians and designed to assess the severity of cellulite into 5 levels.

Except as otherwise provided herein, “collagenase” means any of the following: (a) collagenase (including mutants) having activity as defined by EC 3.4.24.3 (https://www.brenda-enzymes.org/enzyme.php?ecno=3.4.24.3 (accessed Jul. 3, 2019); (b) collagenase produced by fermentation of Clostridium histolyticum (also known as Hathewaya histolytica); (c) CCH (as defined above); (d) collagenase having at least 50% sequence alignment with AUX-I as determined by BLAST; (e) collagenase having at least 50% sequence alignment with AUX-II as determined by BLAST; (f) collagenase produced by fermentation of other source organisms (i.e., non-Clostridium histolyticum), e.g., mammalian, crustacean, fungal, bacterial or microbial collagenase; (g) collagenase obtained by recombinant techniques; (h) collagenase with a molecular mass from about 65 kDa to about 130 kDa; (i) collagenase designated as class I or class II (also referred to as collagenase I (or 1), collagenase II (or 2), Type I collagenase, Type 2 collagenase); (j) mixtures of collagenases I and II; (k) collagenase from strain JCM 1403 (ATCC 19401) or derivatives thereof, (1) collagenase from strain ATCC 21000 or derivatives thereof; (m) collagenase from ATCC 69334 or derivatives thereof, (n) collagenase from C. perfringens; (o) collagenase from Vibrio alginolyticus; (p) collagenase from Streptomyces; (q) collagenase from Pseudomonas; (r) collagenase from Achromobacter iophagus (s) collagenase described by Worthington Biochemical Corp. (www.Worthington-biochem.com; “Product Highlights”); (t) collagenase described by Sigma-Aldrich (www.sigma-aldrich.com); (u) collagenase having one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay);
(v) collagenase described by Nordmark Arzneimittel GmbH & Co. KG; (w) collagenase from strain 004; or (x) equivalents or mixtures of any of the foregoing. Non-limiting examples of collagenases that may be used in the disclosure herein are described in U.S. Pat. Nos. 7,811,560; 9,757,435; 9,744,138; and WO2012/125948.

“Dimple analysis” as used herein means an analysis of one or more selected dimples wherein parameters, such as dimple volume, length, width and surface area are measured. Measurements may be performed by various known methods such as those described in Eckhouse et al. WO 2018/116304 and WO 2018/116305, and from Cherry Imaging (www.cherryimaging.com) and Canfield Scientific, Inc. See also Salameh et al., “Novel Stereoscopic Optical System for Objectively Measuring Above-Surface Scar Volume First-Time Quantification of Responses to Various Treatment Modalities,” Dermatol. Surg. 00:1-7 (2017); and U.S. Pat. No. 9,996,923. Such measurements of volume, length, width and surface area may be calculated using digital 3-D greyscale images (with X and Y axis rotation feature) and digital 3-D textured and lit images (with X and Y rotation feature) together with a computer program that analyzes such images. As an example, for a buttock treatment area, images may be taken of the left treated buttock and/or right treated buttock for each patient before and after treatment. For a thigh treatment area, images may be taken of each of the thigh treated areas at 0 degrees, 45 degrees and 90 degrees before and after treatment. For a thigh treatment area, images taken using the method by Canfield Scientific may be taken of each of the thigh treated areas at 0 degrees, 45 degrees and 90 degrees before and after treatment.

“Durability” as used herein means the period of time in which there is a persistence of a treatment effect. This period of time can range from about 3 months to about 20 years, or about 1 to 19 years, or about 2 to 18 years, or about 3 to 17 years, or about 4 to 16 years, or about 5 to 15 years, or about 6 to 14 years, or about 7 to 13 years, or about 8 to 12 years, or about 9 to 11 years. The period may be for about 6 months, about 1 year, about 2 years, about 3 years, about 4 years, about 5 years, about 10 years, about 15 years, or about 20 years.

“Early Termination Visit” as used herein means for any subject that terminates the study, her final visit is considered the Early Termination Visit and the assessments that would be typically done on Day 71 for a subject who completed the study would be performed at the Early Termination Visit.

“Fitzpatrick scale” as used herein means a scale is used to assess a subject's skin type as shown in Table 1.

TABLE 1
Fitzpatrick Scale

| | | |
|---|---|---|
| I | Pale white skin, blue/hazel eyes, | Always burns, does not tan |
| | blond/red hair | |
| II | Fair skin, blue eyes | Burns easily, tans poorly |
| III | Darker white skin | Tans after initial burn |
| IV | Light brown skin | Burns minimally, tans easily |
| V | Brown skin | Rarely burns, tans darkly easily |
| VI | Dark brown or black skin | Never burns, always tans darkly |

“Hexsel Cellulite Severity Scale” or “Hexsel CSS” or “Cellulite Severity Scale (CSS)” as used herein means the following photonumeric scale that evaluates 5 key morphologic features of cellulite (Table 2):

TABLE 2
Hexsel Cellulite Severity Scale

| | | |
|---|---|---|
| A | Number of | 0 = no depressions |
| | evident | 1 = small amount: 1-4 depressions are visible |
| | depressions | 2 = Moderate amount: 5-9 depressions |
| | | 3 = large amount: 10 or more depressions |
| B | Depth of | 0 = no depressions |
| | depressions | 1 = superficial depressions |
| | | 2 = medium depth depressions |
| | | 3 = deep depressions |
| C | Morphological | 0 = no raised areas |
| | appearance of | 1 = orange peel appearance |
| | skin surface | 2 = cottage cheese appearance |
| | alterations | 3 = mattress appearance |
| D | Grade of laxity, | 0 = absence of laxity, flaccidity, or sagging skin |
| | flaccidity, or | 1 = slight draped appearance |
| | sagging skin | 2 = moderate draped appearance |
| | | 3 = severe draped appearance |
| E | Classification | Stage 0 = No dimpling when the subject is standing |
| | scale by | and lying. The pinch test reveals “folds and |
| | Nürnberger | furrows”, but there is no mattress-like appearance. |
| | and Müller^{a} | Stage 1 = No dimpling while the subject is standing |
| | | or lying, but the pinch test reveals the mattress-like |
| | | appearance. Stage 2 = Dimpling appears |
| | | spontaneously when standing and not lying down. |
| | | Stage 3 = Dimpling is spontaneously positive |
| | | standing and lying down. |

The sum of points results in the following classification.

| Points | Classification of Cellulite |
|---|---|
| 1-5 | Mild |
| 6-10 | Moderate |
| 11-15 | Severe |

Hexsel et al., J. Eur. Acad., Dermatol. Venereol. 2009; 23(5): 523-528.
a. Nürnberger and Müller, J. Dermatol. Surg. Oncol. 1978; 4(3): 221-229. Subjects were evaluated in the standing position with relaxed gluteus muscles. However, if the subject had no evident depressions, they were asked to contract their gluteus muscles or the pinch test was applied (by pinching the skin between the thumb and index finger) in order to differentiate between scores/grades of zero or 1.

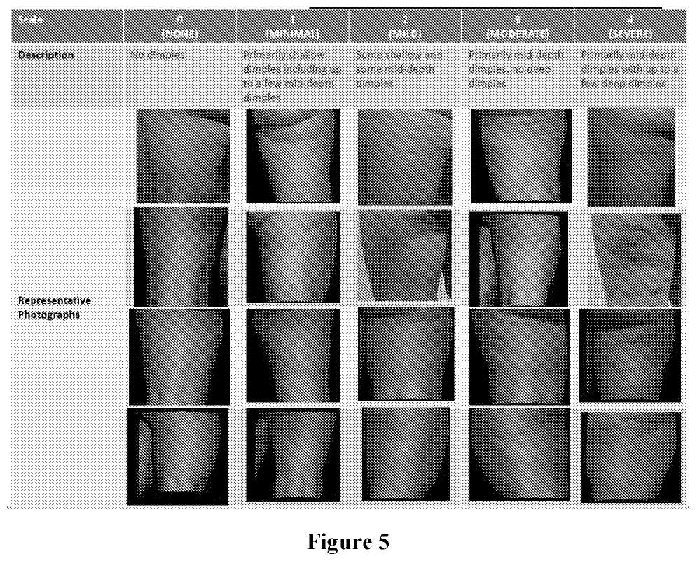

“Hexsel Depression Depth Score” as used herein means an assessment of only (B) depth of depressions from the Hexsel CSS (FIG. 4):
0=no depressions
1=superficial depressions
2=medium depth depressions
3=deep depressions.

“Images” or “Imagery” as used herein means photographs, illustrations, drawings, models, 3-D models, computer-generated images, MRI images and the like.

“Likert Scale score” as used herein means the score identified by an independent blinded assessor (or patient) of the change in the treated area (buttock or thigh) at each post-treatment visit by comparing the photographs (2-D color, 3-D color and 3-D greyscale) of cellulite from the Day 1 pretreatment (Baseline) with photographs for the post-treatment visit. The score is captured in the following 5-point Likert Scale:

| −1 | 0 | 1 | 2 | 3 |
|---|---|---|---|---|
| Worse | No change | Improved | Much improved | Very much |
| | | | | improved |
| The treated area | The treated area | Obvious | Marked | Optimal |
| appearance is | appearance is | improvement in | improvement in | cosmetic result |
| worse than | essentially the | the treated area | the treated area | from treatment |
| before treatment | same as before | appearance from | appearance from | of the treated |
| | treatment | before treatment, but | before treatment, | dimples |
| | | additional treatment | but not completely | |
| | | is indicated | optimal | |

The term “non-target thigh” or “non-target buttock,” as used herein, means the thigh or buttock that is not selected for evaluating the primary efficacy endpoint(s). Such non-target areas may still receive treatment and be used to evaluate secondary efficacy endpoints.

“Optional” or “optionally” means that the subsequently described element, component or circumstance may or may not occur, so that the description includes instances where the element, component, or circumstance occurs and instances where it does not.

“Patient Reported Cellulite Impact Scale (PR-CIS)” as used herein means an assessment of the visual and emotional impact of cellulite (happy with the appearance of cellulite, bothered, self-conscious, embarrassed, looking older, or looking overweight or out of shape) using a 6-question survey, with each question rated on a numerical rating scale from 0 (not at all) to 10 (extremely). More specifically, the PR-CIS is a 6-item static questionnaire assessing the visual and emotional impact of cellulite (happy with the appearance of cellulite, bothered, self-conscious, embarrassed, looking older or looking overweight or out of shape); each item is answered by the subject on an 11-level numerical rating (or interval) scale from 0 (not at all) to 10 (extremely) while viewing digital images of their buttocks or thighs. This assessment may be of all thighs and/or buttocks together rather than each individual area separately. A PR-CIS total score can be derived from 6 individual questions:
Question 1-Thinking about the areas selected for treatment, how happy are you with the appearance of your cellulite?
Question 2-Thinking about the areas selected for treatment, how bothered are you by the appearance of your cellulite?
Question 3-Thinking about the areas selected for treatment, how self-conscious are you about the appearance of your cellulite?
Question 4-Thinking about the areas selected for treatment, how embarrassed are you about the appearance of your cellulite?
Question 5-Thinking about the areas selected for treatment, how much older do you look because of your cellulite?
Question 6-Thinking about the areas selected for treatment, how overweight or out of shape do you look because of your cellulite?

“Patient Reported Cellulite Impact Scale (Abbreviated)” (PR-CIS Abbreviated) as used herein means an assessment of the visual and emotional impact of cellulite (happy with the appearance of cellulite, bothered, self-conscious, embarrassed, or looking overweight or out of shape) using a 5-question survey, with each question rated on a numerical rating scale from 0 (not at all) to 10 (extremely). More specifically, the PR-CIS Abbreviated is a 5-item static questionnaire assessing the visual and emotional impact of cellulite (happy with the appearance of cellulite, bothered, self-conscious, embarrassed, or looking overweight or out of shape); each item is answered by the subject on an 11-level numerical rating (or interval) scale from 0 (not at all) to 10 (extremely) while viewing digital images of their buttocks or thighs. This assessment may be of all thighs and/or buttocks together rather than each individual area separately. In a non-limiting example, a PR-CIS Abbreviated total score can be derived from 5 individual questions:
Question 1-Thinking about the areas selected for treatment, how happy are you with the appearance of your cellulite?
Question 2-Thinking about the areas selected for treatment, how bothered are you by the appearance of your cellulite?
Question 3-Thinking about the areas selected for treatment, how self-conscious are you about the appearance of your cellulite?
Question 4-Thinking about the areas selected for treatment, how embarrassed are you about the appearance of your cellulite?
Question 5-Thinking about the areas selected for treatment, how overweight or out of shape do you look because of your cellulite?
   A PR-CIS Abbreviated total score can be derived from other sets of 5 questions from the full PR-CIS.

“Patient Reported Photonumeric Cellulite Severity Scale (PR-PCSS)” as used herein are the photonumeric scales described in PCT Patent Application PCT/US2018/020551 (published as WO2018/160905 on Sep. 7, 2018) used by patients and designed to assess the severity of cellulite into 5 levels.

“Photonumeric” as used herein means using a series of photographs, illustrations, drawings, models, 3-D models, computer-generated images, MRI images, images and the like each assigned a different level of cellulite severity in a scale.

“Sequential visit” as used herein means two or more clinician visits or times where cellulite changes are assessed by a scale. The time between visits may be about two weeks, three weeks, about one month, about two months, about three months, about fourth months, about five months, about six months, about one year, about eighteen months, about two years, about three years, about 4 years, or about five years or longer.

“Serious Adverse Events” as used herein means an adverse event that results in death, is immediately life-threatening, results in or prolongs an inpatient hospitalization, results in permanent or substantial disability, is a congenital anomaly/birth defect, or is considered an important medical event.

“Statistically significant” as used herein means statistical data having a “P” value generally of less than 0.05. In context of the present disclosure, clinical trials are generally designed to test the superiority of an intervention (e.g., in this case, a treatment) as compared with a control. Given that clinical trials involve people, each of whom are physiologically different from one another, variations in the results occur naturally. Statistics are therefore used to determine whether any observed differences are caused by chance or by the intervention itself. Measures of statistical significance quantify the probability of a study's result being due to chance. The “P” value, frequently used to measure statistical significance, is the probability that the study results are due to chance rather than to a real treatment effect. Generally, the conventional cut off for the “P” value to be considered statistically significant is 0.05, or 5% although it may change depending on study design and outcomes. If the “P” value is less than 0.05, this means that the possibility of the results in the study being due to chance is less than 5%. If the “P” value is greater than 0.05 (5%), any difference between the treated group and control group is not statistically significant-meaning that the difference cannot likely be attributed to the treatment, but instead may be due to chance.

The terms “subject” or “patient” is used interchangeably herein and refers to a human or other mammal.

“Subject Global Aesthetic Improvement Scale (S-GAIS)” and “Investigator Global Aesthetic Improvement Scale (I-GAIS)” as used herein mean the following scales to assess cellulite severity and/or improvement. The subject is asked the following introductory question: “How would you rate the appearance of your treated cellulite after treatment?” The rating ranges from −3 (Very much worse) to +3 (Very much improved) depending on the subject's response, as shown in Table 3.

TABLE 3
Subject Global Aesthetic Improvement Scale (S-GAIS) and
Investigator Global Aesthetic Improvement Scale (I-GAIS)

| Rating | Response Option | Description (S-GAIS) | Description (I-GAIS) |
|---|---|---|---|
| +3 | Very much improved | My treated cellulite looks very much | Optimal cosmetic result from |
| | | better. | treatment of the treated dimples |
| +2 | Much improved | My treated cellulite looks much | Marked improvement in the treated |
| | | better, but additional treatment | area appearance from |
| | | would slightly improve the result. | before treatment, but not completely |
| | | | optimal |
| +1 | Improved | My treated cellulite looks better, but | Obvious improvement in the treated |
| | | additional treatment is necessary. | area appearance from |
| | | | before treatment, but additional |
| | | | treatment is indicated |
| 0 | No change | My treated cellulite looks essentially | The treated area appearance is |
| | | the same as it did originally. | essentially the same as before |
| | | | treatment |
| −1 | Worse | My treated cellulite looks worse than | The treated area appearance is worse |
| | | it did originally. | than before treatment |
| −2 | Much worse | My treated cellulite looks much | Marked worsening in appearance |
| | | worse than it did originally. | from the initial condition |
| −3 | Very much worse | My treated cellulite looks very | Obvious worsening in appearance |
| | | much worse than it did originally. | from the initial condition |

“Subject Satisfaction with Cellulite Treatment” (SSCT) as used herein means a subject satisfaction rating ranging from −2 to +2. As an example, Table 4 below provides such assessment for cellulite treatment on the buttock. The patients are asked: “Today, how satisfied are you with the results of the cellulite treatment you received on the specific area or areas on your buttocks that were treated?” They then choose an answer/rating as shown in Table 4.

TABLE 4
Subject Satisfaction with Cellulite
Treatment Assessment - Buttocks

| Rating | Description |
|---|---|
| +2 | I am very satisfied with the cellulite treatment on my buttocks. |
| +1 | I am satisfied with the cellulite treatment on my buttocks. |
| 0 | I am neither dissatisfied nor satisfied with the cellulite |
| | treatment on my buttocks. |
| −1 | I am dissatisfied with the cellulite treatment on my buttocks. |
| −2 | I am very dissatisfied with the cellulite treatment on my |
| | buttocks. |

Table 5 provides such assessment for cellulite treatment on the thighs. The patients are asked: “Today, how satisfied are you with the results of the cellulite treatment you received on the specific area or areas on your thighs that were treated?” They then choose an answer/rating as shown in Table 5.

TABLE 5
Subject Satisfaction with Cellulite Treatment Assessment - Thighs

| Rating | Description |
|---|---|
| +2 | I am very satisfied with the cellulite treatment on my thighs. |
| +1 | I am satisfied with the cellulite treatment on my thighs. |
| 0 | I am neither dissatisfied nor satisfied with the cellulite treatment |
| | on my thighs. |
| −1 | I am dissatisfied with the cellulite treatment on my thighs. |
| −2 | I am very dissatisfied with the cellulite treatment on my thighs. |

“Subject Self-Rating Scale (SSRS)” as used herein is a scale used by a subject to assess his/her satisfaction with appearance in association with cellulite using whole numbers on a 7-level scale that ranges from 0 (extremely dissatisfied) to 6 (extremely satisfied) as shown in Table 6.

TABLE 6
Subject Self-Rating Scale (SSRS)

| Rating | Response Option |
|---|---|
| 6 | Extremely satisfied |
| 5 | Satisfied |
| 4 | Slightly satisfied |
| 3 | Neither satisfied nor dissatisfied |
| 2 | Slightly dissatisfied |
| 1 | Dissatisfied |
| 0 | Extremely dissatisfied |

The term “target thigh” or “target buttock,” as used herein, means the thigh or buttock that is selected for evaluating the primary efficacy endpoint(s).

The term “therapeutically effective amount,” as used herein, refers to the amount of collagenase needed to reduce the severity of cellulite in a patient or a statistically significant population of patients. The amount collagenase composition employed will be that amount necessary to deliver an amount of collagenase needed to achieve the desired result. In practice, this will vary depending upon the collagenase being injected, the injection technique, and the enzymatic activity at the treatment area.

The term “treatment course,” as used herein, comprises three treatment sessions (i.e., each a visit to the clinician to receive treatment).

The term “treatment-emergent adverse event” or “TEAE” as used herein is any condition that was not present prior to treatment with study medication but appeared following treatment, was present at treatment initiation but worsened during treatment, or was present at treatment initiation but resolved and then reappeared while the individual was on treatment (regardless of the intensity of the AE when the treatment was initiated).

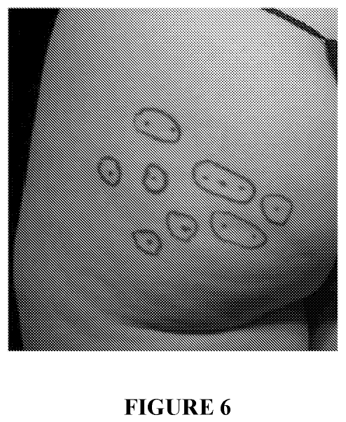

The term “Thigh Cellulite Severity-Patient” (“TCS-P”) and “Thigh Cellulite Severity-Clinician” (“TCS-C”) as used herein means the photonumeric scale shown in FIG. 5 (or a substantially similar scale) used by patients (TCS-P) or clinicians (TCS-C) to assess thigh cellulite severity, improvement, and/or patient satisfaction and assist in assessing collagenase efficacy. The patient-reported use of the scale is referred to as TCS-P; the clinician-reported use of the scale is referred to as TCS-C.

The term “treatment visit” or “treatment” or “treatment session” as used herein means one or more injections or treatments to affected area(s) with a therapeutically effective amount of at least one active agent useful in treating cellulite in a single office visit.

The terms “validated,” “validity” or “validation” as used herein mean a process by which a particular scale is demonstrated to be accurate and reliable, including the repeatability of visual assessments to ensure that the same result can be consistently obtained. Validation further examines the precision, accuracy and sensitivity of the scale to confirm the measurements taken by it are reliable, reproducible and robust.

### B. INTRODUCTION

The present disclosure relates to methods of treating cellulite, comprising the administration of a therapeutically effective amount of one or more collagenases to a subject having the appearance of cellulite, through the use of certain injection techniques described below.

Generally, there are four phases of treatment: (1) The clinician and patient perform pretreatment assessments to determine a pretreatment baseline, and the clinician selects dimples to be treated; (2) the clinician marks the dimples to be treated at the nadir, if a nadir is present; (3) the clinician treats the patient with collagenase; and (4) the clinician and patient perform post-treatment assessments. These phases are detailed below. The phases, and steps within them, are optional and the order of steps is not intended to be limiting as the order may vary yet achieve comparable results.

### C. PHASE 1—PRETREATMENT ASSESSMENTS

In a first phase of the methods of treating cellulite described herein, the clinician performs a selection of cellulite dimples to be treated based on the following criteria:
Dimples should be well-defined and evident naturally when the patient is standing in a relaxed pose (standing position with relaxed gluteus muscles) as confirmed by photographs
Dimples chosen should be the ones the clinician believes is most likely to improve aesthetic appearance of each entire buttock, thigh or other affected area
Photographs of affected areas are taken before treatment when the patient is standing in a relaxed pose
Before injection, an assessment is performed, i.e., the clinician and/or patient independently assess the photographs and score the result using one or more of the following scales or assessment methods:
   Hexsel Cellulite Severity Scale (Hexsel CSS)
   Hexsel Depression Depth Score
   Likert Scale
   Dimple Analysis
   Clinician Reported Photonumeric Cellulite Severity Scale (CR-PCSS)
   Patient Reported Photonumeric Cellulite Severity Scale (PR-PCSS)
   Investigator Global Aesthetic Improvement Scale (I-GAIS)
   Subject Global Aesthetic Improvement Scale (S-GAIS)
   Patient Reported Cellulite Impact Scale (PR-CIS)
   Subject Self-Rating Scale (SSRS)
   Subject Satisfaction with Cellulite Treatment (SSCT)
   Thigh Cellulite Severity-Patient (TCS-P); Thigh Cellulite Severity-Clinician (TCS-C)
   Body-Q
   Fitzpatrick scale
   Any validated photonumeric or other scale used by clinicians and/or patients to assess cellulite severity, improvement, and/or patient satisfaction (e.g., Hexsel-Merz scale)

### D. PHASE 2-MARKING OF DIMPLES TO BE TREATED

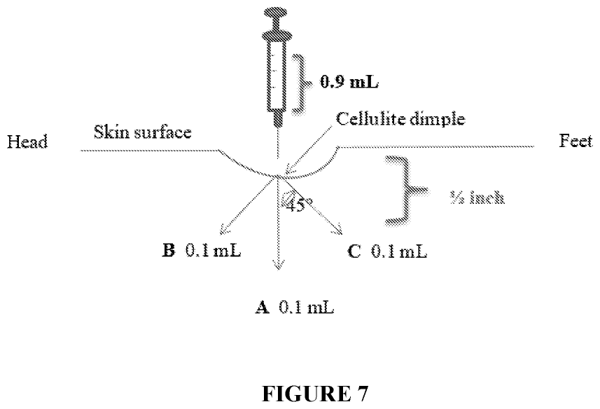

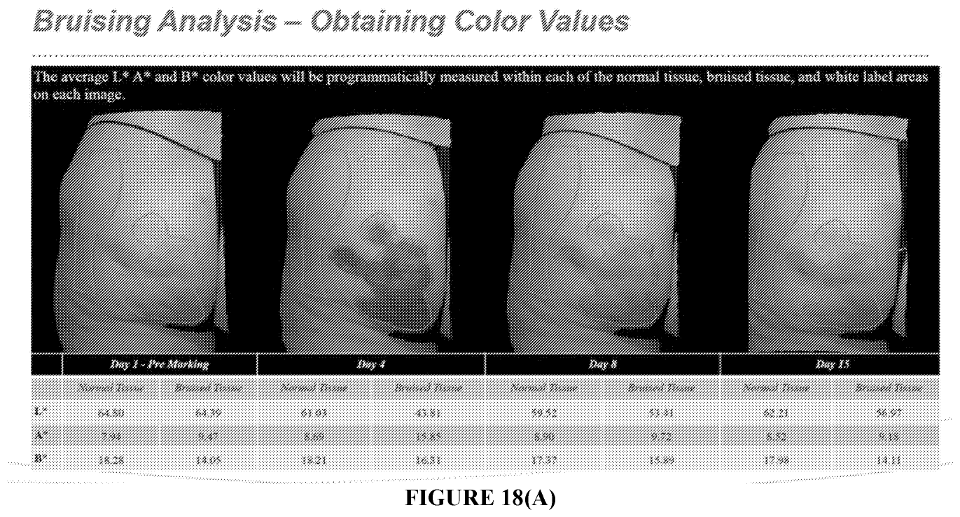

In a second phase of the treatments described herein, dimples to be treated can be marked with a dot(s) by the clinician. More photographs may be taken. See, e.g., FIGS. 6 and 15.

### E. PHASE 3-COLLAGENASE INJECTIONS

In a third phase of treatment, a clinician treats the patient with collagenase injections.

### 1. Types of Collagenases

The collagenases useful in the present disclosure include any of the collagenases as defined above. By way of further background, matrix metalloproteinases (MMPs) can be comprised of collagenases falling within the definition herein. For example, MMP-1 comprises collagenase 1; MMP-8 comprises collagenase 2/neutrophil collagenase; MMP-13 comprises collagenase 3; and MMP-18 comprises collagenases 4. Further, cathepsins can be classified as collagenases.

### 2. Collagenase Enzyme Kinetics

Collagenases useful in the present disclosure may also be characterized by their enzyme kinetics. Here, the approximate kinetic values of the one or more collagenases effective to treat cellulite include the following:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
   Vₘₐₓ=maximal rate
   K_{M}=[Substrate] at 50% of Vₘₐₓ
   K_{cat}=molecules of substrate cleaved per second
   1/K_{cat}=The microseconds required to cleave a molecule of substrate.
   These values may be determined experimentally using the microplate assays described below, but with different substrates and times. Other assays and parameters may be employed.

These values reflect a quantitative expression of enzyme behavior based on the Michaelis-Menten Equation:

$${V}_{0} = {V}_{ma x} [ S ]$$

$${K}_{M} + [ S ]$$

Wherein V₀ is the reaction rate (velocity) at a substrate concentration [S], Vₘₐₓ is the maximum rate that can be observed, and K_{M} is the Michaelis constant, which correlates to the concentration of substrate that yields 50% of Vₘₐₓ.

$${K}_{M} = {k}_{2} + {k}_{- 1}$$

$${k}_{1}$$

Wherein k₁, k₋₁ and k₂ are rate constants for the following steps:

$${\begin{matrix}\begin{matrix}\begin{matrix}\begin{matrix}E + S & {k}_{1}\end{matrix} & ES\end{matrix} & {k}_{2}\end{matrix} & E + P\end{matrix}}_{{⟷}_{k - 1} ⟶}$$

Wherein E is the enzyme, S is the substrate, ES is the enzyme-substrate complex, and P is the product.

The catalytic constant K_{cat} refers to the turnover number, i.e., how fast the ES complex proceeds to E+P. It reflects the number of catalytic cycles that each active site undergoes per unit time.

In certain embodiments, AUX-I and AUX-II have the following characteristics:
AUX I
   Assay: SRC microplate
   Vmax, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
AUX II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934

### Assumptions:

$${K}_{c a t} = {V}_{max} / [ AUX ] = ( nmoles Substrate / nG AUX * min - 1 ) / nG AUX$$

Catalytic efficiency (K_{cat}/K_{M}) generally represents the enzyme's overall ability to convert substrate to product, and reflects both binding and catalytic events. In another embodiment, AUX-I and AUX-II comprise the following characteristics.

| | AUX-I (SRC assay)* | AUX-II (GPA assay)+ |
|---|---|---|
| Vₘₐₓ, min⁻¹ | 3.8 | 15.4 |
| K_{M}, mM | 2.07 × 10⁻⁴ | 1.5 |
| K_{cat}, sec⁻¹ | 53 | 4,636 |
| 1/K_{cat}, microseconds | 18,799 | 216 |
| K_{cat}/K_{M}, mM⁻¹ sec⁻¹ | 256,977 | 2,997 |

Vₘₐₓ=maximal rate
K_{M}=[Substrate] at 50% of Vmax
K_{cat}=molecules of substrate cleaved per second
1/K_{cat}=The time required to cleave one molecule of substrate
K_{cat}/K_{M} is often used to represent catalytic efficiency of the enzyme
y SRC microplate assay
By GPA microplate assay

### 3. Potency (Specific Activity) of Collagenase(s)

Assays have been developed and used to determine the specific activity (potency) of collagenase. Such assays are described in subsections a. to c. and characterize collagenase by its ability to convert substrate to product within a given time period with a pre-determined enzyme concentration. In certain non-limiting embodiments, these assays are used to determine the potency of each of AUX-I and AUX-II, and the combined CCH drug product (1:1 ratio of AUX-I and AUX-II). The SRC assays (described below) use collagen as substrate for the reaction. The SRC assays use soluble rat (tail) collagen (SRC) as substrate, and are used to measure Type I collagenases activity, with Type II collagenases contributing approximately 20% of the observed activity of a collagenase mixture. The SRC assay is fluorometric and utilizes fluorescamine to detect the peptides produced by the Type I digestion of SRC. The reaction is run at pH 7.2 in 4-(2-hydroxyethyl)-1-piperazineethanesulfonic acid (HEPES) buffer containing 15 mM divalent calcium ion for 2.5 h at 25° C.

The bovine tendon collagen (BTC) assay (described below) is based on the procedures of Mandl et al., Arch. Biochem. Biophys. 74: 465-475 (1958), as modified by Keller and Mandl, Arch. Biochem. Biophys. 101: 81-88 (1963). See also Rosen, Arch. Biochem. Biophys. 67: 10-15 (1957). The BTC assay uses insoluble bovine tendon collagen as substrate and measures both Type I and Type II activity (such as AUX-I and II collagenases). The BTC assay is colorimetric and utilizes ninhydrin to detect the peptides produced by Type I and Type II degradation of BTC. This reaction is also run at pH 7.2, but for 22 h at 37° C. in tris (hydroxymethyl) aminomethane (TRIS) buffer containing 10 mM divalent calcium ion.

The third collagenase type of assay, the GPA assays (described below), utilize a soluble, derivatized hexapeptide (carbobenzoxy-GPGGPA (SEQ ID NO: 7)) as substrate. The GPA assay is used to measure primarily Type II activity, with Type I contributing approximately 10% of observed activity. Type II collagenase cleaves the hexapeptide into two tripeptides, one of which (GPA) has a free amino terminus which reacts with fluorescamine to provide a fluorescent product. The GPA assay is run at pH 7.2 in HEPES buffer containing 100 mM divalent calcium ion for 10 min at 25° C.

The SRC and BTC assays both degrade a natural substrate (collagen), which more closely approximates what collagenase injection is designed to do therapeutically. The GPA assays have the advantage that they utilizes a well-defined, small molecular weight hexapeptide as substrate and two well-defined tripeptides are produced. The GPA assays produce a fluorescent signal and is quite sensitive. Finally, the GPA assay are amenable to Michaelis-Menten kinetic analysis because it uses a single substrate, and reaction conditions (10 minutes incubation), which approximate initial enzyme velocities. The SRC assay is well-suited to collagen-degrading enzymes with collagen binding domains, whereas the GPA assay is well-suited to collagen-degrading enzymes without collagen binding domains, which are often referred to as gelatinases.

### A. Gpa Unit Assay Methods and Specific Activity Units

i. Collagenase Potency as Measured by GPA Assay (Cuvette)

The GPA assay is primarily used to measure the potency of a class II collagenase. The first step of the assay involves an enzymatic reaction involving the digestion of the substrate carbobenzoxy-glycyl-L-prolyl-glycyl-glycyl-prolyl-L-alanine (zGPGGPA) (SEQ ID NO: 7) by a collagenase sample into two peptides: carbobenzoxy-glycyl-L-prolyl-glycine (zGPG) and glycyl-prolyl-L-alanine (GPA). The second step involves the subsequent measurement of liberated GPA with the fluorogenic derivative fluorescamine. The assay follows the methodology below, but a person of ordinary skill in the art will appreciate that certain modifications (e.g., dilution concentrations and times) may be made yet carry out the purpose of the assay.

The general methodology is as follows. Leucine standards are prepared. A collagenase sample is obtained and solutions are prepared to be used in the first step for the enzymatic cleavage of zGPGGPA (SEQ ID NO: 7) (hereafter “substrate”) by collagenase. Following this step, the collagenase-treated samples (containing the liberated GPA) and leucine standards are treated at room temperature for a period of time with fluorescamine in order to fluorescently tag the free amino groups of the generated GPA and leucine molecules, respectively. The fluorescence emission of each solution at 480 nm is measured following excitation at 392 nm. The resulting slopes of the leucine and collagenase sample curves are then used to calculate potency units as follows:

$$Potency ( f - GPA units / mg = ( {M}_{Sample} / {M}_{L e u c i n e} ) \times ( DF / T )$$

Where:
M_{Sample}=Slope of the collagenase sample potency curve
M_{Leucine}=Slope of the leucine standard curve
DF=Dilution Factor
T=Reaction time

Additional, non-limiting details regarding the GPA assay methodology are set forth below.

### Buffers and Reagents

1. f-Appel's Buffer, pH 7.2 (55 mM HEPES, 100 mM calcium acetate)
2. 1 mM Leucine Working Stock Solution
3. 200 mM Borate, pH 9.0
4. 0.5 mM Fluorescamine Solution in Acetone
5. 2 mg/mL zGPGGPA (SEQ ID NO: 7) Substrate in f-Appel's Buffer

### Solution Preparation

Solutions are prepared as follows:

f-Appel's Buffer: Dissolve 13.0 g HEPES and 17.6 g calcium acetate in approximately 800 mL of water. Adjust pH to 7.2 with sodium hydroxide and QS to 1 L with water. Store at 2-8 degrees C.

10 mM Leucine Stock Solution: Dissolve 65.5 mg of leucine in 50 mL of water. Leucine must be weighed directly into a 100 mL (or equivalent) glass beaker on the scale. Weigh out approximately 65 mg (target weight) of leucine into the beaker. Based on the weight of leucine weighed, calculate the amount of water to add to the beaker using the equation below. Add the calculated volume of water to the beaker and mix thoroughly to ensure the leucine is fully dissolved. Dispense in to 1 mL aliquots. Store at less than or equal to 20 degrees C.

$$V 2 ( mL ) = C 2 ( mg ) \times \frac{V 1 ( 50 mL )}{C 1 ( 65.5 mg )}$$

Where:
C2=mass of leucine weighed (mg)
V1=50 (mL of water)
C1=65.5 (mg of leucine)
V2=volume of water needed to produce a 10 mM stock solution (mL)

1 mM Leucine Working Stock Solution: Thaw a vial of 10 mM Leucine Stock Solution and dilute to 1 mM by combining 150 μL with 1350 μL water. Mix well prior to use.

0.5 N HCl: Dilute HCl to 0.5 N with water and mix well. Store at room temperature. Alternatively, commercially available 0.5 N HCl may be used.

200 nM Borate, pH 9.0: Dissolve 2.4 g boric acid in approximately 150 ML water. Adjust the pH to 9.0 using sodium hydroxide. QS to 200 mL with water and mix well. Store at 2-8 degrees C.

0.5 mM Fluorescamine Solution: Mix 15 mg of fluorescamine with 100 mL acetone and swirl to dissolve. Store at 2-8 degrees C. protected from light.

Substrate Solution (2 mg/mL zGPGGPA) (SEQ ID NO: 7): Prepare substrate at 2 mg/mL with f-Appel's buffer. Dissolve on a mechanical shaker/rotator, allowing sufficient time for complete dissolution (about 15 minutes).

### Leucine Standard Curve

The leucine standard curve is prepared according to Table 7.

TABLE 7
Preparation of the leucine standard curve.

| Standard | L1 | L2 | L3 | L4 | L5 | L6 |
|---|---|---|---|---|---|---|
| Leucine Conc. (μM) | 0 | 70 | 140 | 210 | 280 | 350 |
| Water (μL) | 500 | 430 | 360 | 290 | 220 | 150 |
| 0.5N HCl (μL) | 500 | 500 | 500 | 500 | 500 | 500 |
| 1 mM Leucine (μL) | 0 | 70 | 140 | 210 | 280 | 350 |

“L1” means Leucine standard sample 1.

100 μL of each Leucine Standard is then transferred into separate tubes for detection of fluorescamine.

### Collagenase Sample Preparation

The collagenase sample is diluted to 0.01 mg/mL with f-Appel's Buffer in two stages and vortexed gently to mix. The following is an example dilution scheme:
1. 100 μL×1.0 mg/mL→1000 μL=0.1 mg/mL
2. 100 μL×0.1 mg/mL→1000 μL=0.01 mg/mL

### Blank Preparations

Blanks are prepared by combining 45 μL of the diluted preparation with 500 μL of 0.5 N hydrochloric acid to inactivate the enzyme. Add 455 μL of zGPGGPA (SEQ ID NO: 7) substrate solution and vortex to mix thoroughly. Transfer 100 μL of each blank into separate tubes for detection of impurities that may react with fluorescamine.

### Potency Curves

A set of potency curves are prepared for each collagenase sample as follows:

| Tubes | 2 mg/mL Substrate Solution | f-Appel's Buffer (μL) |
|---|---|---|
| 1-2 | 1 | 45 |
| 3-4 | 1 | 30 |
| 5-6 | 1 | 15 |

Warm the tubes containing substrate and buffer in a water bath at 25 degrees C. for a minimum of 15 minutes. Label a second set of tubes and add 50 μL of 0.5 N hydrochloric acid to each. Add the diluted collagenase sample preparations (0.01 mg/mL) to the tubes according to Table 8 for a 10 minute incubation, mix and return to the 25 degrees C. water bath. Start the incubation period upon addition of the first preparation to the pre-warmed substrate.

TABLE 8
Sample Preparation

| Preparation | Tubes | Sample (μL) |
|---|---|---|
| Potency Curve | 1-2 | 55 |
| Potency Curve | 3-4 | 70 |
| Potency Curve | 5-6 | 85 |

Remove the preparations from the water bath with 1-2 minutes remaining on the 10 minute incubation and vortex gently to mix. Ten minutes after addition of the first preparation to the substrate, transfer 50 μL from each tube into the tubes containing 50 μL of 0.5 N HCl. The preparations should be added directly to the acid to quench the digestion. Vortex each tube to mix well after quenching all preparations.

### Detection

Add 400 μL of 200 mM Borate Buffer and 500 μL of 0.5 mM Fluorescamine Solution to all detection tubes containing 100 μL of each preparation (blanks, collagenase sample potency curves, and leucine standards). Vortex thoroughly to mix. Allow the tubes to incubate at room temperature for a minimum of 10 minutes.

### Fluorometer Setup

Set up the fluorometer with the following instrument parameters and read the fluorescence of each preparation with 1 hour of derivatization.

| Parameter | Setting | |
|---|---|---|
| Excitation Wavelength | 392 | nm |
| Emission Wavelength | 480 | nm |
| Integration | 5.0 | sec. |
| Slits (Ex & Em) | 5.0 nm (band pass) | |
| Path Length | 3 | mm |

### Calculations

Plot the concentration of each leucine standard (X-axis) against the fluorescence response at 480 nm (Y-axis). Determine the slope (m) and coefficient of determination (R²). Determine the mean fluorescence of each potency curve preparation. Prepare collagenase sample and leucine potency curves by plotting the concentration of each preparation (X-axis) against the mean fluorescent response at 480 nm (Y-axis). Determine the slope (m) and coefficient of determination (R²) for the resulting linear curves.

### Determine Potency of Collagenase Sample

$$Potency ( f - GPA units / mg = ( {M}_{Sample} / {M}_{L e u c i n e} ) \times ( DF / T )$$

Where:
M_{Sample}=Slope of the collagenase sample potency curve
M_{Leucine}=Slope of the leucine standard curve
DF=Dilution Factor (1100 μL/50 μL=22)
T=Reaction time (10 minutes)
   ii. GPA Microplate Assay for the Determination of Class II Collagenase Activity in a Collagenase Sample

This method is similar to the GPA assay above, except is performed in a microplate. Like the assay above, the microplate assay measures the proteolytic activity of collagenase samples in the enzymatic cleavage of the substrate carbenzoxy-glycyl-L-prolyl-glycyl-glycyl-L-propyl-L-alanine (zGPGGPA) (SEQ ID NO: 7) (hereafter, “substrate”). The assay follows the methodology below, but a person of ordinary skill in the art will appreciate that certain modifications (e.g., dilution concentrations and times) may be made yet carry out the purpose of the assay.

### Reagents

1. Peptide substrate (zGPGGPA) (SEQ ID NO: 7) (Bachem M1260 or equivalent)
2. Tripeptide GPA (Bachem H3615 or equivalent)
3. Fluorescamine (Acros 191675000 or equivalent)
4. Purified Water (Milli-Q-Plus 18.2 M9 system or equivalent)
5. 1 M HEPES buffer (Gibco 15630-080 or equivalent)
6. Surfact-Amps 20™ (10% Tween solution) (Pierce Cat. #28320 or equivalent)
7. 1 M Calcium Acetate (Ca(C₂H₃O₂)₂) (Emerald Biosciences Cat. #EBS-100-CAAC or equivalent)
8. Boric acid (Sigma B7660 or equivalent)
9. 2.5 N NaOH (J.T Baker 5666-02 or equivalent)
10. 0.5 N Hydrochloric Acid (VWR 101223-134 or equivalent)
11. Acetone (Sigma 270725 or equivalent)

### Preparation of Solutions

(i) Preparation of assay buffer (50 mM HEPES pH 7.1/0.05% Tween 20/5 mM (Ca(C₂H₃O₂)₂): An amount of 50 mL 1 M HEPES is pipetted into 800 mL DI water. 5 mL 1 M (Ca(C₂H₃O₂)₂) and 5 mL Surfact-Amps (10% Tween 20) are added. The pH is checked and adjusted to 7.1±0.05 if necessary. A sufficient quantity of water is added to adjust the volume to 1 L and the solution is filtered through a 0.22 micron filter. This assay buffer can be stored at room temperature for up to 3 months.
(ii) Preparation of 0.1 N NaOH: An amount of 2 mL of 2.5 N NaOH is added into 48 mL DI water. This solution can be stored at room temperature for up to 3 months.
(iii) Preparation of 4 mg/mL tripeptide GPA stock solution: An amount of 400 mg (±1 mg) of tripeptide GPA is dissolved into 10 mL 0.1 N NaOH and vortexed until totally dissolved. A sufficient quantity of assay buffer is added to make the volume 100 mL and the solution is dispensed into 0.5 mL aliquots and stored at −70° C. The 4 mg/mL tripeptide GPA stock can be stored at −70° C. for up to one year.
(iv) Preparation of 4 mg/mL (6.8 mM) peptide substrate zGPGGPA (SEQ ID NO: 7): An amount of 400 mg (+1 mg) of the peptide substrate zGPGGPA (SEQ ID NO: 7) is dissolved into 10 mL 0.1N NaOH, vortex until totally dissolved. A sufficient quantity of assay buffer is added to make the volume 100 mL. This solution can be stored at 4° C. for up to 3 months.
(v) Preparation of 120 mM Boric Acid pH 9.0: An amount of 7.4 g (0.5 g) of the boric acid is dissolved into 800 mL DI water. The solution is titrated with NaOH to pH 9.0. a sufficient quantity of DI water is added to adjust the volume to 1 liter. This solution can be stored at room temperature for up to 3 months.
(vi) Preparation of 1 mM Fluorescamine in Acetone: An amount of 28±2 mg Fluorescamine is dissolved in 100 mL acetone. This solution needed to be freshly prepared and protected from light and moisture.

### Preparation of Tripeptide GPA Standard and Serial Dilution

A 0.08 mg/mL (329 μM) tripeptide GPA standard is prepared by making a 50-fold dilution of the 4 mg/mL tripeptide GPA stock in assay buffer (for example, 20 μL 4 mg/mL tripeptide GPA in 980 μL assay buffer). In the assay plate, row A, 200 μL of 329 μM tripeptide GPA standard is pipetted into A1 and A7. An amount of 100 μL assay buffer is pipetted into A2-A6 and A8-A12.

For the tripeptide GPA standard serial dilution, an amount of 100 μL is transferred from A1 into A2, mixed, an amount of 100 μL is transferred from A2 into A3, and repeated until A5. An amount of 100 μL is taken out from A5 well so that its final volume is 100 μL. The A6 well contains buffer only.

For the second tripeptide GPA standard serial dilution, an amount of 100 μL is transferred from A7 into A8, mixed, an amount of 100 μL is transferred from A8 into A9, and repeated until well A11. An amount of 100 μL is taken out from A11 well so that its final volume is 100 μL. The A12 well contains buffer only.

Preparation of Collagenase Samples

For collagenase samples (e.g., a lyophilized collagenase drug product), the sample is allowed to come to room temperature for at least 10 minutes and reconstituted to form a 500 ng/mL stock solution. Different concentrations may be used. A test collagenase sample (T1A) is prepared from the stock solution by diluting with assay buffer. The procedure is repeated to prepare triplicate test samples (T1A, T1B, T1C).

Discussion

In this method, 50 μL of increasing concentrations of the collagenase test samples are mixed with 50 μL of excess substrate (2.0 mg/mL final concentration) in a 96-well plate. An amount of 50 μL of assay buffer is added to rows C-G in a U-bottomed, 96 well polypropylene reaction plate. 150 μL of collagenase samples are pipetted into row B. Then, a 1/1.5 serial dilution is performed using a multi-channel pipette, by transferring 100 μL of collagenase sample from row B into row C, mixing and repeating the process until row G is reached. An amount of 100 μL is removed and discarded from row G. Table 9 contains the final collagenase concentrations after adding 50 μL substrate to row B through row H.

The Blank is prepared in row H by pipetting 50 μL assay buffer to row H. This row contains no enzyme. Exemplary concentrations are shown in Table 9.

TABLE 9
Assay Target Concentrations After Substrate Addition

| Row | Dilution | Collagenase (ng/ml) |
|---|---|---|
| A | N/A | N/A |
| B | Stock | 250 |
| C | 1/1.5 | 167 |
| D | 1/2.3 | 111 |
| E | 1/3.4 | 74 |
| F | 1/5.1 | 49 |
| G | 1/7.6 | 33 |
| H-Blank | N/A | 0 |

### Collagenase Reaction

The zGPGGPA (SEQ ID NO: 7) substrate is cleaved by class II collagenases into zGPG and GPA during a 15 minute incubation time at room temperature. The incubator and temperature probe are turned on (temperature 22±1° C. prior to the addition of substrate to the plates). To column 1-12 in row B-H, 50 μL 4 mg/mL (6.8 mM) zGPGGPA (SEQ ID NO: 7) substrate is added column by column, then mixed. The reaction start time begins after the substrate is added to the first column. The plate is covered and placed in the 22±1° C. incubator for a total reaction time of 15±1 minutes.

After incubation, the reaction is quenched by the addition of hydrochloric acid, and the amount of released GPA peptide is quantitated after reacting the free amino terminus of the peptide with the fluorogenic reagent, fluorescamine. To quench the reaction, 100 μL of 0.5 N HCl is added into each well from row A to row H, added column by column, and then mixed. Reaction time ends after the HCl is added to the first column.

### Detection

An amount of 195 μL of 120 mM Borate pH 9.0 is added to each well of a Microplate Greiner polypropylene black reading plate. 30 μL of the quenched reaction mixture is transferred from the reaction plate into the corresponding wells of the reading plate and mixed well. Then, 75 μL of 1 mM Fluorescamine is added to each well of the reading plate (using a polypropylene tray to dispense Fluorescamine/acetone) and mixed immediately after every addition. The plate is read within 15 minutes after Fluorescamine addition with a Molecular Devices M2 fluorescence plate reader using the following settings: Excitation 380 nm, Emission 473 nm, cutoff 455 nm, 6 reads/well, PMT medium.

The concentration of GPA (μM) versus the emission at 473 nm and the concentration of collagenase (ng/mL) versus the emission at 473 nm are plotted. For each plot, a linear regression is fitted with no fixed parameters. For collagenase test samples, the zero point data are excluded from the linear fit and the entire triplicate data set for each sample is used to generate the plot. The slopes for the tripeptide GPA standard and collagenase samples are determined.

### Potency Determination

The collagenase sample specific activity can by calculated as follows:

GPA Microplate Assay Units=((Slope of Collagenase Sample)/(Slope of Tripeptide GPA×incubation time))×10⁶.

The specific activity of the collagenase test sample is determined from the slope of the tripeptide GPA standard and calculated by the curve-fitting program. Using the microplate method, different concentrations of substrate and different times may be used to calculate enzyme kinetics according to Michaelis-Menton.
iii. Collagenase Potency as Measured by GPA Assays

The collagenases useful in the present disclosure may have a potency of about 100,000 to about 300,000 GPA units/mg, or about 175,000 to about 300,000 f-GPA units/mg. In other embodiments, the potency may be about 70,000 to about 400,000 GPA units/mg, or about 100,000 to about 375,000 GPA units/mg, or about 125,000 to about 350,000 GPA units/mg, or about 150,000 to about 325,000 GPA units/mg, or about 175,000 to about 300,000 GPA units/mg, or about 200,000 to about 275,000 GPA units/mg. Alternatively, the potency may be about 70,000 to about 400,000 f-GPA units/mg, or about 100,000 to about 375,000 f-GPA units/mg, or about 125,000 to about 350,000 f-GPA units/mg, or about 150,000 to about 325,000 f-GPA units/mg, or about 175,000 to about 300,000 f-GPA units/mg, or about 230,000 to about 430,000 f-GPA units/mg, or about 200,000 to about 275,000 f-GPA units/mg. The collagenases may also have a potency of about 30,100 to 87,100, or about 43,000 to 67,000 GPA Microplate Assay Units. The above GPA assays may be employed to analyze the specific activity of any collagenase.

b. Src Unit Assay Methods and Specific Activity Units
i. Collagenase Potency as Measured by SRC Assay (Cuvette)

The SRC assay is primarily used to measure the potency of a class I collagenase. The general methodology is as follows. Leucine standards and collagenase sample solutions are prepared. The first step of the assay involves an enzymatic reaction involving the digestion of soluble rat-tail tendon collagen (SRC) by the collagenase. The second step involves the subsequent measurement of liberated peptide fragments/amino acids with the fluorogenic derivative fluorescamine. The assay follows the methodology below, but a person of ordinary skill in the art will appreciate that certain modifications (e.g., dilution concentrations and times) may be made yet carry out the purpose of the assay.

Such collagenase and leucine standard samples are treated with reagents in order to tag the generated GPA with fluorescamine. The leucine standards and collagenase samples are allowed to incubate at room temperature for 10 minutes prior to determining the fluorescence of each solution at 392 and 480 nm excitation and emission wavelengths, respectively. The resulting slopes of the leucine and collagenase sample curves are then used to calculate potency units as follows:

$$Potency ( f - SRC units / mg = ( {M}_{Sample} / {M}_{L e u c i n e} ) \times ( DF / T ) \times CF$$

Where:
M_{Sample}=Slope of the collagenase sample potency curve
M_{Leucine}=Slope of the leucine standard curve
DF=Dilution Factor (1500 μL/100 mL=15)
T=Reaction time (2.5 hr×60 min/1 hr=150 min)
CF=Conversion factor (1000 μg/1 mg=1000)

Additional, non-limiting details regarding the SRC assay methodology are set forth below.

### Buffers and Reagents

1. F-TC Assay Buffer, pH 7.2 (22 g HEPES [4-(2-Hydroxyethyl)-1-piperazineethanesulfonic acid], 4.4 g calcium acetate)
2. F-Enzyme Buffer, pH 7.2
3. 200 mM Borate, pH 9.0
4. 10 mM Leucine Stock Solution
5. 1 mM Leucine Working Stock Solution
6. 1 mM Fluorescamine Solution in Acetone
7. 2 mg/mL Rat Tail Collagen in 0.02N acetic acid

### Solution Preparation

Solutions are prepared as follows:

F-TC Assay Buffer: Dissolve 22 g HEPES and 4.4 g calcium acetate in approximately 900 mL of water. Adjust pH to 7.2 with sodium hydroxide and QS to 1 L with water. Store at 2-8° C.

F-Enzyme Buffer: Dilute F-TC Assay Buffer by combining 4 mL with 16 mL water. Store at 2-8° C.

10 mM Leucine Stock Solution: Dissolve 65.5 mg of leucine in 50 mL of water. Leucine must be weighed directly into a 100 mL (or equivalent) glass beaker on the scale. Weigh out approximately 65 mg (target weight) of leucine into the beaker. Based on the weight of leucine weighed, calculate the amount of water to add to the beaker using the equation below. Add the calculated volume of water to the beaker and mix thoroughly to ensure the leucine is fully dissolved. Dispense in to 1 mL aliquots. Store at less than or equal to −20° C.

$$V 2 ( mL ) = C 2 ( mg ) \times \frac{V 1 ( 50 mL )}{C 1 ( 65.5 mg )}$$

Where:
C2=mass of leucine weighed (mg)
V1=50 (mL of water)
C1=65.5 (mg of leucine)
V2=volume of water needed to produce a 10 mM stock solution (mL)

1 mM Leucine Working Stock Solution: Thaw a vial of 10 mM Leucine Stock Solution and dilute to 1 mM by combining 150 μL with 1350 μL water. Mix well prior to use.

0.5 N HCl: Dilute HCl to 0.5 N with water and mix well. Store at room temperature. Alternatively, commercially available 0.5 N HCl may be used.

0.02 N Acetic Acid: Combine 1 mL of 1 N Acetic Acid with 49 mL of water and mix well. Store at room temperature.

200 mM Borate, pH 9.0: Dissolve 2.4 g boric acid in approximately 150 mL water. Adjust the pH to 9.0 using sodium hydroxide. QS to 200 mL with water and mix well. Store at 2-8° C.

1 mM Fluorescamine Solution: Dissolve 15 mg of fluorescamine with 50 mL acetone and swirl to dissolve. Store at 2-8° C. protected from light.

Substrate Solution (2 mg/mL Rat Tail Collagen): Dilute sock rat tail collagen to 2 mg/mL with 0.02 N acetic acid. Store at 2-8° C.

### Leucine Standard Curve

The leucine standard curve is prepared according to Table 10.

TABLE 10
Preparation of the leucine standard curve

| Reagent | L1 | L2 | L3 | L4 | L5 | L6 |
|---|---|---|---|---|---|---|
| Leucine Conc. (μM) | 0 | 70 | 140 | 210 | 280 | 350 |
| Water (μL) | 1000 | 930 | 860 | 790 | 720 | 650 |
| 1 mM Leucine (μL) | 0 | 70 | 140 | 210 | 280 | 350 |

100 μL of each Leucine Standard is then transferred into separate centrifuge tubes for detection of fluorescamine.

### Collagenase Sample and Blanks Preparation

The sample is diluted to 0.01 mg/mL with F-Enzyme Buffer in two stages vortexed gently to mix. The following is an example dilution scheme:
1. 100 μL×1.0 mg/mL→1000 μL=0.1 mg/mL
2. 100 μL×0.1 mg/mL→1000 μL=0.01 mg/mL Maintain the diluted samples at room temperature until use.

Blanks are prepared according to Table 11 by first combining the sample and 0.5 N hydrochloric acid to inactivate the enzyme prior to addition of buffers and substrate.

Collagenase samples in labeled tubes according to Table 11. Tubes 1, 2, 4 and 6 are prepared from one preparation and tubes 3, 5 and 7 from the duplicate preparation.

TABLE 11
Blank and Collagenase Sample Preparations

| | | 0.5N HCl | F-Enzyme | F-TC | 2 mg/ml | Sample |
|---|---|---|---|---|---|---|
| Preparation | Tube(s) | (μl) | Buffer (μl) | Buffer (μl) | RTC (μl) | (μl) |
| Blank | 1 | 750 | 137.5 | 375 | 187.5 | 50.0 |
| Potency | 2-3 | — | 167.5 | 375 | 187.5 | 20.0 |
| Curve | 4-5 | — | 152.5 | 375 | 187.5 | 35.0 |
| | 6-7 | — | 137.5 | 375 | 187.5 | 50.0 |

The tubes are capped and vortexed gently to mix. The potency curve preparations are incubated in a 25° C.+3° C. water bath for 2.5 hours. At the end of incubation, the potency curve tubes are removed from the water bath. 750 μL of 0.5 N HCl is added to each preparation and vortexed thoroughly to mix. The preparations may be stored at 2-8° C. for up to 22 hours prior to detection.

### Detection/Fluorometer Setup

The leucine standards are prepared as described above.

Set up the luminescence spectrometer with the following instrument parameters and read the fluorescence of each preparation with 1 hour of derivatization.

| Parameter | Setting | |
|---|---|---|
| Excitation Wavelength | 392 | nm |
| Emission Wavelength | 480 | nm |
| Integration | 5.0 | sec. |
| Slits (Ex & Em) | 5.0 nm (band pass) | |
| Path Length | 3 | mm |

### Calculations

Plot the concentration of each leucine standard (X-axis) against the fluorescence response at 480 nm (Y-axis). Determine the slope (m) and coefficient of determination (R²). Do not force through zero. Determine the mean fluorescence of each duplicate preparation. Calculate the net fluorescence of each collagenase sample preparation.

$$F ( net ) = Mean Collagenase Sample ( {EM}_{480} ) - Blank ( {EM}_{480} )$$

Plot the amount of the collagenase sample in each preparation (X-axis) against the net fluorescence (Y-axis). Determine the slopes (m) and coefficient of determination (R²). Do not force through zero.

### Determine Potency of Collagenase Sample

$$Potency ( f - SRC units / mg = ( {M}_{Sample} / {M}_{L e u c i n e} ) \times ( DF / T ) \times CF$$

Where:
M_{Sample}=Slope of the collagenase sample potency curve
M_{Leucine}=Slope of the leucine standard curve
DF=Dilution Factor (1500 μL/100 mL=15)
T=Reaction time (2.5 hr×60 min/1 hr=150 min)
CF=Conversion factor (1000 μg/1 mg=1000)

The above SRC assay may be employed to analyze the specific activity of any collagenase.

ii. SRC Microplate Assay for the Determination of Class I Collagenase Activity in a Collagenase Sample

This method is similar to the SRC assay above, except is performed in a microplate. Like the SRC assay above, the microplate assay measures the collagenase activity towards soluble rat-tail collagen (SRC) substrate (hereafter, “substrate”). The assay follows the methodology below, but a person of ordinary skill in the art will appreciate that certain modifications may be made yet carry out the purpose of the assay.

### Reagents

1. Soluble Rat Collagen Substrate (BD Biosciences 354236)
2. Tripeptide GPA (Bachem H3615 or equivalent)
3. Fluorescamine (Acros 191675000 or equivalent)
4. Purified Water (Millipore, Milli-Q-Plus 18.2 M9 system or equivalent)
5. 1 M HEPES buffer (Gibco 15630-080 or equivalent)
6. 1 M Calcium Acetate (Ca(C2H302)2) (Emerald Biosciences EBS-100-CAAC or equivalent)
7. Surfact-Amps 20™ (10% Tween solution) (Pierce Cat. #28320 or equivalent)
8. 1.0 N Acetic acid (Sigma 318590 or equivalent)
9. 0.5 N Hydrochloric Acid (VWR 101223-134 or equivalent)
10. Boric acid (Sigma B7660 or equivalent)
11. 2.5 N Sodium hydroxide (J.T Baker 5666-02 or equivalent)
12. Acetone (Sigma 270725 or equivalent)

### Preparation of Solutions

(i) Preparation of assay buffer (50 mM HEPES pH 7.1/0.05% Tween 20/5 mM (Ca(C2H302)2): An amount of 50 mL 1 M HEPES is pipetted into 800 mL DI water. 5 mL 1 M (Ca(C2H302)2) and 5 mL Surfact-Amps (10% Tween 20) are added. The pH is checked and adjusted to 7.1±0.1 if necessary. A sufficient quantity of water is added to adjust the volume to 1 L and the solution is filtered through a 0.22 micron filter. This assay buffer can be stored at room temperature for up to 3 months.
(ii) Preparation of 0.1 N NaOH: An amount of 2 mL of 2.5 N NaOH is added into 48 mL DI water. This solution can be stored at room temperature for up to 3 months.
(iii) Preparation of 4 mg/mL tripeptide GPA stock: An amount of 400 mg (±1 mg) of GPA tripeptide is dissolved into 10 mL 0.1 N NaOH and vortexed until totally dissolved. A sufficient quantity of assay buffer is added to make the volume 100 mL and the solution is dispensed into 0.5 mL aliquots and stored at −70° C. The 4 mg/mL tripeptide GPA stock can be stored at −70° C. for up to one year.
(iv) Preparation of 0.02 N acetic acid: An amount of 1 mL of 1.0 N acetic acid is added to 40 mL of purified water. A sufficient amount of purified water is added to adjust the volume to 50 mL. This solution can be stored at room temperature for up to 1 year.
(v) Preparation of 2 mg/mL SRC substrate stock solution: An amount of 23.3 mL 0.02 N acetic acid is added directly to the vial in which substrate is supplied (supplied in one non-limiting example as 100 mg SRC at 3.75 mg/mL). Other concentrations of SRC substrate may be used. The calculation is below:

$$100 mg \div 375 mg / mL = 26.7 mL ;$$

Total vol (mL)=(3.75 mg/mL×26.7 mL)/2 mg/mL;

Total vol (50.0mL)−26.7 mL=23.3 mL

The solutions are mixed thoroughly by inversion and can be stored at 2-8° C. for up to 3 months.

(vi) Preparation of 0.6 mg/mL SRC substrate working solution: An amount of 4.2 mL of assay buffer is added to a 15 mL conical tube. Then, 1.8 mL of 2 mg/mL SRC substrate stock solution is added and the solution is mixed by inversion. This solution should be prepared immediately before addition to plate.
(vii) Preparation of 120 mM Boric Acid pH 9.0: An amount of 7.4 g (±0.5 g) of the boric acid is dissolved in 800 mL DI water. The solution is titrated with NaOH to pH 9.0 and sufficient DI water is added to adjust the volume to 1 L. This solution can be stored at room temperature for up to 3 months.
(viii) Preparation of 1 mM Fluorescamine in Acetone: An amount of 28±2 mg Fluorescamine is dissolved in 100 mL acetone. This solution needed to be freshly prepared and protected from light and moisture.

### Preparation of Tripeptide GPA Standard and Serial Dilution

A 0.08 mg/mL (329 μM) tripeptide GPA standard is prepared by making a 50-fold dilution of the 4 mg/mL tripeptide GPA stock in assay buffer (for example, 20 μL 4 mg/mL GPA in 980 μL assay buffer). In the assay plate, row A, 200 μL of 329 μM tripeptide GPA standard is pipetted into A1 and A7. An amount of 100 μL assay buffer is pipetted into A2-A6 and A8-A12.

For the tripeptide GPA standard serial dilution, an amount of 100 μL is transferred from A1 into A2, mixed, an amount of 100 μL is transferred from A2 into A3, and repeated until A5. An amount of 100 μL is taken out from A5 well so that its final volume is 100 μL. The A6 well contains buffer only.

For the second tripeptide GPA standard serial dilution, an amount of 100 μL is transferred from A7 into A8, mixed, an amount of 100 μL is transferred from A8 into A9, and repeated until A11. An amount of 100 μL is taken out from A11 well so that its final volume is 100 L. The A12 well contains buffer only.

### Preparation of Collagenase Test Samples

For collagenase samples (e.g., a lyophilized collagenase drug product), the sample is allowed to come to room temperature for at least 10 minutes and reconstituted to form a 3.0 μg/mL stock solution. Different concentrations may be used. A test collagenase sample (T1A) is prepared from the stock solution by diluting with assay buffer. The procedure is repeated to prepare triplicate test samples (T1A, T1B, T1C).

### Discussion

In this method, 50 μL of increasing concentrations of the test collagenase samples are mixed with 50 μL of excess substrate (0.2 mg/mL final concentration) in a 96-well plate. An amount of 50 μL of assay buffer is added to rows C-G in a U-bottomed, 96 well polypropylene reaction plate. 150 μL of collagenase samples are pipetted into row B. Then, a 1/1.5 serial dilution is performed using a multi-channel pipette, by transferring 100 μL of collagenase sample from row B into row C, mixing and repeating the process until row G is reached. An amount of 100 μL is removed and discarded from row G. The Blank is prepared in row H by pipetting 50 μL assay buffer to row H. This row contains no enzyme. Table 12 contains the final collagenase concentrations after adding 50 μL substrate to row B through row H.

TABLE 12
Assay Target Concentrations After Substrate Addition

| Row | Dilution | Collagenase (ng/ml) |
|---|---|---|
| A | N/A | N/A |
| B | Stock | 1500 |
| C | 1/1.5 | 100 |
| D | 1/2.3 | 667 |
| E | 1/3.4 | 444 |
| F | 1/5.1 | 296 |
| G | 1/7.6 | 198 |
| H-Blank | N/A | 0 |

### Collagenase Reaction

The incubator and temperature probe are turned on (temperature 22±1° C. prior to the addition of substrate to the plates). An amount of 50 μL 0.6 mg·mL SRC substrate is added to each well from row B to row H, added column by column then mixed. The reaction start time begins after the substrate is added to the first column. The plate is covered and placed in the 22±1° C. incubator for a total reaction time of 45±5 minutes. To quench the reaction, 100 μL of 0.5 N HCl is added into each well of the dilution plate, column by column, and mixed. Reaction time ends after the HCl is added to the first column.

### Detection

An amount of 195 μL of 120 mM Borate pH 9.0 is added to each well of a Microplate Greiner polypropylene black reading plate. 30 μL of the quenched reaction mixture is transferred from the reaction plate into the corresponding wells of the reading plate and mixed well. Then, 75 μL of 1 mM Fluorescamine is added to each well of the reading plate (using a polypropylene tray to dispense Fluorescamine/acetone) and mixed immediately after every addition. The plate is read within 15 minutes after Fluorescamine addition with a Molecular Devices M2 fluorescence plate reader using the following settings: Excitation 380 nm, Emission 473 nm, cutoff 455 nm, 6 reads/well, PMT medium.

The concentration of GPA (μM) versus the emission at 473 nm and the concentration of collagenase (ng/mL) versus the emission at 473 nm are plotted. For each plot, a linear regression is fitted with no fixed parameters. For collagenase samples, the zero point data are excluded from the linear fit and the entire triplicate data set for each sample is used to generate the plot. The slopes for the tripeptide GPA standard and collagenase samples are determined.

### Specific Activity and Relative Potency Determination

The collagenase sample specific activity can by calculated as follows:

$$SRC Microplate Assay Units = ( ( Slope of Collagenase Sample ) / ( Slope of Tripeptide GPA \times incubation time ) ) \times {10}^{6}$$

The specific activity of the collagenase test sample is determined from the slope of the tripeptide GPA standard and calculated by the curve-fitting program. Using the microplate method, different concentrations of substrate and different times may be used to calculate enzyme kinetics according to Michaelis-Menton.

iii. Collagenase Potency as Measured by SRC Assays

The collagenases useful in the present disclosure may have a potency of about 500 to about 15,000 SRC units/mg. In certain embodiments, the potency is about 500 to about 12,500 SRC units/mg, or about 700 to about 10,000 SRC units/mg, or about 1,000 to about 7,500 SRC units/mg, or 1,500 to about 6,000 SRC units/mg, or about 2,500 to about 5,000 SRC units/mg. Alternatively, the potency may be about 5,000 to about 35,000 f-SRC units/mg, or about 10,000 to about 30,000 f-SRC units/mg, or about 13,000 to about 23,000 f-SRC units/mg, or about 15,000 to about 25,000 f-SRC units/mg. The collagenases may also have a potency of about 980 to 3,510, or about 1,400 to 2,700 SRC Microplate Assay Units.

c. Collagenase Potency in Btc Unit Assay

The Bovine Tendon Collagen Assay for Collagenase is based on the procedure of Mandl et al. (1958), as modified by Keller and Mandl (1963). Since bovine tendon collagen is an insoluble substrate, it is important that it be finely divided. Trypsin is run as a control in order to account for the presence of denatured collagen or other protein impurities. The assay is run in the presence of calcium ions, which are required for collagenase activity. The number of peptides solubilized is determined by reacting the N-terminal amino group of the peptides with ninhydrin and measuring colorimetrically the amount of adjunct formed (Rosen 1957).

The purpose of this procedure is to test the specific activity of collagenase enzyme using a collagen substrate.

### Reagents and Solutions

1. Collagen Substrate (collagen)
2. Deionized Water (water)
3. Tris Assay Buffer
4. Trypsin Stock Solution
5. 0.5 M HCl
6. Leucine Standard Assay Solution (1 mM leucine)
7. Rosen Buffer
8. 3% Ninhydrin
9. 50% Isopropanol

### Incubation

Set up and label reaction tubes as follows: three tubes for the trypsin controls, six tubes for the Reference Solution and six tubes for each sample under test. Label and uncap each tube. Weigh out 10±1 mg collagen in the order of Table 13 and place the weighed collagen into each reaction tube.

TABLE 13
Order of Weighing and Reaction Tube Numbers

| Order of Weighing | Reaction Tube # |
|---|---|
| 1 | 1 |
| 2 | 4 |
| 3 | 6 |
| 4 | 8 |
| 5 | 10 |
| 6 | 12 |
| 7 | 14 |
| 8 | 16 |
| 9 | 18 |
| 10 | 20 |
| 11 | 2 |
| 12 | 3 |
| 13 | 5 |
| 14 | 7 |
| 15 | 9 |
| 16 | 11 |
| 17 | 13 |
| 18 | 15 |
| 19 | 17 |
| 20 | 19 |
| 21 | 21 |

For samples under test the amount of enzyme should contain an activity between 1.6 to 5.7 nmol leu eq/min per reaction tube (ACT). Undissolved samples should first be dissolved in Tris assay buffer before they are used in the assay. The concentration (before adding to the reaction tubes) should be no less than 0.0065 mg/mL.

Set the reaction tubes according to Table 14 to have a matrix pattern. The following table assumes 2 under test samples. If more or less samples are run, adjust the number of reaction tubes, but retain the pattern. Where volumes are constant, they are listed in Table 14.

TABLE 14
The Matrix Pattern

| | | | S3 | | |
|---|---|---|---|---|---|
| Reaction | S1 | S4 | Standard | | |
| Tube # | Tris Buffer | Trypsin | Solution | Samples*1 | Samples*2 |
| 1 | 1960 μL | 40 μL | | | |
| 2 | 1960 μL | 40 μL | | | |
| 3 | 1960 μL | 40 μL | | | |
| 4 | 1940 μL | | 60 μL | | |
| 5 | 1940 μL | | 60 μL | | |
| 6 | 1920 μL | | 80 μL | | |
| 7 | 1920 μL | | 80 μL | | |
| 8 | 1900 μL | | 100 μL | | |
| 9 | 1900 μL | | 100 μL | | |
| 10 | 1970 μL | | | 30 μL | |
| 11 | 1970 μL | | | 30 μL | |
| 12 | 1960 μL | | | 40 μL | |
| 13 | 1960 μL | | | 40 μL | |
| 14 | 1950 μL | | | 50 μL | |
| 15 | 1950 μL | | | 50 μL | |
| 16 | 1970 μL | | | | 30 μL |
| 17 | 1970 μL | | | | 30 μL |
| 18 | 1960 μL | | | | 40 μL |
| 19 | 1960 μL | | | | 40 μL |
| 20 | 1950 μL | | | | 50 μL |
| 21 | 1950 μL | | | | 50 μL |

*Suggested maximum number of samples is 3.

Cap the reaction tubes. Mix the contents gently but thoroughly. Place the reaction tubes in a 37° C. water bath. Incubate for 22±0.5 hours. Record the actual time incubation started a, 37° C., the number of the water bath used, the lot number of the collagen Lipid, and the collagen correction factor for the lot used and the lot numbers of all solutions used.

### Quenching And Filtration

Label a filtrate tube to correspond to each reaction tube incubated. Place a funnel containing and folded filter paper onto each labeled filtrate tube. At the end of the incubation period, remove the reaction tubes from the water bath. Record the actual time incubation ended.

Uncap the reaction tubes and discard the caps. Quench the reaction by dispensing 2 mL 0.5 M HCl into each reaction tube. Mix the contents of the tubes thoroughly. Filter the contents of each reaction tube into the appropriate filtrate tube.

The previous two steps need to be finished as quickly as possible because undigested collagen could be dissolved by HCl in a short time. The filtrate may be stored refrigerated in covered filtrate tubes for up to 95.5 hours before color development. Record the refrigeration and time stored.

### Color Development

Set up and label boiling tubes as follows: six tubes for the water and the leucine controls (Step 1) and two tubes for each filtrate tub (Step 1). Place the following amounts of water and leucine standard assay solution into the six leucine control tubes.

| Tube # | 1 | 2 | 3 | 4 | 5 | 6 |
|---|---|---|---|---|---|---|
| Water (μL) | 1000 | 900 | 850 | 800 | 750 | 700 |
| Leu (μL) | 0 | 100 | 150 | 200 | 250 | 300 |
| Leu (nmol) | 0 | 100 | 150 | 200 | 250 | 300 |

Pipette 0.8 mL of water into each boiling tube (Step 2). Pipette 0.2 mL of filtrate from each sample into the appropriately labeled boiling tubes. Dispense 0.5 mL of Rosen buffer into each boiling tube. Under a containment hood, dispense 0.5 mL of 3% ninhydrin into each boiling tube. Mix the contents of each tube thoroughly on a vortex mixer. Place the boiling tubes in a boiling water bath in a fume hood. Boil for 15±1 minutes. At the end of the boiling period, remove the boiling tubes from the water bath. Under a containment hood, dispense 5.0 mL of 50% Isopropanol into each boiling tube and mix the contents thoroughly. Allow the boiling tubes to reach ambient temperature (at least 10 minutes) before reading the absorbances.

### Reading Of Absorbances

Read the absorbances of the tubes while working under a containment hood. Turn on the spectrophotometer and allow it to warm up. Set the wavelength of the spectrophotometer to 570 nm. Zero the spectrophotometer against 50% Isopropanol. Read the absorbances (A₅₇₀) of the water, leucine, trypsin controls and the samples under test. Record the time that the first sample is read, in hours. Record the readings as 1000×A₅₇₀ and record the time that the last sample is read, in hours. All readings are to be done within a 1-hour interval.

### Calculations Principles

Calculate, in minutes, the total reading time and the total time of incubation. The total reading time should be less than 60 minutes and the total time of incubation should be between 1290-1350 minutes. Using the linear least square method, calculate the slope “b” and correlation coefficient “r” for leucine standards (x=nmol leucine vs y=A₅₇₀ reading). The unit for “b” value is A₅₇₀/nmol leucine. Record “b” value to two decimal places. b value for leucine should be between 2.88-3.33. Calculate the average reading for the trypsin controls (T). The average reading for the trypsin controls (T) should be 221-338. Record this average to the nearest whole number (Step A). Average duplicated sample A₅₇₀ reading for each reaction tube. Record this number to the nearest whole number. Subtract average trypsin (Step A) from the average sample A₅₇₀ reading to get the net sample reading.

Calculate the activity (ACT) per tube, in nmol leu eq/min, as follows:

$$ACT ( nmol leu eq / min ) = \frac{( Net sample reading ) ( 20 )}{( b ) ( Time in minutes )}$$

where 20 is the dilution factor for the amount of reaction mixture developed and “b” is the slope of the leucine standard curve. Record this number to one decimal point. The activity per tube of the samples under test should be 1.6-5.7 nmol leu eq/min.

Calculate the activity in BTC units, as follows:

BTC units=activity in nmol leu eq/min×collagen correction factor.

Calculate the activity in BTC units/mL of the sample as follows:

$$BTC unit / mL = \frac{Activity in BTC units}{Sample volume used in mL}$$

Calculate the specific activity of the sample in BTC units/mm follows:

$$BTC unit / mg = \frac{Activity in BTC units / mL}{Protein Concentration in mg / mL}$$

The conversion of BTC units to ABC units is:

$$ABC units = BTC units \times 1.09$$

i. BTC Units and ABC Units

Various collagenase compositions may be employed wherein the collagenase has a specific activity of about 5,000 BTC units/mg to about 25,000 BTC units/mg, or about 10,000 BTC units/mg to about 25,000 BTC units/mg, or about 15,000 BTC units/mg, or about 17,500 BTC units/mg, or about 20,000 BTC units/mg, or about 22,500 BTC units/mg, or about 9,175 BTC units/0.58 mg, or 15,817 BTC units/mg wherein “mg” refers to the amount of collagenase(s) present in a composition (as distinct from excipients and other constituents).

Further, various collagenase compositions may be employed wherein the collagenase has a specific activity of about 5,000 ABC units/mg to about 25,000 ABC units/mg, or about 10,000 ABC units/mg to about 25,000 ABC units/mg, or about 15,000 ABC units/mg, or about 17,500 ABC units/mg, or about 20,000 ABC units/mg, or about 22,500 ABC units/mg, or about 10,000 ABC units/0.58 mg, or 17,241 ABC units/mg wherein “mg” refers to the amount of collagenase(s) present in a composition (as distinct from excipients and other constituents).

d. Other Assays

Assay methods utilizing labelled collagen have been reported by Gisslow et al., Anal. Biochem., 68: 70-78 (1975); Robertson et al., Clinica Chimica Acta, 42:43-45 (1972); Sakamoto et al., A New Method for the Assay of Tissue Collagenase (36297) (1972). One other assay is the Worthington Biochemical Corp. Assay (http://www.worthington-biochem.com/CLS/assay.html) (accessed Jul. 3, 2019).

### 4. Doses of Collagenase

As for collagenase doses employed herein, the present disclosure provides for therapeutically effective amounts of collagenase sufficient to bind and lyse the septae upon subcutaneous injection to result in a decreased appearance of cellulite compared to pretreatment baseline.

In one embodiment, the collagenase may be injected in an amount of about 0.01 mg to about 20 mg in a single or divided doses. In another embodiment, the collagenase may be injected in an amount of about 0.05 mg to about 15 mg in a single or divided doses. In another embodiment, the collagenase may be injected in an amount of about 0.10 mg to about 10 mg in a single or divided doses. In another embodiment, the collagenase may be injected in an amount of about 0.15 mg to about 5 mg in a single or divided doses. In another embodiment, the collagenase may be injected in an amount of about 0.20 mg to about 3 mg in a single or divided doses. In another embodiment, the collagenase may be injected in an amount of about 0.25 mg to about 2 mg in a single or divided doses. In yet another embodiment, the collagenase may be injected in an amount of about 0.05 mg, about 0.10 mg, about 0.15 mg, about 0.20 mg, about 0.25 mg, about 0.30 mg, about 0.35 mg, about 0.40 mg, about 0.45 mg, about 0.50 mg, about 0.55 mg, about 0.60 mg, about 0.65 mg, about 0.70 mg, about 0.75 mg, about 0.80 mg, about 0.85 mg, about 0.90 mg, about 0.95 mg, about 1.00 mg, 1.05 mg, about 1.10 mg, about 1.15 mg, about 1.20 mg, about 1.25 mg, about 1.30 mg, about 1.35 mg, about 1.40 mg, about 1.45 mg, about 1.50 mg, about 1.55 mg, about 1.60 mg, about 1.65 mg, about 1.70 mg, about 1.75 mg, about 1.80 mg, about 1.85 mg, about 1.90 mg, about 1.95 mg, about 2.00 mg, 2.05 mg, about 2.10 mg, about 2.15 mg, about 2.20 mg, about 2.25 mg, about 2.30 mg, about 2.35 mg, about 2.40 mg, about 2.45 mg, about 2.50 mg, about 2.55 mg, about 2.60 mg, about 2.65 mg, about 2.70 mg, about 2.75 mg, about 2.80 mg, about 2.85 mg, about 2.90 mg, about 2.95 mg, about 3.00 mg, 3.05 mg, about 3.10 mg, about 3.15 mg, about 3.20 mg, about 3.25 mg, about 3.30 mg, about 3.35 mg, about 3.40 mg, about 3.45 mg, about 3.50 mg, about 3.55 mg, about 3.60 mg, about 3.65 mg, about 3.70 mg, about 3.75 mg, about 3.80 mg, about 3.85 mg, about 3.90 mg, about 3.95 mg, about 4.00 mg, 4.05 mg, about 4.10 mg, about 4.15 mg, about 4.20 mg, about 4.25 mg, about 4.30 mg, about 4.35 mg, about 4.40 mg, about 4.45 mg, about 4.50 mg, about 4.55 mg, about 4.60 mg, about 4.65 mg, about 4.70 mg, about 4.75 mg, about 4.80 mg, about 4.85 mg, about 4.90 mg, about 4.95 mg, about 5.00 mg, 5.05 mg, about 5.10 mg, about 5.15 mg, about 5.20 mg, about 5.25 mg, about 5.30 mg, about 5.35 mg, about 5.40 mg, about 5.45 mg, about 5.50 mg, about 5.55 mg, about 5.60 mg, about 5.65 mg, about 5.70 mg, about 5.75 mg, about 5.80 mg, about 5.85 mg, about 5.90 mg, about 5.95 mg, or about 6.00 mg.

In one embodiment, the collagenase may have a Vₘₐₓ of about 2.6 min⁻¹ to 5.2 min⁻¹, as measured using the SRC assay. In another embodiment, the collagenase may have a Vₘₐₓ of about 3.0 min⁻¹ to 5.0 min⁻¹, as measured using the SRC assay. In another embodiment, the collagenase may have a Vₘₐₓ of about 3.4 min⁻¹ to 4.8 min⁻¹, as measured using the SRC assay. In still another embodiment, the collagenase may have a Vₘₐₓ of about 3.5 min⁻¹ to 4.5 min⁻¹, as measured using the SRC assay. In yet another embodiment, the collagenase may have a Vₘₐₓ of about 2.0 min⁻¹, about 2.1 min⁻¹, about 2.2 min⁻¹, about 2.3 min⁻¹, about 2.4 min⁻¹, about 2.5 min⁻¹, about 2.6 min⁻¹, about 2.7 min⁻¹, about 2.8 min⁻¹, about 2.9 min⁻¹, about 3.0 min⁻¹, about 3.1 min⁻¹, about 3.2 min⁻¹, about 3.3 min⁻¹, about 3.4 min⁻¹, about 3.5 min⁻¹, about 3.6 min⁻¹, about 3.7 min⁻¹, about 3.8 min⁻¹, about 3.9 min⁻¹, about 4.0 min⁻¹, about 4.1 min⁻¹, about 4.2 min⁻¹, about 4.3 min⁻¹, about 4.4 min⁻¹, about 4.5 min⁻¹, about 4.6 min⁻¹, about 4.7 min⁻¹, about 4.8 min⁻¹, about 4.9 min⁻¹, about 5.0 min⁻¹, about 5.1 min⁻¹, about 5.2 min⁻¹, about 5.3 min⁻¹, about 5.4 min⁻¹, about 5.5 min⁻¹, about 5.6 min⁻¹, about 5.7 min⁻¹, about 5.8 min⁻¹, about 5.9 min⁻¹, or about 6.0 min⁻¹, as measured using the SRC assay. In another embodiment, the collagenase may have a Vₘₐₓ of about 0.7 min⁻¹ to 7.6 min⁻¹, as measured using the SRC assay, or about 1 to 6, or about 2 to 5, or about 3 to 4 min⁻¹, as measured using the SRC assay.

In one embodiment, the collagenase may have a Vₘₐₓ of about 135 min⁻¹ to 268 min⁻¹, as measured using the GPA assay. In another embodiment, the collagenase may have a Vₘₐₓ of about 150 min⁻¹ to 250 min⁻¹, as measured using the GPA assay. In another embodiment, the collagenase may have a Vₘₐₓ of about 175 min⁻¹ to 225 min⁻¹, as measured using the GPA assay. In still another embodiment, the collagenase may have a Vₘₐₓ of about 130 min⁻¹, about 135 min-1, about 140 min⁻¹, about 145 min⁻¹, about 150 min⁻¹, about 155 min⁻¹, about 160 min⁻¹, about 165 min⁻¹, about 170 min⁻¹, about 175 min⁻¹, about 180 min⁻¹, about 185 min⁻¹, about 190 min⁻¹, about 195 min⁻¹, about 200 min⁻¹, about 205 min⁻¹, about 210 min⁻¹, about 215 min⁻¹, about 220 min⁻¹, about 225 min⁻¹, about 230 min⁻¹, about 235 min⁻¹, about 240 min⁻¹, about 245 min⁻¹, about 250 min⁻¹, about 255 min⁻¹, about 260 min⁻¹, about 265 min⁻¹, about 270 min⁻¹, about 275 min⁻¹, or about 280 min⁻¹, as measured using the GPA assay. In another embodiment, the collagenase may have a Vₘₐₓ of about 4 min⁻¹ to 400 min⁻¹, as measured using the GPA assay, or about 0.3 to 30.5, or about 10 to 375, or about 20 to 350, or about 50 to 300, or about 100 to 275 min⁻¹, as measured using the GPA assay.

In one embodiment, the collagenase may have a Kₘ of about 75 mM to 147 mM, as measured using the SRC assay. In another embodiment, the collagenase may have a Kₘ of about 80 mM to 140 mM, as measured using the SRC assay. In another embodiment, the collagenase may have a Kₘ of about 85 mM to 130 mM, as measured using the SRC assay. In another embodiment, the collagenase may have a Kₘ of about 90 mM to 120 mM, as measured using the SRC assay. In yet another embodiment, the collagenase may have a Kₘ of about 70 mM, about 72 mM, about 75 mM, about 77 mM, about 80 mM, about 82 mM, about 85 mM, about 87 mM, about 90 mM, about 92 mM, about 95 mM, about 97 mM, about 100 mM, about 102 mM, about 105 mM, about 107 mM, about 110 mM, about 112 mM, about 115 mM, about 117 mM, about 120 mM, about 122 mM, about 125 mM, about 127 mM, about 130 mM, about 132 mM, about 135 mM, about 137 mM, about 140 mM, about 142 mM, about 145 mM, about 147 mM, about 150 mM, about 152 mM, about 155 mM, or about 157 mM, as measured using the SRC assay. In another embodiment, the collagenase may have a Kₘ of about 4.4 mM to 437 mM, as measured using the SRC assay, or about 5 to 400, or about 20 to 375, or about 50 to 325, or about 100 to 275, or about 150 to 250 mM, or about 4.1 to 410 nanoMolar as measured using the SRC assay.

In one embodiment, the collagenase may have a Kₘ of about 0.03 mM to 3.1 mM, as measured using the GPA assay. In another embodiment, the collagenase may have a Kₘ of about 1.00 mM to 1.60 mM, as measured using the GPA assay. In another embodiment, the collagenase may have a Kₘ of about 1.10 mM to 1.50 mM, as measured using the GPA assay. In another embodiment, the collagenase may have a Kₘ of about 1.15 mM to 1.40 mM, as measured using the GPA assay. In yet another embodiment, the collagenase may have a Kₘ of about 0.80 mM, about 0.82 mM, about 0.85 mM, about 0.87 mM, about 0.90 mM, about 0.92 mM, about 0.95 mM, about 0.97 mM, about 1.00 mM, about 1.02 mM, about 1.05 mM, about 1.07 mM, about 1.10 mM, about 1.12 mM, about 1.15 mM, about 1.17 mM, about 1.20 mM, about 1.22 mM, about 1.25 mM, about 1.27 mM, about 1.30 mM, about 1.32 mM, about 1.35 mM, about 1.37 mM, about 1.40 mM, about 1.42 mM, about 1.45 mM, about 1.47 mM, about 1.50 mM, about 1.52 mM, about 1.55 mM, about 1.57 mM, about 1.60 mM, about 1.62 mM, about 1.65 mM, or about 1.67 mM, as measured using the GPA assay. In another embodiment, the collagenase may have a Kₘ of about 0.027 mM to 2.7 mM, as measured using the GPA assay, or about 0.1 to 2, or about 0.5 to 1.5, or about 1 to 1.35 mM, as measured using the GPA assay.

In one embodiment, the collagenase may have a K_{cat} of about 36 sec⁻¹ to 671 sec⁻¹, as measured using the SRC assay. In another embodiment, the collagenase may have a K_{cat} of about 50 sec⁻¹ to 600 sec⁻¹, as measured using the SRC assay. In another embodiment, the collagenase may have a K_{cat} of about 60 sec⁻¹ to 500 sec⁻¹, as measured using the SRC assay. In another embodiment, the collagenase may have a K_{cat} of about 70 sec⁻¹ to 400 sec⁻¹, as measured using the SRC assay. In still another embodiment, the collagenase may have a K_{cat} of about 100 sec⁻¹ to 350 sec⁻¹, as measured using the SRC assay. In another embodiment, the collagenase may have a K_{cat} of about 30 sec⁻¹, about 40 sec⁻¹, about 50 sec⁻¹, about 60 sec⁻¹, about 70 sec⁻¹, about 80 sec⁻¹, about 90 sec⁻¹, about 100 sec⁻¹, about 110 sec⁻¹, about 120 sec⁻¹, about 130 sec⁻¹, about 140 sec⁻¹, about 150 sec⁻¹, about 160 sec⁻¹, about 170 sec⁻¹, about 180 sec⁻¹, about 190 sec⁻¹, about 200 sec⁻¹, about 210 sec⁻¹, about 220 sec⁻¹, about 230 sec⁻¹, about 240 sec⁻¹, about 250 sec⁻¹, about 260 sec⁻¹, about 270 sec⁻¹, about 280 sec⁻¹, about 290 sec⁻¹, about 300 sec⁻¹, about 310 sec⁻¹, about 320 sec⁻¹, about 330 sec⁻¹, about 340 sec⁻¹, about 350 sec⁻¹, about 360 sec⁻¹, about 370 sec⁻¹, about 380 sec⁻¹, about 390 sec⁻¹, about 400 sec⁻¹, about 410 sec⁻¹, about 420 sec⁻¹, about 430 sec⁻¹, about 440 sec⁻¹, about 450 sec⁻¹, about 460 sec⁻¹, about 470 sec⁻¹, about 480 sec⁻¹, about 490 sec⁻¹, about 500 sec⁻¹, about 510 sec⁻¹, about 520 sec⁻¹, about 530 sec⁻¹, about 540 sec⁻¹, about 550 sec⁻¹, about 560 sec⁻¹, about 570 sec⁻¹, about 580 sec⁻¹, about 590 sec⁻¹, about 600 sec⁻¹, about 610 sec⁻¹, about 620 sec⁻¹, about 630 sec⁻¹, about 640 sec⁻¹, about 650 sec⁻¹, about 660 sec⁻¹, about 670 sec⁻¹, about 680 sec⁻¹, about 690 sec⁻¹, about 700 sec⁻¹, about 710 sec⁻¹, about 720 sec⁻¹, about 730 sec⁻¹, about 740 sec⁻¹, about 750 sec⁻¹, or about 760 sec⁻¹, as measured using the SRC assay. In another embodiment, the collagenase may have a K_{cat} of about 1 sec⁻¹ to 107 sec⁻¹, as measured using the SRC assay, or about 10 to 100, or about 20 to 80, or about 30 to 70, or about 40 to 60 sec⁻¹, as measured using the SRC assay.

In one embodiment, the collagenase may have a K_{cat} of about 90 to 10,000, or about 41,000 sec⁻¹ to about 81,000 sec⁻¹, as measured using the GPA assay. In another embodiment, the collagenase may have a K_{cat} of about 45,000 sec⁻¹ to about 75,000 sec⁻¹, as measured using the GPA assay. In another embodiment, the collagenase may have a K_{cat} of about 50,000 sec⁻¹ to about 70,000 sec⁻¹, as measured using the GPA assay. In another embodiment, the collagenase may have a K_{cat} of about 55,000 sec⁻¹ to about 65,000 sec⁻¹, as measured using the GPA assay. In still another embodiment, the collagenase may have a K_{cat} of about 35,000 sec⁻¹, about 37,500 sec⁻¹, about 40,000 sec⁻¹, about 42,500 sec⁻¹, about 45,000 sec⁻¹, about 47,500 sec⁻¹, about 50,000 sec⁻¹, about 52,500 sec⁻¹, about 55,000 sec⁻¹, about 57,500 sec⁻¹, about 60,000 sec⁻¹, about 62,500 sec⁻¹, about 65,000 sec⁻¹, about 67,500 sec⁻¹, about 70,000 sec⁻¹, about 72,500 sec⁻¹, about 75,000 sec⁻¹, about 77,500 sec⁻¹, about 80,000 sec⁻¹, about 82,500 sec⁻¹, or about 85,000 sec-, as measured using the GPA assay. In another embodiment, the collagenase may have a K_{cat} of about 1215 sec⁻¹ to about 120,000 sec⁻¹, as measured using the GPA assay, or about 2,000 to 100,000, or about 10,000 to 90,000, or about 20,000 to 80,000, or about 30,000 to 70,000, or about 40,000 to 60,000 sec⁻¹, as measured using the GPA assay.

In one embodiment, the collagenase may have 1/K_{cat} of about 376 to 38,000 μsec, or about 14,000 μsec to about 28,000 μsec, as measured using the SRC assay. In another embodiment, the collagenase may have 1/K_{cat} of about 16,000 μsec to about 26,000 μsec, as measured using the SRC assay. In one embodiment, the collagenase may have 1/K_{cat} of about 18,000 μsec to about 24,000 μsec, as measured using the SRC assay. In one embodiment, the collagenase may have 1/K_{cat} of about 20,000 μsec to about 22,000 μsec, as measured using the SRC assay. In still another embodiment, the collagenase may have 1/K_{cat} of about 12,500 μsec, about 12,750 μsec, about 13,000 μsec, about 13,250 μsec, about 13,500 μsec, about 13,750 μsec, about 14,000 μsec, about 14,250 μsec, about 14,750 μsec, about 15,000 μsec, about 15,250 μsec, about 15,500 μsec, about 15,750 μsec, about 16,000 μsec, about 16,250 μsec, about 16,500 μsec, about 16,750 μsec, about 17,000 μsec, about 17,250 μsec, about 17,500 μsec, about 17,750 μsec, about 18,000 μsec, about 18,250 μsec, about 18,500 μsec, about 18,750 μsec, about 19,000 μsec, about 19,250 μsec, about 19,500 μsec, about 19,750 μsec, about 20,000 μsec, about 20,250 μsec, about 20,500 μsec, about 20,750 μsec, about 21,000 μsec, about 21,250 μsec, about 21,500 μsec, about 21,750 μsec, about 22,000 μsec, about 22,250 μsec, about 22,500 μsec, about 22,750 μsec, about 23,000 μsec, about 23,250 μsec, about 23,500 μsec, about 23,750 μsec, about 24,000 μsec, about 24,250 μsec, about 24,500 μsec, about 24,750 μsec, about 25,000 μsec, about 25,250 μsec, about 25,500 μsec, about 25,750 μsec, about 26,000 μsec, about 26,250 μsec, about 26,500 μsec, about 26,750 μsec, about 27,000 μsec, about 27,250 μsec, about 27,500 μsec, about 27,750 μsec, about 28,000 μsec, about 28,250 μsec, about 28,500 μsec, about 28,750 μsec, about 29,000 μsec, or about 29,250 μsec, as measured using the SRC assay. In another embodiment, the collagenase may have 1/K_{cat} of about 370 μsec to about 36,700 μsec, as measured using the SRC assay, or about 750 to 30,000, or about 2,500 to 25,000, or about 5,000 to 20,000, or about 10,000 to 18,000, or about 15,000 μsec, as measured using the SRC assay.

In one embodiment, the collagenase may have 1/K_{cat} of about 4 μsec to about 430 μsec, as measured using the GPA assay. In another embodiment, the collagenase may have 1/K_{cat} of about 14 μsec to about 23 μsec, as measured using the GPA assay. In another embodiment, the collagenase may have 1/K_{cat} of about 16 μsec to about 21 μsec, as measured using the GPA assay. In still another embodiment, the collagenase may have 1/K_{cat} of about 10.0 μsec, about 10.2 μsec, about 10.4 μsec, about 10.6 μsec, about 10.8 μsec, about 11.0 μsec, about 11.2 μsec, about 11.4 μsec, about 11.6 μsec, about 11.8 μsec, about 12.0 μsec, about 12.2 μsec, about 12.4 μsec, about 12.6 μsec, about 12.8 μsec, about 13.0 μsec, about 13.2 μsec, about 13.4 μsec, about 13.6 μsec, about 13.8 μsec, about 14.0 μsec, about 14.2 μsec, about 14.4 μsec, about 14.6 μsec, about 14.8 μsec, about 15.0 μsec, about 15.2 μsec, about 15.4 μsec, about 15.6 μsec, about 15.8 μsec, about 16.0 μsec, about 16.2 μsec, about 16.4 μsec, about 16.6 μsec, about 16.8 μsec, about 17.0 μsec, about 17.2 μsec, about 17.4 μsec, about 17.6 μsec, about 17.8 μsec, about 18.0 μsec, about 18.2 μsec, about 18.4 μsec, about 18.6 μsec, about 18.8 μsec, about 19.0 μsec, about 19.2 μsec, about 19.4 μsec, about 19.6 μsec, about 19.8 μsec, about 20.0 μsec, about 20.2 μsec, about 20.4 μsec, about 20.6 μsec, about 20.8 μsec, about 21.0 μsec, about 21.2 μsec, about 21.4 μsec, about 21.6 μsec, about 21.8 μsec, about 22.0 μsec, about 22.2 μsec, about 22.4 μsec, about 22.6 μsec, about 22.8 μsec, about 23.0 μsec, about 23.2 μsec, about 23.4 μsec, about 23.6 μsec, about 23.8 μsec, about 24.0 μsec, about 24.2 μsec, about 24.4 μsec, about 24.6 μsec, about 24.8 μsec, about 25.0 μsec, about 25.2 μsec, about 25.4 μsec, about 25.6 μsec, about 25.8 μsec, about 26.0 μsec, about 26.2 μsec, about 26.4 μsec, about 26.8 μsec, about 27.0 μsec, about 27.2 μsec, or about 27.4 μsec, as measured using the GPA assay. In another embodiment, the collagenase may have 1/K_{cat} of about 0.3 μsec to about 32 μsec, as measured using the GPA assay, or about 1 to 30, or about 5 to 25, or about 10 to 20, or about 15 μsec, as measured using the GPA assay.

In one embodiment, the collagenase may have a K_{cat}/Kₘ of about 5,140 mM⁻¹sec⁻¹ to about 508,814 mM⁻¹sec⁻¹, as measured using the SRC assay. In another embodiment, the collagenase may have a K_{cat}/Kₘ of about 0.50 mM⁻¹sec⁻¹ to about 7.75 mM⁻¹sec⁻¹, as measured using the SRC assay. In another embodiment, the collagenase may have a K_{cat}/Kₘ of about 0.75 mM⁻¹sec⁻¹ to about 7.00 mM⁻¹sec⁻¹, as measured using the SRC assay. In still another embodiment, the collagenase may have a K_{cat}/Kₘ of about 1.00 mM⁻¹sec⁻¹ to about 6.00 mM⁻¹sec⁻¹, as measured using the SRC assay. In still another embodiment, the collagenase may have a K_{cat}/Kₘ of about 0.10 mM⁻¹sec⁻¹, about 0.20 mM⁻¹sec⁻¹, about 0.30 mM⁻¹sec⁻¹, about 0.40 mM⁻¹sec⁻¹, about 0.50 mM⁻¹sec⁻¹, about 0.60 mM⁻¹sec⁻¹, about 0.70 mM⁻¹sec⁻¹, about 0.80 mM⁻¹sec⁻¹, about 0.90 mM⁻¹sec⁻¹, about 1.00 mM⁻¹sec⁻¹, about 1.10 mM⁻¹sec⁻¹, about 1.20 mM⁻¹sec⁻¹, about 1.30 mM⁻¹sec⁻¹, about 1.40 mM⁻¹sec⁻¹, about 1.50 mM⁻¹sec⁻¹, about 1.60 mM⁻¹sec⁻¹, about 1.70 mM⁻¹sec⁻¹, about 1.80 mM⁻¹sec⁻¹, about 1.90 mM⁻¹sec⁻¹, about 2.00 mM⁻¹sec⁻¹, about 2.10 mM⁻¹sec⁻¹, about 2.20 mM⁻¹sec⁻¹, about 2.30 mM⁻¹sec⁻¹, about 2.40 mM⁻¹sec⁻¹, about 2.50 mM⁻¹sec⁻¹, about 2.60 mM⁻¹sec⁻¹, about 2.70 mM⁻¹sec⁻¹, about 2.80 mM⁻¹sec⁻¹, about 2.90 mM⁻¹sec⁻¹, about 3.00 mM⁻¹sec⁻¹, about 3.10 mM⁻¹sec⁻¹, about 3.20 mM⁻¹sec⁻¹, about 3.30 mM⁻¹sec⁻¹, about 3.40 mM⁻¹sec⁻¹, about 3.50 mM⁻¹sec⁻¹, about 3.60 mM⁻¹sec⁻¹, about 3.70 mM⁻¹sec⁻¹, about 3.80 mM⁻¹sec⁻¹, about 3.90 mM⁻¹sec⁻¹, about 4.00 mM⁻¹sec⁻¹, about 4.10 mM⁻¹sec⁻¹, about 4.20 mM⁻¹sec⁻¹, about 4.30 mM⁻¹sec⁻¹, about 4.40 mM⁻¹sec⁻¹, about 4.50 mM⁻¹sec⁻¹, about 4.60 mM⁻¹sec⁻¹, about 4.70 mM⁻¹sec⁻¹, about 4.80 mM⁻¹sec⁻¹, about 4.90 mM⁻¹sec⁻¹, about 5.00 mM⁻¹sec⁻¹, about 5.10 mM⁻¹sec⁻¹, about 5.20 mM⁻¹sec⁻¹, about 5.30 mM⁻¹sec⁻¹, about 5.40 mM⁻¹sec⁻¹, about 5.50 mM⁻¹sec⁻¹, about 5.60 mM⁻¹sec⁻¹, about 5.70 mM⁻¹sec⁻¹, about 5.80 mM⁻¹sec⁻¹, about 5.90 mM⁻¹sec⁻¹, about 6.00 mM⁻¹sec⁻¹, about 6.10 mM⁻¹sec⁻¹, about 6.20 mM⁻¹sec⁻¹, about 6.30 mM⁻¹sec⁻¹, about 6.40 mM⁻¹sec⁻¹, about 6.50 mM⁻¹sec⁻¹, about 6.60 mM⁻¹sec⁻¹, about 6.70 mM⁻¹sec⁻¹, about 6.80 mM⁻¹sec⁻¹, about 6.90 mM⁻¹sec⁻¹, about 7.00 mM⁻¹sec⁻¹, about 7.10 mM⁻¹sec⁻¹, about 7.20 mM⁻¹sec⁻¹, about 7.30 mM⁻¹sec⁻¹, or about 7.40 mM⁻¹sec⁻¹, as measured using the SRC assay. In another embodiment, the collagenase may have a K_{cat}/Kₘ of about 0.0048 mM⁻¹sec⁻¹ to about 0.47 mM⁻¹sec⁻¹, as measured using the SRC assay, or about 0.009 to about 0.3, or about 0.01 to about 0.25, or about 0.1 to 0.25 mM⁻¹sec⁻¹, as measured using the SRC assay.

In one embodiment, the collagenase may have a K_{cat}/Kₘ of about 60 mM⁻¹sec⁻¹ to about 6,000 mM⁻¹sec⁻¹, as measured using the GPA assay. In another embodiment, the collagenase may have a K_{cat}/Kₘ of about 30,000 mM⁻¹sec⁻¹ to about 85,000 mM⁻¹sec⁻¹, as measured using the GPA assay. In another embodiment, the collagenase may have a K_{cat}/Kₘ of about 36,000 mM⁻¹sec⁻¹ to about 77,000 mM⁻¹sec⁻¹, as measured using the GPA assay. In yet another embodiment, the collagenase may have a K_{cat}/Kₘ of about 40,000 mM⁻¹sec⁻¹ to about 70,000 mM⁻¹sec⁻¹, as measured using the GPA assay. In still another embodiment, the collagenase may have a K_{cat}/Kₘ of about 40,000 mM⁻¹sec⁻¹, about 42,000 mM⁻¹sec⁻¹, about 44,000 mM⁻¹sec⁻¹, about 46,000 mM⁻¹sec⁻¹, about 48,000 mM⁻¹sec⁻¹, about 50,000 mM⁻¹sec⁻¹, about 52,000 mM⁻¹sec⁻¹, about 54,000 mM⁻¹sec⁻¹, about 56,000 mM⁻¹sec⁻¹, about 58,000 mM⁻¹sec⁻¹, about 60,000 mM⁻¹sec⁻¹, about 62,000 mM⁻¹sec⁻¹, about 64,000 mM⁻¹sec⁻¹, about 66,000 mM⁻¹sec⁻¹, about 68,000 mM⁻¹sec⁻¹, about 70,000 mM⁻¹sec⁻¹, about 72,000 mM⁻¹sec⁻¹, about 74,000 mM⁻¹sec⁻¹, about 76,000 mM⁻¹sec⁻¹, about 78,000 mM⁻¹sec⁻¹, about 80,000 mM⁻¹sec⁻¹, about 82,000 mM⁻¹sec⁻¹, about 84,000 mM⁻¹sec⁻¹, about 86,000 mM⁻¹sec⁻¹, about 88,000 mM⁻¹sec⁻¹, about 90,000 mM⁻¹sec⁻¹, about 92,000 mM⁻¹sec⁻¹, about 94,000 mM⁻¹sec⁻¹, or about 96,000 mM⁻¹sec⁻¹, as measured using the GPA assay. In another embodiment, the collagenase may have a K_{cat}/Kₘ of about 900 mM^{-l}sec⁻¹ to about 90,000 mM⁻¹sec⁻¹, as measured using the GPA assay, or about 2,000 to 80,000, or about 10,000 to 70,000, or about 20,000 to 60,000, or about 30,000 to 50,000, or about 40,000 to 45,000 mM⁻¹sec⁻¹, as measured using the GPA assay.

In one embodiment, the collagenase may have a molecular mass of about 60 kDa to about 130 kDa. In another embodiment, the collagenase may have a molecular mass of about 70 kDa to about 130 kDa. In another embodiment, the collagenase may have a molecular mass of about 80 kDa to about 120 kDa. In still another embodiment, the collagenase may have a molecular mass of about 90 kDa to about 120 kDa. In another embodiment, the collagenase may have a molecular mass of about 100 kDa to about 110 kDa. In yet another embodiment, the collagenase may have a molecular mass of about 55 kDa, about 57 kDa, about 60 kDa, about 62 kDa, about 65 kDa, about 67 kDa, about 70 kDa, about 72 kDa, about 75 kDa, about 77 kDa, about 80 kDa, about 82 kDa, about 85 kDa, about 87 kDa, about 90 kDa, about 92 kDa, about 95 kDa, about 97 kDa, about 100 kDa, about 102 kDa, about 105 kDa, about 107 kDa, about 110 kDa, about 112 kDa, about 115 kDa, about 117 kDa, about 120 kDa, about 122 kDa, about 125 kDa, about 127 kDa, about 130 kDa, about 132 kDa, about 135 kDa, or about 137 kDa.

In one embodiment, the collagenase may have a purity of at least 80%, as measured by reverse phase HPLC. In another embodiment, the collagenase may have a purity of about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, as measured by reverse phase HPLC. In still another embodiment, the collagenase may comprise less than or equal to 1% by area of clostripain. In another embodiment, the collagenase may comprise less than or equal to 1% by area of gelatinase. In another embodiment, the collagenase may comprise less than or equal to 1% by area of leupeptin. In still another embodiment, the collagenase may comprise less than or equal to 1 cfu/mL bioburden.

In one embodiment, the collagenase may comprise a potency (i.e., specific activity) of about 500 to about 30,000 SRC units/mg. In another embodiment, the collagenase may comprise a potency of about 2,500 to about 25,000 SRC units/mg. In another embodiment, the collagenase may comprise a potency of about 5,000 to about 20,000 SRC units/mg. In still another embodiment, the collagenase may comprise a potency of about 500, about 1,000, about 1,500, about 2,000, about 2,500, about 3,000, about 3,500, about 4,000, about 4,500, about 5,000, about 5,500, about 6,000, about 6,500, about 7,000, about 7,500, about 8,000, about 8,500, about 9,000, about 9,500, about 10,000, about 10,500, about 11,000, about 11,500, about 12,000, about 12,500, about 13,000, about 13,500, about 14,000, about 14,500, about 15,000, about 15,500, about 16,000, about 16,500, about 17,000, about 17,500, about 18,000, about 18,500, about 19,000, about 19,500, about 20,000, about 20,500, about 21,000, about 21,500, about 22,000, about 22,500, about 23,000, about 23,500, about 24,000, about 24,500, about 25,000, about 25,500, about 26,000, about 26,500, about 27,000, about 27,500, about 28,000, about 28,500, about 29,000, about 29,500, or about 30,000 SRC units/mg.

In one embodiment, the collagenase may comprise a potency (i.e., specific activity) of about 5,000 to about 30,000 f-SRC units/mg. In another embodiment, the collagenase may comprise a potency of about 7,500 to about 25,000 f-SRC units/mg. In another embodiment, the collagenase may comprise a potency of about 10,000 to about 20,000 f-SRC units/mg. In still another embodiment, the collagenase may comprise a potency of about 2,500, about 3,000, about 3,500, about 4,000, about 4,500, about 5,000, about 5,500, about 6,000, about 6,500, about 7,000, about 7,500, about 8,000, about 8,500, about 9,000, about 9,500, about 10,000, about 10,500, about 11,000, about 11,500, about 12,000, about 12,500, about 13,000, about 13,500, about 14,000, about 14,500, about 15,000, about 15,500, about 16,000, about 16,500, about 17,000, about 17,500, about 18,000, about 18,500, about 19,000, about 19,500, about 20,000, about 20,500, about 21,000, about 21,500, about 22,000, about 22,500, about 23,000, about 23,500, about 24,000, about 24,500, about 25,000, about 25,500, about 26,000, about 26,500, about 27,000, about 27,500, about 28,000, about 28,500, about 29,000, about 29,500, or about 30,000 f-SRC units/mg.

In one embodiment, the collagenase may comprise a potency of about 100,000 to about 400,000 GPA units/mg. In another embodiment, the collagenase may comprise a potency of about 150,000 to about 350,000 GPA units/mg. In another embodiment, the collagenase may comprise a potency of about 200,000 to about 300,000 GPA units/mg. In still another embodiment, the collagenase may comprise a potency of about 100,000, about 110,000, about 120,000, about 130,000, about 140,000, about 150,000, about 160,000, about 170,000, about 180,000, about 190,000, about 200,000, about 210,000, about 220,000, about 230,000, about 240,000, about 250,000, about 260,000, about 270,000, about 280,000, about 290,000, about 300,000, about 310,000, about 320,000, about 330,000, about 340,000, about 350,000, about 360,000, about 370,000, about 380,000, about 390,000, or about 400,000 GPA units/mg.

In one embodiment, the collagenase may comprise a potency of about 175,000 to about 500,000 f-GPA units/mg. In another embodiment, the collagenase may comprise a potency of about 250,000 to about 450,000 f-GPA units/mg. In another embodiment, the collagenase may comprise a potency of about 300,000 to about 400,000 GPA units/mg. In still another embodiment, the collagenase may comprise a potency of about 175,000, about 185,000, about 195,000, about 205,000, about 215,000, about 225,000, about 235,000, about 245,000, about 255,000, about 265,000, about 275,000, about 285,000, about 295,000, about 305,000, about 315,000, about 325,000, about 335,000, about 345,000, about 355,000, about 365,000, about 375,000, about 385,000, about 395,000, about 405,000, about 415,000, about 425,000, about 435,000, about 445,000, about 455,000, about 465,000, about 475,000, about 485,000, or about 495,000 f-GPA units/mg.

In one embodiment, the collagenase may comprise a potency of about 5,000 to about 25,000 ABC units/mg. In one embodiment, the collagenase may comprise a potency of about 7,500 to about 20,000 ABC units/mg. In one embodiment, the collagenase may comprise a potency of about 10,000 to about 17,500 ABC units/mg. In another embodiment, the collagenase may comprise about 5,000, about 5,500, about 6,000, about 6,500, about 7,000, about 7,500, about 8,000, about 8,500, about 9,000, about 9,500, about 10,000, about 10,500, about 11,000, about 11,500, about 12,000, about 12,500, about 13,000, about 13,500, about 14,000, about 14,500, about 15,000, about 15,500, about 16,000, about 16,500, about 17,000, about 17,500, about 18,000, about 18,500, about 19,000, about 19,500, about 20,000, about 20,500, about 21,000, about 21,500, about 22,000, about 22,500, about 23,000, about 23,500, about 24,000, about 24,500, or about 25,000 ABC units/mg.

In some embodiments, the collagenase present in the composition comprises collagenase I and collagenase II in a ratio of approximately 1:1. Other ratios of collagenase I and collagenase II may be employed such as 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1. Each of collagenase I and collagenase II may have a purity by area of at least 80%, or 85%, or 90%, or 91%, or 92%, or 93%, or 94%, or 95%, or 96%, or 97%, or 98%, or 99%, or 100% as measured by reverse phase HPLC.

In another embodiment, the collagenase composition comprises CCH having an AUX I and AUX II ratio of approximately 1:1. Other ratios of AUX I and AUX II may be employed such as 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1. Each of AUX I and AUX II may have a purity by area of at least 80%, or 85%, or 90%, or 91%, or 92%, or 93%, or 94%, or 95%, or 96%, or 97%, or 98%, or 99%, or 100% as measured by reverse phase HPLC.

In other examples, the collagenase composition may be a liquid or is reconstituted from a lyophilized solid form with a diluent. The dose of the mixture is measured by the amount of collagenase present without regard to diluent, and may comprise about 0.1 mg to about 20 mg in one or more injections. In another embodiment, the dose administered is about 0.06 mg, 0.48 mg, 0.84 mg, 1.68 mg, 2.52 mg, 3.36 mg, 4.2 mg, 5.04 mg, 5.88 mg, 6.72 mg, 7.56 mg, or 8.4 mg in one or more injections.

For instance, about 0.06 mg, 0.48 mg, 0.84 mg, or 1.68 mg is administered in about 12 divided injections. The volume of collagenase composition injected may range from 0.01 mL to 3 mL per injection, or total about 0.2 mL to 150 mL per treatment visit. In a specific embodiment, the above doses are to a collagenase composition comprising CCH. In another embodiment, the above doses are to a collagenase composition having one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another embodiment, about 0.84 mg of CCH is injected in about 12 equally divided injections per treatment area (about 0.07 mg×12 injections=about 0.84 mg). In some cases, such treatment with 0.84 mg occurs every 10-40 days for 2, 3, 4 or 5 treatment visits. In other cases, more than one treatment area is injected with 0.84 mg in one treatment visit or every 10-40 days for 2, 3, 4 or 5 treatment visits. In other embodiments, there are more than 5 treatment visits.

In another aspect, the amount of collagenase that may be injected to a treatment area(s) is about 0.001 mg to 20 mg collagenase per treatment visit in one or more injections, e.g., the dose is divided equally into about 3 to about 100 injections. The collagenase is in liquid form, or is reconstituted from a lyophilized solid with a diluent. The dose of collagenase is measured by the amount of collagenase without regard to diluent, and may comprise about 0.1 mg to 1 mg, or 0.25 mg to 0.75 mg, or 0.1 mg to 2 mg, or 0.25 mg to 1.75 mg, or 0.5 mg to 1 mg, 0.1 mg to 3 mg, or 0.25 mg to 2.75 mg, or 0.5 mg to 2.5 mg, or 0.75 mg to 2.25 mg, or 1 mg to 2 mg, or 0.1 mg to 4 mg, or 0.25 mg to 3.75 mg, or 0.5 mg to 3.5 mg, or 0.75 mg to 3 mg, or 1 mg to 3 mg. In other embodiments, the dose is about 0.001 mg, 0.01 mg, 0.04 mg, 0.05 mg, 0.07 mg, 0.1 mg, 0.2 mg, 0.3 mg, 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.25 mg, 2.5 mg, 2.75 mg, 3 mg, 3.25 mg, 3.5 mg, 3.75 mg, 4.0 mg, 4.25 mg, 4.5 mg, 4.75 mg, 5.0 mg, 5.25 mg, 5.5 mg, 5.75 mg, 6 mg, 6.25 mg, 6.5 mg, 6.75 mg, 7 mg, 7.25 mg, 7.5 mg, 7.75 mg, 8 mg, 8.25 mg, 8.5 mg, 8.75 mg, 9 mg, 9.25 mg, 9.5 mg, 9.75 mg, 10 mg, 11 mg, 12, mg, 13 mg, 14 mg, 15 mg, 16 mg, 17 mg, 18 mg, 19 mg, or 20 mg in one or more injections.

In another embodiment, the dose administered is about 0.06 mg, 0.48 mg, 0.84 mg, 1.68 mg, 2.52 mg, 3.36 mg, 4.2 mg, or 5.04 mg in one or more injections. In another example, about 0.06 mg, 0.48 mg, 0.84 mg, 1.68 mg, 2.52 mg, 3.36 mg, 4.2 mg, or 5.04 mg is administered in about 12 divided injections to a treatment area. In other examples, the dose of collagenase is divided into 3 or more injections. The volume of collagenase composition injected may range from 0.01 mL to 3 mL per injection, or total about 1 mL to 150 mL per treatment visit.

In one aspect, the AUX I and II mixture described above (“CCH”) may be injected in an amount of about 0.01 mg to 10 mg collagenase per treatment visit in one or more injections, e.g., the dose is divided equally into about 3 to about 50 injections. The collagenase may be a liquid or may be reconstituted from a lyophilized form with a diluent. The dose of the mixture is measured by the amount of collagenase without regard to diluent, and may comprise about 0.1 mg to 1 mg, or 0.25 mg to 0.75 mg, or 0.1 mg to 2 mg, or 0.25 mg to 1.75 mg, or 0.5 mg to 1 mg, 0.1 mg to 3 mg, or 0.25 mg to 2.75 mg, or 0.5 mg to 2.5 mg, or 0.75 mg to 2.25 mg, or 1 mg to 2 mg, or 0.1 mg to 4 mg, or 0.25 mg to 3.75 mg, or 0.5 mg to 3.5 mg, or 0.75 mg to 3 mg, or 1 mg to 3 mg, or about 0.05 mg, 0.1 mg, 0.2 mg, 0.3 mg, 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.25 mg, 2.5 mg, 2.75 mg, 3 mg, 3.25 mg, 3.5 mg, 3.75 mg, 4.0 mg, 4.25 mg, 4.5 mg, 4.75 mg, 5.0 mg, 5.25 mg, 5.5 mg, 5.75 mg, 6 mg, 6.25 mg, 6.5 mg, 6.75 mg, 7 mg, 7.25 mg, 7.5 mg, 7.75 mg, 8 mg, 8.25 mg, 8.5 mg, 8.75 mg, 9 mg, 9.25 mg, 9.5 mg, 9.75 mg or 10 mg in one or more injections. In another embodiment, the dose of CCH administered is about 0.06 mg, 0.48 mg, 0.84 mg, or 1.68 mg 2.52 mg, 3.36 mg, 4.2 mg, or 5.04 mg in one or more injections. For instance, about 0.06 mg, 0.48 mg, 0.84 mg, or 1.68 mg 2.52 mg, 3.36 mg, 4.2 mg, or 5.04 mg is administered in 12 injections. The volume of collagenase composition injected may range from 0.01 mL to 3 mL per injection, or total about 1 mL to 80 mL per treatment visit.

The doses of collagenase can also be expressed in mg per injection (again without regard to diluent) such as from about 0.001 mg to 0.5 mg per injection, about 0.01 mg to about 5 mg per injection, or about 0.005 mg to about 0.1 mg, or about 0.005 mg, 0.04 mg, or 0.07 mg per injection.

In certain aspects, the present disclosure contemplates injecting about 500 ABC units to about 50,000 ABC units per treatment visit, or about 10,000 ABC units to about 25,000 ABC units per treatment visit. In another embodiment, the dose of collagenase per injection is about 50 ABC units to about 2,500 ABC units, or about 85 ABC units to about 2,000 ABC units, or about 150 ABC units to about 1,750 ABC units, or about 200 ABC units to about 1,500 ABC units, or about 300 ABC units to about 1,250 ABC units, or about 500 ABC units to about 1,000 ABC units.

In certain embodiments, the doses based on various specific activities are as follows:

| | Doses | | | | | | | | | | | |
|---|---|---|---|---|---|---|---|---|---|---|---|---|
| Specific | 500 SRC | | 700 SRC | | 1000 SRC | | 1500 SRC | | 10,000 | | 15,000 | |
| Activities | Units | | Units | | Units | | Units | | ABC Units | | SRC Units | |
| 500 SRC | 1.00 | mg* | 1.40 | mg | 2.00 | mg | 3.00 | mg | 3.17 | mg | 30.00 | mg |
| Units/mg | | | | | | | | | | | | |
| 700 SRC | 0.71 | mg | 1.00 | mg | 1.43 | mg | 2.14 | mg | 2.27 | mg | 21.43 | mg |
| Units/mg | | | | | | | | | | | | |
| 1000 SRC | 0.50 | mg | 0.70 | mg | 1.00 | mg | 1.50 | mg | 1.59 | mg | 15.00 | mg |
| Units/mg | | | | | | | | | | | | |
| 1500 SRC | 0.33 | mg | 0.47 | mg | 0.67 | mg | 1.00 | mg | 1.06 | mg | 10.00 | mg |
| Units/mg | | | | | | | | | | | | |
| 10,000 ABC | 0.18 | mg^{†} | 0.25 | mg | 0.36 | mg | 0.55 | mg | 0.58 | mg | 5.48 | mg |
| Units/0.58 mg | | | | | | | | | | | | |
| 15,000 SRC | 0.033 | mg | 0.047 | mg | 0.067 | mg | 0.100 | mg | 0.106 | mg | 1.00 | mg |
| Units/mg | | | | | | | | | | | | |

*Milligram calculation from SRC units and specific activity in SRC units/mg is achieved by multiplying the SRC units by the inverse of the specific activity in SRC units/mg. For example, when the dose is 500 SRC units and the specific activity is 500 SRC units/mg, the amount in milligrams equivalent to the 500 SRC units dose is (500 SRC units) * (1/(500 SRC units/mg)) = 1.00 mg.
^{†}Milligram calculation from SRC units and specific activity in ABC units/mg is achieved by multiplying the SRC units by 6.3 ABC units/SRC unit, and then multiplying by the inverse of the specific activity in ABC units/mg. For example, when the dose is 500 SRC units and the specific activity is 10,000 ABC units/0.58 mg, the amount in milligrams equivalent to the 500 SRC units is (500 SRC units) * (6.3 ABC units/SRC unit) * (1/(10,000 ABC units/0.58 mg)) = 0.18 mg.

In certain aspects, the present disclosure contemplates injecting collagenase in an amount of about 5,000 BTC units to about 25,000 BTC units, or about 10,000 BTC units to about 25,000 BTC units, or about 15,000 BTC units, or about 17,500 BTC units, or about 20,000 BTC units, or about 22,500 BTC units, or about 9,175 BTC units, or about 15,817 BTC units.

### 5. Formulations

The CCH or other collagenase may be in the form of a pharmaceutical formulation comprising the CCH or collagenase and pharmaceutically acceptable excipients. Such excipients may include sterile water or sodium chloride/calcium chloride for injection, pH adjusting agents and stabilizers.

One non-limiting example is XIAFLEX®, supplied commercially by Applicant as single-use glass vials containing 0.9 mg of CCH as a sterile, lyophilized powder for reconstitution. Sterile diluent for reconstitution is also provided in a single-use glass vial. Inactive ingredients include hydrochloric acid, sucrose, and tromethamine. The diluent contains calcium chloride dihydrate in 0.9% sodium chloride. XJAFLEX® Prescribing Information (2018).

In another embodiment, CCH for cellulite is a sterile lyophilized powder comprising 0.92 mg CCH, sucrose, Tris, mannitol, and hydrochloric acid, in a 5-mL vial. A sterile diluent for reconstitution may comprise water for injection, normal saline, or 0.6% sodium chloride and 0.03% calcium chloride dehydrate in water for injection filled into individual 5 mL vials.

The collagenase or CCH may be filled into other size vials, e.g., 10 mL, 15 mL, 20 mL, or 30 mL. Other pH adjusting agents, sugars, polyols and stabilizing agents may be found in Rowe et al., Handbook of Pharmaceutical Excipients (5^{th} Ed.).

### 6. Methods of Treatment: Injection Techniques and Dosing Regimens

The foregoing collagenase compositions are useful in methods to treat or reduce the severity of cellulite in human subjects. The present disclosure relates to a method to reduce the severity of cellulite in a human patient, comprising: providing a composition comprising at least one collagenase; and injecting a therapeutically effective amount of the composition to one or more dimples, wherein the patient demonstrates a reduction in the severity of cellulite compared to a pretreatment baseline level of severity. As further detailed below, the composition may be administered by various injection techniques and the efficacy measured by a number of scales and other measurement tools.

The administration of the collagenase compositions described herein may be bilaterally (two thighs or two buttocks) or to all 4 quadrants (both buttocks and both thighs) in a single subject during a treatment visit. Such treatment visits may occur every 10-40 days for 2, 3, 4, 5 or 6 treatment visits over a one-year period. And, even when collagenase is injected to all 4 quadrants at high cumulative doses, the bioanalyses to detect plasma AUX-I or AUX-II concentrations after subcutaneous CCH administration up to and including 3.36 mg indicate that there was no quantifiable levels of CCH in systemic circulation (i.e. below the assay's lower level of quantitation) indicating no systemic absorption and further indicating overall safety. For example, humans dosed at 3.36 mg and rats dosed at 43 times the human equivalent dose (HED) showed that these amounts were well-tolerated. Applicant's studies of the pharmacokinetics of collagenase therapy are set forth in its U.S. Provisional Application Ser. No. 62/733,046 filed on Sep. 18, 2018, U.S. Provisional Application Ser. No. 62/788,916 filed on Jan. 6, 2019, U.S. Provisional Application Ser. No. 62/812,036 filed on Feb. 28, 2019, U.S. Provisional Application Ser. No. 62/823,596 filed on Mar. 25, 2019, International Appl. Ser. No. PCT/US2019/041494 filed on Jul. 11, 2019, and International Appl. Ser. No. PCT/US2019/41718 filed on Jul. 12, 2019, which are incorporated herein by reference.

A general overview of the various injection parameters for collagenase and related techniques for treatment of patients is provided in Table 15.

TABLE 15
An Overview of the Various Injection Parameters for Collagenase
and Related Techniques for Treatment of Patients

| Parameters | Approximate Ranges and Descriptions |
|---|---|
| Total dose per treatment visit (mg) | 1 mg to 20 mg collagenase |
| Total dose per treatment visit (fSRC units) | Collagenase or CCH: 5,000 to 600,000 |
| Total dose per treatment visit (fGPA units) | Collagenase or CCH: 175,000 to 10 million |
| Total dose per treatment visit (ABC units) | Collagenase or CCH: 5,000 to 500,000 |
| Total dose per treatment visit (SRC units) | Collagenase or CCH: 500 to 600,000 |
| Total dose per treatment visit (GPA units) | Collagenase or CCH: 100,000 to 8 million |
| Total dose per treatment area (mg) | Collagenase or CCH: 0.07 mg to 5 mg |
| Total dose per treatment area (fSRC units) | Collagenase or CCH: 1,250 to 150,000 |
| Total dose per treatment area (fGPA units) | Collagenase or CCH: 43,750 to 2.5 million |
| Total dose per treatment area (ABC units) | Collagenase or CCH: 1,250 to 125,000 |
| Total dose per treatment area (SRC units) | Collagenase or CCH: 1,250 to 150,000 |
| Total dose per treatment area (GPA units) | Collagenase or CCH: 25,000 to 2 million |
| Dose per injection | 0.0001 mg to 1 mg, or 0.0002 mg to 0.5 mg, |
| | or 0.00029 mg to 0.01 mg, or 0.01 mg to 2 |
| | mg, or 0.025 mg to 1.5 mg, or 0.05 mg to 1 |
| | mg, or 0.07 mg to 0.25 mg |
| Total injection volume per treatment visit | 0.1 mL to 150 mL, or 0.2 mL to 7.2 mL; or 5 |
| (mL) | mL to 50 mL, or 20 mL to 40 mL, or 28 mL |
| | to 36 mL |
| Total injection volume per treatment area | 0.1 mL to 36 mL |
| (mL) | |
| Total injection volume per dimple (mL) | 0.01 mL to 10 mL, or 1.2 mL, or 1.5 mL, or |
| | 0.3 mL. |
| Number of injection aliquots per injection | 1 to 12 |
| Number of injections per dimple | 1 to 5 |
| Number of aliquots per dimple | 1 to 60 |
| Volume of each aliquot | 0.01 mL to 36 mL |
| Collagenase concentration per injection | 0.01 mg/mL to 1 mg/mL, or 0.04 mg/mL to |
| | 0.9 mg/mL, or 0.06 mg/mL to 0.75 mg/mL, or |
| | 0.23 mg/mL, or 0.001 mg/mL to 10 mg/mL |
| Angle of subcutaneous injection into the | 5 to 175 degrees, or 30 to 150 degrees, or 45 |
| dimple | to 135 degrees, or 75 to 105 degrees, or 90 |
| | degrees |
| Depth of injection from skin surface | ⅛ inch to 2 inches |
| Needle gauge and length | 25 to 32 gauge; ⅛ inch to 3 inches needle |
| Number of injections per treatment visit | 3 to 175 injections |
| Length of time between treatment sessions | 7 to 100 days |
| (days) | |
| Dilution factor | 1x, 2x, 3x, 4x, 5x, 6x, 7x, 8x, 9x or 10x |
| Amount lyophilized collagenase in vial | 1 mg to 50 mg |
| Diluent | 0.9% NaCl/0.03% CaCl₂, or 0.6% NaCl/ |
| | 0.03% CaCl₂ |

| | Approximate Ranges and |
|---|---|
| Parameters | Descriptions |
| Osmolality of reconstituted | 50 to 1,000 mOsm/kg |
| product | |
| Amount of collagenase in vial | 1 mg to 50 mg |
| (liquid product) | |
| Vial size | 2 mL to 50 mL, or 5 mL, or 7.5 |
| | mL, or 10 mL, or 15 mL, or 20 mL, |
| | or 30 m, or 40 mL, or 50 mL |
| pH of collagenase | 4 to 9 |

In certain embodiments, about 0.84 mg of CCH is injected in about 12 equally divided injections to an affected area such as a quadrant (i.e., the right or left buttock or right or left thigh) (about 0.07 mg×12 injections=about 0.84 mg CCH). In some cases, such treatment with 0.84 mg occurs every 10-40 days for 2, 3, 4, 5 or 6 treatments. In other cases, more than one affected area or quadrant is injected with 0.84 mg every 10-40 days for 2, 3, 4, 5 or 6 treatments.

In one embodiment, a surgical marker is used to circle each of the dimples selected for treatment. In another embodiment, the circles in the selected treatment area do not overlap. In yet another embodiment, the circles in the selected treatment area overlap.

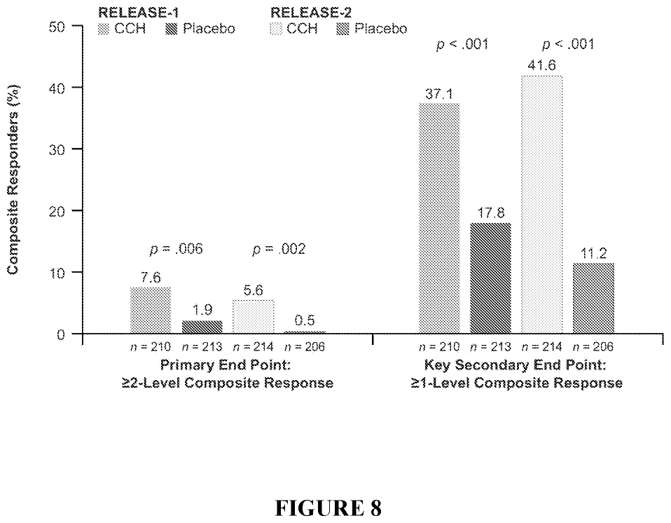

In certain embodiments, patients are administered collagenase as shown in Table 16 using the procedure according to Treatment I (FIG. 7).

TABLE 16
Collagenase Dose and Volume

| Dose per Each | Injection | | | Injection | |
|---|---|---|---|---|---|
| Injection^{a}/ | Volume | Number of | | Volume (mL) | |
| Number of | per Each | Injections at Each | Dose (mg) at Each | per Each | Cumulative |
| Subjects | Injection | Treatment Visit | Treatment Visit | Treatment Visit | EFP Dose |
| 0.07 mg | 0.3 mL | 12 per treatment | 0.84 mg per | 3.6 mL per | 5.04 mg (3 |
| | | area × 2 treatment | treatment area × 2 | treatment area × 2 | treatment visits × |
| | | areas = 24 | treatment areas = | treatment area = | 0.84 mg per |
| | | injections | 1.68 mg | 7.2 mL | treatment area × |
| | | | (12 injections per | (24 injections × | 2 treatment areas) |
| | | | treatment area × | 0.3 mL) | |
| | | | 0.07 mg/injection × | | |
| | | | 2 treatment areas) | | |

^{a}Each injection of collagenase is 0.3 mL administered as three 0.1 mL aliquots.

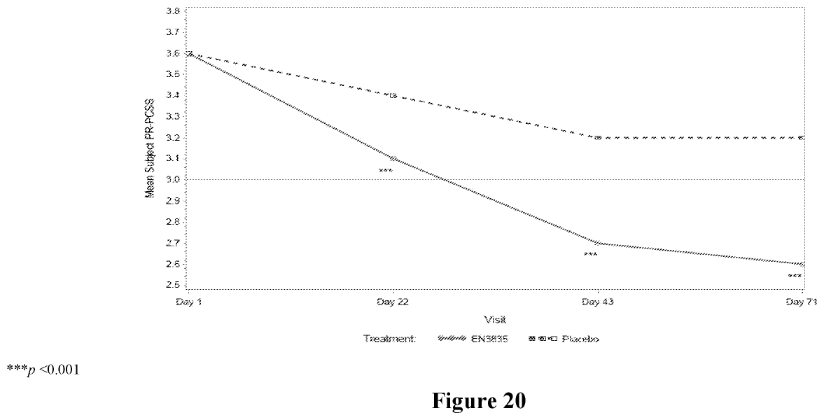

In this example, the clinician may select dimples within each buttock that are well-defined, evident when the subject is standing, and suitable for treatment. The clinician is not limited in his or her choice of dimples to treat. Treatment comprises 12 injections per buttock (24 injections total in 2 buttocks) per treatment visit. Because the goal of treatment is to improve the aesthetic appearance of each entire buttock, the clinician is instructed to select dimples that in his or her opinion would most improve the aesthetic appearance of each entire buttock. The same dimples within a buttock or different dimples from those previously treated within a buttock may be treated at each treatment visit, but injections are preferably within the buttocks (12 injections per buttock) for each of the 3 visits. Each buttock receives all 3 treatments unless the buttock has no treatable EFP dimples and the clinician rates the buttock a score of 0 on the CR-PCSS. If no injections in a particular buttock (right or left) are given at Treatment Visit 2, subjects are still assessed for treatment in the contralateral buttock at Treatment Visit 2 and return for Treatment Visit 3 and each of the buttocks is again evaluated by the subject (PR PCSS) and investigator (CR-PCSS). If the investigator rates either or both of the buttocks greater than 0 on the CR PCSS, injections at Treatment Visit 3 are given. Additionally, the collagenase treatment may include one or more of the following:
Treat 1 or more quadrants;
Treat dimples regardless of size;
Treat women older than 45;
Treat dimples devoid of skin laxity, flaccidity or sagging skin;
Treat different dimples at different treatment visits;
Use 1% inch or longer needle;
Not limit the distance between the dimples injected;
Not rely on a spacer, ruler, paper or other device to limit the location of the injections;
Ensure that at least one injection occur at the nadir of the dimple;
Treat dimples that are less than about 1 cm long or more than about 2 cm long;
Use injections within about 2 cm of each other;
Use injection within less than about 2 cm of each other; and/or
Measure efficacy using one or more of the scales and methods described herein.
   In certain embodiments, when the treatment adheres to the above aspects, patients experience a rapid rate of response to therapy. See FIGS. 20-23.

Further, in certain specific embodiments, the parameters for treatment patients are provided in Tables 17 and 18.

TABLE 17
CCH Lyophilized Formulation Parameters

| | Cellulite - Buttock/ | Cellulite- thigh/ |
|---|---|---|
| | treatment area | treatment area |
| Amount cake in vial | 0.92 mg CCH | 0.92 mg CCH |
| Syringe needle | 30 gauge × ½ | 30 gauge × 1 inch and |
| | inch | 30 gauge × ½ inch |
| Diluent | 0.6% NaCl/0.03% | 0.6% NaCl/0.03% |
| | CaCl₂ | CaCl₂ |
| Osmolality of | ~235-315 | ~227 mOsm |
| Reconstituted Product | mOsm/kg | |
| Dose applied per | 0.84 mg per | 0.84 mg per |
| treatment visit | treatment area | treatment area |
| Concentration to be | 0.233 mg/mL | 0.05 mg/mL |
| injected | | |
| Vial size | 5 mL or 10 mL | 5 mL or 10 mL |

TABLE 18
CCH Dilution Parameters

| | | Target | | |
|---|---|---|---|---|
| | | Concentration | | Osmolality |
| | Sample | mg/mL | pH | (mOsM/kg) |
| Diluent A | CCH 1X | 0.23 | 8.1 | 362 to 402 |
| (0.9% NaCl, | reconstitution | | | |
| 0.03% | CCH 2X | 0.12 | 7.8 | 329 to 358 |
| CaCl₂) | dilution | | | |
| | CCH 4X | 0.06 | 7.4 | 310 to 336 |
| | dilution | | | |
| | CCH 8X | 0.03 | 7.2 | 301 to 325 |
| | dilution | | | |
| Diluent B | CCH 1X | 0.23 | 8.0 | 267 to 300 |
| (0.6% NaCl, | reconstitution | | | |
| 0.03% | CCH 2X | 0.12 | 7.8 | 235 to 256 |
| CaCl₂) | dilution | | | |
| | CCH 4X | 0.06 | 7.5 | 216 to 234 |
| | dilution | | | |
| | CCH 8X | 0.03 | 7.3 | 208 to 223 |
| | dilution | | | |

In one embodiment, the osmolality of reconstitution product is about 50 to about 1,000, about 100 to about 900, about 200 to about 800, about 300 to about 700, about 400 to about 600, about 50, about 100, about 150, about 200, about 250, about 300, about 350, about 400, about 450, about 500, about 550, about 600, about 650, about 700, about 750, about 800, about 850, about 900, about 950, or about 1,000 mOsm/kg. In yet another embodiment, the osmolality of reconstitution product is about 512 mOsm/kg, about 275 mOsm/kg, about 281 mOsm/kg, or about 227 mOsm/kg.

In addition to the methods above, the current disclosure provides a method of treating or reducing EFP in a subject in need thereof, wherein the method provides at least one of the following advantages over common procedures and treatments of EFP:
a. ease of use by a physician;
b. shorter time to treat;
c. unexpected efficacy in light of physicians' general view that improvements for aesthetic conditions is difficult to obtain;
d. no need to use hyaluronidase;
e. no need to use heat;
f. no need to use lasers;
g. no subscision;
h. no need for anesthetic (despite bruising);
i. no need for compressive garments; and
j. no vacuum is used.

In another embodiment, the method of treatment or reducing cellulite places no cap on the severity of the cellulite to be treated, e.g., the treatment with collagenase is safe and effective regardless of the prevalence or severity of cellulite.

### F. PHASE 4-THERAPEUTIC ENDPOINTS AND MEASUREMENTS OF EFFICACY

The treatments described herein are effective in treating cellulite by a number of measures described below. Generally as used herein, the term “Day” means the study day measured sequentially day-by-day from the first day of collagenase treatment in a particular treatment area from the first treatment in a treatment course, except as otherwise provided in Example 5 below, which measures the days sequentially from Day 71 of prior studies. For instance, as explained in Example 5, “Day 180” means 180 days after the Day 71 reported in Examples 2 and 3. Thus, outside of the context of Example 5, Day 1 is the first day of treatment; Day 71 is 70 days after Day 1; Day 180 is 179 days after Day 1 (unless “Day 180” is used as in Example 5, which means it is 180 days after Day 71, or 251 days after the first day of treatment).

In certain embodiments, in a population of patients who all have CR-PCSS and/or PR-PCSS ratings of moderate or severe:
At least 50% of the patients show improvement in severity at Day 22, 43, or 71 from baseline of at least 1 level of severity in the CR-PCSS as assessed live by the clinician of the target treatment area.
At least 50% of the patients show improvement in severity at Day 22, 43, or 71 from baseline of at least 1 level of severity in the PR-PCSS as assessed by the subject while viewing the digital image of the target treatment area.
At least 5% of the patients show improvement in severity at Day 22, 43, or 71 from baseline of at least 2 levels of severity in the CR-PCSS as assessed live by the clinician of the target treatment area.
At least 5% of the patients show improvement in severity at Day 22, 43, or 71 from baseline of at least 2 levels of severity in the PR-PCSS as assessed by the subject while viewing the digital image of the target treatment area.
At least 5% of the patients experience a significant decrease in dimple size (volume, length, width, depth), e.g., at least a 5% decrease, or at least a 10% decrease, or at least a 20% decrease at Day 22, 43, or 71 from baseline.
   These and other efficacy parameters and benchmarks are detailed below. Further, as shown in the Examples below, collagenase injections significantly improved cellulite appearance, demonstrated durability, and was generally well-tolerated.

### 1. Efficacy as Measured by CR-PCSS and PR-PCSS

An improvement for an individual patient at any visit is an improvement of at least 1 level, or 1 rating, from baseline or any previous score or rating. An improvement for a group of patients at any visit is an improvement of about 0.1 in the mean score or rating from the baseline or any previous mean score or rating. A responder is any patient showing at least a 25% improvement of maximum total score or rating from baseline. In certain embodiments, the treatment methods detailed herein result in one or more of the following efficacy endpoints as measured by CR-PCSS and/or PR-PCSS:

1. An improvement of at least 0.1 in CR-PCSS and/or PR-PCSS rating over baseline.
2. An improvement in severity at Day 22, 43, 71, 90, 180, 251, 360, 431, 720, 3 years, 4 years, or 5 years from baseline (pretreatment “Day 1”) of at least 2 levels of severity in the CR-PCSS as assessed live by the clinician of the treatment area.
3. An improvement in severity at Day 22, 43, 71, 90, 180, 251, 360, 431, 720, 3 years, 4 years, or 5 years from baseline (Day 1) of at least 2 levels of severity in the PR-PCSS as assessed by the subject while viewing the digital image of the treatment area.
4. An improvement demonstrated by a 2-level composite response at Day 22, 43, 71, 90, 180, 251, 360, 431, 720, 3 years, 4 years, or 5 years defined as a subject with an improvement from baseline of at least 2 levels of severity in the CR-PCSS and an improvement from baseline of at least 2 levels of severity in the PR-PCSS.
5. An improvement in severity at Day 22, 43, 71, 90, 180, 251, 360, 431, 720, 3 years, 4 years, or 5 years from baseline of at least 1 level of severity in the CR-PCSS as assessed live by the clinician of the treatment area.
6. An improvement in severity at Day 22, 43, 71, 90, 180, 251, 360, 431, 720, 3 years, 4 years, or 5 years from baseline of at least 1 level of severity in the PR-PCSS as assessed by the subject while viewing the digital image of the treatment area.
7. An improvement demonstrated by a 1-level composite response at Day 22, 43, 71, 90, 180, 251, 360, 431, 720, 3 years, 4 years, or 5 years defined as a subject with an improvement from baseline of at least 1 level of severity in the CR-PCSS and an improvement from baseline of at least 1 level of severity in the PR-PCSS.
8. In a population of patients who all had CR-PCSS and/or PR-PCSS ratings of moderate or severe, the improvement in at least one treatment area was statistically significant compared to placebo wherein the improvement is one or more of Nos. 1 to 7 above.
9. The treatment resulted in at least 5% of patients maintaining their level of improvement versus pretreatment baseline for at least 71 days after the initial dose. In certain cases, at least 10%, or 20%, or 30% or, 40% or 50% of patients maintained such level for at least 6 months, or 9 months, or 12 months, or 18 months, or 2 years or 3 years, or 4 years, or 5 years after the initial dose. In other cases, the treatments resulted in at least 5% of patients demonstrating improvement versus pretreatment baseline and showing an additional increase in improvement over time. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial dose. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial treatment, or second treatment, or third treatment.
10. At Day 180, the improvement seen in the CR-PCSS rating from baseline was consistent on the right and left treatment areas.
11. In a population of patients who all have CR-PCSS and/or PR-PCSS ratings of moderate or severe, the median time to the earliest 2-level CR-PCSS and/or PR-PCSS improvement in at least one treatment area is about 50 days, or 60 days, or 70 days, or 80 days, or 90 days.
12. In a population of patients who all have CR-PCSS and/or PR-PCSS ratings of moderate or severe, the median time to the earliest 1-level CR-PCSS and/or PR-PCSS improvement in at least one treatment area is about 15 days, or 20 days, or 30 days, or 40 days, or 50 days, or 60 days, or 70 days, or 80 days, or 90 days.
13. In a population of patients who all have CR-PCSS and/or PR-PCSS ratings of moderate or severe, the mean subject CR-PCSS and/or PR-PCSS scores separate from placebo 21 days after the first treatment and demonstrate continuous and significant improvement after subsequent treatments.
14. In a population of patients who all have CR-PCSS and/or PR-PCSS ratings of moderate or severe, the percentage of the subjects who have a 2-level composite response as measured by CR-PCSS and/or PR-PCSS in at least one treatment area at Day 71 is from about 1% to 10%, 10% to 20%, 20% to 30%, 30% to 40%, 40% to 50%, or greater than 50%.
15. In a population of patients who all have CR-PCSS and/or PR-PCSS ratings of moderate or severe, the percentage of the subjects who have a 1-level composite response as measured by CR-PCSS and/or PR-PCSS in at least one treatment area at Day 71 is from about 1% to 10%, 10% to 20%, 20% to 30%, 30% to 40%, 40% to 50%, or greater than 50%.
16. In a population of patients who all have CR-PCSS and/or PR-PCSS ratings of moderate or severe, over one-third, or one-half, or two-thirds, or three-fourths of the patients have at least a 1-level CR-PCSS and/or at least a 1-level PR-PCSS responses in at least one treatment area by Day 71 post-treatment wherein the CR-PCSS results are independent of age, BMI or skin color.
17. The reduction in the severity of cellulite occurs rapidly within about 7, or 14, or 21, or 30, or 35, or 40, or 45, or 50 days after the first treatment visit.
18. In a population of patients who all have CR-PCSS and/or PR-PCSS ratings of moderate or severe, the maximum decrease in the CR-PCSS and/or PR-PCSS rating from the baseline visit (screening visit) is first observed at Day 90.
19. In a population of patients who all have CR-PCSS and/or PR-PCSS ratings of moderate or severe, the improvement in cellulite severity (i.e., a negative change) at a given time point before Day 180 is maintained at the Day 180 visit.
20. In a population of patients who all have CR-PCSS and/or PR-PCSS ratings of moderate or severe, at Day 90, the mean (SD) change in the CR-PCSS and/or PR-PCSS rating from baseline of the left buttock and left thigh is about −0.8 (0.58) and about −0.6 (0.62), respectively, and in the right buttock and right thigh is about −0.7 (0.73) and about −0.5 (0.70), respectively.
21. In a population of patients who all have CR-PCSS and/or PR-PCSS ratings of moderate or severe, at Day 180, the decrease in the CR-PCSS and/or PR-PCSS rating from baseline (screening visit) is consistent on the right and left sides.
22. In a population of patients who all have CR-PCSS and/or PR-PCSS ratings of moderate or severe, a 2-level improvement in the CR-PCSS and/or PR-PCSS rating in at least 1 area is observed in at least 10% of subjects at Day 90 and at least 15% of subjects at Day 180.
22. In a population of patients who all have CR-PCSS and/or PR-PCSS ratings of moderate or severe, a 2-level improvement in the CR-PCSS and/or PR-PCSS rating in at least 1 area is observed in at least 10% of subjects at Day 90 and at least 15% of subjects at Day 180, and the response is similar for the buttock and thigh regions and for left and right sides.
23. In a population of patients who all have CR-PCSS and/or PR-PCSS ratings of moderate or severe, a 1-level improvement in the CR-PCSS and/or PR-PCSS rating in at least 1 treatment area is observed in at least 60% of subjects at Day 90 and at least 65% of subjects at Day 180.
24. In a population of patients who all have CR-PCSS and/or PR-PCSS ratings of moderate or severe, a 1-level improvement in the CR-PCSS and/or PR-PCSS rating in at least 1 treatment area is observed in at least 60% of subjects at Day 90 and at least 65% of subjects at Day 180, and the response is similar for the buttock and thigh regions and for left and right sides.
25. In a population of patients who all have CR-PCSS and/or PR-PCSS ratings of moderate or severe, the median time to the earliest 2-level CR-PCSS and/or PR-PCSS response in at least 1 treatment area is observed at about 80 days.
26. In a population of patients who all have CR-PCSS and/or PR-PCSS ratings of moderate or severe, the median time to the earliest 1 level CR-PCSS and/or PR-PCSS response in at least 1 treatment area is about 40 days.
27. In a population of patients who all have CR-PCSS and/or PR-PCSS ratings of moderate or severe, at least 50% of the patients are 1-level PR-PCSS and/or PR-PCSS responders in the target buttock at Day 71.
28. In a population of patients who all have CR-PCSS and/or PR-PCSS ratings of moderate or severe, at least 20% of the patients are 2-level PR-PCSS and/or PR-PCSS responders in the target buttock at Day 71.
29. In a population of patients who all have CR-PCSS and/or PR-PCSS ratings of moderate or severe, at least 35% of patients are 1-level CR-PCSS and/or PR-PCSS composite responders in the target buttock at Day 71.
30. In a population of patients who all have CR-PCSS and/or PR-PCSS ratings of moderate or severe, at least 5% of patients are 2-level CR-PCSS and/or PR-PCSS composite responders in the non-target buttock at Day 71.
31. In a population of patients who all have CR-PCSS and/or PR-PCSS ratings of moderate or severe, the mean change from baseline in PR-PCSS and/or PR-PCSS was greater in subjects treated with collagenase than in those treated with placebo in the target buttock (about 0.9 vs. about 0.5, respectively) and the non-target buttock (about 0.9 vs about 0.5, respectively) at Day 71.
32. In a population of patients who all have CR-PCSS and/or PR-PCSS ratings of moderate or severe, the proportion of 1-level PR-PCSS and/or PR-PCSS responders was greater in subjects treated with collagenase than in placebo treated subjects in the target buttock (about 62% vs about 40%, respectively) and in the non-target buttock (about 65% vs 40%, respectively) at Day 71.
33. In a population of patients who all have CR-PCSS and/or PR-PCSS ratings of moderate or severe, the mean change from baseline in CR-PCSS and/or PR-PCSS was greater in subjects treated with CCH than in those treated with placebo in the target buttock (about 0.7 vs. about 0.4 [0.72], respectively) and the non-target buttock (about 0.8 vs. about 0.3, respectively) at Day 71.
34. In a population of patients who all have CR-PCSS and/or PR-PCSS ratings of moderate or severe, the proportion of 1-level CR-PCSS and/or PR-PCSS responders was greater in subjects treated with collagenase than in placebo treated subjects in the target buttock (about 58% vs. about 32%, respectively), and in the non-target buttock (about 60% vs. about 27%, respectively) at Day 71.
35. In a population of patients who all have CR-PCSS and/or PR-PCSS ratings of moderate or severe, the proportion of 1-level CR-PCSS and/or PR-PCSS composite responders was greater in subjects treated with collagenase than in placebo treated subjects in the target buttock (about 42% vs. about 20%, respectively) and in the non-target buttock (about 44% vs. about 13%, respectively) at Day 71.
36. In a population of patients who all have CR-PCSS and/or PR-PCSS ratings of moderate or severe, the collagenase treatments as described herein result in greater than or equal to 2-level composite responders in a population of patients of at least 5% higher than placebo, or at least 7.5% higher than placebo, or at least 10% higher than placebo, or at least 12.5% higher than placebo, or at least 15% higher than placebo, or at least 20% higher than placebo.
37. In a population of patients who all have CR-PCSS and/or PR-PCSS ratings of moderate or severe, the collagenase treatments as described herein result in greater than or equal to 1-level composite responders in a population of patients of at least 5% higher than placebo, or at least 7.5% higher than placebo, or at least 10% higher than placebo, or at least 12.5% higher than placebo, or at least 15% higher than placebo, or at least 20% higher than placebo, or at least 25% higher than placebo, or at least 30% higher than placebo, or at least 35% higher than placebo, or at least 40% higher than placebo.
38. In a population of patients who all have CR-PCSS and/or PR-PCSS ratings of moderate or severe, the maximum decrease in the CR-PCSS and/or PR-PCSS rating from the baseline visit (screening visit) is first observed at Day 22, Day 71, or earlier.
39. In a population of patients who all have CR-PCSS and/or PR-PCSS ratings of moderate or severe, at base line (pretreatment), the decrease in the CR-PCSS and/or PR-PCSS rating from baseline (screening visit) is consistent on the right and left sides.

In another embodiment, the injection of about 1 mg to about 20 mg of collagenase to at least one treatment area during at least one treatment visit results in one or more of the results Nos. 1 to 39 above, wherein the collagenase has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In other cases, the injection of about 1 mg to about 20 mg of collagenase according to Treatment I results in one or more of the results Nos. 1 to 39 above.

In another instance, the injection of about 1 mg to about 20 mg of collagenase according to Treatment I results in one or more of the results Nos. 1 to 39 above, wherein the collagenase has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another example, the injection of about 1 mg to about 20 mg of CCH according to Treatment I results in one or more of the results Nos. 1 to 39 above.

In certain embodiments, about 1 mg to about 20 mg of an approximate 1:1 ratio of collagenase Type I and collagenase Type II is injected to at least one treatment area during at least one treatment visit and results in one or more of the results Nos. 1 to 39 above, wherein the collagenases I and II have the following characteristics:
Type I
   Assay: SRC microplate
   Vmax, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934
      Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In certain embodiments, about 1 mg to about 20 mg of an approximate 1:1 ratio of collagenase Type I and collagenase Type II is injected employing Treatment I and results in one or more of the results Nos. 1 to 39 above, wherein the collagenases I and II have the following characteristics:
Type I
   Assay: SRC microplate
   Vmax, min⁻¹: About 0.08 to 7.70
   K_{M}: About 4.1 to 410 nanoMolar
   K_{cat}, sec-1: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934

Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In other embodiments, in response to the above treatments, a patient is a 2 level CR-PCSS responder who shows improvement in CR-PCSS rating of at least 2 levels from baseline (change of −2, −3, or −4) at an evaluation time point. A 1 level CR-PCSS responder is a patient exhibiting improvement in CR-PCSS rating of at least 1 level from baseline (change of −1, −2, −3, or −4) at an evaluation time point. A patient is a 2 level PR-PCSS responder who shows improvement in PR-PCSS rating of at least 2 levels from baseline (change of −2, −3, or −4) at an evaluation time point. A 1 level PR-PCSS responder is a patient exhibiting improvement in PR-PCSS rating of at least 1 level from baseline (change of −1, −2, −3, or −4) at an evaluation time point. In other aspects, a 2-level composite responder is a patient who is both a 2-level PR-PCSS responder and a 2-level CR-PCSS responder at an evaluation time point. A 1-level composite responder is a patient who is both a 1-level PR-PCSS responder and a 1-level CR-PCSS responder at an evaluation time point.

### 2. Efficacy as Measured by Hexsel Cellulite Severity Scale (Hexsel CSS)

In Hexsel CSS, an improvement for the individual patient at any visit is an improvement of at least 1 level or 1 rating from baseline or any previous score. An improvement for a group of patients at any visit is an improvement of about 0.1 in the mean Hexsel CSS score or rating from the baseline or any previous mean Hexsel CSS score or rating. A responder is any patient showing at least a 25% improvement of maximum total score or rating from baseline. In certain embodiments, the treatment methods detailed above result in one or more of the following efficacy endpoints as measured by Hexsel CSS:

1. In a population of patients who all had baseline Day 1 Hexsel CSS ratings, the injection of collagenase to at least one treatment area during at least one treatment visit resulted in a statistically significant number of such patients meeting one or more of the following efficacy endpoints:
Shift from severe to moderate (from score of 11-15 to 6-10)
Shift from severe to mild (from score of 11-15 to 1-5)
Shift from severe to zero (from score of 11-15 to 0)
Shift from moderate to mild (from score of 6-10 to 1-5)
Shift from moderate to zero (from score of 6-10 to 0)
Shift from mild to zero (from score of 1-5 to 0)
   2. A change in severity at Day 22, 43, 71, 90, 180, 251, 360, 431, 720, 3 years, 4 years, or 5 years from baseline (pretreatment “Day 1”) of at least 2 levels of severity in the Hexsel CSS as assessed live by the clinician of the treatment area.
   3. A change in severity at Day 22, 43, 71, 90, 180, 251, 360, 431, 720, 3 years, 4 years, or 5 years from baseline (Day 1) of at least 2 levels of severity in the Hexsel CSS as assessed by the clinician while viewing the digital image of the treatment area.
   4. A change demonstrated by a 2-level response at Day 22, 43, 71, 90, 180, 251, 360, 431, 720, 3 years, 4 years, or 5 years defined as a subject with an improvement from baseline of at least 2 levels of severity in the Hexsel CSS by clinician assessment.
   5. A change in severity at Day 22, 43, 71, 90, 180, 251, 360, 431, 720, 3 years, 4 years, or 5 years from baseline (Day 1) of at least 1 level of severity in the Hexsel CSS as assessed live by the clinician of the treatment area.
   6. A change in severity at Day 22, 43, 71, 90, 180, 251, 360, 431, 720, 3 years, 4 years, or 5 years from baseline (Day 1) of at least 1 level of severity in the Hexsel CSS as assessed by the clinician while viewing the digital image of the treatment area.
   7. A change demonstrated by a 1-level response at Day 22, 43, 71, 90, 180, 251, 360, 431, 720, 3 years, 4 years, or 5 years defined as a subject with an improvement from baseline of at least 1 level of severity in the Hexsel CSS by clinician assessment.
   8. In a population of patients who all had Hexsel CSS ratings of moderate or severe, the improvement in at least one treatment area was statistically significant compared to placebo wherein the change is one or more of Nos. 2 to 7 above.
   9. The treatment resulted in at least 5% of patients maintaining their level of improvement versus pretreatment baseline for at least 71 days after the initial dose. In certain cases, at least
   10%, or 20%, or 30% or, 40% or 50% of patients maintained such level for at least 6 months, or 9 months, or 12 months, or 18 months, or 2 years or 3 years, or 4 years, or 5 years after the initial dose. In other cases, the treatments resulted in at least 5% of patients demonstrating improvement versus pretreatment baseline and showing an additional increase in improvement over time. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial dose. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial treatment, or second treatment, or third treatment.
   10. At day 180, the improvement seen in the Hexsel CSS rating from baseline was consistent on the right and left treatment areas.
   11. In a population of patients who all have Hexsel CSS ratings of moderate or severe, the median time to the earliest 2-level Hexsel CSS improvement in at least one treatment area is about 50 days, or 60 days, or 70 days, or 80 days, or 90 days.
   12. In a population of patients who all have Hexsel CSS ratings of moderate or severe, the median time to the earliest 1-level Hexsel CSS improvement in at least one treatment area is about 15 days, or 20 days, or 30 days, or 40 days, or 50 days, or 60 days, or 70 days, or 80 days, or 90 days.
   13. In a population of patients who all have Hexsel CSS ratings of moderate or severe, the mean subject Hexsel CSS scores separates from placebo 21 days after the first treatment and demonstrate continuous and significant improvement after subsequent treatments.
   14. In a population of patients who all have Hexsel CSS ratings of moderate or severe, the percentage of the subjects who have a 2-level response as measured by Hexsel CSS in at least one treatment area at Day 71 is from about 1% to 10%, 10% to 20%, 20% to 30%, 30% to 40%, 40% to 50%, or greater than 50%.
   15. In a population of patients who all have Hexsel CSS ratings of moderate or severe, the percentage of the subjects who have a 1-level response as measured by Hexsel CSS in at least one treatment area at Day 71 is from about 1% to 10%, 10% to 20%, 20% to 30%, 30% to 40%, 40% to 50%, or greater than 50%.
   16. In a population of patients who all have Hexsel CSS ratings of moderate or severe, over one-third, or one-half, or two-thirds, or three-fourths of the patients have at least a 1-level Hexsel CSS response in at least one treatment area by Day 71 post-treatment wherein the Hexsel CSS results are independent of age, BMI or skin color.
   17. The reduction in the severity of cellulite occurs rapidly within about 7, or 14, or 21, or 30, or 35, or 40, or 45, or 50 days after the first treatment visit.

In another embodiment, the injection of about 1 mg to about 20 mg of collagenase to at least one treatment area during at least one treatment visit results in one or more of the results Nos. 1 to 17 above, wherein the collagenase has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In other cases, the injection of about 1 mg to about 20 mg of collagenase according to Treatment I results in one or more of the results Nos. 1 to 17 above.

In another instance, the injection of about 1 mg to about 20 mg of collagenase according to Treatment I results in one or more of the results Nos. 1 to 17 above, wherein the collagenase has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another example, the injection of about 1 mg to about 20 mg of CCH according to Treatment I results in one or more of the results Nos. 1 to 17 above.

In certain embodiments, about 1 mg to about 20 mg of an approximate 1:1 ratio of collagenase Type I and collagenase Type II is injected to at least one treatment area during at least one treatment visit and results in one or more of the results Nos. 1 to 17 above, wherein the collagenases I and II have the following characteristics:
Type I
   Assay: SRC microplate
   Vmax, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934
      Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In certain embodiments, about 1 mg to about 20 mg of an approximate 1:1 ratio of collagenase Type I and collagenase Type II is injected employing Treatment I and results in one or more of the results Nos. 1 to 17 above, wherein the collagenases I and II have the following characteristics:
Type I
   Assay: SRC microplate
   Vmax, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934
      Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

### 3. Efficacy as Measured by Hexsel Depression Depth Score

In Hexsel Depression Depth Score, an improvement for the individual patient at any visit is an improvement of at least 1 level or 1 rating from baseline or any previous score. An improvement for a group of patients at any visit is an improvement of about 0.1 in the mean Hexsel Depression Depth score or rating from the baseline or any previous mean Hexsel Depression Depth score or rating. A responder is any patient showing at least a 25% improvement of maximum total score or rating from baseline. In certain embodiments, the treatment methods detailed above result in one or more of the following efficacy endpoints as measured by Hexsel Depression Depth Score:

1. In a population of patients who all had baseline Day 1 Hexsel Depression Depth Score ratings, the injection of collagenase to at least one treatment area during at least one treatment visit resulted in a statistically significant number of such patients meeting one or more of the following efficacy endpoints:
Shift from deep depressions (3) to medium depth (2)
Shift from deep depressions (3) to superficial depressions (1)
Shift from deep depressions (3) to no depressions (0)
Shift from medium depth (2) to superficial depressions (1)
Shift from medium depth (2) to no depressions (0)
Shift from superficial depressions (1) to no depressions (0)
   2. A change in severity at Day 22, 43, 71, 90, 180, 251, 360, 431, 720, 3 years, 4 years, or 5 years from baseline (pretreatment “Day 1”) of at least 2 levels of severity in the Hexsel Depression Depth Score as assessed live by the clinician of the treatment area.
   3. A change demonstrated by a 2-level response at Day 22, 43, 71, 90, 180, 251, 360, 431, 720, 3 years, 4 years, or 5 years defined as a subject with an improvement from baseline of at least 2 levels of severity in the Hexsel Depression Depth Score by clinician assessment.
   4. A change in severity at Day 22, 43, 71, 90, 180, 251, 360, 431, 720, 3 years, 4 years, or 5 years from baseline (Day 1) of at least 1 level of severity in the Hexsel Depression Depth Score as assessed live by the clinician of the treatment area.
   5. A change demonstrated by a 1-level response at Day 22, 43, 71, 90, 180, 251, 360, 431, 720, 3 years, 4 years, or 5 years defined as a subject with an improvement from baseline of at least 1 level of severity in the Hexsel Depression Depth Score by clinician assessment.
   6. In a population of patients who all had Hexsel CSS ratings of medium or deep depressions, the improvement in at least one treatment area was statistically significant compared to placebo wherein the change is one or more of Nos. 2 to 7 above.
   7. The treatment resulted in at least 5% of patients maintaining their level of improvement versus pretreatment baseline for at least 71 days after the initial dose. In certain cases, at least 10%, or 20%, or 30% or, 40% or 50% of patients maintained such level for at least 6 months, or 9 months, or 12 months, or 18 months, or 2 years or 3 years, or 4 years, or 5 years after the initial dose. In other cases, the treatments resulted in at least 5% of patients demonstrating improvement versus pretreatment baseline and showing an additional increase in improvement over time. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial dose. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial treatment, or second treatment, or third treatment.
   8. At Day 180, the improvement seen in the Hexsel Depression Depth Score rating from baseline was consistent on the right and left treatment areas.
   9. In a population of patients who all have Hexsel Depression Depth Score ratings of medium or deep depressions, the median time to the earliest 2-level Hexsel Depression Depth Score improvement in at least one treatment area is about 50 days, or 60 days, or 70 days, or 80 days, or 90 days.
   10. In a population of patients who all have Hexsel Depression Depth Score ratings of medium or deep depressions, the median time to the earliest 1-level Hexsel Depression Depth Score improvement in at least one treatment area is about 15 days, or 20 days, or 30 days, or 40 days, or 50 days, or 60 days, or 70 days, or 80 days, or 90 days.
   11. In a population of patients who all have Hexsel Depression Depth Score ratings of medium or deep depressions, the mean subject Hexsel Depression Depth Scores separates from placebo 21 days after the first treatment and demonstrate continuous and significant improvement after subsequent treatments.
   12. In a population of patients who all have Hexsel Depression Depth Score ratings of medium or deep depressions, the percentage of the subjects who have a 2-level response as measured by Hexsel Depression Depth Score in at least one treatment area at Day 71 is from about 1% to 10%, 10% to 20%, 20% to 30%, 30% to 40%, 40% to 50%, or greater than 50%.
   13. In a population of patients who all have Hexsel Depression Depth Score ratings of medium or deep depressions, the percentage of the subjects who have a 1-level response as measured by Hexsel Depression Depth Score in at least one treatment area at Day 71 is from about 1% to 10%, 10% to 20%, 20% to 30%, 30% to 40%, 40% to 50%, or greater than 50%.
   14. In a population of patients who all have Hexsel Depression Depth Score ratings of medium or deep depressions, over one-third, or one-half, or two-thirds, or three-fourths of the patients have at least a 1-level Hexsel Depression Depth Score response in at least one treatment area by day 71 post-treatment wherein the Hexsel Depression Depth Score results are independent of age, BMI or skin color.
   15. The reduction in the severity of cellulite occurs rapidly within about 7, or 14, or 21, or 30, or 35, or 40, or 45, or 50 days after the first treatment visit.
   16. A change in score from baseline to Day 71 of about −0.1 to about −2.0 one or more treatment areas.
   17. In a statistically significant population of patients, the least squares (LS) mean is from about −0.1 to about −1.5 (95% confidence interval (CI)) for one or more treatment areas.

In another embodiment, the injection of about 1 mg to about 20 mg of collagenase to at least one treatment area during at least one treatment visit results in one or more of the results Nos. 1 to 19 above, wherein the collagenase has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In other cases, the injection of about 1 mg to about 20 mg of collagenase according to Treatment I results in one or more of the results Nos. 1 to 19 above.

In another instance, the injection of about 1 mg to about 20 mg of collagenase according to Treatment I results in one or more of the results Nos. 1 to 19 above, wherein the collagenase has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another example, the injection of about 1 mg to about 20 mg of CCH according to Treatment I results in one or more of the results Nos. 1 to 19 above.

In certain embodiments, about 1 mg to about 20 mg of an approximate 1:1 ratio of collagenase Type I and collagenase Type II is injected to at least one treatment area during at least one treatment visit and results in one or more of the results Nos. 1 to 19 above, wherein the collagenases I and II have the following characteristics:
Type I
   Assay: SRC microplate
   Vmax, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934
      Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In certain embodiments, about 1 mg to about 20 mg of an approximate 1:1 ratio of collagenase Type I and collagenase Type II is injected employing Treatment I and results in one or more of the results Nos. 1 to 19 above, wherein the collagenases I and II have the following characteristics:
Type I
   Assay: SRC microplate
   Vmax, min⁻¹: About 0.08 to 7.70
   K_{M}: About 4.1 to 410 nanoMolar
   K_{cat}, sec-1: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934

Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

### 4. Efficacy as Measured by Likert Scale for Aesthetic Appearance

In the Likert Scale, an improvement for the individual patient at any visit is an improvement of at least 1 level or 1 rating from before treatment or any previous score. An improvement for a group of patients at any visit is an improvement of about 0.1 in the mean Likert score or rating from before treatment or any previous mean Likert score or rating. In certain embodiments, the treatment methods detailed above result in one or more of the following efficacy endpoints as measured by Likert Scale Score:

1. In a population of patients with cellulite, the injection of collagenase to at least one treatment area during at least one treatment visit resulted in a statistically significant number of such patients meeting one or more of the following efficacy endpoints:
A Likert Scale Score of “Improved” (1)
A Likert Scale Score of “Much Improved” (2)
A Likert Scale Score of “Very Much Improved” (3)
   2. An improvement in the treatment area appearance at Day 22, 43, 71, 90, 180, 251, 360, 431, 720, 3 years, 4 years, or 5 years from before treatment of at least 2 levels in the Likert Scale Score as assessed live by the clinician of the treatment area.
   3. An improvement in the treatment area appearance at Day 22, 43, 71, 90, 180, 251, 360, 431, 720, 3 years, 4 years, or 5 years from before treatment of at least 2 levels in the Likert Scale Score as assessed by the subject while viewing the digital image of the treatment area.
   4. An improvement in the treatment area appearance at Day 22, 43, 71, 90, 180, 251, 360, 431, 720, 3 years, 4 years, or 5 years from before treatment of at least 1 level in the Likert Scale Score as assessed live by the clinician of the treatment area.
   5. An improvement in the treatment area appearance at Day 22, 43, 71, 90, 180, 251, 360, 431, 720, 3 years, 4 years, or 5 years from before treatment of at least 1 level in the Likert Scale Score as assessed by the subject while viewing the digital image of the treatment area.
   6. In a population of patients with cellulite, the improvement in Likert Scale Scores in at least one treatment area was statistically significant wherein the improvement is one or more of Nos. 2 to 7 above.
   7. The treatment resulted in at least 5% of patients maintaining their level of improvement versus pretreatment baseline for at least 71 days after the initial dose. In certain cases, at least 10%, or 20%, or 30% or, 40% or 50% of patients maintained such level for at least 6 months, or 9 months, or 12 months, or 18 months, or 2 years or 3 years, or 4 years, or 5 years after the initial dose. In other cases, the treatments resulted in at least 5% of patients demonstrating improvement versus pretreatment baseline and showing an additional increase in improvement over time. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial dose. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial treatment, or second treatment, or third treatment.
   8. At Day 180, the improvement seen in the Likert Scale Score rating from baseline was consistent on the right and left treatment areas.
   9. In a population of patients who have cellulite, the median time to the earliest 2-level Likert Scale Score improvement in at least one treatment area is about 50 days, or 60 days, or 70 days, or 80 days, or 90 days.
   10. In a population of patients who have cellulite, the median time to the earliest 1-level Likert Scale Score improvement in at least one treatment area is about 15 days, or 20 days, or 30 days, or 40 days, or 50 days, or 60 days, or 70 days, or 80 days, or 90 days.
   11. In a population of patients who have cellulite, the mean subject Likert Scale Scores separates from placebo 21 days after the first treatment and demonstrate continuous and significant improvement after subsequent treatments.
   12. In a population of patients who have cellulite, the percentage of the subjects who have a 2-level response as measured by Likert Scale Score in at least one treatment area at Day 71 is from about 1% to 10%, 10% to 20%, 20% to 30%, 30% to 40%, 40% to 50%, or greater than 50%.
   13. In a population of patients who have cellulite, the percentage of the subjects who have a 1-level response as measured by Likert Scale Score in at least one treatment area at Day 71 is from about 1% to 10%, 10% to 20%, 20% to 30%, 30% to 40%, 40% to 50%, or greater than 50%.
   14. In a population of patients who have cellulite, over one-third, or one-half, or two-thirds, or three-fourths of the patients have at least a 1-level Likert Scale Score response in at least one treatment area by Day 71 post-treatment wherein the Likert Scale Score results are independent of age, BMI or skin color.
   15. The reduction in the severity of cellulite occurs rapidly within about 7, or 14, or 21, or 30, or 35, or 40, or 45, or 50 days after the first treatment visit.

In another embodiment, the injection of about 1 mg to about 20 mg of collagenase to at least one treatment area during at least one treatment visit results in one or more of the results Nos. 1 to 15 above, wherein the collagenase has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In other cases, the injection of about 1 mg to about 20 mg of collagenase according to Treatment I results in one or more of the results Nos. 1 to 15 above.

In another instance, the injection of about 1 mg to about 20 mg of collagenase according to Treatment I results in one or more of the results Nos. 1 to 15 above, wherein the collagenase has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another example, the injection of about 1 mg to about 20 mg of CCH according to Treatment I results in one or more of the results Nos. 1 to 15 above.

In certain embodiments, about 1 mg to about 20 mg of an approximate 1:1 ratio of collagenase Type I and collagenase Type II is injected to at least one treatment area during at least one treatment visit and results in one or more of the results Nos. 1 to 15 above, wherein the collagenases I and II have the following characteristics:
Type I
   Assay: SRC microplate
   Vmax, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934
      Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In certain embodiments, about 1 mg to about 20 mg of an approximate 1:1 ratio of collagenase Type I and collagenase Type II is injected employing Treatment I and results in one or more of the results Nos. 1 to 15 above, wherein the collagenases I and II have the following characteristics:
Type I
   Assay: SRC microplate
   Vmax, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934
      Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

### 5. Dimple Analysis

In certain embodiments, the treatment of cellulite with collagenase(s) decreases dimple size parameters as follows:
Depth: By about 100%, 90%, 80%, 70%, 60%, 50%, 40%, 30%, 25%, or 20%, or 15%, or 10%, or 5%, or 2.5%, or 2%, or 1%
Width: By about 100%, 90%, 80%, 70%, 60%, 50%, 40%, 30%, 25%, or 20%, or 15%, or 10%, or 5%, or 2.5%, or 2%, or 1%
Length: By about 100%, 90%, 80%, 70%, 60%, 50%, 40%, 30%, 25%, or 20%, or 15%, or 10%, or 5%, or 2.5%, or 2%, or 1%
Overall Volume: By about 100%, 90%, 80%, 70%, 60%, 50%, 40%, 30%, 25%, or 20%, or 15%, or 10%, or 5%, or 2.5%, or 2%, or 1%
Surface Area: By about 100%, 90%, 80%, 70%, 60%, 50%, 40%, 30%, 25%, or 20%, or 15%, or 10%, or 5%, or 2.5%, or 2%, or 1%

In some embodiments, the treatments resulted in at least 5% of patients maintaining their level of improvement versus pretreatment baseline for at least 71 days after the initial dose. In certain cases, at least 10%, or 20%, or 30% or, 40% or 50% of patients maintained such level for at least 6 months, or 9 months, or 12 months, or 18 months, or 2 years or 3 years, or 4 years, or 5 years after the initial dose. In other embodiments, the treatments resulted in at least 5% of patients demonstrating improvement versus pretreatment baseline and showing an additional increase in improvement over time. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial dose. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial treatment, or second treatment, or third treatment.

In another embodiment, the injection of about 1 mg to about 20 mg of collagenase to at least one treatment area during at least one treatment visit results in a reduction in at least one of the dimple size parameters by at least 5%, or at least 10% or at least 20%, wherein the collagenase has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In other cases, the injection of about 1 mg to about 20 mg of collagenase according to Treatment I results in a reduction in at least one of the dimple size parameters by at least 5%, or at least 10% or at least 20%.

In another instance, the injection of about 1 mg to about 20 mg of collagenase according to Treatment I results in a reduction in at least one of the dimple size parameters by at least 5%, or at least 10% or at least 20%, wherein the collagenase has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another example, the injection of about 1 mg to about 20 mg of CCH according to Treatment I results in a reduction in at least one of the dimple size parameters by at least 5%, or at least 10% or at least 20%.

In certain embodiments, about 1 mg to about 20 mg of an approximate 1:1 ratio of collagenase Type I and collagenase Type II is injected to at least one treatment area during at least one treatment visit and results in a reduction in at least one of the dimple size parameters by at least 5%, or at least 10% or at least 20%, wherein the collagenases I and II have the following characteristics:
Type I
   Assay: SRC microplate
   Vmax, min⁻¹: About 0.08 to 7.70
   K_{M}: About 4.1 to 410 nanoMolar
   K_{cat}, sec-1: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934
      Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In certain embodiments, about 1 mg to about 20 mg of an approximate 1:1 ratio of collagenase Type I and collagenase Type II is injected employing Treatment I and results in a reduction in at least one of the dimple size parameters by at least 5%, or at least 10% or at least 20%, wherein the collagenases I and II have the following characteristics:
Type I
   Assay: SRC microplate
   Vmax, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934
      Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

### 6. Efficacy as Measured by Subject Global Aesthetic Improvement Scale (S-GAIS) and Investigator Global Aesthetic Improvement Scale (I-GAIS)

The treatment methods detailed above result in improved responses as measured by S-GAIS and I-GAIS. A 2-level S-GAIS responder is a subject with an S-GAIS rating of at least 2 (+2 or +3) at an evaluation time point. A 1-level S-GAIS responder is a subject with an S-GAIS rating of at least 1 (+1, +2, or +3) at an evaluation time point. A 2-level I-GAIS responder is a subject with an I-GAIS rating of at least 2 (+2 or +3) at an evaluation time point. A 1-level I-GAIS responder is a subject with an I-GAIS rating of at least 1 (+1, +2, or +3) at an evaluation time point. An improvement for the individual patient at any visit is an improvement of at least 1 level or 1 rating from baseline or any previous score. An improvement for a group of patients at any visit is an improvement of about 0.1 in the mean score or rating from the baseline or any previous mean score or rating.

In certain embodiments, the treatment methods detailed above result in one or more of the following efficacy endpoints as measured by S-GAIS and/or I-GAIS:

1. In a population of patients with cellulite, the injection of collagenase to at least one treatment area during at least one treatment visit results in a statistically significant number of such patients meeting one or more of the following efficacy endpoints:
S-GAIS and/or I-GAIS score of “Improved” (+1)
S-GAIS and/or I-GAIS score of “Much Improved” (+2)
S-GAIS and/or I-GAIS score of “Very Much Improved” (+3)
   2. An improvement at Day 22, 43, 71, 90, 180, 365, or 730 of at least 2 levels in the I-GAIS as assessed live by the clinician of the treatment area.
   3. An improvement at Day 22, 43, 71, 90, 180, 365, or 730 of at least 2 levels in the S-GAIS as assessed by the subject while viewing the digital image of the treatment area.
   4. An improvement demonstrated by a 2-level composite response at Day 22, 43, 71, 90, 180, 365, or 730 defined as a subject with an improvement of at least 2 levels in the I-GAIS by clinician assessment and an improvement of at least 2 levels in the S-GAIS by patient assessment.
   5. An improvement at Day 22, 43, 71, 90, 180, 365, or 730 of at least 1 level in the I-GAIS as assessed live by the clinician of the treatment area.
   6. An improvement at Day 22, 43, 71, 90, 180, 365, or 730 of at least 1 level in the S-GAIS as assessed by the subject while viewing the digital image of the treatment area.
   7. An improvement demonstrated by a 1-level composite response at Day 22, 43, 71, 90, 180, 365, or 730 defined as a subject with an improvement of at least 1 level in the I-GAIS by clinician assessment and an improvement of at least 1 level in the S-GAIS by patient assessment.
   8. In a population of patients with cellulite, the improvement in I-GAIS and/or S-GAIS in at least one treatment area was statistically significant compared to placebo wherein the improvement is one or more of Nos. 2 to 7 above.
   9. The treatment resulted in at least 5% of patients maintaining their level of improvement for at least 71 days after the initial dose. In certain cases, at least 10%, or 20%, or 30% or, 40% or 50% of patients maintained such level for at least 6 months, or 9 months, or 12 months, or 18 months, or 2 years or 3 years, or 4 years, or 5 years after the initial dose. In other cases, the treatments resulted in at least 5% of patients demonstrating improvement and showing an additional increase in improvement over time. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial dose. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial treatment, or second treatment, or third treatment.
   10. At Day 180, the improvement seen in the I-GAIS and S-GAIS rating was consistent on the right and left treatment areas.
   11. In a population of patients who have cellulite, the median time to the earliest 2-level I-GAIS and/or S-GAIS improvement in at least one treatment area is about 50 days, or 60 days, or 70 days, or 80 days, or 90 days.
   12. In a population of patients who have cellulite, the median time to the earliest 1-level I-GAIS and/or S-GAIS improvement in at least one treatment area is about 15 days, or 20 days, or 30 days, or 40 days, or 50 days, or 60 days, or 70 days, or 80 days, or 90 days.
   13. In a population of patients who have cellulite, the mean subject I-GAIS and/or S-GAIS separates from placebo 21 days after the first treatment and demonstrate continuous and significant improvement after subsequent treatments.
   14. In a population of patients who have cellulite, the percentage of the subjects who have a 2-level composite response as measured by I-GAIS and/or S-GAIS in at least one treatment area at Day 71 is from about 1% to 10%, 10% to 20%, 20% to 30%, 30% to 40%, 40% to 50%, or greater than 50%.
   15. In a population of patients who have cellulite, the percentage of the subjects who have a 1-level composite response as measured by I-GAIS and/or S-GAIS in at least one treatment area at Day 71 is from about 1% to 10%, 10% to 20%, 20% to 30%, 30% to 40%, 40% to 50%, or greater than 50%.
   16. In a population of patients who have cellulite, over one-third, or one-half, or two-thirds, or three-fourths of the patients have at least a 1-level I-GAIS and/or S-GAIS responses in at least one treatment area by Day 71 post-treatment wherein the GAIS results are independent of age, BMI or skin color.
   17. The reduction in the severity of cellulite occurs rapidly within about 7, or 14, or 21, or 30, or 35, or 40, or 45, or 50 days after the first treatment visit.
   18. In a population of patients who all have CR-PCSS and/or PR-PCSS ratings of moderate or severe, at least 60% of patients are 1-level S-GAIS responders in the target buttock at Day 71.
   19. In a population of patients who all have CR-PCSS and/or PR-PCSS ratings of moderate or severe, at least 20% of patients are 2-level S-GAIS responders in the target buttock at Day 71.
   20. In a population of patients who all have CR-PCSS and/or PR-PCSS ratings of moderate or severe, at Day 71, the mean S-GAIS was greater in subjects treated with collagenase in the target buttock when compared to subjects treated with placebo (about 1.0 vs. about 0.5, respectively). The results are similar for the non-target buttock (about 1.0 in collagenase treated subjects vs. about 0.5 in placebo treated subjects).
   21. In a population of patients who all have CR-PCSS and/or PR-PCSS ratings of moderate or severe, at Day 71, the proportion of 1-level S-GAIS responders are greater in subjects treated with collagenase than in placebo treated subjects in the target buttock (about 70% vs. about 40%, respectively) and the non-target buttock (about 70% vs. about 40%, respectively).
   22. In a population of patients who all have CR-PCSS and/or PR-PCSS ratings of moderate or severe, at Day 71, the mean I-GAIS is statistically significantly greater in subjects treated with collagenase in the target buttock when compared to subjects treated with placebo (about 1.0 vs. 0.3, respectively). The results are similar for the non-target buttock (about 0.6 in collagenase treated subjects vs. about 0.1 in placebo treated subjects).
   23. In a population of patients who all have CR-PCSS and/or PR-PCSS ratings of moderate or severe, at Day 71, the proportion of 1-level I-GAIS responders is greater in subjects treated with collagenase than in placebo treated subjects in the target buttock (about 70% vs 25%, respectively) and the non-target buttock (70% vs 25%, respectively).
   24. In a population of patients who all have CR-PCSS and/or PR-PCSS ratings of moderate or severe, a series of cross-tabulations show consistency between PR-PCSS and S-GAIS. A 1 level change in the PR-PCSS was associated with similar changes in S-GAIS.

In another embodiment, the injection of about 1 mg to about 20 mg of collagenase to at least one treatment area during at least one treatment visit results in one or more of the results Nos. 1 to 24 above, wherein the collagenase has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In other cases, the injection of about 1 mg to about 20 mg of collagenase according to Treatment I results in one or more of the results Nos. 1 to 24 above.

In another instance, the injection of about 1 mg to about 20 mg of collagenase according to Treatment I results in one or more of the results Nos. 1 to 24 above, wherein the collagenase has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another example, the injection of about 1 mg to about 20 mg of CCH according to Treatment I results in one or more of the results Nos. 1 to 24 above.

In certain embodiments, about 1 mg to about 20 mg of an approximate 1:1 ratio of collagenase Type I and collagenase Type II is injected to at least one treatment area during at least one treatment visit and results in one or more of the results Nos. 1 to 24 above, wherein the collagenases I and II have the following characteristics:
Type I
   Assay: SRC microplate
   Vmax, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934

Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In certain embodiments, about 1 mg to about 20 mg of an approximate 1:1 ratio of collagenase Type I and collagenase Type II is injected employing Treatment I and results in one or more of the results Nos. 1 to 24 above, wherein the collagenases I and II have the following characteristics:
Type I
   Assay: SRC microplate
   Vmax, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934
      Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

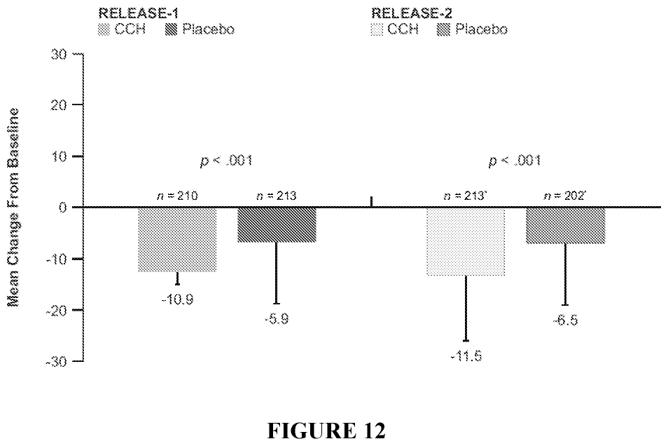

As further described in Examples 2 and 3, patients were rated for improvement after treatment compared with baseline using S-GAIS for the target and non-target buttock. As shown in FIG. 11, treatment with CCH was significantly better than placebo as shown by the ≥2-level and ≥1-level responses measured by S-GAIS.

### 7. Efficacy as Measured by PR-CIS

The treatment methods detailed above result in improved responses as measured by PR-CIS. The PR-CIS total score is the sum of the six items on the scales. The PR-CIS total score can range from 0 to 60 with higher numbers reflecting a more negative impact from the cellulite. Item #1 on the PR-CIS asking how happy the subject is about their appearance of cellulite is reversed by subtracting the subject's reported assessment from 10. For PR-CIS total score, a responder is a subject with a reduction in the PR-CIS total score of at least 12 from baseline at an evaluation time point. For individual PR-CIS impact scores, response is an improvement from baseline of at least 2 score intervals at each time point. Further, a responder is any patient showing at least a 20% improvement of maximum total score from baseline. An improvement for the individual patient at any visit is an improvement of at least 1 level or 1 rating from baseline or any previous score. An improvement for a group of patients at any visit is an improvement of about 0.1 in the mean score or rating from the baseline or any previous mean score or rating. Further, an improvement is a change from baseline of at least 1 level out of 60.

In certain embodiments, the treatment methods detailed above result in one or more of the following efficacy endpoints as measured by PR-CIS:

1. In a population of patients with cellulite, the injection of collagenase to at least one treatment area during at least one treatment visit results in a statistically significant number of such patients meeting one or more of the following efficacy endpoints:
The PR-CIS shows improvement across at least one domain selected from the group consisting of happiness with the appearance of cellulite, bother, self-consciousness, embarrassment, looking older, and looking overweight/out of shape
A reduction in the PR-CIS total score of at least 12 from baseline at one or more evaluation time points
PR-CIS impact scores showing improvement from baseline of at least 2 score intervals at one or more evaluation time points
An improvement is a change from baseline of at least 1 level out of 60
   2. An improvement in severity at Day 22, 43, 71, 90, 180, 251, 360, 431, 720, 3 years, 4 years, or 5 years from baseline (pretreatment “Day 1”) of at least 12 points in the PR-CIS for the treatment area.
   3. In a population of patients with cellulite, a statistically significant improvement in severity over placebo at Day 22, 43, 71, 90, 180, 251, 360, 431, 720, 3 years, 4 years, or 5 years from baseline (pretreatment Day 1) of at least 12 points in the PR-CIS for the treatment area.
   4. The treatment resulted in at least 5% of patients maintaining their level of improvement versus pretreatment baseline for at least 71 days after the initial dose. In certain cases, at least 10%, or 20%, or 30% or, 40% or 50% of patients maintained such level for at least 6 months, or 9 months, or 12 months, or 18 months, or 2 years or 3 years, or 4 years, or 5 years after the initial dose. In other cases, the treatments resulted in at least 5% of patients demonstrating improvement versus pretreatment baseline and showing an additional increase in improvement over time. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial dose. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial treatment, or second treatment, or third treatment.
   5. At Day 180, the improvement seen in the PR-CIS rating from baseline was consistent on the right and left treatment areas.
   6. In a population of patients who have cellulite, the median time to a reduction in the PR-CIS total score of at least 12 from baseline at one or more evaluation time points for at least one treatment area is about 15 days, or 20 days, or 30 days, or 40 days, or 50 days, or 60 days, or 70 days, or 80 days, or 90 days.
   7. In a population of patients who have cellulite, the mean subject PR-CIS score separates from placebo 21 days after the first treatment and demonstrate continuous and significant improvement after subsequent treatments.
   8. In a population of patients who have cellulite, over one-third, or over one-half, or over two-thirds, or over three-fourths of the patients have a reduction in the PR-CIS total score of at least 12 from baseline at one or more evaluation time points in at least one treatment area by Day 71 post-treatment wherein the PR-CIS results are independent of age, BMI or skin color.
   9. The reduction in the severity of cellulite occurs rapidly within about 7, or 14, or 21, or 30, or 35, or 40, or 45, or 50 days after the first treatment visit.
   10. In a population of patients who all have CR-PCSS and/or PR-PCSS ratings of moderate or severe, the mean change from baseline of PR-CIS Total Score at Day 71 is about −10 in collagenase treated subjects vs. about −5 in placebo treated subjects.
   11. In a population of patients who all have CR-PCSS and/or PR-PCSS ratings of moderate or severe, the mean PR-CIS change from baseline at Day 71 is statistically significantly favorable for collagenase treated subjects vs. placebo treatment subjects in total score (about −12 vs. −6, respectively) and abbreviated score (about −10 vs. 5, respectively), as well as individual impact scores (happiness with the appearance of cellulite, bothersome, self-consciousness, embarrassment, old appearance, and body shape concern).
   12. In a population of patients who all have CR-PCSS and/or PR-PCSS ratings of moderate or severe, at Day 71, the proportion of PR-CIS responders is greater in subjects treated with collagenase than in those treated with placebo for total score (about 45% vs. 20%, respectively) and for the abbreviated score (about 50% vs. 25%, respectively). In addition, the proportion of responders for each individual impact score was greater in collagenase treated subjects than in placebo treated subjects. These differences were also statistically significant.

In another embodiment, the injection of about 1 mg to about 20 mg of collagenase to at least one treatment area during at least one treatment visit results in one or more of the results Nos. 1 to 12 above, wherein the collagenase has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In other cases, the injection of about 1 mg to about 20 mg of collagenase according to Treatment I results in one or more of the results Nos. 1 to 12 above.

In another instance, the injection of about 1 mg to about 20 mg of collagenase according to Treatment I results in one or more of the results Nos. 1 to 12 above, wherein the collagenase has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another example, the injection of about 1 mg to about 20 mg of CCH according to Treatment I results in one or more of the results Nos. 1 to 12 above.

In certain embodiments, about 1 mg to about 20 mg of an approximate 1:1 ratio of collagenase Type I and collagenase Type II is injected to at least one treatment area during at least one treatment visit and results in one or more of the results Nos. 1 to 12 above, wherein the collagenases I and II have the following characteristics:
Type I
   Assay: SRC microplate
   Vₘₐₓ, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹ sec⁻¹: About 60 to 5,934

Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In certain embodiments, about 1 mg to about 20 mg of an approximate 1:1 ratio of collagenase Type I and collagenase Type II is injected employing Treatment I and results in one or more of the results Nos. 1 to 12 above, wherein the collagenases I and II have the following characteristics:
Type I
   Assay: SRC microplate
   Vmax, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vmax, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934
      Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

As further described in Examples 2 and 3, patients were rated for improvement after treatment compared with baseline using PR-CIS for CCH versus placebo. As shown in FIG. 11, treatment with CCH was significantly better than placebo.

### 8. Efficacy as Measured by PR-CIS Abbreviated

The treatment methods detailed above result in improved responses as measured by PR-CIS Abbreviated. The PR-CIS Abbreviated total score will be the sum of the five items on the scales. The PR-CIS Abbreviated total score can range from 0 to 50 with higher numbers reflecting a more negative impact from the cellulite. Item #1 on the PR-CIS asking how happy the subject is about their appearance of cellulite will be reversed by subtracting the subject's reported assessment from 10. For PR-CIS Abbreviated total score, a responder is a subject with a reduction in the PR-CIS total score of at least 10 from baseline at an evaluation time point. For individual PR-CIS Abbreviated impact scores, response is an improvement from baseline of at least 2 score intervals at each time point. Further, a responder is any patient showing at least a 20% improvement of maximum total score from baseline. An improvement for the individual patient at any visit is an improvement of at least 1 level or 1 rating from baseline or any previous score. An improvement for a group of patients at any visit is an improvement of about 0.1 in the mean score or rating from the baseline or any previous mean score or rating. Further, an improvement is a change from baseline of at least 1 level out of 50.

In certain embodiments, the treatment methods detailed above result in one or more of the following efficacy endpoints as measured by PR-CIS Abbreviated:

1. In a population of patients with cellulite, the injection of collagenase to at least one treatment area during at least one treatment visit results in a statistically significant number of such patients meeting one or more of the following efficacy endpoints:
The PR-CIS Abbreviated shows improvement across at least one domain selected from the group consisting of happiness with the appearance of cellulite, bother, self-consciousness, embarrassment, looking older, and looking overweight/out of shape
A reduction in the PR-CIS Abbreviated total score of at least 10 from baseline at one or more evaluation time points
PR-CIS Abbreviated impact scores showing improvement from baseline of at least 2 score intervals at one or more evaluation time points
Further, an improvement is a change from baseline of at least 1 level out of 50
   2. An improvement in severity at Day 22, 43, 71, 90, 180, 251, 360, 431, 720, 3 years, 4 years, or 5 years from baseline (pretreatment “Day 1”) of at least 12 points in the PR-CIS Abbreviated for the treatment area.
   3. In a population of patients with cellulite, a statistically significant improvement in severity over placebo at Day 22, 43, 71, 90, 180, 251, 360, 431, 720, 3 years, 4 years, or 5 years from baseline (pretreatment Day 1) of at least 10 points in the PR-CIS Abbreviated for the treatment area.
   4. The treatment resulted in at least 5% of patients maintaining their level of improvement versus pretreatment baseline for at least 71 days after the initial dose. In certain cases, at least 10%, or 20%, or 30% or, 40% or 50% of patients maintained such level for at least 6 months, or 9 months, or 12 months, or 18 months, or 2 years or 3 years, or 4 years, or 5 years after the initial dose. In other cases, the treatments resulted in at least 5% of patients demonstrating improvement versus pretreatment baseline and showing an additional increase in improvement over time. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial dose. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial treatment, or second treatment, or third treatment.
   5. At Day 180, the improvement seen in the PR-CIS Abbreviated rating from baseline was consistent on the right and left treatment areas.
   6. In a population of patients who have cellulite, the median time to a reduction in the PR-CIS Abbreviated total score of at least 10 from baseline at one or more evaluation time points for at least one treatment area is about 15 days, or 20 days, or 30 days, or 40 days, or 50 days, or 60 days, or 70 days, or 80 days, or 90 days.
   7. In a population of patients who have cellulite, the mean subject PR-CIS Abbreviated score separates from placebo 21 days after the first treatment and demonstrate continuous and significant improvement after subsequent treatments.
   8. In a population of patients who have cellulite, over one-third, or over one-half, or over two-thirds, or over three-fourths of the patients have a reduction in the PR-CIS Abbreviated total score of at least 10 from baseline at one or more evaluation time points in at least one treatment area by Day 71 post-treatment wherein the PR-CIS results are independent of age, BMI or skin color.
   9. The reduction in the severity of cellulite occurs rapidly within about 7, or 14, or 21, or 30, or 35, or 40, or 45, or 50 days after the first treatment visit.
   10. In a population of patients who all have CR-PCSS and/or PR-PCSS ratings of moderate or severe, the mean change from baseline of PR-CIS Abbreviated Total Score at Day 71 is about −10 in collagenase treated subjects vs. about −5 in placebo treated subjects.
   11. In a population of patients who all have CR-PCSS and/or PR-PCSS ratings of moderate or severe, the mean PR-CIS Abbreviated change from baseline at Day 71 is statistically significantly favorable for collagenase treated subjects vs. placebo treatment subjects in total score (about −12 vs. −6, respectively) and abbreviated score (about −10 vs. 5, respectively), as well as individual impact scores (happiness with the appearance of cellulite, bothersome, self-consciousness, embarrassment, old appearance, and body shape concern).
   12. In a population of patients who all have CR-PCSS and/or PR-PCSS ratings of moderate or severe, at Day 71, the proportion of PR-CIS Abbreviated responders is greater in subjects treated with collagenase than in those treated with placebo for total score (about 45% vs. 20%, respectively) and for the abbreviated score (about 50% vs. 25%, respectively). In addition, the proportion of responders for each individual impact score was greater in collagenase treated subjects than in placebo treated subjects. These differences were also statistically significant.

In another embodiment, the injection of about 1 mg to about 20 mg of collagenase to at least one treatment area during at least one treatment visit results in one or more of the results Nos. 1 to 12 above, wherein the collagenase has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In other cases, the injection of about 1 mg to about 20 mg of collagenase according to Treatment I results in one or more of the results Nos. 1 to 9 above.

In another instance, the injection of about 1 mg to about 20 mg of collagenase according to Treatment I results in one or more of the results Nos. 1 to 12 above, wherein the collagenase has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another example, the injection of about 1 mg to about 20 mg of CCH according to Treatment I results in one or more of the results Nos. 1 to 12 above.

In certain embodiments, about 1 mg to about 20 mg of an approximate 1:1 ratio of collagenase Type I and collagenase Type II is injected to at least one treatment area during at least one treatment visit and results in one or more of the results Nos. 1 to 12 above, wherein the collagenases I and II have the following characteristics:
Type I
   Assay: SRC microplate
   Vmax, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934

Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In certain embodiments, about 1 mg to about 20 mg of an approximate 1:1 ratio of collagenase Type I and collagenase Type II is injected employing Treatment I and results in one or more of the results Nos. 1 to 12 above, wherein the collagenases I and II have the following characteristics:
Type I
   Assay: SRC microplate
   Vmax, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934

Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

### 9. Efficacy as Measured by Subject Self-Rating Scale (SSRS)

The treatment methods detailed above result in improvements as measured by SSRS. A SSRS responder is a subject who is at least slightly satisfied (slightly satisfied [4], very satisfied [5], or extremely satisfied [6]) with the appearance of the cellulite on an affected area at an evaluation time point. Further, a responder is any patient showing at least a 17% improvement of maximum total score from baseline. An improvement for the individual patient at any visit is an improvement of at least 1 level or 1 rating from baseline or any previous score. An improvement for a group of patients at any visit is an improvement of about 0.1 in the mean score or rating from the baseline or any previous mean score or rating. In certain embodiments, the treatment methods result in one or more of the following efficacy endpoints as measured by SSRS Rating:

1. In a population of patients with cellulite, the injection of collagenase to at least one treatment area during at least one treatment visit resulted in a statistically significant number of such patients meeting one or more of the following efficacy endpoints:
A SSRS Rating of “Slightly satisfied”
A SSRS Rating of “Satisfied”
A SSRS Rating of “Extremely satisfied”
   2. An improvement at Day 22, 43, 71, 90, 180, 251, 360, 431, 720, 3 years, 4 years, or 5 years days from baseline (pretreatment “Day 1”) of at least 2 levels in the SSRS Rating of the treatment area.
   3. An improvement at Day 22, 43, 71, 90, 180, 251, 360, 431, 720, 3 years, 4 years, or 5 years days from baseline (Day 1) of at least 1 level in the SSRS Rating of the treatment area.
   4. In a population of patients with cellulite, the improvement in SSRS Ratings in at least one treatment area was statistically significant compared to placebo wherein the improvement is one or more of Nos. 2 to 3 above.
   5. The treatment resulted in at least 5% of patients maintaining their level of improvement versus pretreatment baseline for at least 71 days after the initial dose. In certain cases, at least 10%, or 20%, or 30% or, 40% or 50% of patients maintained such level for at least 6 months, or 9 months, or 12 months, or 18 months, or 2 years or 3 years, or 4 years, or 5 years after the initial dose. In other cases, the treatments resulted in at least 5% of patients demonstrating improvement versus pretreatment baseline and showing an additional increase in improvement over time. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial dose. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial treatment, or second treatment, or third treatment.
   6. At Day 180 after the first injection, the improvement seen in the SSRS Rating from baseline was consistent on the right and left treatment areas.
   7. In a population of patients who have cellulite, the median time to the earliest 2-level SSRS Rating improvement in at least one treatment area is about 50 days, or 60 days, or 70 days, or 80 days, or 90 days.
   8. In a population of patients who have cellulite, the median time to the earliest 1-level SSRS Rating improvement in at least one treatment area is about 15 days, or 20 days, or 30 days, or 40 days, or 50 days, or 60 days, or 70 days, or 80 days, or 90 days.
   9. In a population of patients who have cellulite, the mean subject SSRS Rating separates from placebo 21 days after the first treatment and demonstrate continuous and significant improvement after subsequent treatments.
   10. In a population of patients who have cellulite, the percentage of the subjects who have a 2-level composite response as measured by SSRS Rating in at least one treatment area at Day 71 is from about 1% to 10%, 10% to 20%, 20% to 30%, 30% to 40%, 40% to 50%, or greater than 50%.
   11. In a population of patients who have cellulite, the percentage of the subjects who have a 1-level composite response as measured by SSRS Rating in at least one treatment area at Day 71 is from about 1% to 10%, 10% to 20%, 20% to 30%, 30% to 40%, 40% to 50%, or greater than 50%.
   12. In a population of patients who have cellulite, over one-third, or one-half, or two-thirds, or three-fourths of the patients have at least a 1-level SSRS Rating responses in at least one treatment area by day 71 post-treatment wherein the SSRS Rating results are independent of age, BMI or skin color.
   13. The reduction in the severity of cellulite occurs rapidly within about 7, or 14, or 21, or 30, or 35, or 40, or 45, or 50 days after the first treatment visit.
   14. In a population of patients who all have CR-PCSS and/or PR-PCSS ratings of moderate or severe, at least 40% of patients are 1-level SSRS responders at Day 71.
   15. In a population of patients who all have CR-PCSS and/or PR-PCSS ratings of moderate or severe, at Day 71, the mean SSRS score are statistically significantly greater in subjects treated with collagenase than in those treated with placebo.
   16. In a population of patients who all have CR-PCSS and/or PR-PCSS ratings of moderate or severe, at least 50% of patients are 1-level SSRS responders.

In another embodiment, the injection of about 1 mg to about 20 mg of collagenase to at least one treatment area during at least one treatment visit results in one or more of the results Nos. 1 to 16 above, wherein the collagenase has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In other cases, the injection of about 1 mg to about 20 mg of collagenase according to Treatment I results in one or more of the results Nos. 1 to 16 above.

In another instance, the injection of about 1 mg to about 20 mg of collagenase according to Treatment I results in one or more of the results Nos. 1 to 16 above, wherein the collagenase has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another example, the injection of about 1 mg to about 20 mg of CCH according to Treatment I results in one or more of the results Nos. 1 to 16 above.

In certain embodiments, about 1 mg to about 20 mg of an approximate 1:1 ratio of collagenase Type I and collagenase Type II is injected to at least one treatment area during at least one treatment visit and results in one or more of the results Nos. 1 to 16 above, wherein the collagenases I and II have the following characteristics:
Type I
   Assay: SRC microplate
   Vmax, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934
      Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In certain embodiments, about 1 mg to about 20 mg of an approximate 1:1 ratio of collagenase Type I and collagenase Type II is injected employing Treatment I and results in one or more of the results Nos. 1 to 16 above, wherein the collagenases I and II have the following characteristics:
Type I
   Assay: SRC microplate
   Vmax, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934

Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

10. Efficacy as Measured by Subject Satisfaction with Cellulite Treatment (SSCT)

The treatment methods detailed above result in improvements as measured by SSCT. A subject with a response of “Satisfied” or “Very Satisfied” on the SSCT assessment at Day 71 is considered a responder showing efficacy. An improvement for the individual patient at any visit is an improvement of at least 1 level or 1 rating from baseline or any previous score or rating. An improvement for a group of patients at any visit is an improvement of about 0.1 in the mean score or rating from the baseline or any previous mean score or rating. Further, for SSCT, an improvement is at least 0.1 as compared to placebo. In certain embodiments, the treatment methods result in one or more of the following efficacy endpoints as measured by SSCT Rating:

1. In a population of patients with cellulite, the injection of collagenase to at least one treatment area during at least one treatment visit resulted in a statistically significant number of such patients meeting one or more of the following efficacy endpoints:
A SSCT Rating of “Satisfied”
A SSCT Rating of “Very Satisfied”
   2. Increase in the SSCT rating (e.g., 1 to 2, etc.) at Day 22, 43, 71, 90, 180, 251, 360, 431, 720, 3 years, 4 years, or 5 years of the treatment area.
   3. In a population of patients with cellulite, an increase in SSCT Ratings in at least one treatment area was statistically significant compared to placebo wherein the improvement is at Day 22, 43, 71, 90, 180, 251, 360, 431, 720, 3 years, 4 years, or 5 years in the SSCT Rating of the treatment area.
   4. The treatment resulted in at least 5% of patients maintaining their level of improvement for at least 71 days after the initial dose. In certain cases, at least 10%, or 20%, or 30% or, 40% or 50% of patients maintained such level for at least 6 months, or 9 months, or 12 months, or 18 months, or 2 years or 3 years, or 4 years, or 5 years after the initial dose. In other cases, the treatments resulted in at least 5% of patients demonstrating improvement and showing an additional increase in improvement over time. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial dose. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial treatment, or second treatment, or third treatment.
   5. At Day 180, the improvement seen in the SSCT Rating is consistent on the right and left treatment areas.
   6. In a population of patients who have cellulite, the median time to the earliest SSCT Rating improvement in at least one treatment area is about 15 days, or 20 days, or 30 days, or 40 days, or 50 days, or 60 days, or 70 days, or 80 days, or 90 days.
   7. In a population of patients who have cellulite, the mean subject SSCT Ratings separate from placebo 21 days after the first treatment and demonstrates continuous and significant improvement after subsequent treatments.
   8. In a population of patients who have cellulite, the percentage of the subjects who have an improved SSCT Rating in at least one treatment area at Day 71 is from about 1% to 10%, 10% to 20%, 20% to 30%, 30% to 40%, 40% to 50%, or greater than 50%.
   9. In a population of patients who have cellulite, over one-third, or one-half, or two-thirds, or three-fourths of the patients have an improved SSCT Rating in at least one treatment area by Day 71 post-treatment wherein the SSCT Rating results are independent of age, BMI or skin color.
   10. The reduction in the severity of cellulite occurs rapidly within about 7, or 14, or 21, or 30, or 35, or 40, or 45, or 50 days after the first treatment visit.
   11. In a population of patients who all have CR-PCSS and/or PR-PCSS ratings of moderate or severe, at Day 180, more than half of patients are either satisfied or very satisfied with treatment.
   12. In a population of patients who all have CR-PCSS and/or PR-PCSS ratings of moderate or severe, the decrease in the CR-PCSS and/or PR-PCSS ratings sustained at Day 180 coupled with the scores on the subject satisfaction survey demonstrates that three treatment sessions of 0.84 mg CCH administered as 12 subcutaneous injections per treatment are (×2 treatment areas) to either bilateral buttocks or bilateral thighs is effective in decreasing cellulite.
   13. In a population of patients who all have CR-PCSS and/or PR-PCSS ratings of moderate or severe, at least 50% of patients treated with collagenase are satisfied or very satisfied with their cellulite treatment.
   14. In a population of patients who all have CR-PCSS and/or PR-PCSS ratings of moderate or severe, there is a statistically significant difference in mean subject satisfaction scores at Day 71 between the collagenase and the placebo treatment groups.

In another embodiment, the injection of about 1 mg to about 20 mg of collagenase to at least one treatment area during at least one treatment visit results in one or more of the results Nos. 1 to 14 above, wherein the collagenase has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In other cases, the injection of about 1 mg to about 20 mg of collagenase according to Treatment I results in one or more of the results Nos. 1 to 14 above.

In another instance, the injection of about 1 mg to about 20 mg of collagenase according to Treatment I results in one or more of the results Nos. 1 to 14 above, wherein the collagenase has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another example, the injection of about 1 mg to about 20 mg of CCH according to Treatment I results in one or more of the results Nos. 1 to 14 above.

In certain embodiments, about 1 mg to about 20 mg of an approximate 1:1 ratio of collagenase Type I and collagenase Type II is injected to at least one treatment area during at least one treatment visit and results in one or more of the results Nos. 1 to 14 above, wherein the collagenases I and II have the following characteristics:
Type I
   Assay: SRC microplate
   Vmax, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934

Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In certain embodiments, about 1 mg to about 20 mg of an approximate 1:1 ratio of collagenase Type I and collagenase Type II is injected employing Treatment I and results in one or more of the results Nos. 1 to 14 above, wherein the collagenases I and II have the following characteristics:
Type I
   Assay: SRC microplate
   Vmax, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934

Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

### 11. Efficacy as Measured by the Thigh Cellulite Severity-Patient (TCS-P); Thigh Cellulite Severity-Clinician (TCS-C)

An improvement for the individual patient at any visit is an improvement of at least 1 level or 1 rating from baseline or any previous score or rating. An improvement for a group of patients at any visit is an improvement of about 0.1 in the mean score or rating from the baseline or any previous mean score or rating. A responder is any patient showing at least a 20% improvement of maximum total score or rating from baseline. In certain embodiments, the treatment methods detailed above result in one or more of the following efficacy endpoints as measured by TCS-C and/or TCS-P:

1. An improvement of at least 0.1 in TCS-C and/or TCS-P rating over baseline.
2. An improvement in severity at Day 22, 43, 71, 90, 180, 251, 360, 431, 720, 3 years, 4 years, or 5 years from baseline (pretreatment “Day 1”) of at least 2 levels of severity in the TCS-C as assessed live by the clinician of the target thigh.
3. An improvement in severity at Day 22, 43, 71, 90, 180, 251, 360, 431, 720, 3 years, 4 years, or 5 years from baseline (Day 1) of at least 2 levels of severity in the TCS-P as assessed by the subject while viewing the digital image of the target thigh.
4. An improvement demonstrated by a 2-level composite response at Day 22, 43, 71, 90, 180, 251, 360, 431, 720, 3 years, 4 years, or 5 years defined as a subject with an improvement from baseline of at least 2 levels of severity in the TCS-C and an improvement from baseline of at least 2 levels of severity in the TCS-P.
5. An improvement in severity at Day 22, 43, 71, 90, 180, 251, 360, 431, 720, 3 years, 4 years, or 5 years baseline (Day 1) of at least 1 level of severity in the TCS-C as assessed live by the clinician of the target thigh.
6. An improvement in severity at Day 22, 43, 71, 90, 180, 251, 360, 431, 720, 3 years, 4 years, or 5 years from baseline (Day 1) of at least 1 level of severity in the TCS-P as assessed by the subject while viewing the digital image of the target thigh.
7. An improvement demonstrated by a 1-level composite response at Day 22, 43, 71, 90, 180, 251, 360, 431, 720, 3 years, 4 years, or 5 years defined as a subject with an improvement from baseline of at least 1 level of severity in the TCS-C and an improvement from baseline of at least 1 level of severity in the TCS-P.
8. In a population of patients who all had TCS-C ratings of moderate or severe, the improvement in at least one treatment area was statistically significant compared to placebo wherein the improvement is one or more of Nos. 1 to 7 above.
9. The treatment resulted in at least 5% of patients maintaining their level of improvement versus pretreatment baseline for at least 71 days after the initial dose. In certain cases, at least 10%, or 20%, or 30% or, 40% or 50% of patients maintained such level for at least 6 months, or 9 months, or 12 months, or 18 months, or 2 years or 3 years, or 4 years, or 5 years after the initial dose. In other cases, the treatments resulted in at least 5% of patients demonstrating improvement versus pretreatment baseline and showing an additional increase in improvement over time. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial dose. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial treatment, or second treatment, or third treatment.
10. At Day 180, the improvement seen in the TCS-C and/or TCS-P rating from baseline was consistent on the right and left thighs.
11. In a population of patients who all have TCS-C ratings of moderate or severe, the median time to the earliest 2-level TCS-C and/or TCS-P improvement in at least one treatment area is about 50 days, or 60 days, or 70 days, or 80 days, or 90 days.
12. In a population of patients who all have TCS-C ratings of moderate or severe, the median time to the earliest 1-level TCS-C and/or TCS-P improvement in at least one treatment area is about 15 days, or 20 days, or 30 days, or 40 days, or 50 days, or 60 days, or 70 days, or 80 days, or 90 days.
13. In a population of patients who all have TCS-C ratings of moderate or severe, the mean subject TCS-C and/or TCS-P scores separates from placebo 21 days after the first treatment and demonstrate continuous and significant improvement after subsequent treatments.
14. In a population of patients who all have TCS-C ratings of moderate or severe, the percentage of the subjects who have a 2-level composite response as measured by TCS-C and/or TCS-P in at least one treatment area at Day 71 is from about 1% to 10%, 10% to 20%, 20% to 30%, 30% to 40%, 40% to 50%, or greater than 50%.
15. In a population of patients who all have TCS-C ratings of moderate or severe, the percentage of the subjects who have a 1-level composite response as measured by TCS-C and/or TCS-P in at least one treatment area at Day 71 is from about 1% to 10%, 10% to 20%, 20% to 30%, 30% to 40%, 40% to 50%, or greater than 50%.
16. In a population of patients who all have TCS-C ratings of moderate or severe, over one-third, or one-half, or two-thirds, or three-fourths of the patients have at least a 1-level TCS-C and/or at least a 1-level TCS-P responses in at least one treatment area by Day 71 post-treatment wherein the TCS-C results are independent of age, BMI or skin color.
17. The reduction in the severity of cellulite occurs rapidly within about 7, or 14, or 21, or 30, or 35, or 40, or 45, or 50 days after the first treatment visit.

In another embodiment, the injection of about 1 mg to about 20 mg of collagenase to at least one treatment area during at least one treatment visit results in one or more of the results Nos. 1 to 17 above, wherein the collagenase has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In other cases, the injection of about 1 mg to about 20 mg of collagenase according to Treatment I results in one or more of the results Nos. 1 to 17 above.

In another instance, the injection of about 1 mg to about 20 mg of collagenase according to Treatment I results in one or more of the results Nos. 1 to 17 above, wherein the collagenase has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another example, the injection of about 1 mg to about 20 mg of CCH according to Treatment I results in one or more of the results Nos. 1 to 17 above.

In certain embodiments, about 1 mg to about 20 mg of an approximate 1:1 ratio of collagenase Type I and collagenase Type II is injected to at least one treatment area during at least one treatment visit and results in one or more of the results Nos. 1 to 17 above, wherein the collagenases I and II have the following characteristics:
Type I
   Assay: SRC microplate
   Vmax, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934

Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In certain embodiments, about 1 mg to about 20 mg of an approximate 1:1 ratio of collagenase Type I and collagenase Type II is injected employing Treatment I and results in one or more of the results Nos. 1 to 17 above, wherein the collagenases I and II have the following characteristics:
Type I
   Assay: SRC microplate
   Vmax, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934

Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

### 12. Efficacy as Measured by Body-Q

The treatment methods detailed above result in improved responses as measured by Body-Q. For cellulite, there are 16 scaled items having patient response options ranging from “not at all” to “extremely bothered” over the timeframe of the past week and assuming a Flesch-Kincaid grade reading level. The score ranges from 16 (extremely bothered) to 64 (not at all). For Body-Q total score, a responder is a subject with an increase in the Body-Q total score of at least 16 from baseline at an evaluation time point. For individual Body-Q impact scores, response is an improvement from baseline of at least 1 score interval at each time point. In alternative embodiments, the scaled items may be more or less than 16, and a responder is any patient showing at least a 25% improvement of the maximal total score from baseline.

In certain embodiments, the treatment methods detailed above result in one or more of the following efficacy endpoints as measured by Body-Q:

1. In a population of patients with cellulite, the injection of collagenase to at least one treatment area during at least one treatment visit results in a statistically significant number of such patients meeting one or more of the following efficacy endpoints:
The Body-Q shows improvement across at least one domain selected from the group consisting of:
   An increase in the Body-Q total score of at least 16 from baseline at one or more evaluation time points;
   for individual Body-Q impact scores, an improvement from baseline of at least 1 score interval at each time point; and
   at least a 25% improvement of the maximal total score from baseline.
An increase in the Body-Q total score of at least 16 from baseline at one or more evaluation time points
Body-Q impact scores showing improvement from baseline of at least 1 score interval at one or more evaluation time points
Mean change from baseline in Body-Q appraisal of cellulite at Day 90 and/or Day 180
   2. An improvement in severity at Day 22, 43, 71, 90, 180, 251, 360, 431, 720, 3 years, 4 years, or 5 years days from baseline (pretreatment “Day 1”) of an increase of at least 16 points in the Body-Q for the target buttock.
   3. In a population of patients with cellulite, a statistically significant improvement in severity over placebo at Day 22, 43, 71, 90, 180, 251, 360, 431, 720, 3 years, 4 years, or 5 years days baseline (pretreatment Day 1) as indicated by an increase of at least 16 points in the Body-Q for the target buttock.
   4. The treatment resulted in at least 5% of patients maintaining their level of improvement versus pretreatment baseline for at least 71 days after the initial dose. In certain cases, at least 10%, or 20%, or 30% or, 40% or 50% of patients maintained such level for at least 6 months, or 9 months, or 12 months, or 18 months, or 2 years or 3 years, or 4 years, or 5 years after the initial dose. In other cases, the treatments resulted in at least 5% of patients demonstrating improvement versus pretreatment baseline and showing an additional increase in improvement over time. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial dose. Some treatments resulted in at least 10%, or 20%, or 30%, or 40% or 50% of patients demonstrating improvement coupled with an additional increase in improvement at 1 month, or 3 months, or 6 months, or 9 months, or 12 months, or 18 months, or 24 months after the initial treatment, or second treatment, or third treatment.
   5. At Day 180 after the first injection, the improvement seen in the Body-Q rating from baseline was consistent on the right and left buttocks.
   6. In a population of patients who have cellulite, the median time to a Body-Q total score increase of at least 16 from baseline at one or more evaluation time points for at least one treatment area is about 15 days, or 20 days, or 30 days, or 40 days, or 50 days, or 60 days, or 70 days, or 80 days, or 90 days.
   7. In a population of patients who have cellulite, the mean subject Body-Q score separates from placebo 21 days after the first treatment and demonstrate continuous and significant improvement after subsequent treatments.
   8. In a population of patients who have cellulite, over one-third, or over one-half, or over two-thirds, or over three-fourths of the patients have a Body-Q total score increase of at least 16 from baseline at one or more evaluation time points in at least one treatment area by day 71 post-treatment wherein the Body-Q results are independent of age, BMI or skin color.
   9. The reduction in the severity of cellulite occurs rapidly within about 7, or 14, or 21, or 30, or 35, or 40, or 45, or 50 days after the first treatment visit.

In another embodiment, the injection of about 1 mg to about 20 mg of collagenase to at least one treatment area during at least one treatment visit results in one or more of the results Nos. 1 to 9 above, wherein the collagenase has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In other cases, the injection of about 1 mg to about 20 mg of collagenase according to Treatment I results in one or more of the results Nos. 1 to 9 above.

In another instance, the injection of about 1 mg to about 20 mg of collagenase according to Treatment I results in one or more of the results Nos. 1 to 9 above, wherein the collagenase has one or more of the following characteristics:
Vₘₐₓ (min⁻¹) of about 0.08 to 7.70 (SRC assay), or about 0.3 to 30.5 (GPA assay)
K_{M}, of about 4.1 to 410 nanoMolar (SRC assay), or about 0.03 to 3.1 mM (GPA assay)
K_{cat} (sec⁻¹) of about 1.1 to 107 (SRC assay), or about 93 to 9,179 (GPA assay)
1/K_{cat}, microseconds of about 376 to 37,222 (SRC assay), or about 4 to 428 (GPA assay)
K_{cat}/K_{M}, mM⁻¹sec⁻¹ of about 5,140 to 508,814 (SRC assay), or about 60 to 5,934 (GPA assay)
A molecular mass from about 60 kDa to about 130 kDa, or about 70 to about 130 kDa, or about 80 to about 120 kDa, or about 90 to about 120 kDa, or about 100 to about 110 kDa.
A purity by area of at least 80% as measured by reverse phase HPLC (high pressure liquid chromatography)
Potency (i.e., specific activity) of about 500 to about 30,000 SRC units/mg
Potency of about 5,000 to about 30,000 f-SRC units/mg
Potency of about 100,000 to about 400,000 GPA units/mg
Potency of about 175,000 to about 500,00 f-GPA units/mg
Potency of about 5,000 to about 25,000 ABC units/mg
Less than or equal to 1% by area of an impurity selected from the group consisting of clostripain, gelatinase, and leupeptin
Less than or equal to 1 cfu/mL bioburden

In another example, the injection of about 1 mg to about 20 mg of CCH according to Treatment I results in one or more of the results Nos. 1 to 9 above.

In certain embodiments, about 1 mg to about 20 mg of an approximate 1:1 ratio of collagenase Type I and collagenase Type II is injected to at least one treatment area during at least one treatment visit and results in one or more of the results Nos. 1 to 9 above, wherein the collagenases I and II have the following characteristics:
Type I
   Assay: SRC microplate
   Vmax, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934
      Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

In certain embodiments, about 1 mg to about 20 mg of an approximate 1:1 ratio of collagenase Type I and collagenase Type II is injected employing Treatment I and results in one or more of the results Nos. 1 to 9 above, wherein the collagenases I and II have the following characteristics:
Type I
   Assay: SRC microplate
   Vmax, min⁻¹: About 0.08 to 7.70
   K_{M}: About 4.1 to 410 nanoMolar
   K_{cat}, sec⁻¹: About 1.1 to 107
   1/K_{cat}, microseconds: About 376 to 37,222
   K_{cat}/K_{M}, mM⁻¹sec⁻¹: About 5,140 to 508,814
Type II
   Assay: GPA microplate
   Vₘₐₓ, min⁻¹: About 0.3 to 30.5
   K_{M}, mM: About 0.03 to 3.1
   K_{cat}, sec⁻¹: About 93 to 9,179
   1/K_{cat}, microseconds: About 4 to 428
   K_{cat}/K_{M}, mM⁻¹sec⁻¹: About 60 to 5,934
      Other ratios may be employed (e.g., 0.1-2:1, or 0.25-2:1, or 0.5-2:1, or 0.75-2:1, or 1:0.1-2, or 1:0.25-2, or 1:0.5-2, or 1:0.75-2, or 1:0, or 0:1). Further, the Type I and Type II collagenases may be AUX-I and AUX-II, respectively.

### 13. Assessment of Treatment Effect as Measured by 3-D Photography/Imagery

Introduction. 3-D photography or other imagery can be used in the assessment of treatment effect, in particular for the dimple and bruising analyses. Dimple analysis (as defined above) using 3-D imagery can include calculations of dimple volume, length, width and area. The calculations may be performed by various known methods such as those described in Eckhouse et al. WO 2018/116304 and WO 2018/116305, Cherry Imaging and those available from Canfield Scientific, Inc. Such measurements of volume, length, width and area may be calculated using digital 3-D greyscale images (with X and Y axis rotation feature) and digital 3-D textured and lit images (with X and Y rotation feature) together with a computer program that analyzes such images. In one embodiment, for a buttock treated area, images may be taken of the left treated buttock and/or right treated buttock for each patient before and after treatment. In another embodiment, for a thigh treated area, images may be taken of each of the thigh treated areas of the subject's posterior, oblique, and lateral sides at, for example, 0 degrees, 45 degrees and 90 degrees before and after treatment.

Clinicians and patients may use photographs and imaging tools to assess the severity of cellulite with or without the scales and other tools described herein. In one embodiment, the clinician/investigator or qualified designee photographs each treatment area (for example, each buttock or each thigh) using a supplied standardized 3-D camera in a standardized manner. The subject stands for each photography session and wears a standardized photographic garment. The clinician/investigator or qualified designee photographs each of the 2 treatment areas (for example, 2 buttocks or 2 thighs) while the subject is standing in a consistent, standardized relaxed pose. These photographs and the imagery described above may be used in the efficacy analyses described below. Additional non-limiting and optional details for the methodology are set forth below.

Image Analysis by 3-D Photography. Assessment of treatment effect is performed by 3-D photography. The investigator or qualified designee photograph each treatment area (both buttocks or both thighs) prior to injections using a standardized digital camera in a standardized manner, before and after marking dimples and injection sites on treatment Day 1, 22, and 43. A single set of photographs are taken of each treatment area at screening and all other non-dosing visits. The photographs taken at Day 4, 8, 15, 22, 43, and 71 (end of study/ET) are reviewed by a central assessor blinded to the treatment arm and study visit day.

The standard Image Analysis (IA) procedures used to evaluate bruising, volume, surface area, and max length/width are discussed below. The camera system may be an IntelliStudio available from Canfield Scientific equipped with custom lighting, a Vectra M1 camera, and Canfield Capture software (v. 3.5). The image types, attributes, views, and visit windows are defined and inputted directly into a Digital Monitoring System (DMS). A study set-up in DMS contained the following:

| Attribute Type | |
|---|---|
| (category) | Attribute Names |
| Image Type | 2-D, 3-D, Contact Sheets |
| View | ID Card, Left Buttock Posterior, Right |
| | Buttock Posterior, Left Thigh Lateral, Left |
| | Thigh Oblique, Left Thigh Posterior, Right |
| | Thigh Posterior, Right Thigh Oblique, Right |
| | Thigh Lateral |
| Real Time Monitoring | Real Time Monitoring |

Images are captured and staged into DMS and available for review. Photographic visits may include: Screening, Day 1/Pre-Marking, Day 1/Post-Marking, Day 4, Day 8, Day 15, Day 22/Pre-Marking, Day 22/Post Marking, Day 43/Pre-Marking, Day 43 Post-Marking, and Day 71/ET.

Dimple Analysis: Images are reviewed to ensure a primary dimple is marked with an “X” as instructed in each quadrant. If a primary dimple is not marked, the following steps are taken:
1. The image(s) and place the images are exported in a PowerPoint.
2. The site is contacted and asked the site's investigator to mark the primary dimple with an X on the image within the PowerPoint.
3. The correspondence is saved and shared with the image analysis (IA) team.

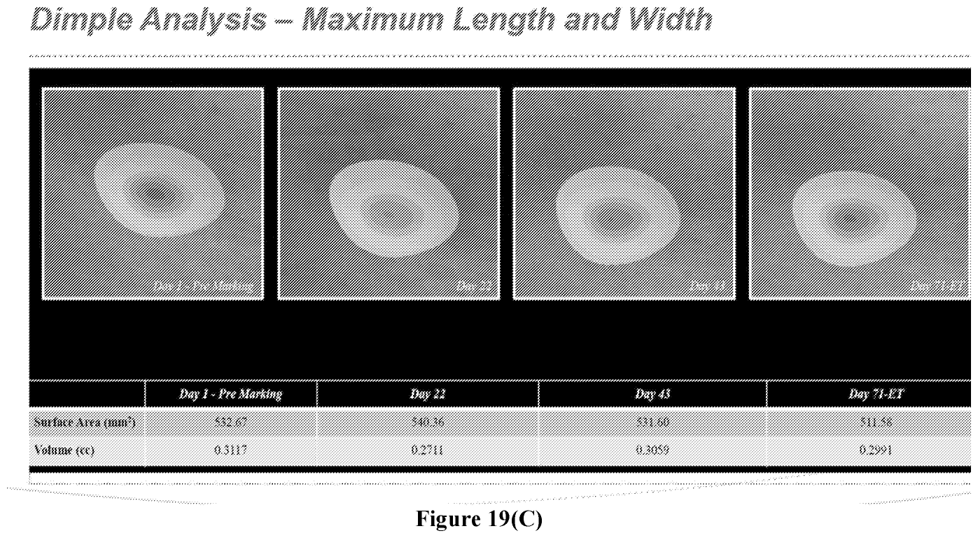

Dimple analysis is performed on Days 1 (pre-marking), 22, 43 and 71. The observed and change from Day 1 pre-marking image in dimple analysis parameters, maximum length, maximum width, surface area, and volume between the dimple base and interpolated surface, are summarized at Day 22, 43, and 71 by treatment area and injection type using descriptive statistics (an exemplary dimple analysis is depicted in FIGS. 19(A)-19(C)). In addition, the volume is analyzed using the linear model.

An image analysis technician (IAT) uses the Day 1-Post marking image as a reference to determine the location of the target dimple on the Day 1-Pre marking image. A tracing is made around the border of concavity of the dimple on the Day 1-Pre marking. The dimple tracing is transposed on to the Day 22-Pre Marking, Day 43-Pre marking, and Day 71/ET images. This tracing is used to measure the (i) maximum length (i.e., the longest straight line distance across the dimple); (ii) maximum width (i.e., the longest straight line distance perpendicular to the maximum length measurement); (iii) surface area of the dimple; and (iv) volume between the base of the dimple and the interpolated surface.

Bruising Analysis: Images are reviewed to ensure a white reference label is present in each image and outside of the bruise area. If the reference label is not present or obstructing the bruised area, a reshoot is requested if applicable for that visit. Bruise analysis is performed on Days 1 (pre-marking), 4, 8, and 15.

Site Marking Analysis: Images are reviewed to ensure dimples are marked at the Day 1 Post-Marking visit. If dimples are not marked, a reshoot is requested if applicable for that visit.

Image Analysis (IA) Workflow: All images selected for analysis are assigned random tracking numbers which blinds the image analysis technicians (IAT) to information, such as investigative site, subject secondary identifier, and/or treatment arm. The analysis procedure performed on a particular visit is pre-determined based on visit name. Dimple analysis is performed on the pre-identified, primary dimple and will include measurements of volume, surface area, and max length/width. Bruise analysis included L*a*b* color measurements in the perceived bruised area and an area outside the perceived bruise on unaffected skin. Site Marking Analysis consists of max length/width in addition to a surface area measurement of the tracing.

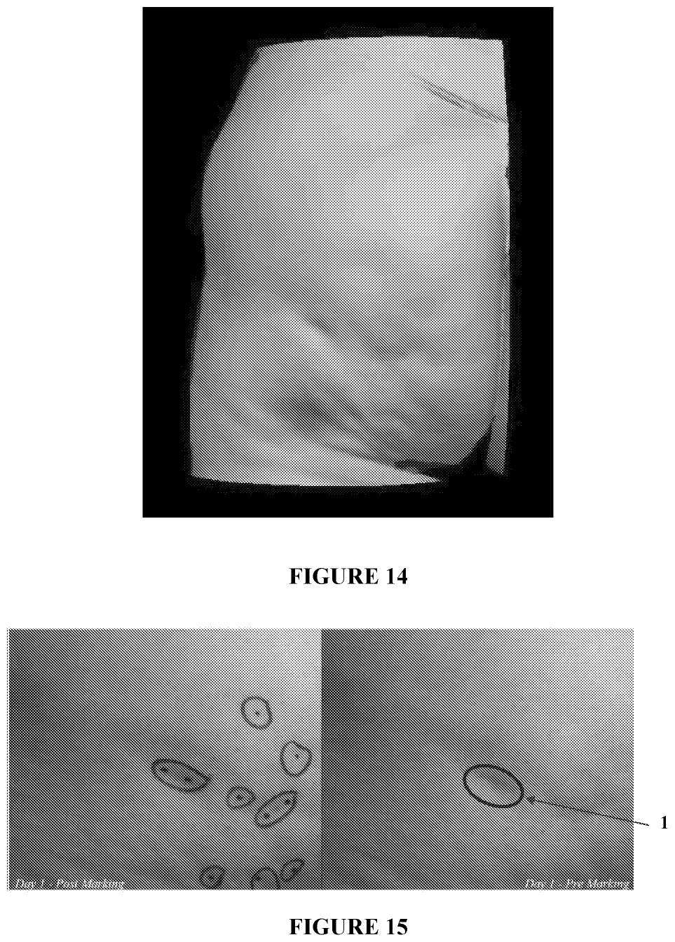

Image Registration: Upon receiving a Day 1 Pre-Marking (Baseline) visit, a IAT opens the pre-marking image in Vectra Analysis Module (VAM) software and centers the image to grid in 3-D space. Using the grid as a reference, the IAT positions the Baseline image so that the approximate center of the image is placed at the grid's origin. The thigh/buttock faces forward in the +z-direction, the upper thigh/buttock points in the +y-direction and the lower thigh/buttock points in the −y-direction (FIG. 13). All subsequent time points, including the Day 1 post-marking image are registered to Baseline image's position in the grid using an algorithmic registration function.

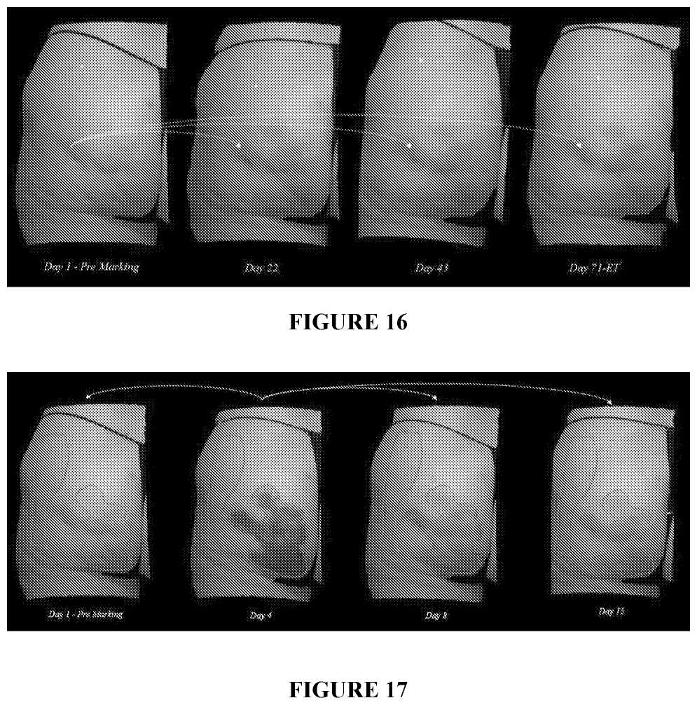

A color-by-distance map is produced within VAM which the IAT uses to review registration. The color-by-distance map represents the distance between the two (2) image models based on a color scale of +5 to −5 millimeter (mm) (FIG. 14). The IAT ensures the majority of the image is colored green, indicating there is a negligible distance between the two images and that they are properly aligned.

| Color | Distance from Baseline |
|---|---|
| Green | 0 or negligible distance |
| Cyan → Blue | Positive Distance (Up to +5 mm) |
| Yellow → Red | Negative Distance (Down to −5 mm) |

Surface Tracking Processing: Following image registration, the Day 1 (Pre-Marking), Day 4, Day 8, Day 15, Day 22, Day 43 and Day 71 images receive surface tracking processing. The IAT first uses a script to create a high-density mesh template of the Day 1, Baseline image. One Follow-Up image at a time is opened in the VAM software along with the Baseline and mesh template. Tracking seed landmarks are placed on identifiable skin features as references. The tracking script is run to programmatically create and save three (3) new 3-D images, a tracked Baseline image, a tracked Follow-Up image, and a Quality Control (QC) image which is the shape of the tracked Follow-Up image and conveys information regarding tracking quality.

A quality check IAT reviews the quality of the tracked Follow-Up image and QC image to verify a successful tracking. For visits that receive dimple analysis, the IAT also overlays the QC image over the tracked Follow-Up image to ensure there are no large holes on the QC image in the region of the primary dimple. A hole in the mesh of the QC image indicates a loss of tracking information in the area of drop out. In the event that the IAT deems tracking unacceptable due to a loss of information within an area for analysis, the tracking seed landmarks are adjusted and the tracking script run again. The IAT notes unavoidable instances of poor surface tracking quality which stem from glare, deep shadow, imprints in skin left by clothing, large changes in subject position or large change in skin pigmentation. Holes in the mesh of the QC image are expected in areas of strong bruising.

Primary Dimple Area of Interest (AOI) Delineation: Following successful surface tracking, an IAT opens the tracked Baseline image and registers Day 1-Post Marking image in VAM. On the Day 1-Post Marking image, the IAT traces the outer border of the primary dimple site marking. This tracing is used to measure surface area of the site marking, the largest axis across the site marking (Max Dimple Length) and the length of its largest perpendicular axis (Max Dimple Width). This tracing is not used to delineate an AOI for the Dimple or Bruise analysis. In cases where a primary dimple is not identified by the site, the IAT traces the dimple he/she perceived to be largest.

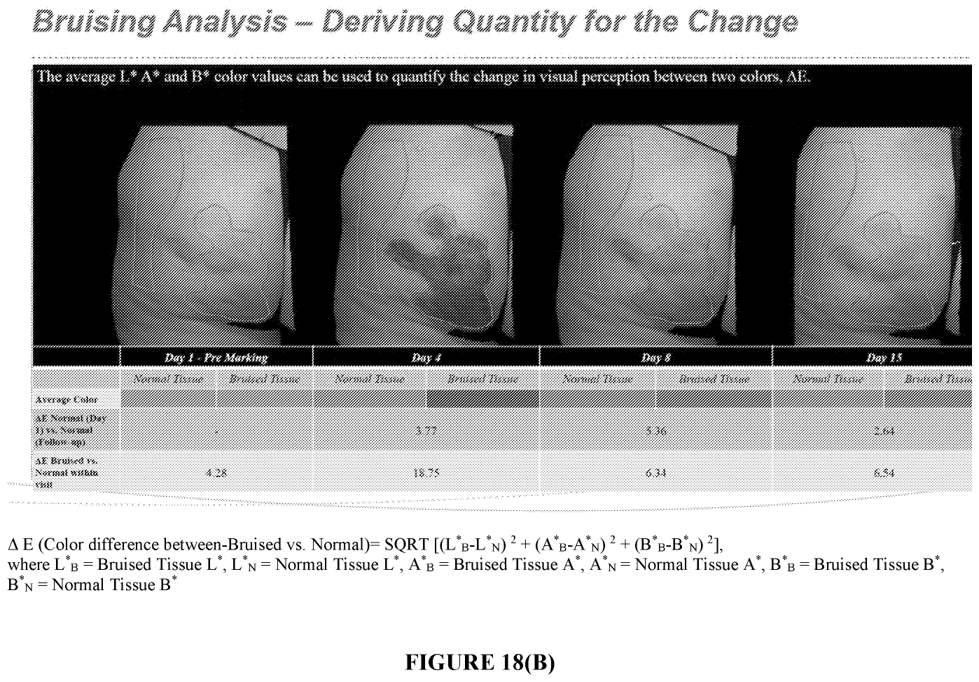

The IAT uses the Day 1-Post Marking image as a reference to locate the target dimple on the Day 1-Pre-Marking image. The IAT then traces the boundary of the primary dimple on the tracked, pre-marking image. The tracing is adjusted as necessary until it is just outside the ridge at which the dimple became concave and delineated the primary dimple AOI for dimple analysis. (FIG. 15). The dimple tracing on the tracked, pre-marking image is transposed onto the Day 22, Day 43 and Day 71 Follow-Up images based on each Follow-Up image's unique surface tracking relationship to the Baseline (FIG. 16). The IAT notes any instances of poor image quality affecting the transposed AOI.

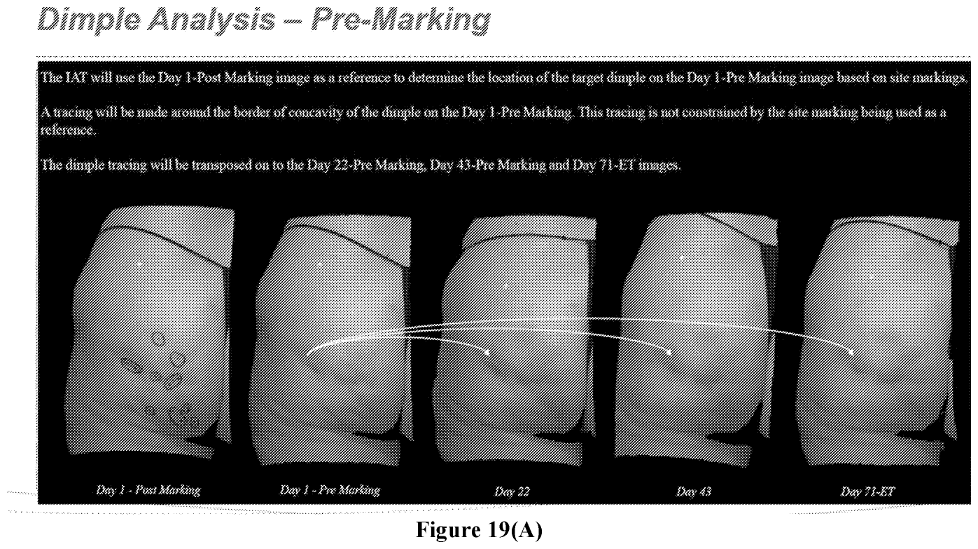

Bruise Area of Interest (AOI) Delineation: The IAT creates a “Bruised Tissue” AOI, a single continuous tracing around the perimeter of the largest perceptible bruise on the Day 4 image (FIG. 17). The IAT traces the perimeter where he/she can determine a difference in skin pigmentation between bruised skin and unaffected skin. Skin is considered bruised if it exhibits blue/purple, green, or yellow/brown pigmentation distinguishable from the surrounding skin tone. In the case where no bruise can be distinguished on the Day 4 image, the subject is failed for the purposes of the bruise analysis.

Additionally, the IAT creates a second tracing, the “Normal Tissue” AOL. The Normal Tissue AOI is created on an area of skin unaffected by skin texture distortions caused by clothing compression lines, abnormal redness, or skin features such as acne or scar tissue. Where possible and applicable, the Normal Tissue AOI attempts to match any shadowing, shine, or glare present across the curvature of the Day 1 Pre-Marking Bruised Tissue AOL. The size and shape of the Normal Tissue AOI varies across subjects depending on the amount of natural appearing skin available. Once traced, the IAT transposes both the Bruised Tissue and Normal Tissue AOI from the Day 4 image to the Day 1-Pre-Marking, Day 8 and Day 15 images based on their surface tracking relation to the Day 4 image. In the event that a Bruised Tissue AOI or Normal Tissue AOI extend beyond the edges of other bruise analysis visits for a particular subject or side of subject, the AOIs are adjusted until consistent across all visits.

The IAT manually traces the white reference label on the Day 1-Pre Marking, Day 4, Day 8 and Day 15 images and labeled the tracing “White.” If the white reference label is placed within the bruised area, the IAT traces the border of the label and subtracts it from the bruise AOI at each affected visit. In the case where no white reference label is present on an image, that image is failed for purposes of the bruise analysis.

Quality Control (QC) Checks: The QC checks are performed by a IAT independent of the IAT who performs analysis on the images. A separate set of QC checks is performed depending on the analysis procedure for a given visit. Any adjustments made by the QC IAT throughout the QC checks are noted and re-QC'd by another IAT before passing to analysis. Prior to beginning QC, the QC IAT reviews any notes made by the original IAT regarding image quality issues.

Analysis Measurements: After images pass QC, the analysis IAT reviews notes from the QC IAT. The analysis IAT then run a scripted analysis procedure. The procedure and analysis end points differ between images depending on the given visit. For dimple volume analysis, an interpolated surface is created across the top of the primary dimple AOL. A volume measurement represents the space between the interpolated surface and base of the dimple.

Surface area is measured as the total surface area of the primary dimple AOI or site tracing AOI in the case of the Day 1-Post Marking image. The Max Dimple Length is measured as the largest point to point axis across the primary dimple AOI. The Max Dimple Width is measured as the largest point-to-point distance perpendicular to the Max Dimple Length Axis.

Bruise analysis consists of two L*a*b* color measurements. L*a*b* color values are measured within the Bruised Tissue and Normal Tissue AOIs (FIG. 18(A)). The following table summarizes the analyses that may be performed (Table 19).

TABLE 19
Summary of the Bruise Analysis

| Analysis | | Analysis | |
|---|---|---|---|
| Category | Analysis Name | Units | Analysis Time Point |
| Dimple | Volume | cc | Day 1 Pre-Marking, Day |
| | | | 22, Day 43 and Day 71 |
| Dimple | Surface Area | mm² | Day 1 Pre-Marking, Day 1- |
| | | | Post Marking, Day 22, Day |
| | | | 43 and Day 71 |
| Dimple | Max Dimple Length | mm | Day 1 Pre-Marking, Day 1 |
| | (Max Length | | Post-Marking, Day 22, Day |
| | Straight Line) | | 43 and Day 71 |
| Dimple | Max Dimple Width | mm | Day 1 Pre-Marking, Day 1 |
| | (Max Perpendicular | | Post-Marking, Day 22, Day |
| | Width Straight Line) | | 43 and Day 71 |
| Color | Bruised Tissue L* | L* color | Day 1-Pre Marking, Day 4, |
| | (BruisedL) | value | Day 8, Day 15 |
| Color | Bruised Tissue a* | a* color | Day 1-Pre Marking, Day 4, |
| | (Bruiseda) | value | Day 8, Day 15 |
| Color | Bruise Tissue b* | b* color | Day 1-Pre Marking, Day 4, |
| | (Bruisedb) | value | Day 8, Day 15 |
| Color | Normal Tissue L* | L* color | Day 1-Pre Marking, Day 4, |
| | (Normal L) | value | Day 8, Day 15 |
| Color | Normal Tissue a* | a* color | Day 1-Pre Marking, Day 4, |
| | (Normala) | value | Day 8, Day 15 |
| Color | Normal Tissue b* | b* color | Day 1-Pre Marking, Day 4, |
| | (Normalb) | value | Day 8, Day 15 |

Accepting Data: Once image analysis is complete, the analyzing IAT either accepts the image or fails the image for analysis. Images can be partially failed, e.g., a Day 1 Pre-Marking visit may fail bruising analysis for no visible bruising at Day 4, but still be acceptable for dimple analysis. All failures receive a mandatory Failure Note detailing why the image model is unacceptable for analysis. Image failure may be due to factors such as poor image quality, poor tracking quality or obstructions within an AOL. Images are recommended for failure by the QC IAT and reviewed by the analyzing IAT. If the analysis IAT agrees with the failure recommendation, the image is failed.

Bruising Analysis. Bruised tissue and normal tissue are assessed on 3-D photographs using two L*A*B* color measurements at Day 1, 4, 8, and 15 (FIG. 18(B)). The greater the L*A*B* color intensity measurement, the worse the bruising. The change in visual perception between two colors of the bruised tissue versus the normal tissue for each treatment area and injection type are determined.

### G. DURABILITY OF THE EFFECT

The collagenase treatments described herein have durability in effectiveness as measured by any of the scales or assessment methods disclosed herein. Such durability may range from about 3 months to 5 years or longer. A single injection or series of injections can maintain an improvement in cellulite or continue to improve the appearance of cellulite or reduce the severity of the appearance of cellulite for a long period of time, e.g., about 6 months, 1 year, 2 years, 3 years, 4 years, or 5 years, or longer.

In a certain embodiment, patients receiving collagenase injections continue to exhibit a ≥1-point improvement from baseline for either or both the CR-PCSS and PR-PCSS score at about 6 months post-treatment, or have a ≥1-point improvement from baseline for either or both the CR-PCSS and PR-PCSS score at about 12 months after treatment. Further, such patients have a ≥1-point improvement from baseline for either or both the CR-PCSS and PR-PCSS score at about 22 days, 43 days, 90 days, or 180 days after treatment. For example, at least about 10%, or 15%, or 20%, or 25%, or 30%, or 35%, or 40%, or 45%, or 50%, or 55%, or 60%, or 65%, or 70%, or 75%, or 80%, or 85%, or 90%, or 95%, or 100% of patients demonstrate such durability.

In another aspect, the treatment method evaluates the durability of the effect of 2-level composite responders (patients that had an improvement of at least 2 levels of cellulite severity in both the PR-PCSS and the CR-PCSS), resulting in a statistically significant number demonstrating durability of effect at 6 months and 12 months post-treatment. In certain embodiments, at least about 10%, or 15%, or 20%, or 25%, or 30%, or 35%, or 40%, or 45%, or 50%, or 55%, or 60%, or 65%, or 70%, or 75%, or 80%, or 85%, or 90%, or 95%, or 100% of patients demonstrate such durability.

Non-limiting examples of durability include:
a. The decrease in the CR-PCSS ratings is maintained until Day 180 coupled with the scores on the Subject Satisfaction with Cellulite Treatment Scale support the effectiveness of 3 treatment sessions of CCH 0.84 mg administered as 12 subcutaneous injections per treatment area (×2 treatment areas) to either bilateral buttocks or bilateral thighs.
b. At Day 180, the decrease in the CR-PCSS rating from Baseline (Screening Visit) are consistent on the right and left sides.
c. A 2-level improvement in the CR-PCSS rating in at least 1 treatment area is observed at Day 180. Response is similar for the buttock and thigh regions and for left and right sides.
d. A 1-level improvement in the CR-PCSS rating in at least 1 area is observed at Day 180. Response is similar for the buttock and thigh regions and for left and right sides.
e. The median time to the earliest 2-level CR-PCSS response in at least 1 area is observed at 83 days.
f. The median time to the earliest 1-level CR-PCSS response is 41 days.
g. At Day 180 more than half of patients were either satisfied or very satisfied with treatment.
h. The period of time a patient's score on the Hexsel CSS was first classified as moderate (6-10) or mild (1-5) and continued to be classified as moderate (6-10) or mild (1-5) as compared to her baseline classification as severe (11-15).
i. The period of time measured from the date of a measured improvement to the date when the patient returns to baseline or worse after having demonstrated improvement as measured by one or more of the CR-PCSS, PR-PCSS, Hexsel CSS, Hexsel depression depth score, Likert Scale, dimple analysis, I-GAIS, S-GAIS, PR-CIS, PR-CIS Abbreviated, SSRS, SSCT, TCS-C, TCS-P, Body-Q, assessment by photography or other imagery, or any other validated photonumeric or other scale used by clinicians and/or patients to assess cellulite severity, improvement, and/or patient satisfaction.
j. A period of time measured from the date of measured improvement to the date when a subject has an observable loss of response to the treatment as measured by one or more of the CR-PCSS, PR-PCSS, Hexsel CSS, Hexsel depression depth score, Likert Scale, dimple analysis, I-GAIS, S-GAIS, PR-CIS, PR-CIS Abbreviated, SSRS, SSCT, TCS-C, TCS-P, Body-Q, assessment by photography or other imagery, or any other validated photonumeric or other scale used by clinicians and/or patients to assess cellulite severity, improvement, and/or patient satisfaction.
k. A period of time measured from the reference time point of response to treatment to when the subject has a change in response (including an improvement over initial response to treatment). Change in response and/or improvement can be measured by one or more of the CR-PCSS, PR-PCSS, Hexsel CSS, Hexsel depression depth score, Likert Scale, dimple analysis, I-GAIS, S-GAIS, PR-CIS, PR-CIS Abbreviated, SSRS, SSCT, TCS-C, TCS-P, Body-Q, assessment by photography or other imagery, or any other validated photonumeric or other scale used by clinicians and/or patients to assess cellulite severity, improvement, and/or patient satisfaction. In some embodiments, the reference time point is baseline (pre-dose, Day 1) or Day 71 after treatment.
l. A period of time measured from the visit date that a subject became a 2-level composite responder according to the CR-PCSS and PR-PCSS until the first date of 2 sequential visits at which the assessment ratings return and are sustained to baseline ratings.
m. A period of time measured from the visit date that a subject became a 1-level composite responder according to the CR-PCSS and PR-PCSS until the first date of 2 sequential visits at which the assessment ratings return and are sustained to baseline ratings.

### H. SAFETY OF COLLAGENASE INJECTIONS

The studies done to date, some of which are detailed in the Examples below, establish the safety of the treatments described herein. For example, these studies confirm the lack of systemic exposure of collagenase following concurrent, subcutaneous administration of 3.36 mg CCH in four treatment areas. In fact, no new concerns in the safety profile of collagenase were observed with concurrent administration in four treatment areas. The commonly reported events were consistent with the currently known adverse event profile of collagenase.

The majority of subjects treated with CCH experienced at least one TEAE that was mild to moderate in severity. There were no notable differences in the subjects experiencing a TEAE by thigh or buttock treated region. The most common type of TEAEs were injection site reactions, specifically injection site bruising, which did not differ between treatment region (buttock or thigh). Most TEAEs resolving within 21 days. There were no clinically meaningful changes in the clinical and hematology laboratory parameters, urinalysis results, vital signs or concomitant medications. There were no clinically relevant findings in subjects with anti-drug antibodies or neutralizing antibodies.

As described herein and shown in FIGS. 16-18, bruising analyses can be performed to measure the extent of bruising over time. In certain embodiments, any bruising caused by the collagenase treatments may resolve or significantly decrease in color intensity at about 3 days, or 4 days, or 5 days, or 6 days, or 7 days, or 8 days, or 9 days, or 10 days, or 11 days, or 12 days, or 13 days, or 14 days, or 15 days, or 20 days after a treatment visit.

### I. EXAMPLES

The following examples are included to demonstrate certain embodiments of the present disclosure. Those of skill in the art should, however, in light of the present disclosure, appreciate that modifications can be made in the specific embodiments that are disclosed and still obtain a like or similar result without departing from the spirit and scope of the invention. Therefore, all matter set forth is to be interpreted as illustrative and not in a limiting sense. For instance, in the studies employing CCH, other collagenases can be used in an amount sufficient (therapeutically effective amount) to produce the activity and response comparable to CCH. In the clinical trial results described below, it is to be understood that each numerical value reported is not intended to be strictly limiting. The scope of the present disclosure includes ranges around each value that are consistent with the facts and principles of the inventions described herein. Accordingly, each value may vary up or down by about 1%, 5%, 10%, 15%, 20%, 25%, 30%, 35%, 40%, 45%, 50%, 55%, 60%, 65%, 70%, 75%, 80%, 85%, 90%, 95%, or 100%. In some instances and in keeping with the context and circumstances of a particular value, it may vary up or down by about 125%, 150%, 175%, 200%, 225%, 250%, 275%, or 300%.

### Example 1-Clinical Study of Cch for the Treatment of Efp (Study 205)

An open-label Phase 2 study was performed in which subjects with mild, moderate, or severe levels of cellulite in at least 2 bilateral treatment regions (i.e., bilateral buttocks or bilateral posterolateral thighs) were administered CCH in the assigned treatment region on Days 1, 22, and 43. Follow-up visits were conducted at Days 90 and 180 for all subjects and in a subset of subjects, at 1, 3, 6, and 13 days after each treatment session. At Days 90 and 180 follow-up visits (i.e., 89 and 179 days after Day 1, respectively) occurred to assess treatment effectiveness (photographic sub-study). Unless otherwise specified in this example, “Days” as used in this study 205 were relative to the initial dose (Day 1) in study 205.

The CCH used was a sterile lyophilized powder comprising 0.9 mg of collagenase clostridium histolyticum, 0.5 mg of hydrochloric acid, 18.5 mg of sucrose, and 1.1 mg of tromethamine in a lyophilized cake. CCH sterile diluent for reconstitution was 0.03% calcium chloride dihydrate in 0.6% sodium chloride solution, 2.0 mL per vial.

Subjects were healthy, non-pregnant females 18 years of age or older with two bilateral treatment areas (i.e., both buttocks or both thighs) at the screening visit with a score of 2 (mild) or greater as reported by the clinician-reported photonumeric cellulite severity score (CR-PCSS), and a Hexsel Cellulite Severity Scale (CSS) score no greater than 13. Subjects were excluded if they exhibited any one of the following conditions: coagulation disorder; evidence or history of malignancy (other than basal-cell carcinoma) unless there has been no recurrence in at least 5 years; history of keloidal scarring or abnormal wound healing; or concurrent diseases or conditions that might have interfered with the conduct of the study, confounded the interpretation of the study results, or endangered the subject's well-being.

Subjects were administered a maximum dose of 1.68 mg of CCH per treatment session. A dose of 0.84 mg CCH was administered as 12 subcutaneous injections per treatment area (i.e., each buttock or each thigh). CCH was administered to each subject during three treatment sessions, each occurring at least 21 days apart. The cumulative dose of CCH was 5.04 mg (i.e., three treatments visits×0.84 mg per treatment area×2 treatment areas).

For each dimple selected for treatment, injection sites were chosen. Each injection site was marked with a dot using a surgical marker. For round dimples, the dot was placed in the center of the dimple. For elongated dimples, dots were spaced out approximately 2 cm along the longer axis of the dimple. If a dimple required more than 1 injection, injection sites within the dimple were spaced approximately 2 cm apart, locating at least one injection site at the nadir of the dimple, if present. The surgical marker was then used to circle each of the dimples selected for treatment. Circles in the selected treatment area did not overlap. See, e.g., FIG. 6.

CCH was injected subcutaneously while the subject was in a prone position using a syringe with a 30-gauge ½-inch needle. As shown in FIG. 7, each injection site received a single skin injection of CCH administered as three 0.1 mL aliquots to Positions A, B, and C, for a total injection volume of 0.3 mL. The depth of injection was 12 inch, corresponding to the length of the treatment needle from the tips of the needle to the base of the needle without downward pressure. At each injection site, the needle was positioned at 900 perpendicular to the skin surface and inserted, and 0.1 mL aliquot of CCH was injected (Position A). The needle was withdrawn slightly (but not removed from the skin) and repositioned 450 off vertical towards the head and above the long axis of the dimple, and 0.1 mL aliquot of CCH was injected (Position B). The needle was again withdrawn slightly and repositioned approximately 45° off vertical towards the feet and below the long axis of the dimple, and 0.1 mL aliquot of CCH was injected (Position C). After injection, the subject remained prone for 5 minutes.

Efficacy. The efficacy of CCH for the treatment of cellulite was analyzed by the clinician using the CR-PCSS rating. The maximum decrease in the CR-PCSS rating from the baseline visit (screening visit) was observed first at Day 90. This improvement in cellulite severity (i.e., a negative change) was maintained at the Day 180 visit. The majority of subjects had mild-to-moderate CR-PCSS rating scores at study entry. At Day 90, the mean (SD) change in the CR-PCSS rating from baseline of the left buttock and left thigh was −0.8 (0.58) and −0.6 (0.62), respectively, and in the right buttock and right thigh was −0.7 (0.73) and −0.5 (0.70), respectively. CR-PCSS scores by region and visit are provided in Table 20.

TABLE 20
CR-PCSS ratings and change from baseline by visit.

CCH (1.68 mg)

| | | | |
|---|---|---|---|
| | | Buttock | Thigh |
| CR-PCSS Rating | Statistic | (N = 62) | (N = 82) |

Left Side
Baseline

| | | | |
|---|---|---|---|
| None (0) | n (%) | 0 (0.0) | 0 (0.0) |
| Almost none (1) | n (%) | 0 (0.0) | 0 (0.0) |
| Mild (2) | n (%) | 23 (37.1) | 43 (52.4) |
| Moderate (3) | n (%) | 33 (53.2) | 34 (41.5) |
| Severe (4) | n (%) | 6 (9.7) | 5 (6.1) |
| Not Done | n (%) | 0 (0.0) | 0 (0.0) |
| | Mean | 2.7 | 2.5 |
| | SD | 0.63 | 0.61 |

Day 22

| | | |
|---|---|---|
| Mean | 2.3 | 2.3 |
| SD | 0.84 | 0.65 |

Change from baseline

| | | |
|---|---|---|
| Mean | −0.4 | −0.3 |
| SD | 0.70 | 0.53 |

Day 43

| | | |
|---|---|---|
| Mean | 2.1 | 2.1 |
| SD | 0.70 | 0.70 |

Change from baseline

| | | |
|---|---|---|
| Mean | −0.6 | −0.5 |
| SD | 0.56 | 0.58 |

Day 90

| | | |
|---|---|---|
| Mean | 2.0 | 1.9 |
| SD | 0.79 | 0.67 |

Change from baseline

| | | |
|---|---|---|
| Mean | −0.8 | −0.6 |
| SD | 0.58 | 0.62 |

Day 180

| | | |
|---|---|---|
| Mean | 1.9 | 1.9 |
| SD | 0.86 | 0.82 |

Change from baseline

| | | |
|---|---|---|
| Mean | −0.8 | −0.6 |
| SD | 0.65 | 0.69 |

Right Side
Baseline

| | | | |
|---|---|---|---|
| None (0) | n (%) | 0 (0.0) | 0 (0.0) |
| Almost none (1) | n (%) | 0 (0.0) | 0 (0.0) |
| Mild (2) | n (%) | 22 (35.5) | 38 (46.3) |
| Moderate (3) | n (%) | 34 (54.8) | 38 (46.3) |
| Severe (4) | n (%) | 6 (9.7) | 6 (7.3) |
| Not Done | n (%) | 0 (0.0) | 0 (0.0) |
| | Mean | 2.7 | 2.6 |
| | SD | 0.63 | 0.62 |

Day 22

| | | |
|---|---|---|
| Mean | 2.4 | 2.3 |
| SD | 0.79 | 0.70 |

Change from baseline

| | | |
|---|---|---|
| Mean | −0.3 | −0.3 |
| SD | 0.54 | 0.54 |

Day 43

| | | |
|---|---|---|
| Mean | 2.1 | 2.1 |
| SD | 0.75 | 0.76 |

Change from baseline

| | | |
|---|---|---|
| Mean | −0.6 | −0.5 |
| SD | 0.56 | 0.62 |

Day 90

| | | |
|---|---|---|
| Mean | 2.0 | 1.9 |
| SD | 0.76 | 0.77 |

Change from baseline

| | | |
|---|---|---|
| Mean | −0.8 | −0.7 |
| SD | 0.64 | 0.61 |

Day 180

| | | |
|---|---|---|
| Mean | 2.0 | 2.1 |
| SD | 0.87 | 0.86 |

Change from baseline

| | | |
|---|---|---|
| Mean | −0.7 | −0.5 |
| SD | 0.73 | 0.70 |

SD = standard deviation

Sixty-nine subjects had their bilateral buttocks treated, and eighty-nine subjects had their bilateral thighs treated. A 2-level improvement in the severity of cellulite on the CR-PCSS score from baseline was observed in at least one treatment in 17 (13.4%) of the evaluable subjects at Day 90 and 20 (15.3%) of the evaluable subjects (62 subjects treated in buttocks and 82 subjects treated in thighs) at Day 180. The median time to achieve the earliest 2-level response in at least one treatment area was 83 days. The proportion of 2-level CR-PCSS responders and the median time for a 2-level CR-PCSS response was similar for subjects treated on the left side and the right side and similar for the thigh and buttock treated regions. These results are provided in Table 21.

TABLE 21
Two-level responders in CR-CPSS ratings.

| | | CCH 1.68 mg | | |
|---|---|---|---|---|
| Two-Level | | Buttock | Thigh | Overall |
| Responders | Statistic | (N = 62) | (N = 82) | (N = 144) |

Left
Day 22

| | | | | | | | |
|---|---|---|---|---|---|---|---|
| Yes | n (%) | 3 | (5.1) | 1 | (1.3) | 4 | (2.9) |
| No | n (%) | 56 | (94.9) | 77 | (98.7) | 133 | (97.1) |

Day 43

| | | | | | | | |
|---|---|---|---|---|---|---|---|
| Yes | n (%) | 1 | (1.7) | 3 | (4.1) | 4 | (3.0) |
| No | n (%) | 58 | (98.3) | 71 | (95.9) | 129 | (97.0) |

Day 90

| | | | | | | | |
|---|---|---|---|---|---|---|---|
| Yes | n (%) | 4 | (7.1) | 4 | (5.6) | 8 | (6.3) |
| No | n (%) | 52 | (92.9) | 67 | (94.4) | 119 | (93.7) |

Day 180

| | | | | | | | |
|---|---|---|---|---|---|---|---|
| Yes | n (%) | 6 | (10.7) | 7 | (9.3) | 13 | (9.9) |
| No | n (%) | 50 | (89.3) | 68 | (90.7) | 118 | (90.1) |

Time to earliest 2-level responder (days)

| | | | |
|---|---|---|---|
| N | 7 | 9 | 16 |
| Mean | 54.0 | 108.8 | 84.8 |
| SD | 33.38 | 72.59 | 63.59 |
| Median | 44.0 | 85.0 | 66.5 |
| Minimum | 21 | 23 | 21 |
| Maximum | 91 | 191 | 191 |

Right
Day 22

| | | | | | | | |
|---|---|---|---|---|---|---|---|
| Yes | n (%) | 2 | (3.4) | 2 | (2.6) | 4 | (2.9) |
| No | n (%) | 57 | (96.6) | 76 | (97.4) | 133 | (97.1) |

Day 43

| | | | | | | | |
|---|---|---|---|---|---|---|---|
| Yes | n (%) | 1 | (1.7) | 5 | (6.8) | 6 | (4.5) |
| No | n (%) | 58 | (98.3) | 69 | (93.2) | 127 | (95.5) |

Day 90

| | | | | | | | |
|---|---|---|---|---|---|---|---|
| Yes | n (%) | 6 | (10.7) | 6 | (8.5) | 12 | (9.4) |

| | | | | | | |
|---|---|---|---|---|---|---|
| No | n (%) | 50 *89.3) | 65 | (91.5) | 115 | (90.6) |

Day 180

| | | | | | | | |
|---|---|---|---|---|---|---|---|
| Yes | n (%) | 8 | (14.3) | 7 | (9.3) | 15 | (11.5) |
| No | n (%) | 50 | (89.3) | 65 | (91.5) | 115 | (90.6) |

Time to earliest 2-level responder (days)

| | | | |
|---|---|---|---|
| N | 11 | 11 | 22 |
| Mean | 97.5 | 80.2 | 88.9 |
| SD | 59.86 | 57.38 | 57.90 |
| Median | 85.0 | 61.0 | 84.5 |
| Minimum | 21 | 21 | 21 |
| Maximum | 184 | 191 | 191 |

At least one of two areas (left and right)
Day 22

| | | | | | | | |
|---|---|---|---|---|---|---|---|
| Yes | n (%) | 3 | (5.1) | 3 | (3.8) | 6 | (4.4) |
| No | n (%) | 56 | (94.9) | 75 | (96.2) | 131 | (95.6) |

Day 43

| | | | | | | | |
|---|---|---|---|---|---|---|---|
| Yes | n (%) | 2 | (3.4) | 6 | (8.1) | 8 | (6.0) |
| No | n (%) | 57 | (96.6) | 68 | (91.9) | 125 | (94.0) |

Day 90

| | | | | | | | |
|---|---|---|---|---|---|---|---|
| Yes | n (%) | 8 | (14.3) | 9 | (12.7) | 17 | (13.4) |
| No | n (%) | 48 | (85.7) | 62 | (87.3) | 110 | (86.6) |

Day 180

| | | | | | | | |
|---|---|---|---|---|---|---|---|
| Yes | n (%) | 11 | (19.6) | 9 | (12.0) | 20 | (15.3) |
| No | n (%) | 45 | (80.4) | 66 | (88.0) | 111 | (84.7) |

Time to earliest 2-level responder (days)

| | | | |
|---|---|---|---|
| N | 13 | 15 | 28 |
| Mean | 80.6 | 80.9 | 80.8 |
| SD | 53.19 | 57.63 | 54.58 |
| Median | 84.0 | 61.0 | 83.0 |
| Minimum | 21 | 21 | 21 |
| Maximum | 184 | 191 | 191 |

Approximately three-quarters of subjects experienced at least a 1-level response in at least one treatment area by Day 90. A 1-level improvement in the severity of cellulite on the CR-PCSS from baseline in at least 1 treatment area was observed in 96 (75.6%) evaluable subjects at Day 90 and 90 (68.7%) evaluable subjects at Day 180. The median time to achieve the earliest 1-level response in at least 1 area was observed at 41 days. The proportion of 1-level CR-PCSS responders for subjects treated on the left and right side was similar and similar for thigh and buttock treated regions. The median time to achieve the earliest 1-level CR-PCSS response on the left and right sides was identical at 43 days. These results are summarized in Table 22.

TABLE 22
One-level responders in CR-CPSS ratings

| | | CCH 1.68 mg | | |
|---|---|---|---|---|
| One-Level | | Buttock | Thigh | Overall |
| Responders | Statistic | (N = 62) | (N = 82) | (N = 144) |

Left
Day 22

| | | | | |
|---|---|---|---|---|
| Yes | n (%) | 21 (35.6) | 22 (28.2) | 43 (31.4) |
| No | n (%) | 38 (64.4) | 56 (71.8) | 94 (68.6) |

Day 43

| | | | | |
|---|---|---|---|---|
| Yes | n (%) | 36 (61.0) | 33 (44.6) | 69 (51.9) |
| No | n (%) | 23 (39.0) | 41 (55.4) | 64 (48.1) |

Day 90

| | | | | |
|---|---|---|---|---|
| Yes | n (%) | 38 (67.9) | 42 (59.2) | 80 (63.0) |
| No | n (%) | 18 (32.1) | 29 (40.8) | 47 (37.0) |

Day 180

| | | | | |
|---|---|---|---|---|
| Yes | n (%) | 39 (69.6) | 43 (57.3) | 82 (62.6) |
| No | n (%) | 17 (30.4) | 32 (42.7) | 49 (37.4) |

Time to earliest 1-level responder (days)

| | | | |
|---|---|---|---|
| N | 51 | 58 | 109 |
| Mean | 56.0 | 61.0 | 58.7 |
| DS | 49.22 | 47.83 | 48.32 |
| Median | 42.0 | 43.0 | 43.0 |
| Minimum | 20 | 20 | 20 |
| Maximum | 205 | 186 | 205 |

Right
Day 22

| | | | | |
|---|---|---|---|---|
| Yes | n (%) | 18 (30.5) | 23 (29.5) | 41 (29.9) |
| No | n (%) | 41 (69.5) | 55 (70.5) | 96 (70.1) |

Day 43

| | | | | |
|---|---|---|---|---|
| Yes | n (%) | 36 (61.0) | 34 (45.9) | 70 (52.6) |
| No | n (%) | 23 (39.0) | 40 (54.1) | 63 (47.4) |

Day 90

| | | | | |
|---|---|---|---|---|
| Yes | n (%) | 36 (64.3) | 45 (63.4) | 81 (63.8) |
| No | n (%) | 20 (35.7) | 26 (36.6) | 46 (36.2) |

Day 180

| | | | | |
|---|---|---|---|---|
| Yes | n (%) | 34 (60.7) | 36 (48.0) | 70 (53.4) |
| No | n (%) | 22 (39.3) | 39 (52.0) | 61 (46.6) |

Time to earliest 1-level responder (days)

| | | | |
|---|---|---|---|
| N | 48 | 58 | 106 |
| Mean | 50.9 | 54.2 | 52.7 |
| DS | 41.78 | 35.37 | 38.25 |
| Median | 42.5 | 43.0 | 43.0 |
| Minimum | 20 | 20 | 20 |
| Maximum | 205 | 186 | 205 |

At least one of two areas (left and right)
Day 22

| | | | | |
|---|---|---|---|---|
| Yes | n (%) | 24 (40.7) | 31 (39.7) | 55 (40.1) |
| No | n (%) | 35 (59.3) | 47 (60.3) | 82 (59.9) |

Day 43

| | | | | |
|---|---|---|---|---|
| Yes | n (%) | 43 (72.9) | 44 (59.5) | 87 (65.4) |
| No | n (%) | 16 (27.1) | 30 (40.5) | 46 (34.6) |

Day 90

| | | | | |
|---|---|---|---|---|
| Yes | n (%) | 42 (75.0) | 54 (76.1) | 96 (75.6) |
| No | n (%) | 14 (25.0) | 17 (23.9) | 31 (24.4) |

Day 180

| | | | | |
|---|---|---|---|---|
| Yes | n (%) | 40 (71.4) | 50 (66.7) | 90 (68.7) |
| No | n (%) | 16 (28.6) | 25 (33.3) | 41 (31.3) |

Time to earliest 1-level responder (days)

| | | | |
|---|---|---|---|
| N | 53 | 67 | 120 |
| Mean | 46.9 | 50.4 | 48.8 |
| DS | 40.76 | 38.85 | 39.57 |
| Median | 41.0 | 42.0 | 41.0 |
| Minimum | 20 | 20 | 20 |
| Maximum | 205 | 186 | 205 |

The efficacy of CCH for the treatment of cellulite was also analyzed by the subjects using subject satisfaction scores (Subject Satisfaction with Cellulite Treatment Scale). At Day 180, of the 130 evaluable responders, more than half (56.10%) were either satisfied or very satisfied with treatment. The proportion of subjects treated that were satisfied or very satisfied with the treatment in the buttocks was 71.5%. The proportion of subjects that were satisfied or very satisfied with the treatment in the thighs was 44.6%. These results are summarized in Table 23.

TABLE 23
Subject satisfaction with CCH treatment
assessed at the end of the study.

| | | CCH 1.68 mg | | | | | |
|---|---|---|---|---|---|---|---|
| | | Buttock | | Thigh | | Overall | |
| Subject Response | Statistic | (N = 62) | | (N = 82) | | (N = 144) | |
| Very satisfied (2) | N (%) | 16 | (28.6) | 9 | (12.2) | 25 | (19.2) |
| Satisfied (1) | N (%) | 24 | (42.9) | 24 | (32.4) | 48 | (36.9) |
| Neither satisfied nor | N (%) | 9 | (16.1) | 23 | (31.1) | 32 | (24.6) |
| dissatisfied (0) | | | | | | | |
| Dissatisfied (−1) | N (%) | 4 | (7.1) | 8 | (10.8) | 12 | (9.2) |
| Very dissatisfied | N (%) | 3 | (5.4) | 10 | (13.5) | 13 | (10.0) |
| (−2) | | | | | | | |

| | | | |
|---|---|---|---|
| Mean | 0.8 | 0.2 | 0.5 |
| SD | 1.10 | 1.20 | 1.20 |

Other conclusions regarding efficacy include the following:

1. At Day 180, the decrease in the CR-PCSS rating from Baseline (Screening Visit) was consistent on the right and left sides. The change in the mean (SD) CR-PCSS rating from Baseline of the left buttock and thigh (−0.8 [0.65] and −0.6 [0.69] versus the right buttock and thigh (−0.7 [0.73] and −0.5 [0.70]) was similar.

2. A 2-level improvement in the CR-PCSS rating in at least 1 area was observed in 17 (13.4%) evaluable subjects at Day 90 and 20 (15.3%) evaluable subjects at Day 180. Response was similar for the buttock and thigh regions and for left and right sides.

3. A 1-level improvement in the CR-PCSS rating in at least 1 treatment area was observed in 96 (75.6%) evaluable subjects at Day 90 and 90 (68.7%) evaluable subjects at Day 180. Response was similar for the buttock and thigh regions and for left and right sides.

4. The median time to the earliest 2-level CR-PCSS response in at least 1 treatment area was observed at 83 days (range: 21, 191). The median time to the earliest 1 level CR PCSS response in at least 1 treatment area was observed at 41 days (range: 20, 205).

5. At Day 180 (end of study/end of treatment), more than half of evaluable responders (56.1%) were either satisfied or very satisfied with treatment (satisfied: 48 [36.9%]) or very satisfied: (25 [19.2%]).

6. The decrease in the CR-PCSS ratings sustained at Day 180 coupled with the scores on the subject satisfaction survey demonstrates that three treatment sessions of 0.84 mg CCH administered as 12 subcutaneous injections per treatment are (×2 treatment areas) to either bilateral buttocks or bilateral thighs was effective.

Safety. The majority of subjects treated with CCH experienced at least one treatment-emergent adverse event that was mild to moderate in severity. There were no notable differences in the subjects experiencing a treatment-emergent adverse event by thigh or buttock treated region. The most common type of adverse events were injection site reactions, specifically injection site bruising, which did not differ between treatment region.

The study also includes a photographic sub-study during which subjects returned to the clinic for photographs at 1, 3, 6, and 13 days after each treatment course to coincide with the follow up visits. Photographic images of the thigh and buttock regions of the 37 subjects participating in the photographic sub-study at the 2 study sites were arranged by site, by subject, chronologically by study visit, and cumulatively as collages of the respective treatment area (left buttock, right buttock, left thigh and right thigh). Investigators completed a questionnaire designed to capture overall observations of the appearance of injection site bruising after review of the images and photo collages. Investigators observed that injection site bruising was severe at 3 and 6 days after the treatment session, resolved before the next treatment session, and generally became less severe with each treatment session. Discoloration that persisted was attributed to hemosiderin staining.

Analysis of treatment-related injection site reaction by treatment sessions showed a trend of a decreasing incidence and duration of injection site reactions with subsequent treatment sessions, with most resolving within 21 days. During Treatment Sessions 1, 2, and 3, the proportion of subject experiencing treatment-emergent adverse events that resolved within 21 days or less was 76.7%, 85.2%, and 71% respectively.

### Example 2—Phase 3 Clinical Study of Cch for the Treatment of Efp (Study 302)

A Phase 3, randomized, double-blind, placebo-controlled study was performed in which subjects with moderate or severe levels of cellulite in each buttock were administered CCH in the assigned treatment region on Days 1, 22, and 43. Unless otherwise specified in this example, “Days” as used in this study 302 were relative to the initial dose (Day 1) in study 302. The CCH for cellulite composition was a sterile lyophilized powder comprising 0.92 mg of collagenase clostridium histolyticum, sucrose, Tris, mannitol, and hydrochloric acid qs to pH 8.5. CCH sterile diluent for reconstitution as 0.6% sodium chloride and 0.03% calcium chloride dehydrate in water. Subjects were assessed at Days 1, 22, 43 and 71. Subjects were healthy, non-pregnant females 18 years of age or older having a score of 3 or 4 (moderate or severe) at the screening visit and at the first treatment session prior to treatment using both the patient-reported photonumeric cellulite severity score (PC-PCSS) and clinician-reported photonumeric cellulite severity score (CR-PCSS).

Subjects were administered with a maximum dose of 1.68 mg of CCH per treatment session or placebo. A dose of 0.84 mg CCH was administered as 12 subcutaneous injections per buttock. CCH was administered to each subject during three treatment sessions, each occurring approximately 21 days apart. The cumulative dose of CCH was 5.04 mg (i.e., three treatments visits×0.84 mg per treatment area×2 treatment areas). CCH was injected in the same manner described in Example 1 and illustrated in FIG. 7. Both buttocks of a subject received either CCH treatment or placebo treatment depending on which treatment group to which they were randomly assigned.

The following subject populations were among the populations that were analyzed:

The Intent-to-Treat (ITT) Population included all randomized subjects who had at least 1 injection of study drug. All demographic and baseline characteristic summaries were based on this population. The primary and key secondary efficacy parameters were based on this population.

The Modified Intent-to-Treat (mITT) Population included all ITT subjects with a baseline and at least 1 post-injection evaluation of both the CR-PCSS and PR-PCSS for both the target and non-target buttocks. All secondary and supportive efficacy evaluations were based on the mITT Population.

The clinician selected dimples within each buttock that were well-defined, evident when the subject was standing, and suitable for treatment. Because the goal of the treatment was to improve the aesthetic appearance of each entire buttock, the clinician was instructed to select dimples that would likely improve the aesthetic appearance of each entire buttock. The same dimple within a buttock or different dimples with a buttock could be treated at each treatment session but injection must have been within the buttocks. Each buttock received all three treatments unless it had no treatable cellulite dimples and the clinician rated the buttock a score of 0 on the CR-PCSS. If no injections in a particular buttock were given at the second treatment sessions, subjects were still assessed for treatment in the contralateral buttock and returned for the third treatment session, and each of the buttocks was again evaluated by the subject and clinician. If the clinician rated either or both of the buttocks greater than 0 on the CR-PCSS, injections during the third treatment session were given.

All subjects received all 12 injections in each buttock during Treatment Session 1. In Treatment Session 2, 88.1% of subjects in the CCH-treated group and 94.8% of placebo-treated subjects received 12 injections in each buttock. In Treatment Session 3, 89.0% of CCH-treated subjects and 90.6% of placebo-treated subjects received 12 injections in each buttock. The number of dimples treated and the mean number of injections per dimple were similar between the two treatment groups and across treatment sessions. Efficacy was analyzed using the following cellulite severity rating systems: (i) PR-PCSS; (ii) CR-PCSS; (iii) S-GAIS; (iv) I-GAIS; (v) PR-CIS; (vi) SSRS; and (vii) Subject Global Satisfaction with Cellulite Treatment (SSCT). Subject, investigator and staff were blinded to both the target buttock and the treatment. Assessments were done independently by subject and investigator and they were blinded to each other's scores. The primary efficacy variable was the proportion of 2-level composite responders at Day 71 defined as subjects with: (1) An improvement in severity from baseline (Day 1) of at least 2 levels of severity in the CR-PCSS as assessed live by the investigator of the target buttock; and (2) an improvement in severity from baseline (Day 1) of at least 2 levels of severity in the PR-PCSS as assessed by the subject while viewing the digital image of the target buttock. A subject was considered a responder if these criteria were met in the randomized target buttock of that subject.

The definitions of responders used in the study were as follows.

PR-PCSS Responders. A 2 level PR-PCSS responder was defined as a subject with improvement in PR-PCSS rating of at least 2 levels from baseline (change of −2, −3 or −4) at an evaluation time point. A 1 level PR-PCSS responder was defined as a subject with improvement in PR-PCSS rating of at least 1 level from baseline (change of −1, −2, −3, or −4) at an evaluation time point.

CR-PCSS Responders. A 2 level CR-PCSS responder was defined as a subject with improvement in CR-PCSS rating of at least 2 levels from baseline (change of −2, −3, or −4) at an evaluation time point. A 1 level CR-PCSS responder was defined as a subject with improvement in CR-PCSS rating of at least 1 level from baseline (change of −1, −2, −3, or −4) at an evaluation time point.

Composite PR-PCSS/CR-PCSS Responders. The PR PCSS responder classification and CR PCSS responder classification for each buttock were used to compute the composite responder classification for that buttock. If the classification was missing for 1 or both components (i.e., the PR PCSS component or the CR PCSS component), then the composite responder classification was missing for that visit. A 2 level composite responder was defined as a subject who is both a 2-level PR-PCSS responder and a 2-level CR-PCSS responder at an evaluation time point. A 1 level composite responder was defined as a subject who was both a 1-level PR-PCSS responder and a 1-level CR-PCSS responder at an evaluation time point.

S-GAIS Responders. A 2-level S-GAIS responder was defined as a subject with an S-GAIS rating of at least 2 (2 or 3) at an evaluation time point. A 1-level S-GAIS responder was defined as a subject with an S-GAIS rating of at least 1 (1, 2, or 3) at an evaluation time point.

I-GAIS Responders. A 2-level I-GAIS responder was defined as a subject with an I-GAIS rating of at least 2 (2 or 3) at an evaluation time point. A 1-level I-GAIS responder was defined as a subject with an I-GAIS rating of at least 1 (1, 2, or 3) at an evaluation time point.

PR-CIS Responders. For PR-CIS total score, a responder was defined as a subject with a reduction in the PR-CIS total score of at least 12 from baseline at an evaluation time point. For PR-CIS abbreviated score, a responder was defined as a subject with a reduction in the PR CIS abbreviated score of at least 10 from baseline at an evaluation time point. For individual PR-CIS impact scores, response was defined as improvement from baseline of at least 2 score intervals at each time point.

SSRS Responders. A 1-level SSRS responder was defined as a subject who was at least slightly satisfied (slightly satisfied [4], very satisfied [5], or extremely satisfied [6]) with the appearance of the cellulite on her buttocks at the Day 71/Early Termination Visit.

Subject Global Satisfaction with Cellulite Treatment (SSCT). A subject with a response of “Satisfied” or “Very Satisfied” on the Subject Satisfaction with Cellulite Treatment assessment at the Day 71/Early Termination Visit was considered a responder.

The primary endpoint was the proportion of 2-level CR-PCSS/PR-PCSS composite responders in the target buttock at Day 71 in the intent to treat (ITT) Population. A 2-level composite responder was defined as a subject with an improvement from baseline of at least 2 levels of severity in the CR-PCSS and an improvement from baseline of at least 2 levels of severity in the PR-PCSS.

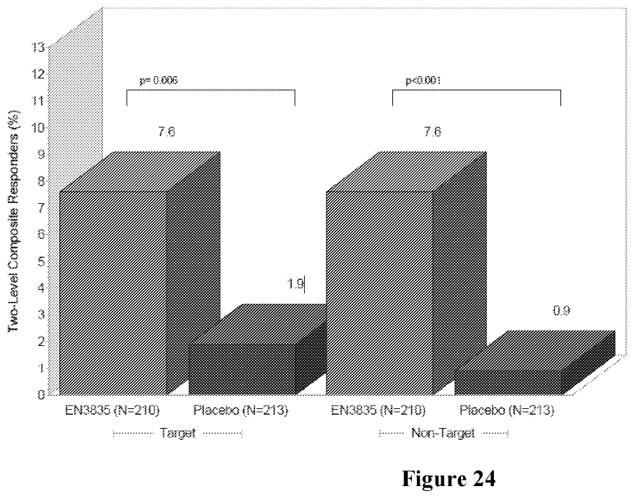

In this study, a statistically significant difference (p=0.006) was seen in the proportion of 2 level CR-PCSS/PR-PCSS composite responders in CCH treated subjects (16 [7.6%]) compared to placebo treated subjects (4 [1.9%]), Table 24 and FIG. 24.

TABLE 24
Two-level Composite Responders for the Target
Buttock on Day 71 (ITT Population)

| | | Study Drug | | | | |
|---|---|---|---|---|---|---|
| | | CCH 0.84 mg per | | | | |
| | | Buttock (1.68 mg | | | | |
| | | Total Dose) | | Placebo | | |
| Endpoint | Statistic | (N = 210) | | (N = 213) | | p-value^{a} |
| Two-level | | | | | | |
| Composite | | | | | | |
| Responder | | | | | | |
| Yes | n (%) | 16 | (7.6) | 4 | (1.9) | 0.006 |
| No | n (%) | 194 | (92.4) | 209 | (98.1) | |

^{a}p-value was based on CMH test adjusted for analysis center.

There were 3 families of endpoints that included 8 key secondary endpoints in this study. The differences between CCH and placebo treated subjects favored CCH in all 8 endpoints, and all differences were statistically significant. These are summarized in Table 25 below.

TABLE 25
Summary of Results for 8 Key Secondary Endpoints (ITT Population)

| | | Frequency of Responders | | | | Treatment Difference | |
|---|---|---|---|---|---|---|---|
| Secondary | | CCH | | Placebo | | | Statistical |
| Family | Endpoint | (N = 210) | | (N = 213) | | p-value^{a} | Significance^{b} |
| 1 | Subject 1-Level PR-PCSS Responders of the Target Buttock at Day 71, n (%) | 114 | (54.3) | 77 | (36.2) | <.001 | * |
| 1 | Subject 2-Level PR-PCSS Responders of the Target Buttock at Day 71, n (%) | 51 | (24.3) | 26 | (12.2) | 0.001 | * |
| 1 | Subject/Investigator 1-Level Composite Responders of the Target Buttock at | 78 | (37.1) | 38 | (17.8) | <.001 | * |
| | Day 71, n (%) | | | | | | |
| 1 | Subject/Investigator 2-Level Composite Responders of the Non-target Buttock | 16 | (7.6) | 2 | (0.9) | <.001 | * |
| | at Day 71 Based on CR-PCSS and PR-PCSS, n (%) | | | | | | |
| 2 | Subject 1-Level SSRS Responders at Day 71, n (%) | 102 | (48.6) | 48 | (22.5) | <.001 | * |
| 2 | Change from Baseline (Day 1) of the PR-CIS Total Score at Day 71, mean (SD) | −10.9 | (12.51) | −5.9 | (11.62) | <.001 | * |
| 3 | Subject 1-Level S-GAIS Responders of Target Buttock at Day 71, n (%) | 135 | (64.3) | 82 | (38.5) | <.001 | * |
| 3 | Subject 2-Level S-GAIS Responders of Target Buttock at Day 71, n (%) | 49 | (23.3) | 13 | (6.1) | <.001 | * |

^{a}p-value from individual analysis without multiplicity adjustment.
^{b}* represents a family-wise statistical significance at significance level of 0.05 after multiplicity adjustment.

PR-PCSS ratings were obtained at each visit and the changes from baseline were examined. A negative change from baseline indicates an improvement in cellulite severity. At baseline, all subjects had PR-PCSS ratings of moderate or severe. As early as Day 22, the mean (SD) change from baseline in PR-PCSS was greater in subjects treated with CCH in the target buttock (−0.4 [0.67]) compared to subjects treated with placebo (−0.1 [0.45]). This difference was statistically significant (p<0.001). Similar results were obtained for the non-target buttock. The observed improvements in subjects treated with CCH versus placebo continued and remained statistically significant in the target and non-target buttock at all study visits, including at Day 71. These results are summarized in Table 26.

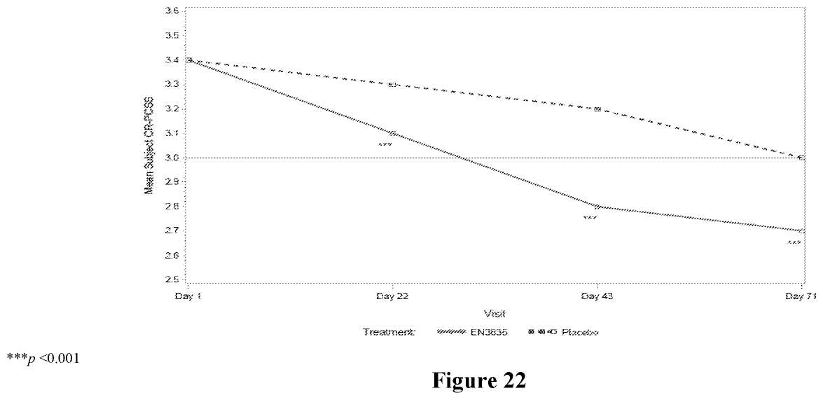

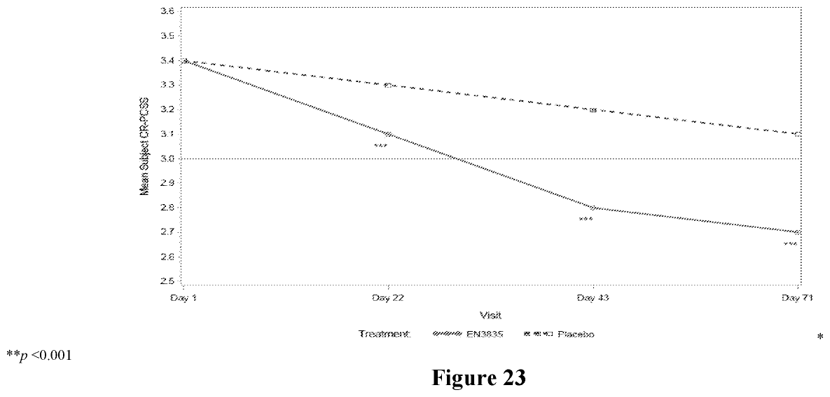

Analyses of PR-PCSS 1-level and 2-level responders for the target and non-target buttocks demonstrated statistically significant differences in improvement for subjects treated with CCH versus placebo. The proportion of 1-level PR-PCSS responders on Day 22 was greater in subjects treated with CCH than in placebo-treated subjects in the target buttock (34.8% vs. 16.8%, respectively) and the non-target buttock (34.8% vs. 18.8%, respectively). These differences were statistically significant (p<0.001) and continued throughout the study and remained statistically significant at each assessment. These results are summarized in Table 27 and shown in FIGS. 22-23.

TABLE 26
PR-PCSS rating and change from baseline for the target and non-target buttock by visit (mITT Population).

| | | Day 1 | | Day 22 | | Day 43 | |
|---|---|---|---|---|---|---|---|
| | | CCH | Placebo | CCH | Placebo | CCH | Placebo |
| PR-PCSS | Statistic | (N = 201) | (N = 205) | (N = 201) | (N = 205) | (N = 201) | (N = 205) |

Target buttock rating

| | | | | | | | | | | | | | |
|---|---|---|---|---|---|---|---|---|---|---|---|---|---|
| None (0) | n (%) | 0 | (0.0) | 0 | (0.0) | 0 | (0.0) | 0 | (0.0) | 0 | (0.0) | 1 | (0.5) |
| Almost none (1) | n (%) | 0 | (0.0) | 0 | (0.0) | 1 | (0.5) | 0 | (0.0) | 13 | (6.9) | 4 | (2.1) |
| Mild (2) | n (%) | 0 | (0.0) | 0 | (0.0) | 29 | (15.5) | 7 | (3.5) | 50 | (26.6) | 24 | (12.3) |
| Moderate (3) | n (%) | 84 | (41.8) | 82 | (40.0) | 79 | (42.2) | 95 | (47.0) | 75 | (39.9) | 95 | (48.7) |
| Severe (4) | n (%) | 117 | (58.2) | 123 | (60.0) | 78 | (41.7) | 100 | (49.5) | 50 | (26.6) | 71 | (36.4) |

| | | | | | | | |
|---|---|---|---|---|---|---|---|
| Not done | n (%) | — | — | 14 | 3 | 13 | 10 |
| | Mean | 3.6 | 3.6 | 3.3 | 3.5 | 2.9 | 3.2 |
| | SD | 0.49 | 0.49 | 0.73 | 0.57 | 0.89 | 0.76 |

Target buttock change from baseline

| | | | | | | | | | | | |
|---|---|---|---|---|---|---|---|---|---|---|---|
| −4 | n (%) | — | — | 0 | (0.0) | 0 | (0.0) | 0 | (0.0) | 1 | (0.5) |
| −3 | n (%) | — | — | 1 | (0.5) | 0 | (0.0) | 5 | (2.7) | 1 | (0.5) |
| −2 | n (%) | — | — | 8 | (4.3) | 1 | (0.5) | 28 | (14.9) | 12 | (6.2) |
| −1 | n (%) | — | — | 56 | (29.9) | 33 | (16.3) | 75 | (39.9) | 54 | (27.7) |
| 0 | n (%) | — | — | 113 | (60.4) | 160 | (79.2) | 72 | (38.3) | 122 | (62.6) |
| +1 | n (%) | — | — | 9 | (4.8) | 8 | (4.0) | 8 | (4.3) | 5 | (2.6) |

| | | | | | | | |
|---|---|---|---|---|---|---|---|
| Not done | n | — | — | 14 | 3 | 13 | 10 |
| | Mean | — | — | −0.4 | −0.1 | −0.7 | −0.4 |
| | SD | — | — | 0.67 | 0.45 | 0.86 | 0.72 |

| | | | | |
|---|---|---|---|---|
| p-value | — | — | <0.001 | <0.001 |

Non-target buttock rating

| | | | | | | | | | | | | | |
|---|---|---|---|---|---|---|---|---|---|---|---|---|---|
| None (0) | n (%) | 0 | (0.0) | 0 | (0.0) | 0 | (0.0) | 0 | (0.0) | 1 | (0.5) | 1 | (0.5) |
| Almost none (1) | n (%) | 0 | (0.0) | 0 | (0.0) | 2 | (1.1) | 0 | (0.0) | 12 | (6.4) | 4 | (2.1) |
| Mild (2) | n (%) | 0 | (0.0) | 0 | (0.0) | 27 | (14.4) | 9 | (4.5) | 50 | (26.6) | 33 | (16.9) |
| Moderate (3) | n (%) | 81 | (40.3) | 83 | (40.5) | 81 | (43.3) | 89 | (44.1) | 80 | (42.6) | 82 | (42.1) |
| Severe (4) | n (%) | 120 | (59.7) | 122 | (59.5) | 77 | (41.2) | 104 | (51.5) | 45 | (23.9) | 75 | (38.5) |

| | | | | | | | |
|---|---|---|---|---|---|---|---|
| Not done | n (%) | — | — | 14 | 3 | 13 | 10 |
| | Mean | 3.6 | 3.6 | 3.2 | 3.5 | 2.8 | 3.2 |
| | SD | 0.49 | 0.49 | 0.74 | 0.58 | 0.89 | 0.81 |

Non-target buttock change from baseline

| | | | | | | | | | | | |
|---|---|---|---|---|---|---|---|---|---|---|---|
| −4 | n (%) | — | — | 0 | (0.0) | 0 | (0.0) | 1 | (0.5) | 1 | (0.5) |
| −3 | n (%) | — | — | 0 | (0.0) | 0 | (0.0) | 2 | (1.1) | 2 | (1.0) |
| −2 | n (%) | — | — | 6 | (3.2) | 0 | (0.0) | 26 | (13.8) | 12 | (6.2) |
| −1 | n (%) | — | — | 59 | (31.6) | 38 | (18.8) | 88 | (46.8) | 56 | (28.7) |
| 0 | n (%) | — | — | 119 | (63.6) | 150 | (74.3) | 67 | (35.6) | 117 | (60.0) |
| +1 | n (%) | — | — | 3 | (1.6) | 14 | (6.9) | 4 | (2.1) | 7 | (3.6) |

| | | | | | | | |
|---|---|---|---|---|---|---|---|
| Not done | n | — | — | 14 | 3 | 13 | 10 |
| | Mean | — | — | −0.4 | −0.1 | −0.8 | −0.4 |
| | SD | — | — | 0.57 | 0.49 | 0.79 | 0.75 |

| | | | | |
|---|---|---|---|---|
| p-value | — | — | <0.001 | <0.001 |

| | | Day 71 | | | | Day 71 (LOCF) | | | |
|---|---|---|---|---|---|---|---|---|---|
| | | CCH | | Placebo | | CCH | | Placebo | |
| PR-PCSS | Statistic | (N = 201) | | (N = 205) | | (N = 201) | | (N = 205) | |
| Target buttock rating | | | | | | | | | |
| None (0) | n (%) | 0 | (0.0) | 2 | (1.1) | 0 | (0.0) | 2 | (1.0) |
| Almost | n (%) | 25 | (13.6) | 5 | (2.6) | 25 | (12.4) | 5 | (2.4) |
| none (1) | | | | | | | | | |
| Mild (2) | n (%) | 54 | (29.3) | 38 | (20.1) | 58 | (28.9) | 40 | (19.5) |
| Moderate (3) | n (%) | 65 | (35.3) | 77 | (40.7) | 72 | (35.8) | 81 | (39.5) |
| Severe (4) | n (%) | 40 | (21.7) | 67 | (35.4) | 46 | (22.9) | 77 | (37.6) |
| Not done | n (%) | 17 | | 16 | | — | | — | |
| | Mean | 2.7 | | 3.1 | | 2.7 | | 3.1 | |
| | SD | 0.97 | | 0.87 | | 0.96 | | 0.87 | |
| Target buttock change from baseline | | | | | | | | | |
| −4 | n (%) | 0 | (0.0) | 2 | (1.1) | 0 | (0.0) | 2 | (1.0) |
| −3 | n (%) | 10 | (5.4) | 2 | (1.1) | 10 | (5.0) | 2 | (1.0) |
| −2 | n (%) | 41 | (22.3) | 22 | (11.6) | 41 | (20.4) | 23 | (11.2) |
| −1 | n (%) | 63 | (34.2) | 51 | (27.0) | 70 | (34.8) | 53 | (25.9) |
| 0 | n (%) | 67 | (36.4) | 102 | (54.0) | 77 | (38.3) | 114 | (55.6) |
| +1 | n (%) | 3 | (1.6) | 10 | (5.3) | 3 | (1.5) | 11 | (5.4) |
| Not done | n | 17 | | 16 | | — | | — | |
| | Mean | −0.9 | | −0.5 | | −0.9 | | −0.5 | |
| | SD | 0.93 | | 0.88 | | 0.92 | | 0.87 | |
| | p-value | <0.001 | | | | <0.001 | | | |
| Non-target buttock rating | | | | | | | | | |
| None (0) | n (%) | 1 | (0.5) | 2 | (1.1) | 1 | (0.5) | 2 | (1.0) |
| Almost | n (%) | 20 | (10.9) | 7 | (3.7) | 21 | (10.4) | 7 | (3.4) |
| none (1) | | | | | | | | | |
| Mild (2) | n (%) | 55 | (29.9) | 34 | (17.9) | 59 | (29.4) | 35 | (17.1) |
| Moderate (3) | n (%) | 63 | (34.2) | 88 | (46.3) | 70 | (34.8) | 93 | (45.4) |
| Severe (4) | n (%) | 45 | (24.5) | 59 | (31.1) | 50 | (24.9) | 68 | (33.2) |
| Not done | n (%) | 17 | | 15 | | — | | — | |
| | Mean | 2.7 | | 3.0 | | 2.7 | | 3.1 | |
| | SD | 0.97 | | 0.86 | | 0.97 | | 0.85 | |
| Non-target buttock change from baseline | | | | | | | | | |
| −4 | n (%) | 1 | (0.5) | 1 | (0.5) | 1 | (0.5) | 1 | (0.5) |
| −3 | n (%) | 7 | (3.8) | 1 | (0.5) | 8 | (4.0) | 1 | (0.5) |
| −2 | n (%) | 32 | (17.4) | 28 | (14.7) | 33 | (16.4) | 29 | (14.1) |
| −1 | n (%) | 80 | (43.5) | 49 | (25.8) | 86 | (42.8) | 50 | (24.4) |
| 0 | n (%) | 59 | (32.1) | 106 | (55.8) | 67 | (33.3) | 118 | (57.6) |
| +1 | n (%) | 5 | (2.7) | 5 | (2.6) | 6 | (3.0) | 6 | (2.9) |

| | | | | | |
|---|---|---|---|---|---|
| Not done | n | 17 | 15 | — | — |
| | Mean | −0.9 | −0.6 | −0.9 | −0.5 |
| | SD | 0.89 | 0.83 | 0.90 | 0.83 |

| | | |
|---|---|---|
| p-value | <0.001 | <0.001 |

TABLE 27
PR-PCSS 1-level and 2-level responders for target buttock, non-target
buttock, at least 1 buttock, and both buttocks (mITT Population).

| | | Day 22 | | Day 43 | |
|---|---|---|---|---|---|
| | | CCH | Placebo | CCH | Placebo |
| PR-PCSS | Statistic | (N = 201) | (N = 205) | (N = 201) | (N = 205) |

Target buttock 1-level responder

| | | | | | | | | | |
|---|---|---|---|---|---|---|---|---|---|
| Yes | n (%) | 65 | (34.8) | 34 | (16.8) | 108 | (57.4) | 68 | (34.9) |
| No | n (%) | 122 | (65.2) | 168 | (83.2) | 80 | (42.6) | 127 | (65.1) |

| | | | | | |
|---|---|---|---|---|---|
| Missing | n (%) | 14 | 3 | 13 | 10 |

| | | |
|---|---|---|
| p-value | <0.001 | <0.001 |

Non-target buttock 1-level responder

| | | | | | | | | | |
|---|---|---|---|---|---|---|---|---|---|
| Yes | n (%) | 65 | (34.8) | 38 | (18.8) | 117 | (62.2) | 71 | (36.4) |
| No | n (%) | 122 | (65.2) | 164 | (81.2) | 71 | (37.8) | 124 | (63.6) |

| | | | | | |
|---|---|---|---|---|---|
| Missing | n (%) | 14 | 3 | 13 | 10 |

| | | |
|---|---|---|
| p-value | <0.001 | <0.001 |

At least buttock 1-level responder

| | | | | | | | | | |
|---|---|---|---|---|---|---|---|---|---|
| Yes | n (%) | 81 | (43.3) | 49 | (24.3) | 128 | (68.1) | 89 | (45.6) |
| No | n (%) | 106 | (56.7) | 153 | (75.7) | 60 | (31.9) | 106 | (54.4) |

| | | | | | |
|---|---|---|---|---|---|
| Missing | n (%) | 14 | 3 | 13 | 10 |

| | | |
|---|---|---|
| p-value | <0.001 | <0.001 |

Both buttocks 1-level responder

| | | | | | | | | | |
|---|---|---|---|---|---|---|---|---|---|
| Yes | n (%) | 49 | (26.2) | 23 | (11.4) | 97 | (51.6) | 50 | (25.6) |
| No | n (%) | 138 | (73.8) | 179 | (88.6) | 91 | (48.4) | 145 | (74.4) |

| | | | | | |
|---|---|---|---|---|---|
| Missing | n (%) | 14 | 3 | 13 | 10 |

| | | |
|---|---|---|
| p-value | <0.001 | <0.001 |

Target buttock 2-level responder

| | | | | | | | | | |
|---|---|---|---|---|---|---|---|---|---|
| Yes | n (%) | 9 | (4.8) | 1 | (0.5) | 33 | (17.6) | 14 | (7.2) |
| No | n (%) | 178 | (95.2) | 201 | (99.5) | 155 | (82.4) | 181 | (92.8) |

| | | | | | |
|---|---|---|---|---|---|
| Missing | n (%) | 14 | 3 | 13 | 10 |

| | | |
|---|---|---|
| p-value | 0.009 | 0.002 |

Non-target buttock 2-level responder

| | | | | | | | | | |
|---|---|---|---|---|---|---|---|---|---|
| Yes | n (%) | 6 | (3.2) | 0 | (0.0) | 29 | (15.4) | 15 | (7.7) |
| No | n (%) | 181 | (96.8) | 202 | (100.0) | 159 | (84.6) | 180 | (92.3) |

| | | | | | |
|---|---|---|---|---|---|
| Missing | n (%) | 14 | 3 | 13 | 10 |

| | | |
|---|---|---|
| p-value | 0.012 | 0.015 |

At least 1 buttock 2-level responder

| | | | | | | | | | |
|---|---|---|---|---|---|---|---|---|---|
| Yes | n (%) | 13 | (7.0) | 1 | (0.5) | 41 | (21.8) | 20 | (10.3) |
| No | n (%) | 174 | (93.0) | 201 | (99.5) | 147 | (78.2) | 175 | (89.7) |

| | | | | | |
|---|---|---|---|---|---|
| Missing | n (%) | 14 | 3 | 13 | 10 |

| | | |
|---|---|---|
| p-value | <0.001 | 0.001 |

Both buttocks 2-level responder

| | | | | | | | | | |
|---|---|---|---|---|---|---|---|---|---|
| Yes | n (%) | 2 | (1.1) | 0 | (0.0) | 21 | (11.2) | 9 | (4.6) |
| No | n (%) | 185 | (98.9) | 202 | (100.0) | 167 | (88.8) | 186 | (95.4) |

| Missing | n (%) | 14 | 3 | 13 | 10 |
|---|---|---|---|---|---|
| | p-value | 0.164 | | 0.018 | |
| | | Day 71 | | Day 71 (LOCF) | |
| | | CCH | Placebo | CCH | Placebo |
| PR-PCSS | Statistic | (N = 201) | (N = 205) | (N = 201) | (N = 205) |

Target buttock 1-level responder

| | | | | | | | | | |
|---|---|---|---|---|---|---|---|---|---|
| Yes | n (%) | 114 | (62.0) | 77 | (40.7) | 121 | (60.2) | 80 | (39.0) |
| No | n (%) | 70 | (38.0) | 112 | (59.3) | 80 | (39.8) | 125 | (61.0) |

| | | | | | |
|---|---|---|---|---|---|
| Missing | n (%) | 17 | 16 | — | — |

| | | |
|---|---|---|
| p- | <0.001 | <0.001 |
| value | | |

Non-target buttock 1-level responder

| | | | | | | | | | |
|---|---|---|---|---|---|---|---|---|---|
| Yes | n (%) | 120 | (65.2) | 79 | (41.6) | 128 | (63.7) | 81 | (39.5) |
| No | n (%) | 64 | (34.8) | 111 | (58.4) | 73 | (36.3) | 124 | (60.5) |

| | | | | | |
|---|---|---|---|---|---|
| Missing | n (%) | 17 | 15 | — | — |

| | | |
|---|---|---|
| p- | <0.001 | <0.001 |
| value | | |

At least buttock 1-level responder

| | | | | | | | | | |
|---|---|---|---|---|---|---|---|---|---|
| Yes | n (%) | 131 | (71.2) | 91 | (48.1) | 139 | (69.2) | 96 | (46.8) |
| No | n (%) | 53 | (28.8) | 98 | (51.9) | 62 | (30.8) | 109 | (53.2) |

| | | | | | |
|---|---|---|---|---|---|
| Missing | n (%) | 17 | 16 | — | — |

| | | |
|---|---|---|
| p- | <0.001 | <0.001 |
| value | | |

Both buttocks 1-level responder

| | | | | | | | | | |
|---|---|---|---|---|---|---|---|---|---|
| Yes | n (%) | 103 | (56.0) | 64 | (33.9) | 110 | (54.7) | 65 | (31.7) |
| No | n (%) | 81 | (44.0) | 125 | (66.1) | 91 | (45.3) | 140 | (68.3) |

| | | | | | |
|---|---|---|---|---|---|
| Missing | n (%) | 17 | 16 | — | — |

| | | |
|---|---|---|
| p- | <0.001 | <0.001 |
| value | | |

Target buttock 2-level responder

| | | | | | | | | | |
|---|---|---|---|---|---|---|---|---|---|
| Yes | n (%) | 51 | (27.7) | 26 | (13.8) | 51 | (25.4) | 27 | (13.2) |
| No | n (%) | 133 | (72.3) | 163 | (86.2) | 150 | (74.6) | 178 | (86.8) |

| | | | | | |
|---|---|---|---|---|---|
| Missing | n (%) | 17 | 16 | — | — |

| | | |
|---|---|---|
| p- | <0.001 | 0.002 |
| value | | |

Non-target buttock 2-level responder

| | | | | | | | | | |
|---|---|---|---|---|---|---|---|---|---|
| Yes | n (%) | 40 | (21.7) | 30 | (15.8) | 42 | (20.9) | 31 | (15.1) |
| No | n (%) | 144 | (78.3) | 160 | (84.2) | 159 | (79.1) | 174 | (84.9) |

| | | | | | |
|---|---|---|---|---|---|
| Missing | n (%) | 17 | 15 | — | — |

| | | |
|---|---|---|
| p- | 0.115 | 0.151 |
| value | | |

At least 1 buttock 2-level responder

| | | | | | | | | | |
|---|---|---|---|---|---|---|---|---|---|
| Yes | n (%) | 57 | (31.0) | 39 | (20.6) | 59 | (29.4) | 40 | (19.5) |
| No | n (%) | 127 | (69.0) | 150 | (79.4) | 142 | (70.6) | 165 | (80.5) |

| | | | | | |
|---|---|---|---|---|---|
| Missing | n (%) | 17 | 16 | — | — |

| | | |
|---|---|---|
| p- | 0.016 | 0.024 |
| value | | |

Both buttocks 2-level responder

| | | | | | | | | | |
|---|---|---|---|---|---|---|---|---|---|
| Yes | n (%) | 34 | (18.5) | 17 | (9.0) | 34 | (16.9) | 18 | (8.8) |
| No | n (%) | 150 | (81.5) | 172 | (91.0) | 167 | (83.1) | 187 | (91.2) |

| | | | | | |
|---|---|---|---|---|---|
| Missing | n (%) | 17 | 16 | — | — |

| | | |
|---|---|---|
| p- | 0.007 | 0.019 |
| value | | |

LOCF =

CR-PCSS ratings by visit and change from baseline were also analyzed. Consistent with the PR-PCSS ratings, they show statistically significant improvements in cellulite severity in patients treated with CCH versus placebo. At Day 71, the mean change from baseline in CR-PCSS was greater in subjects treated with CCH versus placebo. A response to treatment was evident as early as Day 22. The proportion of 1-level CR-PCSS responders on Day 22 was greater in subjects treated with CCH than placebo-treated subjects in both the target buttock (33.2% vs. 16.8%, respectively) and non-target buttock (36.4% vs. 16.8%). These differences were statistically significant with p<0.001. At Day 71, the proportion of 1-level CR-PCSS responders was greater in subjects treated with CCH versus placebo. The results are summarized in Tables 28-29 and FIGS. 22-23.

TABLE 28
CR-PCSS rating and change from baseline for the target and non-target buttocks by visit (mITT Population).

| | | Day 1 | | Day 22 | | Day 43 | |
|---|---|---|---|---|---|---|---|
| | | CCH | Placebo | CCH | Placebo | CCH | Placebo |
| CR-PCSS | Statistic | (N = 201) | (N = 205) | (N = 201) | (N = 205) | (N = 201) | (N = 205) |

Target buttock rating

| | | | | | | | | | | | | | |
|---|---|---|---|---|---|---|---|---|---|---|---|---|---|
| None (0) | n (%) | 0 | (0.0) | 0 | (0.0) | 0 | (0.0) | 0 | (0.0) | 0 | (0.0) | 0 | (0.0) |
| Almost none (1) | n (%) | 0 | (0.0) | 0 | (0.0) | 2 | (1.1) | 2 | (1.0) | 14 | (7.4) | 3 | (1.5) |
| Mild (2) | n (%) | 0 | (0.0) | 0 | (0.0) | 29 | (15.5) | 6 | (3.0) | 54 | (28.7) | 20 | (10.3) |
| Moderate (3) | n (%) | 122 | (60.7) | 125 | (61.0) | 108 | (57.8) | 128 | (63.4) | 79 | (42.0) | 102 | (52.3) |
| Severe (4) | n (%) | 79 | (39.3) | 80 | (39.0) | 48 | (25.7) | 66 | (32.7) | 41 | (21.8) | 70 | (35.9) |

| | | | | | | | |
|---|---|---|---|---|---|---|---|
| Not done | n (%) | — | — | 14 | 3 | 13 | 10 |
| | Mean | 3.4 | 3.4 | 3.1 | 3.3 | 2.8 | 3.2 |
| | SD | 0.49 | 0.49 | 0.67 | 0.57 | 0.87 | 0.69 |

Target buttock change from baseline

| | | | | | | | | | | | |
|---|---|---|---|---|---|---|---|---|---|---|---|
| −4 | n (%) | — | — | 0 | (0.0) | 0 | (0.0) | 0 | (0.0) | 0 | (0.0) |
| −3 | n (%) | — | — | 0 | (0.0) | 0 | (0.0) | 0 | (0.0) | 0 | (0.0) |
| −2 | n (%) | — | — | 4 | (2.1) | 2 | (1.0) | 28 | (14.9) | 7 | (3.6) |
| −1 | n (%) | — | — | 58 | (31.0) | 32 | (15.8) | 68 | (36.2) | 31 | (15.9) |
| 0 | n (%) | — | — | 120 | (64.2) | 154 | (76.2) | 84 | (44.7) | 143 | (73.3) |
| +1 | n (%) | — | — | 5 | (2.7) | 14 | (6.9) | 8 | (4.3) | 14 | (7.2) |

| | | | | | | | |
|---|---|---|---|---|---|---|---|
| Not done | n | — | — | 14 | 3 | 13 | 10 |
| | Mean | — | — | −0.3 | −0.1 | −0.6 | −0.2 |
| | SD | | | 0.56 | 0.51 | 0.79 | 0.59 |

| | | |
|---|---|---|
| p-value | <0.001 | <0.001 |

Non-target buttock rating

| | | | | | | | | | | | | | |
|---|---|---|---|---|---|---|---|---|---|---|---|---|---|
| None (0) | n (%) | 0 | (0.0) | 0 | (0.0) | 0 | (0.0) | 0 | (0.0) | 0 | (0.0) | 0 | (0.0) |
| Almost | n (%) | 0 | (0.0) | 0 | (0.0) | 7 | (3.7) | 0 | (0.0) | 15 | (8.0) | 2 | (1.0) |
| none (1) | | | | | | | | | | | | | |
| Mild (2) | n (%) | 0 | (0.0) | 0 | (0.0) | 26 | (13.9) | 10 | (5.0) | 52 | (27.7) | 21 | (10.8) |
| Moderate (3) | n (%) | 115 | (57.2) | 126 | (61.5) | 102 | (54.5) | 122 | (60.4) | 71 | (37.8) | 110 | (56.4) |
| Severe (4) | n (%) | 86 | (42.8) | 79 | (38.5) | 52 | (27.8) | 70 | (34.7) | 50 | (26.6) | 62 | (31.8) |

| | | | | | | | |
|---|---|---|---|---|---|---|---|
| Not done | n (%) | — | — | 14 | 3 | 13 | 10 |
| | Mean | 3.4 | 3.4 | 3.1 | 3.3 | 2.8 | 3.2 |
| | SD | 0.50 | 0.49 | 0.75 | 0.56 | 0.91 | 0.66 |

Non-target buttock change from baseline

| | | | | | | | | | | | |
|---|---|---|---|---|---|---|---|---|---|---|---|
| −4 | — | — | — | 0 | (0.0) | 0 | (0.0) | 0 | (0.0) | 0 | (0.0) |
| −3 | n (%) | — | — | 0 | (0.0) | 0 | (0.0) | 1 | (0.5) | 0 | (0.0) |
| −2 | n (%) | — | — | 10 | (5.3) | 0 | (0.0) | 24 | (12.8) | 5 | (2.6) |
| −1 | n (%) | — | — | 58 | (31.0) | 34 | (16.8) | 71 | (37.8) | 37 | (19.0) |
| 0 | n (%) | — | — | 111 | (59.4) | 15 | (74.8) | 83 | (44.1) | 143 | (73.3) |
| +1 | n (%) | — | — | 8 | (4.3) | 17 | (8.4) | 9 | (4.8) | 10 | (5.1) |

| | | | | | | | |
|---|---|---|---|---|---|---|---|
| Not done | n | — | — | 14 | 3 | 13 | 10 |
| | Mean | — | — | −0.4 | −0.1 | −0.6 | −0.2 |
| | SD | | | 0.65 | 0.50 | 0.79 | 0.56 |

| | | |
|---|---|---|
| p-value | <0.001 | <0.001 |

| | | Day 71 | | | | Day 71 (LOCF) | | | |
|---|---|---|---|---|---|---|---|---|---|
| | | CCH | | Placebo | | CCH | | Placebo | |
| CR-PCSS | Statistic | (N = 201) | | (N = 205) | | (N = 201) | | (N = 205) | |
| Target buttock rating | | | | | | | | | |
| None (0) | n (%) | 0 | (0.0) | 2 | (1.0) | 0 | (0.0) | 2 | (1.0) |
| Almost | n (%) | 25 | (13.7) | 6 | (3.1) | 27 | (13.4) | 6 | (2.9) |
| none (1) | | | | | | | | | |
| Mild (2) | n (%) | 52 | (28.4) | 32 | (16.8) | 56 | (27.9) | 32 | (15.6) |
| Moderate (3) | n (%) | 66 | (36.1) | 96 | (50.3) | 73 | (36.3) | 104 | (50.7) |
| Severe (4) | n (%) | 40 | (21.9) | 55 | (28.8) | 45 | (22.4) | 61 | (29.8) |
| Not done | n (%) | 18 | | 14 | | — | | — | |
| | Mean | 2.7 | | 3.0 | | 2.7 | | 3.1 | |
| | SD | 0.97 | | 0.82 | | 0.97 | | 0.81 | |
| Target buttock change from baseline | | | | | | | | | |
| −4 | n (%) | 0 | (0.0) | 0 | (0.0) | 0 | (0.0) | 0 | (0.0) |
| −3 | n (%) | 3 | (1.6) | 3 | (1.6) | 3 | (1.5) | 3 | (1.5) |
| −2 | n (%) | 32 | (17.5) | 9 | (4.7) | 35 | (17.4) | 9 | (4.4) |
| −1 | n (%) | 72 | (39.3) | 50 | (26.2) | 76 | (37.8) | 53 | (25.9) |
| 0 | n (%) | 66 | (36.1) | 119 | (62.3) | 76 | (37.8) | 129 | (62.9) |
| +1 | n (%) | 10 | (5.5) | 10 | (5.2) | 11 | (5.5) | 11 | (5.4) |
| Not | n | 18 | | 14 | | — | | — | |
| done | Mean | −0.7 | | −0.4 | | −0.7 | | −0.3 | |
| | SD | 0.87 | | 0.72 | | 0.87 | | 0.71 | |
| | p-value | <0.001 | | | | <0.001 | | | |
| Non-target buttock rating | | | | | | | | | |
| None (0) | n (%) | 0 | (0.0) | 0 | (0.0) | 0 | (0.0) | 0 | (0.0) |
| Almost | n (%) | 21 | (11.5) | 7 | (3.7) | 22 | (10.9) | 7 | (3.4) |
| none (1) | | | | | | | | | |
| Mild (2) | n (%) | 58 | (31.7) | 27 | (14.1) | 62 | (30.8) | 27 | (13.2) |
| Moderate (3) | n (%) | 66 | (36.1) | 96 | (50.3) | 73 | (36.3) | 105 | (51.2) |
| Severe (4) | n (%) | 38 | (20.8) | 61 | (31.9) | 44 | (21.9) | 66 | (32.2) |
| Not | n (%) | 18 | | 14 | | — | | — | |
| done | Mean | 2.7 | | 3.1 | | 2.7 | | 3.1 | |
| | SD | 0.93 | | 0.77 | | 0.94 | | 0.76 | |
| Non-target buttock change from baseline | | | | | | | | | |
| −4 | — | 0 | (0.0) | 0 | (0.0) | 0 | (0.0) | 0 | (0.0) |
| −3 | n (%) | 0 | (0.0) | 0 | (0.0) | 0 | (0.0) | 0 | (0.0) |
| −2 | n (%) | 38 | (20.8) | 12 | (6.3) | 40 | (19.9) | 12 | (5.9) |
| −1 | n (%) | 73 | (39.9) | 41 | (21.5) | 78 | (38.8) | 42 | (20.5) |
| 0 | n (%) | 64 | (35.0) | 27 | (66.5) | 73 | (36.3) | 139 | (67.8) |
| +1 | n (%) | 8 | (4.4) | 11 | (5.8) | 10 | (5.0) | 12 | (5.9) |

| | | | | | |
|---|---|---|---|---|---|
| Not | n | 18 | 14 | — | — |
| done | Mean | −0.8 | −0.3 | −0.7 | −0.3 |
| | SD | 0.83 | 0.67 | 0.83 | 0.66 |

| | | |
|---|---|---|
| p- | <0.001 | <0.001 |
| value | | |

TABLE 29
CR-PCSS 1-level and 2-level responders for target buttock, non-target
buttock, at least one buttock, and both buttocks (mITT Population).

| | | Day 22 | | Day 43 | |
|---|---|---|---|---|---|
| | | CCH | Placebo | CCH | Placebo |
| CR-PCSS | Statistic | (N = 201) | (N = 205) | (N = 201) | (N = 205) |

Target buttock 1-level responder

| | | | | | | | | | |
|---|---|---|---|---|---|---|---|---|---|
| Yes | n (%) | 62 | (33.2) | 34 | (16.8) | 96 | (51.1) | 38 | (19.5) |
| No | n (%) | 125 | (66.8) | 168 | (83.2) | 92 | (48.9) | 157 | (80.5) |

| | | | | | |
|---|---|---|---|---|---|
| Missing | n (%) | 14 | 3 | 13 | 10 |

| | | |
|---|---|---|
| p-value | <0.001 | <0.001 |

Non-target buttock 1-level responder

| | | | | | | | | | |
|---|---|---|---|---|---|---|---|---|---|
| Yes | n (%) | 68 | (36.4) | 34 | (16.8) | 96 | (51.1) | 42 | (21.5) |
| No | n (%) | 119 | (63.6) | 168 | (83.2) | 92 | (48.9) | 153 | (78.5) |

| | | | | | |
|---|---|---|---|---|---|
| Missing | n (%) | 14 | 3 | 13 | 10 |

| | | |
|---|---|---|
| p-value | <0.001 | <0.001 |

At least buttock 1-level responder

| | | | | | | | | | |
|---|---|---|---|---|---|---|---|---|---|
| Yes | n (%) | 89 | (47.6) | 51 | (25.2) | 114 | (60.6) | 54 | (27.7) |
| No | n (%) | 98 | (52.4) | 151 | (74.8) | 74 | (39.4) | 141 | (72.3) |

| | | | | | |
|---|---|---|---|---|---|
| Missing | n (%) | 14 | 3 | 13 | 10 |

| | | |
|---|---|---|
| p-value | <0.001 | <0.001 |

Both buttocks 1-level responder

| | | | | | | | | | |
|---|---|---|---|---|---|---|---|---|---|
| Yes | n (%) | 41 | (21.9) | 17 | (8.4) | 78 | (41.5) | 26 | (13.3) |
| No | n (%) | 146 | (78.1) | 185 | (91.6) | 110 | (58.5) | 169 | (86.7) |

| | | | | | |
|---|---|---|---|---|---|
| Missing | n (%) | 14 | 3 | 13 | 10 |

| | | |
|---|---|---|
| p-value | <0.001 | <0.001 |

Target buttock 2-level responder

| | | | | | | | | | |
|---|---|---|---|---|---|---|---|---|---|
| Yes | n (%) | 4 | (2.1) | 2 | (1.0) | 28 | (14.9) | 7 | (3.6) |
| No | n (%) | 183 | (97.9) | 200 | (99.0) | 160 | (85.1) | 188 | (96.4) |

| | | | | | |
|---|---|---|---|---|---|
| Missing | n (%) | 14 | 3 | 13 | 10 |

| | | |
|---|---|---|
| p-value | 0.311 | <0.001 |

Non-target buttock 2-level responder

| | | | | | | | | | |
|---|---|---|---|---|---|---|---|---|---|
| Yes | n (%) | 10 | (5.3) | 0 | (0.0) | 25 | (13.3) | 5 | (2.6) |
| No | n (%) | 177 | (94.7) | 202 | (100.0) | 163 | (86.7) | 190 | (97.4) |

| | | | | | |
|---|---|---|---|---|---|
| Missing | n (%) | 14 | 3 | 13 | 10 |

| | | |
|---|---|---|
| p-value | <0.001 | <0.001 |

At least 1 buttock 2-level responder

| | | | | | | | | | |
|---|---|---|---|---|---|---|---|---|---|
| Yes | n (%) | 12 | (6.4) | 2 | (1.0) | 36 | (19.1) | 10 | (5.1) |
| No | n (%) | 175 | (93.6) | 200 | (99.0) | 152 | (80.9) | 185 | (94.9) |

| | | | | | |
|---|---|---|---|---|---|
| Missing | n (%) | 14 | 3 | 13 | 10 |

| | | |
|---|---|---|
| p-value | 0.003 | <0.001 |

Both buttocks 2-level responder

| | | | | | | | | | |
|---|---|---|---|---|---|---|---|---|---|
| Yes | n (%) | 2 | (1.1) | 0 | (0.0) | 17 | (9.0) | 2 | (1.0) |
| No | n (%) | 185 | (98.9) | 202 | (100.0) | 171 | (91.0) | 193 | (99.0) |

| Missing | n (%) | 14 | 3 | 13 | 10 |
|---|---|---|---|---|---|
| | p-value | 0.119 | | <0.001 | |
| | | Day 71 | | Day 71 (LOCF) | |
| | | CCH | Placebo | CCH | Placebo |
| CR-PCSS | Statistic | (N = 201) | (N = 205) | (N = 201) | (N = 205) |

Target buttock 1-level responder

| | | | | | | | | | |
|---|---|---|---|---|---|---|---|---|---|
| Yes | n (%) | 107 | (58.5) | 62 | (32.5) | 114 | (56.7) | 65 | (31.7) |
| No | n (%) | 76 | (41.5) | 29 | (67.5) | 87 | (43.3) | 140 | (68.3) |

| | | | | | |
|---|---|---|---|---|---|
| Missing | n (%) | 18 | 14 | — | — |

| | | |
|---|---|---|
| p-value | <0.001 | <0.001 |

Non-target buttock 1-level responder

| | | | | | | | | | |
|---|---|---|---|---|---|---|---|---|---|
| Yes | n (%) | 111 | (60.7) | 53 | (27.7) | 118 | (58.7) | 54 | (26.3) |
| No | n (%) | 72 | (39.3) | 138 | (72.3) | 83 | (41.3) | 151 | (73.7) |

| | | | | | |
|---|---|---|---|---|---|
| Missing | n (%) | 18 | 14 | — | — |

| | | |
|---|---|---|
| p-value | <0.001 | <0.001 |

At least buttock 1-level responder

| | | | | | | | | | |
|---|---|---|---|---|---|---|---|---|---|
| Yes | n (%) | 125 | (68.3) | 75 | (39.3) | 133 | (66.2) | 78 | (38.0) |
| No | n (%) | 58 | (31.7) | 116 | (60.7) | 68 | (33.8) | 127 | (62.0) |

| | | | | | |
|---|---|---|---|---|---|
| Missing | n (%) | 18 | 14 | — | — |

| | | |
|---|---|---|
| p-value | <0.001 | <0.001 |

Both buttocks 1-level responder

| | | | | | | | | | |
|---|---|---|---|---|---|---|---|---|---|
| Yes | n (%) | 93 | (50.8) | 40 | (20.9) | 99 | (49.3) | 41 | (20.0) |
| No | n (%) | 90 | (49.2) | 151 | (79.1) | 102 | (50.7) | 164 | (80.0) |

| | | | | | |
|---|---|---|---|---|---|
| Missing | n (%) | 18 | 14 | — | — |

| | | |
|---|---|---|
| p-value | <0.001 | <0.001 |

Target buttock 2-level responder

| | | | | | | | | | |
|---|---|---|---|---|---|---|---|---|---|
| Yes | n (%) | 35 | (19.1) | 12 | (6.3) | 38 | (18.9) | 12 | (5.9) |
| No | n (%) | 148 | (80.9) | 179 | (93.7) | 163 | (81.1) | 193 | (94.1) |

| | | | | | |
|---|---|---|---|---|---|
| Missing | n (%) | 18 | 14 | — | — |

| | | |
|---|---|---|
| p-value | <0.001 | <0.001 |

Non-target buttock 2-level responder

| | | | | | | | | | |
|---|---|---|---|---|---|---|---|---|---|
| Yes | n (%) | 38 | (20.8) | 12 | (6.3) | 40 | (19.9) | 12 | (5.9) |
| No | n (%) | 145 | (79.2) | 179 | (93.7) | 161 | (80.1) | 193 | (94.1) |

| | | | | | |
|---|---|---|---|---|---|
| Missing | n (%) | 18 | 14 | — | — |

| | | |
|---|---|---|
| p-value | <0.001 | <0.001 |

At least 1 buttock 2-level responder

| | | | | | | | | | |
|---|---|---|---|---|---|---|---|---|---|
| Yes | n (%) | 47 | (25.7) | 16 | (8.4) | 50 | (24.9) | 16 | (7.8) |
| No | n (%) | 136 | (74.3) | 175 | (91.6) | 151 | (75.1) | 189 | (92.2) |

| | | | | | |
|---|---|---|---|---|---|
| Missing | n (%) | 18 | 14 | — | — |

| | | |
|---|---|---|
| p-value | <0.001 | <0.001 |

Both buttocks 2-level responder

| | | | | | | | | | |
|---|---|---|---|---|---|---|---|---|---|
| Yes | n (%) | 26 | (14.2) | 8 | (4.2) | 28 | (13.9) | 8 | (3.9) |
| No | n (%) | 157 | (85.8) | 183 | (95.8) | 173 | (86.1) | 197 | (96.1) |

| | | | | | |
|---|---|---|---|---|---|
| Missing | n (%) | 18 | 14 | — | — |

| | | |
|---|---|---|
| p-value | <0.001 | <0.001 |

S-GAIS ratings were analyzed. As early as Day 22, the mean S-GAIS was greater in subjects treated with CCH versus placebo. This improvement was statistically significant (p<0.001). The improvement was still observed at Day 71. Analyses of S-GAIS 1-level and 2-level responders for target and non-target buttocks demonstrate statistically significant differences in improvement over the course of the study for subjects treated with CCH versus placebo. These results are summarized in Tables 30-31. Similar findings were observed using I-GAIS ratings, which are summarized in Tables 32-33.

TABLE 30
S-GAIS ratings of the target and non-target buttock by visit (mITT Population).

| | | Day 22 | | Day 43 | |
|---|---|---|---|---|---|
| | | CCH | Placebo | CCH | Placebo |
| S-GAIS | Statistic | (N = 201) | (N = 205) | (N = 201) | (N = 205) |

Target buttock

| Very much improved (3) | n (%) | 3 | (1.6) | 0 | (0.0) | 6 | (3.2) | 0 | (0.0) |
|---|---|---|---|---|---|---|---|---|---|
| Much improved (2) | n (%) | 5 | (2.7) | 2 | (1.0) | 32 | (17.0) | 8 | (4.1) |
| Improved (1) | n (%) | 91 | (48.9) | 51 25.2) | | 94 | (50.0) | 66 | (33.8) |
| No change (0) | n (%) | 79 | (42.5) | 140 | (69.3) | 44 | (23.4) | 109 | (55.9) |
| Worse (−1) | n (%) | 6 | (3.2) | 7 | (3.5) | 8 | (4.3) | 7 | (3.6) |
| Much worse (−2) | n (%) | 2 | (1.1) | 1 | (0.5) | 3 | (1.6) | 3 | (1.5) |
| Very much worse (−3) | n (%) | 0 | (0.0) | 1 | (0.5) | 1 | (0.5) | 2 | (1.0) |
| Missing | N | 15 | | 3 | | 13 | | 10 | |
| | Mean (SD) | 0.5 | (0.73) | 0.2 | (0.59) | 0.8 | (0.95) | 0.3 | (0.77) |

| | | |
|---|---|---|
| P = value | <0.001 | <0.001 |

Non-target buttock

| Very much improved (3) | n (%) | 3 | (1.6) | 0 | (0.0) | 8 | (4.3) | 0 | (0.0) |
|---|---|---|---|---|---|---|---|---|---|
| Much improved (2) | n (%) | 6 | (3.2) | 4 | (2.0) | 36 | (19.1) | 12 | (6.2) |
| Improved (1) | n (%) | 87 | (46.8) | 50 | (24.8) | 97 | (51.6) | 62 | (31.8) |
| No change (0) | n (%) | 81 | (43.5) | 139 | (68.8) | 38 | (20.2) | 108 | (55.4) |
| Worse (−1) | n (%) | 9 | (4.8) | 8 | (4.0) | 5 | (2.7) | 9 | (4.6) |
| Much worse (−2) | n (%) | 0 | (0.0) | 0 | (0.0) | 2 | (1.1) | 2 | (1.0) |
| Very much worse (−3) | n (%) | 0 | (0.0) | 1 | (0.5) | 2 | (1.1) | 2 | (1.0) |
| Missing | N | 15 | | 3 | | 13 | | 10 | |
| | Mean (SD) | 0.5 | (0.71) | 0.2 | (0.60) | 0.9 | (0.97) | 0.3 | (0.79) |

| | P = value | <0.001 | | <0.001 | |
|---|---|---|---|---|---|
| | | Day 71 | | Day 71 (LOCF) | |
| | | CCH | Placebo | CCH | Placebo |
| S-GAIS | Statistic | (N = 201) | (N = 205) | (N = 201) | (N = 205) |

Target buttock

| Very much improved (3) | n (%) | 12 | (6.5) | 3 | (1.6) | 12 | (6.0) | 3 | (1.5) |
|---|---|---|---|---|---|---|---|---|---|
| Much improved (2) | n (%) | 37 | (20.1) | 10 | (5.3) | 38 | (18.9) | 10 | (4.9) |
| Improved (1) | n (%) | 86 | (46.7) | 69 | (36.3) | 93 | (46.3) | 71 | (34.6) |
| No change (0) | n (%) | 38 | (20.7) | 100 | (52.6) | 43 | (21.4) | 113 | (55.1) |
| Worse (−1) | n (%) | 10 | (5.4) | 5 | (2.6) | 12 | (6.0) | 5 | (2.4) |
| Much worse (−2) | n (%) | 0 | (0.0) | 2 | (1.1) | 1 | (0.5) | 2 | (1.0) |
| Very much worse (−3) | n (%) | 1 | (0.5) | 1 | (0.5) | 2 | (1.0) | 1 | (0.5) |
| Missing | N | 17 | | 15 | | — | | — | |
| | Mean (SD) | 1.0 | (0.99) | 0.5 | (0.79) | 0.9 | (1.04) | 0.4 | (0.77) |

| | | |
|---|---|---|
| P = value | <0.001 | <0.001 |

Non-target buttock

| Very much improved (3) | n (%) | 12 | (6.5) | 3 | (1.6) | 12 | (6.0) | 3 | (1.5) |
|---|---|---|---|---|---|---|---|---|---|
| Much improved (2) | n (%) | 32 | (17.4) | 11 | (5.8) | 33 | (16.4) | 11 | (5.4) |
| Improved (1) | n (%) | 92 | (50.0) | 67 | (35.3) | 99 | (49.3) | 67 | (32.7) |
| No change (0) | n (%) | 41 | (22.3) | 102 | (53.7) | 47 | (23.4) | 116 | (56.6) |
| Worse (−1) | n (%) | 6 | (3.3) | 5 | (2.6) | 7 | (3.5) | 5 | (2.4) |
| Much worse (−2) | n (%) | 0 | (0.0) | 2 | (1.1) | 0 | (0.0) | 3 | (1.5) |
| Very much worse (−3) | n (%) | 1 | (0.5) | 0 | (0.0) | 3 | (1.5) | 0 | (0.0) |
| Missing | N | 17 | | 15 | | — | | — | |
| | Mean (SD) | 1.0 | (0.94) | 0.5 | (0.76) | 0.9 | (1.01) | 0.4 | (0.76) |

| | | |
|---|---|---|
| P = value | <0.001 | <0.001 |

TABLE 31
S-GAIS 1-level and 2-level responders for target buttock, non-target buttock, at least 1 buttock, and both buttocks (mITT Population).

| | | Day 22 | | Day 43 | | Day 71 | | Day 71 (LOCF) | |
|---|---|---|---|---|---|---|---|---|---|
| | | CCH | Placebo | CCH | Placebo | CCH | Placebo | CCH | Placebo |
| S-GAIS | Statistic | (N = 201) | (N = 205) | (N = 201) | (N = 205) | (N = 201) | (N = 205) | (N = 201) | (N = 205) |

Target buttock 1-level responder

| | | | | | | | | | | | | | | | | | |
|---|---|---|---|---|---|---|---|---|---|---|---|---|---|---|---|---|---|
| Yes | n (%) | 99 | (53.2) | 53 | (26.2) | 132 | (70.2) | 74 | (37.9) | 135 | (73.4) | 82 | (43.2) | 143 | (71.1) | 84 | (41.0) |

| | | | | | | | | | |
|---|---|---|---|---|---|---|---|---|---|
| Missing | n | 15 | 3 | 13 | 10 | 17 | 15 | — | — |

| | | | | |
|---|---|---|---|---|
| p-value | <0.001 | <0.001 | <0.001 | <0.001 |

Non-target buttock 1-level responder

| | | | | | | | | | | | | | | | | | |
|---|---|---|---|---|---|---|---|---|---|---|---|---|---|---|---|---|---|
| Yes | n (%) | 96 | (51.6) | 54 | (26.7) | 141 | (75.0) | 74 | (37.9) | 136 | (73.9) | 81 | (42.6) | 144 | (71.6) | 81 | (39.5) |

| | | | | | | | | | |
|---|---|---|---|---|---|---|---|---|---|
| Missing | n | 15 | 3 | 13 | 10 | 17 | 15 | — | — |

| | | | | |
|---|---|---|---|---|
| p-value | <0.001 | <0.001 | <0.001 | <0.001 |

At least 1 buttock 1-level responder

| | | | | | | | | | | | | | | | | | |
|---|---|---|---|---|---|---|---|---|---|---|---|---|---|---|---|---|---|
| Yes | n (%) | 108 | (58.1) | 64 | (31.7) | 146 | (77.7) | 84 | (43.1) | 144 | (78.3) | 90 | (47.4) | 153 | (76.1) | 92 | (44.9) |

| | | | | | | | | | |
|---|---|---|---|---|---|---|---|---|---|
| Missing | n | 15 | 3 | 13 | 10 | 17 | 15 | — | — |

| | | | | |
|---|---|---|---|---|
| p-value | <0.001 | <0.001 | <0.001 | <0.001 |

Both buttocks 1-level responder

| | | | | | | | | | | | | | | | | | |
|---|---|---|---|---|---|---|---|---|---|---|---|---|---|---|---|---|---|
| Yes | n (%) | 87 | (46.8) | 43 | (21.3) | 127 | (67.6) | 64 | (32.8) | 127 | (69.0) | 73 | (38.4) | 134 | (66.7) | 73 | (35.6) |

| | | | | | | | | | |
|---|---|---|---|---|---|---|---|---|---|
| Missing | n | 15 | 3 | 13 | 10 | 17 | 15 | — | — |

| | | | | |
|---|---|---|---|---|
| p-value | <0.001 | <0.001 | <0.001 | <0.001 |

Target buttock 2-level responder

| | | | | | | | | | | | | | | | | | |
|---|---|---|---|---|---|---|---|---|---|---|---|---|---|---|---|---|---|
| Yes | n (%) | 8 | (4.3) | 2 | (1.0) | 38 | (20.2) | 8 | (4.1) | 49 | (26.6) | 13 | (6.8) | 50 | (24.9) | 13 | (6.3) |

| | | | | | | | | | |
|---|---|---|---|---|---|---|---|---|---|
| Missing | n | 15 | 3 | 13 | 10 | 17 | 15 | — | — |

| | | | | |
|---|---|---|---|---|
| p-value | 0.0.058 | <0.001 | <0.001 | <0.001 |

Non-target buttock 2-level responder

| | | | | | | | | | | | | | | | | | |
|---|---|---|---|---|---|---|---|---|---|---|---|---|---|---|---|---|---|
| Yes | n (%) | 9 | (4.8) | 4 | (2.0) | 44 | (23.4) | 12 | (6.2) | 44 | (23.9) | 14 | (7.4) | 45 | (22.4) | 14 | (6.8) |

| | | | | | | | | | |
|---|---|---|---|---|---|---|---|---|---|
| Missing | n | 15 | 3 | 13 | 10 | 17 | 15 | — | — |

| | | | | |
|---|---|---|---|---|
| p-value | 0.139 | <0.001 | <0.001 | <0.001 |

At least 1 buttock 2-level responder

| | | | | | | | | | | | | | | | | | |
|---|---|---|---|---|---|---|---|---|---|---|---|---|---|---|---|---|---|
| Yes | n (%) | 10 | (5.4) | 4 | (2.0) | 46 | (24.5) | 13 | (6.7) | 52 | (28.3) | 17 | (8.9) | 53 | (26.4) | 17 | (8.3) |

| | | | | | | | | | |
|---|---|---|---|---|---|---|---|---|---|
| Missing | n | 15 | 3 | 13 | 10 | 17 | 15 | — | — |

| | | | | |
|---|---|---|---|---|
| p-value | 0.093 | <0.001 | <0.001 | <0.001 |

Both buttocks 2-level responder

| | | | | | | | | | | | | | | | | | |
|---|---|---|---|---|---|---|---|---|---|---|---|---|---|---|---|---|---|
| Yes | n (%) | 7 | (3.8) | 2 | (1.0) | 36 | (19.1) | 7 | (3.6) | 41 | (22.3) | 10 | (5.3) | 42 | (20.9) | 10 | (4.9) |

| | | | | | | | | | |
|---|---|---|---|---|---|---|---|---|---|
| Missing | n | 15 | 3 | 13 | 10 | 17 | 15 | — | — |

| | | | | |
|---|---|---|---|---|
| p-value | 0.092 | <0.001 | <0.001 | <0.001 |

TABLE 32
I-GAIS ratings of the target and non-target buttocks by visit (mITT Population).

| | | Day 22 | | Day 43 | |
|---|---|---|---|---|---|
| | | CCH | Placebo | CCH | Placebo |
| I-GAIS | Statistic | (N = 201) | (N = 205) | (N = 201) | (N = 205) |

Target buttock

| Very much | n (%) | 0 | (0.0) | 0 | (0.0) | 1 | (0.5) | 0 | (0.0) |
|---|---|---|---|---|---|---|---|---|---|
| improved (3) | | | | | | | | | |
| Much | n (%) | 13 | (7.0) | 2 | (1.0) | 32 | (17.0) | 5 | (2.6) |
| improved (2) | | | | | | | | | |
| Improved (1) | n (%) | 85 | (45.5) | 34 | (17.0) | 97 | (51.6) | 48 | (24.6) |
| No change (0) | n (%) | 85 | (45.5) | 158 | (79.0) | 55 | (29.3) | 137 | (70.3) |
| Worse (−1) | n (%) | 4 | (2.1) | 5 | (2.5) | 2 | (1.1) | 4 | (2.1) |
| Much | n (%) | 0 | (0.0) | 1 | (0.5) | 1 | (0.5) | 1 | (0.5) |
| worse (−2) | | | | | | | | | |
| Very much | n (%) | 0 | (0.0) | 0 | (0.0) | 0 | (0.0) | 0 | (0.0) |
| worse (−3) | | | | | | | | | |
| Missing | N | 14 | | 5 | | 13 | | 10 | |
| | Mean (SD) | 0.6 | (0.65) | 0.2 | (0.48) | 0.9 | (0.75) | 0.3 | (0.57) |

| | | |
|---|---|---|
| P = value | <0.001 | <0.001 |

Non-target buttock

| Very much | n (%) | 0 | (0.0) | 0 | (0.0) | 3 | (1.6) | 0 | (0.0) |
|---|---|---|---|---|---|---|---|---|---|
| improved (3) | | | | | | | | | |
| Much | n (%) | 13 | (7.0) | 1 | (0.5) | 29 | (15.4) | 5 | (2.6) |
| improved (2) | | | | | | | | | |
| Improved (1) | n (%) | 79 | (42.2) | 32 | (15.9) | 103 | (54.8) | 43 | (22.1) |
| No change (0) | n (%) | 94 | (50.3) | 165 | (82.1) | 51 | (27.1) | 145 | (74.4) |
| Worse (−1) | n (%) | 1 | (0.5) | 1 | (0.5) | 1 | (0.5) | 1 | (0.5) |
| Much | n (%) | 0 | (0.0) | 1 | (0.5) | 1 | (0.5) | 1 | (0.5) |
| worse (−2) | | | | | | | | | |
| Very much | n (%) | 0 | (0.0) | 1 | (0.5) | 0 | (0.0) | 0 | (0.0) |
| worse (−3) | | | | | | | | | |
| Missing | N | 14 | | 4 | | 13 | | 10 | |
| | Mean (SD) | 0.6 | (0.63) | 0.1 | (0.48) | 0.9 | (0.74) | 0.3 | (0.53) |

| | P = value | <0.001 | | <0.001 | |
|---|---|---|---|---|---|
| | | Day 71 | | Day 71 (LOCF) | |
| | | CCH | Placebo | CCH | Placebo |
| I-GAIS | Statistic | (N = 201) | (N = 205) | (N = 201) | (N = 205) |

Target buttock

| | | | | | | | | | |
|---|---|---|---|---|---|---|---|---|---|
| Very much | n (%) | 9 | (4.9) | 2 | (1.0) | 9 | (4.5) | 2 | (1.0) |
| improved (3) | | | | | | | | | |
| Much | n (%) | 38 | (20.8) | 10 | (5.2) | 40 | (19.9) | 10 | (4.9) |
| improved (2) | | | | | | | | | |
| Improved (1) | n (%) | 81 | (44.3) | 42 | (22.0) | 90 | (44.8) | 47 | (22.9) |
| No change (0) | n (%) | 50 | (27.3) | 135 | (70.7) | 57 | (28.4) | 144 | (70.2) |
| Worse (−1) | n (%) | 5 | (2.7) | 2 | (1.0) | 5 | (2.5) | 2 | (1.0) |
| Much | n (%) | 0 | (0.0) | 0 | (0.0) | 0 | (0.0) | 0 | (0.0) |
| worse (−2) | | | | | | | | | |
| Very much | n (%) | 0 | (0.0) | 0 | (0.0) | 0 | (0.0) | 0 | (0.0) |
| worse (−3) | | | | | | | | | |

| | | | | | |
|---|---|---|---|---|---|
| Missing | N | 18 | 14 | — | — |

| | | | | | | | | |
|---|---|---|---|---|---|---|---|---|
| Mean (SD) | 1.0 | (0.89) | 0.3 | (0.65) | 1.0 | (0.87) | 0.3 | (0.64) |

| | | |
|---|---|---|
| P = value | <0.001 | <0.001 |

Non-target buttock

| | | | | | | | | | |
|---|---|---|---|---|---|---|---|---|---|
| Very much | n (%) | 6 | (3.3) | 0 | (0.0) | 7 | (3.5) | 0 | (0.0) |
| improved (3) | | | | | | | | | |
| Much | n (%) | 41 | (22.4) | 15 | (7.9) | 43 | (21.4) | 15 | (7.3) |
| improved (2) | | | | | | | | | |
| Improved (1) | n (%) | 90 | (49.2) | 37 | (19.4) | 98 | (48.8) | 41 | (20.0) |
| No change (0) | n (%) | 43 | (23.5) | 134 | (70.2) | 50 | (24.9) | 144 | (70.2) |
| Worse (−1) | n (%) | 3 | (1.6) | 5 | (2.6) | 3 | (1.5) | 5 | (2.4) |
| Much | n (%) | 0 | (0.0) | 0 | (0.0) | 0 | (0.0) | 0 | (0.0) |
| worse (−2) | | | | | | | | | |
| Very much | n (%) | 0 | (0.0) | 0 | (0.0) | 0 | (0.0) | 0 | (0.0) |
| worse (−3) | | | | | | | | | |

| | | | | | |
|---|---|---|---|---|---|
| Missing | N | 18 | 14 | — | — |

| | | | | | | | | |
|---|---|---|---|---|---|---|---|---|
| Mean (SD) | 1.0 | (0.81) | 0.3 | (0.66) | 1.0 | (0.82) | 0.3 | (0.64) |

| | | |
|---|---|---|
| P = value | <0.001 | <0.001 |

TABLE 33
I-GAIS 1-level and 2-level responders for target buttock, non-target buttock, at least 1 buttock, and both buttocks (mITT Population).

| | | Day 22 | | Day 43 | | Day 71 | | Day 71 (LOCF) | |
|---|---|---|---|---|---|---|---|---|---|
| | | CCH | Placebo | CCH | Placebo | CCH | Placebo | CCH | Placebo |
| I-GAIS | Statistic | (N = 201) | (N = 205) | (N = 201) | (N = 205) | (N = 201) | (N = 205) | (N = 201) | (N = 205) |

Target buttock 1-level responder

| | | | | | | | | | | | | | | | | | |
|---|---|---|---|---|---|---|---|---|---|---|---|---|---|---|---|---|---|
| Yes | n (%) | 98 | (52.4) | 36 | (18.0) | 130 | (69.1) | 53 | (27.2) | 128 | (69.9) | 54 | (28.3) | 139 | (69.2) | 59 | (28.8) |

| | | | | | | | | | |
|---|---|---|---|---|---|---|---|---|---|
| Missing | n | 14 | 5 | 13 | 10 | 18 | 14 | — | — |

| | | | | |
|---|---|---|---|---|
| p-value | <0.001 | <0.001 | <0.001 | <0.001 |

Non-target buttock 1-level responder

| | | | | | | | | | | | | | | | | | |
|---|---|---|---|---|---|---|---|---|---|---|---|---|---|---|---|---|---|
| Yes | n (%) | 92 | (49.2) | 33 | (16.4) | 135 | (71.8) | 48 | (24.6) | 137 | (74.9) | 52 | (27.2) | 148 | (73.6) | 56 | (27.3) |

| | | | | | | | | | |
|---|---|---|---|---|---|---|---|---|---|
| Missing | n | 14 | 4 | 13 | 10 | 18 | 14 | — | — |

| | | | | |
|---|---|---|---|---|
| p-value | <0.001 | <0.001 | <0.001 | <0.001 |

At least 1 buttock 1-level responder

| | | | | | | | | | | | | | | | | | |
|---|---|---|---|---|---|---|---|---|---|---|---|---|---|---|---|---|---|
| Yes | n (%) | 108 | (57.8) | 38 | (19.0) | 145 | (77.1) | 62 | (31.8) | 146 | (79.8) | 59 | (30.9) | 158 | (78.6) | 64 | (31.2) |

| | | | | | | | | | |
|---|---|---|---|---|---|---|---|---|---|
| Missing | n | 14 | 5 | 13 | 10 | 18 | 14 | — | — |

| | | | | |
|---|---|---|---|---|
| p-value | <0.001 | <0.001 | <0.001 | <0.001 |

Both buttocks 1-level responder

| | | | | | | | | | | | | | | | | | |
|---|---|---|---|---|---|---|---|---|---|---|---|---|---|---|---|---|---|
| Yes | n (%) | 82 | (43.9) | 31 | (15.4) | 120 | (63.8) | 39 | (20.0) | 119 | (65.0) | 47 | (24.6) | 129 | (64.2) | 51 | (24.9) |

| | | | | | | | | | |
|---|---|---|---|---|---|---|---|---|---|
| Missing | n | 14 | 4 | 13 | 10 | 18 | 14 | — | — |

| | | | | |
|---|---|---|---|---|
| p-value | <0.001 | <0.001 | <0.001 | <0.001 |

Target buttock 2-level responder

| | | | | | | | | | | | | | | | | | |
|---|---|---|---|---|---|---|---|---|---|---|---|---|---|---|---|---|---|
| Yes | n (%) | 13 | (7.0) | 2 | (1.0) | 33 | (17.6) | 5 | (2.6) | 4 | 7(25.7) | 12 | (6.3) | 49 | (24.4) | 12 | (5.9) |

| | | | | | | | | | |
|---|---|---|---|---|---|---|---|---|---|
| Missing | n | 14 | 5 | 13 | 10 | 18 | 14 | — | — |

| | | | | |
|---|---|---|---|---|
| p-value | <0.001 | <0.001 | <0.001 | <0.001 |

Non-target buttock 2-level responder

| | | | | | | | | | | | | | | | | | |
|---|---|---|---|---|---|---|---|---|---|---|---|---|---|---|---|---|---|
| Yes | n (%) | 13 | (7.0) | 1 | (0.5) | 32 | (17.0) | 5 | (2.6) | 47 | (25.7) | 15 | (7.9) | 50 | (24.9) | 15 | (7.3) |

| | | | | | | | | | |
|---|---|---|---|---|---|---|---|---|---|
| Missing | n | 14 | 4 | 13 | 10 | 18 | 14 | — | — |

| | | | | |
|---|---|---|---|---|
| p-value | <0.001 | <0.001 | <0.001 | <0.001 |

At least 1 buttock 2-level responder

| | | | | | | | | | | | | | | | | | |
|---|---|---|---|---|---|---|---|---|---|---|---|---|---|---|---|---|---|
| Yes | n (%) | 19 | (10.2) | 3 | (1.5) | 40 | (21.3) | 7 | (3.6) | 59 | (32.2) | 19 | (9.9) | 62 | (30.8) | 19 | (9.3) |

| | | | | | | | | | |
|---|---|---|---|---|---|---|---|---|---|
| Missing | n | 14 | 5 | 13 | 10 | 18 | 14 | — | — |

| | | | | |
|---|---|---|---|---|
| p-value | <0.001 | <0.001 | <0.001 | <0.001 |

Both buttocks 2-level responder

| | | | | | | | | | | | | | | | | | |
|---|---|---|---|---|---|---|---|---|---|---|---|---|---|---|---|---|---|
| Yes | n (%) | 7 | (3.7) | 0 | (0.0) | 25 | (13.3) | 3 | (1.5) | 35 | (19.1) | 8 | (4.2) | 37 | (18.4) | 8 | (3.9) |

| | | | | | | | | | |
|---|---|---|---|---|---|---|---|---|---|
| Missing | n | 14 | 4 | 13 | 10 | 18 | 14 | — | — |

| | | | | |
|---|---|---|---|---|
| p-value | <0.001 | <0.001 | <0.001 | <0.001 |

As shown in Table 34, mean PR-CIS change from baseline at Day 71 was statistically significantly favorable for CCH-treated subjects versus placebo-treated subjects in total score (10.9 versus −5.7) and abbreviated score (−10.9 versus −5.7) as well as individual impact scores.

TABLE 34
PR-CIS change from baseline (mITT Population).

| | | Baseline | | Day 71 | | Day 71 (LOCF) | |
|---|---|---|---|---|---|---|---|
| | | CCH | Placebo | CCH | Placebo | CCH | Placebo |
| PR-CIS | Statistic | (N = 201) | (N = 205) | (N = 201) | (N = 205) | (N = 201) | (N = 205) |

Total score

| | | | | | | |
|---|---|---|---|---|---|---|
| Mean | 51.3 | 51.5 | 38.7 | 44.7 | 38.7 | 44.8 |
| (SD) | (8.83) | (9.91) | (13.24) | (13.15) | (13.10) | (13.16) |
| Mean | — | — | −12.5 | −6.7 | −12.5 | −6.6 |
| (SD) | | | (12.63) | (12.11) | (12.63) | (12.09) |
| change | | | | | | |
| from | | | | | | |
| baseline | | | | | | |

| | | | |
|---|---|---|---|
| p-value | — | <0.001 | <0.001 |

Abbreviated score

| | | | | | | |
|---|---|---|---|---|---|---|
| Mean | 43.6 | 43.8 | 32.6 | 38.0 | 32.6 | 38.1 |
| (SD) | (7.28) | (8.14) | (10.95) | (10.94) | (10.82) | (10.94) |
| Mean | — | — | −10.9 | −5.7 | −10.9 | −5.7 |
| (SD) | | | (10.72) | (10.23) | (10.71) | (10.22) |
| change | | | | | | |
| from | | | | | | |
| baseline | | | | | | |

| | | | |
|---|---|---|---|
| p-value | — | <0.001 | <0.001 |

#1 impact of happiness

| | | | | | | |
|---|---|---|---|---|---|---|
| Mean | 1.1 | 0.9 | 4.6 | 2.4 | 4.5 | 2.4 |
| (SD) | (2.40) | (2.17) | (3.06) | (2.89) | (3.06) | (2.89) |
| Mean | — | — | 3.7 | 1.5 | 3.4 | 1.5 |
| (SD) | | | (3.67) | (3.46) | (3.88) | (3.45) |
| change | | | | | | |
| from | | | | | | |
| baseline | | | | | | |

| | | | |
|---|---|---|---|
| p-value | — | <0.001 | <0.001 |

#2 impact of bothersome

| | | | | | | |
|---|---|---|---|---|---|---|
| Mean | 8.3 | 8.4 | 6.3 | 7.1 | 6.2 | 7.1 |
| (SD) | (2.98) | (3.02) | (2.90) | (3.24) | (2.93) | (3.24) |
| Mean | — | — | −2.0 | −1.2 | −2.1 | −1.2 |
| (SD) | | | (3.92) | (4.28) | (3.90) | (4.27) |
| change | | | | | | |
| from | | | | | | |
| baseline | | | | | | |

| | | | |
|---|---|---|---|
| p-value | — | 0.009 | 0.003 |

#3 impact of self-consciousness

| | | | | | | |
|---|---|---|---|---|---|---|
| Mean | 8.9 | 8.8 | 7.0 | 7.8 | 7.0 | 7.8 |
| (SD) | (1.95) | (2.29) | (2.76) | (2.68) | (2.73) | (2.68) |
| Mean | — | — | −1.9 | −1.0 | −1.9 | −1.0 |
| (SD) | | | (2.67) | (2.45) | (2.66) | (2.45) |
| change | | | | | | |
| from | | | | | | |
| baseline | | | | | | |

| | | | |
|---|---|---|---|
| p-value | — | <0.001 | <0.001 |

#4 impact of embarrassment

| | | | | | | |
|---|---|---|---|---|---|---|
| Mean | 8.9 | 8.9 | 6.9 | 7.6 | 6.9 | 7.6 |
| (SD) | (1.97) | (2.19) | (2.87) | (2.83) | (2.84) | (2.83) |
| Mean | — | — | −2.0 | −1.2 | −2.0 | −1.2 |
| (SD) | | | (2.87) | (2.68) | (2.84) | (2.68) |
| change | | | | | | |
| from | | | | | | |
| baseline | | | | | | |

| | | | |
|---|---|---|---|
| p-value | — | 0.004 | 0.003 |

#5 impact of old appearance

| | | | | | | |
|---|---|---|---|---|---|---|
| Mean | 7.7 | 7.7 | 6.1 | 6.8 | 6.1 | 6.8 |
| (SD) | (2.57) | (2.65) | (3.06) | (3.19) | (3.04) | (3.19) |
| Mean | — | — | −1.6 | −1.0 | −1.6 | −0.9 |
| (SD) | | | (3.09) | (3.11) | (3.07) | (3.10) |
| change | | | | | | |
| from | | | | | | |
| baseline | | | | | | |

| | | | |
|---|---|---|---|
| p-value | — | 0.024 | 0.015 |

#6 impact of body shape concern

| | | | | | | |
|---|---|---|---|---|---|---|
| Mean | 8.6 | 8.6 | 7.1 | 7.8 | 7.1 | 7.9 |
| (SD) | (2.03) | (2.05) | (2.71) | (2.58) | (2.67) | (2.58) |
| Mean | — | — | −1.4 | −0.8 | −1.4 | −0.8 |
| (SD) | | | (2.68) | (2.56) | (2.63) | (2.55) |
| change | | | | | | |
| from | | | | | | |
| baseline | | | | | | |

| | | | |
|---|---|---|---|
| p-value | — | 0.005 | 0.005 |

There was no statistically significant difference between the treatment groups in mean SSRS at baseline. At Day 71, the mean score was greater in subjects treated with CCH compared to those treated with placebo. This difference was statistically significant (p<0.001). The results are provided Table 35.

TABLE 35
Subject self-rating score (SSRS) by visit (mITT Population).

| | | Baseline | | | | Day 71 | | | | Day 71 (LOCF) | | | |
|---|---|---|---|---|---|---|---|---|---|---|---|---|---|
| | | CCH | | Placebo | | CCH | | Placebo | | CCH | | Placebo | |
| SSRS Score | Statistic | (N = 201) | | (N = 205) | | (N = 201) | | (N = 205) | | (N = 201) | | (N = 205) | |
| Extremely | n (%) | 0 | (0.0) | 1 | (0.5) | 13 | (7.1) | 4 | (2.1) | 13 | (6.7) | 4 | (2.1) |
| satisfied (6) | | | | | | | | | | | | | |
| Satisfied | n (%) | 1 | (0.5) | 0 | (0.0) | 42 | (22.8) | 15 | (7.9) | 46 | (23.6) | 15 | (7.9) |
| (5) | | | | | | | | | | | | | |
| Slightly | n (%) | 4 | (2.0) | 4 | (2.0) | 47 | (25.5) | 29 | (15.3) | 48 | (24.6) | 29 | (15.2) |
| satisfied (4) | | | | | | | | | | | | | |
| Neither | n (%) | 4 | (2.0) | 1 | (0.5) | 22 | (12.0) | 38 | (20.0) | 24 | (12.3) | 38 | (19.9) |
| satisfied | | | | | | | | | | | | | |
| nor | | | | | | | | | | | | | |
| dissatisfied | | | | | | | | | | | | | |
| (3) | | | | | | | | | | | | | |
| Slightly | n (%) | 11 | (5.5) | 15 | (7.3) | 14 | (7.6) | 15 | (7.9) | 16 | (8.2) | 15 | (7.9) |
| dissatisfied | | | | | | | | | | | | | |
| (2) | | | | | | | | | | | | | |
| Dissatisfied | n (%) | 67 | (33.3) | 62 | (30.2) | 31 | (16.8) | 43 | (22.6) | 31 | (15.9) | 43 | (22.5) |
| (1) | | | | | | | | | | | | | |
| Extremely | n (%) | 114 | (56.7) | 122 | (59.5) | 15 | (8.2) | 46 | (24.2) | 17 | (8.7) | 47 | (24.6) |
| dissatisfied | | | | | | | | | | | | | |
| (0) | | | | | | | | | | | | | |
| Missing | N | 0 | | 0 | | 17 | | 15 | | 6 | | 14 | |
| | Mean (SD) | 0.6 | (0.90) | 0.6 | (0.90) | 3.3 | (1.80) | 2.1 | (1.75) | 3.3 | (1.80) | 2.1 | (1.75) |

| | | | |
|---|---|---|---|
| p-value | 0.617 | <0.001 | <0.001 |

One-level SSRS responder

| | | | | | | | | |
|---|---|---|---|---|---|---|---|---|
| n (%) | 102 | (55.4) | 48 | (25.3) | 107 | (54.9) | 48 | (25.1) |

| | | |
|---|---|---|
| p-value | <0.001 | <0.001 |

With respect to SSCT scores, a greater proportion of subjects treated with CCH were satisfied or very satisfied with their cellulite treatment than those treated with placebo (54.3% vs 25.8%, respectively, p<0.001). There was a statistically significant (p<0.001) difference in mean (SD) subject satisfaction scores at Day 71 between the CCH (0.4 [1.12]) and the placebo (−0.4 [1.17]) treatment groups. This is shown in Table 36 below.

TABLE 36
Subjects Satisfaction with Cellulite Treatment at Day 71 (mITT Population)

| | | Day 71 | | | | Day 71 (LOCF)^{a} | | | |
|---|---|---|---|---|---|---|---|---|---|
| | | CCH | | Placebo | | CCH | | Placebo | |
| Satisfaction Score | Statistic | (N = 201) | | (N = 205) | | (N = 201) | | (N = 205) | |
| Very Satisfied (+2) | n (%) | 24 | (13.0) | 7 | (3.7) | 24 | (12.3) | 7 | (3.7) |
| Satisfied (+1) | n (%) | 76 | (41.3) | 42 | (22.1) | 80 | (41.0) | 42 | (22.0) |
| Neither Satisfied Nor Dissatisfied (0) | n (%) | 46 | (25.0) | 56 | (29.5) | 52 | (26.7) | 56 | (29.3) |
| Dissatisfied (−1) | n (%) | 23 | (12.5) | 42 | (22.1) | 23 | (11.8) | 42 | (22.0) |
| Very Dissatisfied (−2) | n (%) | 15 | (8.2) | 43 | (22.6) | 16 | (8.2) | 44 | (23.0) |
| Missing | n | 17 | | 15 | | 6 | | 14 | |
| | Mean (SD) | 0.4 | (1.12) | −0.4 | (1.17) | 0.4 | (1.10) | −0.4 | (1.17) |
| | p-value^{b} | <0.001 | | | | <0.001 | | | |
| Subject Satisfaction Responder (Score ≥1) | n (%) | 100 | (54.3) | 49 (25.8) | | 104 | (53.3) | 49 | (25.7) |

| | | |
|---|---|---|
| p-value^{c} | <0.001 | <0.001 |

^{a}If Day 71 Subject Satisfaction with Cellulite Treatment assessment was missing, the last assessment after the initial injection was carried forward to Day 71.
^{b}p-value for Subject Satisfaction with Cellulite Treatment was based on Wilcoxon rank sum test. Percentages are based on total number of subjects evaluated in each treatment group.
^{c}p-value for 1-level subject satisfaction responder was based on a CMH test adjusted for analysis center. Percentages are based on total number of subjects evaluated in each treatment group.

### Efficacy Conclusions

The primary endpoint was the proportion of 2-level CR-PCSS/PR-PCSS composite responders in the target buttock at Day 71 in the ITT Population. A 2-level CR-PCSS/PR-PCSS composite responder was defined as a subject with an improvement from baseline of at least 2 levels of severity in the CR-PCSS and an improvement from baseline in at least 2 levels of severity in the PR-PCSS.

In this study, a statistically significant difference (p=0.006) was seen in the proportion of 2 level PR-PCSS/CR-PCSS composite responders in CCH treated subjects (16 [7.6%]) compared to placebo treated subjects (4 [1.9%]).

In order to confirm the drug effect using orthogonal scales, a series of secondary endpoints were evaluated. There were statistically significant differences in response that favored subjects treated with CCH over subjects treated with placebo in all 8 key secondary endpoints:
Proportion of 1-level PR-PCSS responders in the target buttock at Day 71 (114 (54.3%] in CCH treated subjects vs 77 [36.2%] in placebo treated subjects, p<0.001).
Proportion of 2-level PR-PCSS responders in the target buttock at Day 71 (51 [24.3%] in CCH treated subjects vs 26 [12.2%] in placebo treated subjects, p=0.001).
Proportion of 1-level CR-PCSS/PR-PCSS composite responders in the target buttock at Day 71 (78 (37.1%] in CCH treated subjects vs 38 [17.8%] in placebo treated subjects, p=<0.001).
Proportion of 2-level CR-PCSS/PR-PCSS composite responders in the nontarget buttock at Day 71 (16 (7.6%] in CCH treated subjects vs 2 [0.9%] in placebo treated subjects, p=<0.001).
Proportion of 1-level SSRS responders at Day 71 (102 (48.6%] in CCH treated subjects vs 28 [22.5%] in placebo treated subjects, p=<0.001).
Mean (SD) change from baseline of PR-CIS Total Score at Day 71 (−10.9 [12.51] in CCH treated subjects vs −5.9 [11.62] in placebo treated subjects, p=<0.001).
Proportion of 1-level S-GAIS responders in the target buttock at Day 71 (135 (64.3%] in CCH treated subjects vs 82 [38.5%] in placebo treated subjects, p=<0.001).
Proportion of 2-level S-GAIS responders in the target buttock at Day 71 (49 (23.3%] in CCH treated subjects vs 13 [6.1%] in placebo treated subjects, p=<0.001).

All sensitivity analyses supported the results for the primary and key secondary endpoints. There were also statistically significant differences between CCH treated subjects and placebo treated subjects in all supportive endpoints by Day 71 of the study (and in many cases statistically significant differences were seen as early as after a single treatment with CCH). In severity assessments, a negative change from baseline indicates improvement in cellulite. Results for the target and nontarget buttocks are outlined below:
At Day 71, the mean (SD) change from baseline in PR-PCSS was greater in subjects treated with CCH than in those treated with placebo in the target buttock (0.9 [0.93] vs 0.5 [0.88], respectively; p<0.001) and the nontarget buttock (0.9 [0.90] vs 0.5 [0.83], respectively; p<0.001).
At Day 71, the proportion of 1-level PR-PCSS responders was greater in subjects treated with CCH than in placebo treated subjects in the target buttock (62.0% vs 40.7%, respectively, p<0.001)) and in the nontarget buttock (65.2% vs 41.6%, respectively; p<0.001).
At Day 71, the mean (SD) change from baseline in CR-PCSS was greater in subjects treated with CCH than in those treated with placebo in the target buttock (0.7 [0.87] vs 0.4 [0.72], respectively; p<0.001) and the nontarget buttock (0.8 [0.83] vs 0.3 [0.67], respectively; p<0.001).
At Day 71 the proportion of 1-level CR-PCSS responders was greater in subjects treated with CCH than in placebo treated subjects in the target buttock (58.5% vs 32.5%, respectively; p<0.001) and in the nontarget buttock (60.7% vs 27.7%, respectively; p<0.001).
At Day 71, the proportion of 1-level CR-PCSS/PR-PCSS composite responders was greater in subjects treated with CCH than in placebo treated subjects in the target buttock (42.9% vs 20.1%, respectively; p<0.001) and in the nontarget buttock (44.5% vs 13.7%, respectively, p<0.001).
At Day 71, the mean (SD) S-GAIS was greater in subjects treated with CCH in the target buttock when compared to subjects treated with placebo (1.0 [0.99] vs 0.5 [0.79], respectively, p<0.001). The results were similar for the nontarget buttock (1.0 [0.94] in CCH treated subjects vs 0.5 [0.76] in placebo treated subjects, p<0.001).
At Day 71, the proportion of 1-level S-GAIS responders was greater in subjects treated with CCH than in placebo treated subjects in the target buttock (73.4% vs 43.2%, respectively; p<0.001) and the nontarget buttock (73.9% vs 42.6%, respectively; p<0.001).
At Day 71, the mean (SD) I-GAIS was statistically significantly (p<0.001) greater in subjects treated with CCH in the target buttock when compared to subjects treated with placebo (1.0 [0.81] vs 0.3 [0.66] respectively). The results were similar for the nontarget buttock (0.6 [0.63] in CCH treated subjects vs 0.1 [0.48] in placebo treated subjects).
At Day 71, the proportion of 1-level I-GAIS responders was greater in subjects treated with CCH than in placebo treated subjects in the target buttock (69.9% vs 28.3%, respectively; p<0.001) and the nontarget buttock (74.9% vs 27.2%, respectively; p<0.001).
Mean (SD) PR-CIS change from baseline at Day 71 was statistically significantly favorable for CCH treated subjects vs placebo treatment subjects in total score (12.5 ([2.63] vs −6.7 [12.11], respectively, p<0.001) and abbreviated score (10.9 [10.72] vs 5.7 [10.23], respectively, p<0.001), as well as individual impact scores (happiness with the appearance of cellulite [p<0.001], bothersome [p=0.009], self-consciousness [p<0.001], embarrassment [p=0.004], old appearance [p=0.024], body shape concern [p=0.005]).
At Day 71, the proportion of PR-CIS responders was greater in subjects treated with CCH than in those treated with placebo for total score (48.4% vs 24.2%, respectively, p<0.001) and for the abbreviated score (52.7% vs 29.5%, respectively; p<0.001). In addition, the proportion of responders for each individual impact score was greater in CCH treated subjects than in placebo treated subjects. These differences were also statistically significant.

Additional supportive results included:
At Day 71, the mean (SD) SSRS score was greater in subjects treated with CCH (3.3 [1.80]) than in those treated with placebo (2.1 [1.75]). This difference was statistically significant (p<0.001). A greater proportion of subjects treated with CCH were 1-level SSRS responders than those treated with placebo: 54.9% vs 25.1%, respectively. This difference was also statistically significant (p<0.001).
A greater proportion of subjects treated with CCH were satisfied or very satisfied with their cellulite treatment than those treated with placebo (54.3% vs 25.8%, respectively, p<0.001). There was a statistically significant (p<0.001) difference in mean (SD) subject satisfaction scores at Day 71 between the CCH (0.4 [1.12]) and the placebo (0.4 [1.17]) treatment groups.
A series of cross-tabulations show consistency between PR-PCSS and S-GAIS. A 1 level change in the PR-PCSS was associated with similar changes in S-GAIS.
By site analyses of the primary and secondary endpoints indicated that response was seen across multiple sites and that no single site markedly impacted the results of any efficacy analysis.

### Conclusions

These clinical results demonstrate that the administration of CCH is effective for treating cellulite in the buttocks of the subject population. This study met its primary endpoint with statistically significant difference in the proportion of 2 level CR-PCSS/PR-PCSS composite responders in CCH treated subjects compared to placebo treated subjects when given as 0.84 mg in each buttock (1.68 mg total) for up to 3 treatment sessions 21 days apart in adult women with EFP of the bilateral buttocks. There were statistically significant differences in response that favored subjects treated with CCH over subjects treated with placebo in all 8 key secondary endpoints and all supportive endpoints. There were no significant or unexpected safety concerns following administration of CCH. The majority of AEs occurred at the site of injection and resolved within 14 days. No clinically meaningful or concerning trends were observed with regard to hematology or chemistry laboratory parameters, or vital signs. Immunogenicity results did not reveal any potential differences from those seen in other clinical studies with this drug. Based on the results of this study, CCH is a safe and effective treatment for EFP in the buttocks of adult females at a dose of 0.84 mg per buttock (total dose 1.68 mg) given at 3 treatment sessions at least 21 days apart.

### Example 3—Phase 3 Clinical Study of Cch for the Treatment of Efp (Study 303)

A second clinical study was performed that was identically designed, randomized, double-blinded, and placebo-controlled as the clinical study described in Example 2. Efficacy was demonstrated and supported by numerous analyses, including the observance of a statistically significant difference (p=0.002) in the proportion of 2-level CR-PCSS/PR-PCSS composite responders in the target buttock of CCH-treated subjects (12 [5.6%])) compared to subjects treated with placebo (1 [0.5%]), consistent with improved cellulite severity in CCH-treated patients.

The following subject populations were among the populations that were analyzed:

The Intent-to-treat (ITT) Population included all randomized subjects who had at least 1 injection of study drug. All demographic and baseline characteristic summaries were based on this population. The primary and key secondary efficacy parameters were based on this population.

The Modified Intent-to-treat (mITT) Population included all ITT Population subjects with a baseline and at least 1 post injection evaluation of both the investigator CR-PCSS and subject PR-PCSS for both the target and nontarget buttocks. All secondary and supportive efficacy evaluations were based on the mITT Population.

In order to confirm the drug effect using orthogonal scales, a series of secondary clinical endpoints were evaluated. There were statistically significant differences in response that favored subjects treated with CCH over subjects treated with placebo, including (i) the proportion of 1-level PR-PCSS responders in the target buttock at Day 71; (ii) the proportion of 2-level PR-PCSS responders in the target buttock at Day 71; (iii) the proportion of 1-level CR-PCSS/PR-PCSS composited responders in the target buttock at Day 71; (iv) the proportion of 1-level SSRS responders at Day 71; (v) the mean change from baseline of PR-CIS Total Score at Day 71; (vi) the proportion of 1-level S-GAIS responders in the target buttock at Day 71; and (vii) the proportion of 2-level S-GAIS responders in the target buttock at Day 71. Unless otherwise specified in this example, “Days” as used in this study 303 were relative to the initial dose (Day 1) in study 303.

Like Example 2, there were 3 families that included 8 key secondary endpoints in this study. The differences between CCH and placebo treated subjects favored CCH in 7 of the 8 endpoints where the differences were statistically significant. For 2-level CR-PCSS/PR-PCSS composite responders in the non-target buttock, the proportion of responders was greater in the CCH-treated subjects compared to placebo, but the difference did not reach statistical significance through the multiplicity test with the family type I error rate of 5%. However, this result with the analyses of secondary endpoints of the non-target buttock strongly support a trend of a difference in the non-target buttock response that favors CCH-treated subjects over placebo-treated subjects. These results are summarized in Table 37 below.

TABLE 37
Summary of Results for 8 Key Secondary Endpoints (ITT Population)

| | | Frequency of Responders | | | | Treatment Difference | |
|---|---|---|---|---|---|---|---|
| Secondary | | CCH | | Placebo | | | Statistical |
| Family | Endpoint | (N = 214) | | (N = 206) | | p-value^{a} | Significance^{b} |
| 1 | Subject 1-Level PR-PCSS Responders of the Target Buttock at Day 71, n (%) | 124 | (57.9) | 61 | (29.6) | <.001 | * |
| 1 | Subject 2-Level PR-PCSS Responders of the Target Buttock at Day 71, n (%) | 45 | (21.0) | 12 | (5.8) | <.001 | * |
| 1 | Subject/Investigator 1-Level Composite Responders of the Target Buttock at | 89 | (41.6) | 23 | (11.2) | <.001 | * |
| | Day 71, n (%) | | | | | | |
| 1 | Subject/Investigator 2-Level Composite Responders of the Non-target Buttock | 13 | (6.1) | 4 | (1.9) | 0.033 | |
| | at Day 71 Based on CR-PCSS and PR-PCSS, n (%) | | | | | | |
| 2 | Subject 1-Level SSRS Responders at Day 71, n (%) | 90 | (42.1) | 31 | (15.0) | <.001 | * |
| 2 | Change from Baseline (Day 1) of the PR-CIS Total Score at Day 71, mean (SD) | −11.5 | (12.74) | −6.5 | (11.74) | <.001 | * |
| 3 | Subject 1-Level S-GAIS Responders of Target Buttock at Day 71, n (%) | 126 | (58.9) | 46 | (22.3) | <.001 | * |
| 3 | Subject 2-Level S-GAIS Responders of Target Buttock at Day 71, n (%) | 38 | (17.8) | 9 | (4.4) | <.001 | * |

^{a}p-value from individual analysis without multiplicity adjustment.
^{eb}* represents a family-wise statistical significance at significance level of 0.05 after multiplicity adjustment.

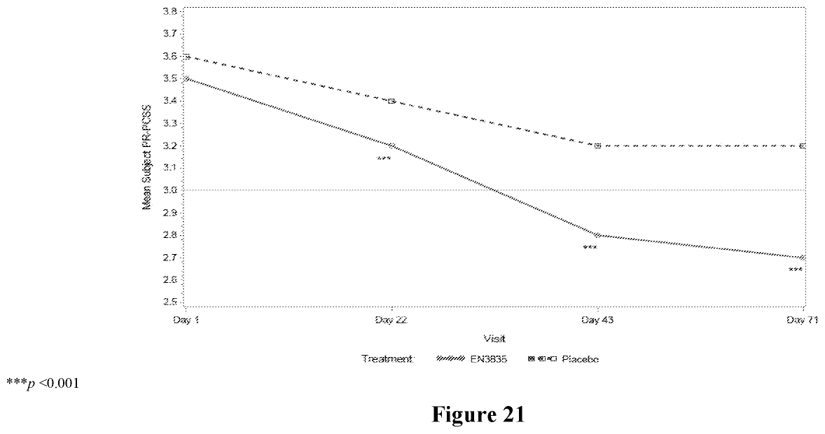

Relevant clinical data are provided in Tables 38-48 below, and shown in FIGS. 20-21.

TABLE 38
PR-PCSS rating and change from baseline for the target and non-target buttock by visit (mITT Population).

| | | Day 1 | | Day 22 | | Day 43 | |
|---|---|---|---|---|---|---|---|
| | | CCH | Placebo | CCH | Placebo | CCH | Placebo |
| PR-PCSS | Statistic | (N = 209) | (N = 201) | (N = 209) | (N = 201) | (N = 209) | (N = 201) |

Target buttock rating

| None (0) | n (%) | 0 | (0.0) | 0 | (0.0) | 0 | (0.0) | 1 | (0.5) | 0 | (0.0) | 0 | (0.0) |
|---|---|---|---|---|---|---|---|---|---|---|---|---|---|
| Almost | n (%) | 0 | (0.0) | 0 | (0.0) | 1 | (0.5) | 1 | (0.5) | 17 | (9.1) | 3 | (1.6) |
| none (1) | | | | | | | | | | | | | |
| Mild (2) | n (%) | 0 | (0.0) | 0 | (0.0) | 45 | (22.8) | 17 | (8.5) | 61 | (32.8) | 26 | (13.6) |
| Moderate | n (%) | 89 | (42.6) | 82 | (40.8) | 78 | (39.6) | 79 | (39.7) | 67 | (36.0) | 85 | (44.5) |
| (3) | | | | | | | | | | | | | |
| Severe | n (%) | 120 | (57.4) | 119 | (59.2) | 73 | (37.1) | 101 | (50.8) | 41 | (22.0) | 77 | (40.3) |

| (4) | | | | | | | |
|---|---|---|---|---|---|---|---|
| Not done | n (%) | — | — | 12 | 2 | 23 | 10 |
| | Mean | 3.6 | 3.6 | 3.1 | 3.4 | 2.7 | 3.2 |
| | SD | 0.50 | 0.49 | 0.78 | 0.71 | 0.91 | 0.74 |

Target buttock change from baseline

| | | | | | | | | | | | |
|---|---|---|---|---|---|---|---|---|---|---|---|
| −4 | n (%) | — | — | 0 | (0.0) | 0 | (0.0) | 0 | (0.0) | 0 | (0.0) |
| −3 | n (%) | — | — | 1 | (0.5) | 1 | (0.5) | 6 | (3.2) | 0 | (0.0) |
| −2 | n (%) | — | — | 9 | (4.6) | 5 | (2.5) | 36 | (19.4) | 13 | (6.8) |
| −1 | n (%) | — | — | 68 | (34.5) | 32 | (16.1) | 75 | (40.3) | 44 | (23.0) |
| 0 | n (%) | — | — | 115 | (58.4) | 155 | (77.9) | 65 | (34.9) | 130 | (68.1) |
| +1 | n (%) | — | — | 4 | (2.0) | 6 | (3.0) | 4 | (2.2) | 4 | (2.1) |

| | | | | | | | |
|---|---|---|---|---|---|---|---|
| Not done | n | — | — | 12 | 2 | 23 | 10 |
| | Mean | — | — | −0.4 | −0.2 | −0.9 | −0.3 |
| | SD | — | — | 0.64 | 0.55 | 0.86 | 0.64 |

| | | |
|---|---|---|
| p-value | <0.001 | <0.001 |

Non-target buttock rating

| None (0) | n (%) | 0 | (0.0) | 0 | (0.0) | 0 | (0.0) | 1 | (0.5) | 0 | (0.0) | 0 | (0.0) |
|---|---|---|---|---|---|---|---|---|---|---|---|---|---|
| Almost | n (%) | 0 | (0.0) | 0 | (0.0) | 3 | (1.5) | 0 | (0.0) | 17 | (9.1) | 4 | (2.1) |
| none (1) | | | | | | | | | | | | | |
| Mild (2) | n (%) | 0 | (0.0) | 0 | (0.0) | 38 | (19.3) | 19 | (9.5) | 55 | (29.6) | 24 | (12.6) |
| Moderate | n (%) | 96 | (45.9) | 71 | (35.3) | 82 | (41.6) | 71 | (35.7) | 67 | (36.0) | 86 | (45.0) |
| (3) | | | | | | | | | | | | | |
| Severe | n (%) | 113 | (54.1) | 130 | (64.7) | 74 | (37.6) | 108 | (54.3) | 47 | (25.3) | 77 | (40.3) |

| (4) | | | | | | | |
|---|---|---|---|---|---|---|---|
| Not done | n (%) | — | — | 12 | 2 | 23 | 10 |
| | Mean | 3.5 | 3.6 | 3.2 | 3.4 | 2.8 | 3.2 |
| | SD | 0.50 | 0.48 | 0.78 | 0.71 | 0.93 | 0.75 |

Non-target buttock change from baseline

| | | | | | | | | | | | |
|---|---|---|---|---|---|---|---|---|---|---|---|
| −4 | n (%) | — | — | 0 | (0.0) | 0 | (0.0) | 0 | (0.0) | 0 | (0.0) |
| −3 | n (%) | — | — | 1 | (0.5) | 1 | (0.5) | 4 | (2.2) | 1 | (0.5) |
| −2 | n (%) | — | — | 9 | (4.6) | 5 | (2.5) | 34 | (18.3) | 11 | (5.8) |
| −1 | n (%) | — | — | 60 | (30.5) | 35 | (17.6) | 73 | (39.2) | 54 | (28.3) |
| 0 | n (%) | — | — | 125 | (63.5) | 153 | (76.9) | 69 | (37.1) | 123 | (64.4) |
| +1 | n (%) | — | — | 2 | (1.0) | 5 | (2.5) | 6 | (3.2) | 2 | (1.0) |

| | | | | | | | |
|---|---|---|---|---|---|---|---|
| Not done | n | — | — | 12 | 2 | 23 | 10 |
| | Mean | — | — | −0.4 | −0.2 | −0.8 | −0.4 |
| | SD | — | — | 0.62 | 0.55 | 0.85 | 0.64 |

| | | |
|---|---|---|
| p-value | <0.001 | <0.001 |

| | | Day 71 | | | | Day 71 (LOCF) | | | |
|---|---|---|---|---|---|---|---|---|---|
| | | CCH | | Placebo | | CCH | | Placebo | |
| PR-PCSS | Statistic | (N = 209) | | (N = 201) | | (N = 209) | | (N = 201) | |
| Target buttock rating | | | | | | | | | |
| None (0) | n (%) | 3 | (1.6) | 0 | (0.0) | 3 | (1.4) | 0 | (0.0) |
| Almost | n (%) | 19 | (10.2) | 2 | (1.0) | 19 | (9.1) | 2 | (1.0) |
| none (1) | | | | | | | | | |
| Mild (2) | n (%) | 61 | (32.8) | 32 | (16.8) | 64 | (30.6) | 32 | (15.9) |
| Moderate | n (%) | 65 | (34.9) | 76 | (39.8) | 76 | (36.4) | 81 | (40.3) |
| (3) | | | | | | | | | |
| Severe | n (%) | 38 | (20.4) | 81 | (42.4) | 47 | (22.5) | 86 | (42.8) |
| (4) | | | | | | | | | |
| Not done | n (%) | 23 | | 10 | | — | | — | |
| | Mean | 2.6 | | 3.2 | | 2.7 | | 3.2 | |
| | SD | 0.97 | | 0.76 | | 0.97 | | 0.75 | |
| Target buttock change from baseline | | | | | | | | | |
| −4 | n (%) | 1 | (0.5) | 0 | (0.0) | 1 | (0.5) | 0 | (0.0) |
| −3 | n (%) | 7 | (3.8) | 0 | (0.0) | 7 | (3.3) | 0 | (0.0) |
| −2 | n (%) | 37 | (19.9) | 12 | (6.3) | 38 | (18.2) | 12 | (6.0) |
| −1 | n (%) | 79 | (42.5) | 49 | (25.7) | 87 | (41.6) | 50 | (24.9) |
| 0 | n (%) | 58 | (31.2) | 125 | (65.4) | 72 | (34.4) | 134 | (66.7) |
| +1 | n (%) | 4 | (2.2) | 5 | (2.6) | 4 | (1.9) | 5 | (2.5) |
| Not done | n | 23 | | 10 | | — | | — | |
| | Mean | −0.9 | | −0.4 | | −0.9 | | −0.3 | |
| | SD | 0.89 | | 0.64 | | 0.88 | | 0.63 | |
| | p-value | <0.001 | | | | <0.001 | | | |
| Non-target buttock rating | | | | | | | | | |
| None (0) | n (%) | 1 | (0.5) | 0 | (0.0) | 1 | (0.5) | 0 | (0.0) |
| Almost | n (%) | 21 | (11.3) | 3 | (1.6) | 21 | (10.0) | 3 | (1.5) |
| none (1) | | | | | | | | | |
| Mild (2) | n (%) | 58 | (31.2) | 32 | (16.8) | 64 | (30.6) | 34 | (16.9) |
| Moderate | n (%) | 66 | (35.5) | 72 | (37.7) | 74 | (35.4) | 75 | (37.3) |
| (3) | | | | | | | | | |
| Severe | n (%) | 40 | (21.5) | 84 | (44.0) | 49 | (23.4) | 89 | (44.3) |
| (4) | | | | | | | | | |
| Not done | n (%) | 23 | | 10 | | — | | — | |
| | Mean | 2.7 | | 3.2 | | 2.7 | | 3.2 | |
| | SD | 0.96 | | 0.78 | | 0.95 | | 0.78 | |
| Non-target buttock change from baseline | | | | | | | | | |
| −4 | n (%) | 0 | (0.0) | 0 | (0.0) | 0 | (0.0) | 0 | (0.0) |
| −3 | n (%) | 5 | (2.7) | 0 | (0.0) | 5 | (2.4) | 0 | (0.0) |
| −2 | n (%) | 38 | (20.4) | 15 | (7.9) | 39 | (18.7) | 15 | (7.5) |
| −1 | n (%) | 77 | (41.4) | 53 | (27.7) | 85 | (40.7) | 56 | (27.9) |
| 0 | n (%) | 63 | (33.9) | 118 | (61.8) | 75 | (35.9) | 125 | (62.2) |
| +1 | n (%) | 3 | (1.6) | 5 | (2.6) | 5 | (2.4) | 5 | (2.5) |

| | | | | | |
|---|---|---|---|---|---|
| Not done | n | 23 | 10 | — | — |
| | Mean | −0.9 | −0.4 | −0.8 | −0.4 |
| | SD | 0.84 | 0.67 | 0.84 | 0.66 |

| | | |
|---|---|---|
| p-value | <0.001 | <0.001 |

TABLE 39
PR-PCSS 1-level and 2-level responders for target buttock, non-target buttock, at least 1 buttock, and both buttocks (mITT Population).

| | | Day 22 | | Day 43 | | Day 71 | | Day 71 (LOCF) | |
|---|---|---|---|---|---|---|---|---|---|
| PR- | Sta- | CCH | Placebo | CCH | Placebo | CCH | Placebo | CCH | Placebo |
| PCSS | tistic | (N = 209) | (N = 201) | (N = 209) | (N = 201) | (N = 209) | (N = 201) | (N = 209) | (N = 201) |

Target buttock 1-level responder

| | | | | | | | | | | | | | | | | | |
|---|---|---|---|---|---|---|---|---|---|---|---|---|---|---|---|---|---|
| Yes | n (%) | 78 | (39.6) | 38 | (19.1) | 117 | (62.9) | 57 | (29.8) | 124 | (66.7) | 61 | (31.9) | 133 | (63.6) | 62 | (30.8) |
| No | n (%) | 119 | (60.4) | 161 | (80.9) | 69 | (37.1) | 134 | (70.2) | 62 | (33.3) | 130 | (68.1) | 76 | (36.4) | 139 | (69.2) |

| | | | | | | | | | |
|---|---|---|---|---|---|---|---|---|---|
| Miss- | n (%) | 12 | 2 | 23 | 10 | 23 | 10 | — | — |
| ing | | | | | | | | | |

| | | | | |
|---|---|---|---|---|
| p-value | <0.001 | <0.001 | <0.001 | <0.001 |

Non-target buttock 1-level responder

| | | | | | | | | | | | | | | | | | |
|---|---|---|---|---|---|---|---|---|---|---|---|---|---|---|---|---|---|
| Yes | n (%) | 70 | (35.5) | 41 | (20.6) | 111 | (59.7) | 66 | (34.6) | 120 | (64.5) | 68 | (35.6) | 129 | (61.7) | 71 | (35.3) |
| No | n (%) | 127 | (64.5) | 158 | (79.4) | 75 | (40.3) | 125 | (65.4) | 66 | (35.5) | 123 | (64.4) | 80 | (38.3) | 130 | (64.7) |

| | | | | | | | | | |
|---|---|---|---|---|---|---|---|---|---|
| Miss- | n (%) | 12 | 2 | 23 | 10 | 23 | 10 | — | — |
| ing | | | | | | | | | |

| | | | | |
|---|---|---|---|---|
| p-value | 0.001 | <0.001 | <0.001 | <0.001 |

At least buttock 1-level responder

| | | | | | | | | | | | | | | | | | |
|---|---|---|---|---|---|---|---|---|---|---|---|---|---|---|---|---|---|
| Yes | n (%) | 92 | (46.7) | 56 | (28.1) | 132 | (71.0) | 79 | (41.4) | 135 | (72.6) | 81 | (42.4) | 147 | (70.3) | 84 | (41.8) |
| No | n (%) | 105 | (53.3) | 143 | (71.9) | 54 | (29.0) | 112 | (58.6) | 51 | (27.4) | 110 | (57.6) | 62 | (29.7) | 117 | (58.2) |

| | | | | | | | | | |
|---|---|---|---|---|---|---|---|---|---|
| Miss- | n (%) | 12 | 2 | 23 | 10 | 23 | 10 | — | — |
| ing | | | | | | | | | |

| | | | | |
|---|---|---|---|---|
| p-value | <0.001 | <0.001 | <0.001 | <0.001 |

Both buttocks 1-level responder

| | | | | | | | | | | | | | | | | | |
|---|---|---|---|---|---|---|---|---|---|---|---|---|---|---|---|---|---|
| Yes | n (%) | 56 | (28.4) | 23 | (11.6) | 96 | (51.6) | 44 | (23.0) | 109 | (58.6) | 48 | (25.1) | 115 | (55.0) | 49 | (24.4) |
| No | n (%) | 141 | (71.6) | 176 | (88.4) | 90 | (48.4) | 147 | (77.0) | 77 | (41.4) | 143 | (74.9) | 94 | (45.0) | 152 | (75.6) |

| | | | | | | | | | |
|---|---|---|---|---|---|---|---|---|---|
| Miss- | n (%) | 12 | 2 | 23 | 10 | 23 | 10 | — | — |
| ing | | | | | | | | | |

| | | | | |
|---|---|---|---|---|
| p-value | <0.001 | <0.001 | <0.001 | <0.001 |

Target buttock 2-level responder

| | | | | | | | | | | | | | | | | | |
|---|---|---|---|---|---|---|---|---|---|---|---|---|---|---|---|---|---|
| Yes | n (%) | 10 | (5.1) | 6 | (3.0) | 42 | (22.6) | 13 | (6.8) | 45 | (24.2) | 12 | (6.3) | 46 | (22.0) | 12 | (6.0) |
| No | n (%) | 187 | (94.9) | 193 | (97.0) | 144 | (77.4) | 178 | (93.2) | 141 | (75.8) | 179 | (93.7) | 163 | (78.0) | 189 | (94.0) |

| | | | | | | | | | |
|---|---|---|---|---|---|---|---|---|---|
| Miss- | n (%) | 12 | 2 | 23 | 10 | 23 | 10 | — | — |
| ing | | | | | | | | | |

| | | | | |
|---|---|---|---|---|
| p-value | 0.313 | <0.001 | <0.001 | <0.001 |

Non-target buttock 2-level responder

| | | | | | | | | | | | | | | | | | |
|---|---|---|---|---|---|---|---|---|---|---|---|---|---|---|---|---|---|
| Yes | n (%) | 10 | (5.1) | 6 | (3.0) | 38 | (20.4) | 12 | (6.3) | 43 | (23.1) | 15 | (7.9) | 44 | (21.1) | 15 | (7.5) |
| No | n (%) | 187 | (94.9) | 193 | (97.0) | 148 | (79.6) | 179 | (93.7) | 143 | (76.9) | 176 | (92.1) | 165 | (78.9) | 186 | (92.5) |

| | | | | | | | | | |
|---|---|---|---|---|---|---|---|---|---|
| Miss- | n (%) | 12 | 2 | 23 | 10 | 23 | 10 | — | — |
| ing | | | | | | | | | |

| | | | | |
|---|---|---|---|---|
| p-value | 0.269 | <0.001 | <0.001 | <0.001 |

At least 1 buttock 2-level responder

| | | | | | | | | | | | | | | | | | |
|---|---|---|---|---|---|---|---|---|---|---|---|---|---|---|---|---|---|
| Yes | n (%) | 15 | (7.6) | 8 | (4.0) | 60 | (32.3) | 19 | (9.9) | 59 | (31.7) | 20 | (10.5) | 61 | (29.2) | 20 | (10.0) |
| No | n (%) | 182 | (92.4) | 191 | (96.0) | 126 | (67.7) | 172 | (90.1) | 127 | (68.3) | 171 | (89.5) | 148 | (70.8) | 181 | (90.0) |

| | | | | | | | | | |
|---|---|---|---|---|---|---|---|---|---|
| Miss- | n (%) | 12 | 2 | 23 | 10 | 23 | 10 | — | — |
| ing | | | | | | | | | |

| | | | | |
|---|---|---|---|---|
| p-value | 0.124 | <0.001 | <0.001 | <0.001 |

Both buttocks 2-level responder

| | | | | | | | | | | | | | | | | | |
|---|---|---|---|---|---|---|---|---|---|---|---|---|---|---|---|---|---|
| Yes | n (%) | 5 | (2.5) | 4 | (2.0) | 20 | (10.8) | 6 | (3.1) | 29 | (15.6) | 7 | (3.7) | 29 | (13.9) | 7 | (3.5) |
| No | n (%) | 192 | (97.5) | 195 | (98.0) | 166 | (89.2) | 185 | (96.9) | 157 | (84.4) | 184 | (96.3) | 180 | (86.1) | 194 | (96.5) |

| | | | | | | | | | |
|---|---|---|---|---|---|---|---|---|---|
| Miss- | n (%) | 12 | 2 | 23 | 10 | 23 | 10 | — | — |
| ing | | | | | | | | | |

| | | | | |
|---|---|---|---|---|
| p-value | 0.708 | 0.003 | <0.001 | <0.001 |

TABLE 40
CR-PCSS rating and change from baseline for the target and non-target buttocks by visit (mITT Population).

| | | Day 1 | | Day 22 | | Day 43 | |
|---|---|---|---|---|---|---|---|
| | | CCH | Placebo | CCH | Placebo | CCH | Placebo |
| CR-PCSS | Statistic | (N = 209) | (N = 201) | (N = 209) | (N = 201) | (N = 209) | (N = 201) |

Target buttock rating

| None (0) | n (%) | 0 | (0.0) | 0 | (0.0) | 0 | (0.0) | 1 | (0.5) | 0 | (0.0) | 0 | (0.0) |
|---|---|---|---|---|---|---|---|---|---|---|---|---|---|
| Almost | n (%) | 0 | (0.0) | 0 | (0.0) | 4 | (2.0) | 0 | (0.0) | 10 | (5.4) | 2 | (1.0) |
| none (1) | | | | | | | | | | | | | |
| Mild (2) | n (%) | 0 | (0.0) | 0 | (0.0) | 36 | (18.4) | 7 | (3.5) | 66 | (35.5) | 17 | (8.9) |
| Moderate (3) | n (%) | 127 | (60.8) | 128 | (63.7) | 115 | (58.7) | 131 | (65.8) | 75 | (40.3) | 115 | (60.2) |
| Severe (4) | n (%) | 82 | (39.2) | 73 | (36.3) | 41 | (20.9) | 60 | (30.2) | 35 | (18.8) | 57 | (29.8) |

| | | | | | | | |
|---|---|---|---|---|---|---|---|
| Not done | n (%) | — | — | 13 | 2 | 23 | 10 |
| | Mean | 3.4 | 3.4 | 3.0 | 3.3 | 2.7 | 3.2 |
| | SD | 0.49 | 0.48 | 0.69 | 0.57 | 0.83 | 0.63 |

Target buttock change from baseline

| | | | | | | | | | | | |
|---|---|---|---|---|---|---|---|---|---|---|---|
| −4 | n (%) | — | — | 0 | (0.0) | 0 | (0.0) | 0 | (0.0) | 0 | (0.0) |
| −3 | n (%) | — | — | 0 | (0.0) | 1 | (0.5) | 0 | (0.0) | 0 | (0.0) |
| −2 | n (%) | — | — | 6 | (3.1) | 0 | (0.0) | 20 | (10.8) | 3 | (1.6) |
| −1 | n (%) | — | — | 71 | (36.2) | 29 | (14.6) | 91 | (48.9) | 33 | (17.3) |
| 0 | n (%) | — | — | 117 | (59.7) | 160 | (80.4) | 72 | (38.7) | 148 | (77.5) |
| +1 | n (%) | — | — | 2 | (1.0) | 9 | (4.5) | 3 | (1.6) | 7 | (3.7) |

| | | | | | | | |
|---|---|---|---|---|---|---|---|
| Not done | n | — | — | 13 | 2 | 23 | 10 |
| | Mean | — | — | −0.4 | −0.1 | −0.7 | −0.2 |
| | SD | — | — | 0.57 | 0.47 | 0.68 | 0.50 |

| | | |
|---|---|---|
| p-value | <0.001 | <0.001 |

Non-target buttock rating

| None (0) | n (%) | 0 | (0.0) | 0 | (0.0) | 0 | (0.0) | 0 | (0.0) | 0 | (0.0) | 0 | (0.0) |
|---|---|---|---|---|---|---|---|---|---|---|---|---|---|
| Almost | n (%) | 0 | (0.0) | 0 | (0.0) | 1 | (0.5) | 0 | (0.0) | 12 | (6.5) | 3 | (1.6) |
| none (1) | | | | | | | | | | | | | |
| Mild (2) | n (%) | 0 | (0.0) | 0 | (0.0) | 49 | (25.0) | 10 | (5.0) | 69 | (37.1) | 13 | (6.8) |
| Moderate (3) | n (%) | 126 | (60.3) | 123 | (61.2) | 95 | (48.5) | 123 | (61.8) | 74 | (39.8) | 115 | (60.2) |
| Severe (4) | n (%) | 83 | (39.7) | 78 | (38.8) | 51 | (26.0) | 66 | (33.2) | 31 | (16.7) | 60 | (31.4) |

| | | | | | | | |
|---|---|---|---|---|---|---|---|
| Not done | n (%) | — | — | 13 | 2 | 23 | 10 |
| | Mean | 3.4 | 3.4 | 3.0 | 3.3 | 2.7 | 3.2 |
| | SD | 0.49 | 0.49 | 0.73 | 0.55 | 0.83 | 0.63 |

Non-target buttock change from baseline

| | | | | | | | | | | | |
|---|---|---|---|---|---|---|---|---|---|---|---|
| −4 | n (%) | — | — | 0 | (0.0) | 0 | (0.0) | 0 | (0.0) | 0 | (0.0) |
| −3 | n (%) | — | — | 0 | (0.0) | 0 | (0.0) | 0 | (0.0) | 0 | (0.0) |
| −2 | n (%) | — | — | 4 | (2.0) | 0 | (0.0) | 24 | (12.9) | 4 | (2.1) |
| −1 | n (%) | — | — | 74 | (37.8) | 35 | (17.6) | 92 | (49.5) | 35 | (18.3) |
| 0 | n (%) | — | — | 114 | (58.2) | 151 | (75.9) | 68 | (36.6) | 142 | (74.3) |
| +1 | n (%) | — | — | 4 | (2.0) | 13 | (6.5) | 2 | (1.1) | 10 | (5.2) |

| | | | | | | | |
|---|---|---|---|---|---|---|---|
| Not done | n | — | — | 13 | 2 | 23 | 10 |
| | Mean | — | — | −0.4 | −0.1 | −0.7 | −0.2 |
| | SD | — | — | 0.57 | 0.48 | 0.69 | 0.54 |

| | | |
|---|---|---|
| p-value | <0.001 | <0.001 |

| | | Day 71 | | | | Day 71 (LOCF) | | | |
|---|---|---|---|---|---|---|---|---|---|
| | | CCH | | Placebo | | CCH | | Placebo | |
| CR-PCSS | Statistic | (N = 209) | | (N = 201) | | (N = 209) | | (N = 201) | |
| Target buttock rating | | | | | | | | | |
| None (0) | n (%) | 1 | (0.5) | 0 | (0.0) | 1 | (0.5) | 0 | (0.0) |
| Almost | n (%) | 16 | (8.6) | 2 | (1.1) | 16 | (7.7) | 2 | (1.0) |
| none (1) | | | | | | | | | |
| Mild (2) | n (%) | 70 | (37.8) | 30 | (15.8) | 75 | (35.9) | 31 | (15.4) |
| Moderate (3) | n (%) | 71 | (38.4) | 103 | (54.2) | 84 | (40.2) | 108 | (53.7) |
| Severe (4) | n (%) | 27 | (14.6) | 55 | (28.9) | 33 | (15.8) | 60 | (29.9) |
| Not done | n (%) | 24 | | 11 | | — | | — | |
| | Mean | 2.6 | | 3.1 | | 2.6 | | 3.1 | |
| | SD | 0.86 | | 0.69 | | 0.86 | | 0.69 | |
| Target buttock change from baseline | | | | | | | | | |
| −4 | n (%) | 0 | (0.0) | 0 | (0.0) | 0 | (0.0) | 0 | (0.0) |
| −3 | n (%) | 1 | (0.5) | 0 | (0.0) | 1 | (0.5) | 0 | (0.0) |
| −2 | n (%) | 31 | (16.8) | 3 | (1.6) | 31 | (14.8) | 3 | (1.5) |
| −1 | n (%) | 89 | (48.1) | 49 | (25.8) | 96 | (45.9) | 51 | (25.4) |
| 0 | n (%) | 63 | (34.1) | 130 | (68.4) | 79 | (37.8) | 138 | (68.7) |
| +1 | n (%) | 1 | (0.5) | 8 | (4.2) | 2 | (1.0) | 9 | (4.5) |
| Not done | n | 24 | | 11 | | — | | — | |
| | Mean | −0.8 | | −0.2 | | −0.8 | | −0.2 | |
| | SD | 0.72 | | 0.55 | | 0.73 | | 0.55 | |
| | p-value | <0.001 | | | | <0.001 | | | |
| Non-target buttock rating | | | | | | | | | |
| None (0) | n (%) | 0 | (0.0) | 0 | (0.0) | 0 | (0.0) | 0 | (0.0) |
| Almost | n (%) | 16 | (8.6) | 3 | (1.6) | 16 | (7.7) | 3 | (1.5) |
| none (1) | | | | | | | | | |
| Mild (2) | n (%) | 75 | (40.5) | 28 | (14.7) | 83 | (39.7) | 29 | (14.4) |
| Moderate (3) | n (%) | 65 | (35.1) | 98 | (51.6) | 72 | (34.4) | 105 | (52.2) |
| Severe (4) | n (%) | 29 | (15.7) | 61 | (32.1) | 38 | (18.2) | 64 | (31.8) |
| Not done | n (%) | 24 | | 11 | | — | | — | |
| | Mean | 2.6 | | 3.1 | | 2.6 | | 3.1 | |
| | SD | 0.86 | | 0.72 | | 0.87 | | 0.71 | |
| Non-target buttock change from baseline | | | | | | | | | |
| −4 | n (%) | 0 | (0.0) | 0 | (0.0) | 0 | (0.0) | 0 | (0.0) |
| −3 | n (%) | 0 | (0.0) | 0 | (0.0) | 0 | (0.0) | 0 | (0.0) |
| −2 | n (%) | 28 | (15.1) | 5 | (2.6) | 29 | (13.9) | 5 | (2.5) |
| −1 | n (%) | 97 | (52.4) | 49 | (25.8) | 105 | (50.2) | 52 | (25.9) |
| 0 | n (%) | 58 | (31.4) | 124 | (65.3) | 72 | (34.4) | 131 | (65.2) |
| +1 | n (%) | 2 | (1.1) | 12 | (6.3) | 3 | (1.4) | 13 | (6.5) |

| | | | | | |
|---|---|---|---|---|---|
| Not done | n | 24 | 11 | — | — |
| | Mean | −0.8 | −0.2 | −0.8 | −0.2 |
| | SD | 0.69 | 0.61 | 0.70 | 0.60 |

| | | |
|---|---|---|
| p-value | <0.001 | <0.001 |

TABLE 41
CR-PCSS 1-level and 2-level responders for target buttock, non-target buttock, at least one buttock, and both buttocks (mITT Population).

| | | Day 22 | | Day 43 | | Day 71 | | Day 71 (LOCF) | |
|---|---|---|---|---|---|---|---|---|---|
| CR- | Sta- | CCH | Placebo | CCH | Placebo | CCH | Placebo | CCH | Placebo |
| PCSS | tistic | (N = 209) | (N = 201) | (N = 209) | (N = 201) | (N = 209) | (N = 201) | (N = 209) | (N-201) |

Target buttock 1-level responder

| | | | | | | | | | | | | | | | | | |
|---|---|---|---|---|---|---|---|---|---|---|---|---|---|---|---|---|---|
| Yes | n (%) | 77 | (39.3) | 30 | (15.1) | 111 | (59.7) | 36 | (18.8) | 121 | (65.4) | 52 | (27.4) | 128 | (61.2) | 54 | (26.9) |
| No | n (%) | 119 | (60.7) | 169 | (84.9) | 75 | (40.3) | 155 | (81.2) | 64 | (34.6) | 138 | (72.6) | 81 | (38.8) | 147 | (73.1) |

| | | | | | | | | | |
|---|---|---|---|---|---|---|---|---|---|
| Miss- | n (%) | 13 | 2 | 23 | 10 | 24 | 11 | — | — |
| ing | | | | | | | | | |

| | | | | |
|---|---|---|---|---|
| p-value | <0.001 | <0.001 | <0.001 | <0.001 |

Non-target buttock 1-level responder

| | | | | | | | | | | | | | | | | | |
|---|---|---|---|---|---|---|---|---|---|---|---|---|---|---|---|---|---|
| Yes | n (%) | 78 | (39.8) | 35 | (17.6) | 116 | (62.4) | 39 | (20.4) | 125 | (67.6) | 54 | (28.4) | 134 | (64.1) | 57 | (28.4) |
| No | n (%) | 118 | (60.2) | 164 | (82.4) | 70 | (37.6) | 152 | (79.6) | 60 | (32.4) | 136 | (71.6) | 75 | (35.9) | 144 | (71.6) |

| | | | | | | | | | |
|---|---|---|---|---|---|---|---|---|---|
| Miss- | n (%) | 13 | 2 | 23 | 10 | 24 | 11 | — | — |
| ing | | | | | | | | | |

| | | | | |
|---|---|---|---|---|
| p-value | <0.001 | <0.001 | <0.001 | <0.001 |

At least buttock 1-level responder

| | | | | | | | | | | | | | | | | | |
|---|---|---|---|---|---|---|---|---|---|---|---|---|---|---|---|---|---|
| Yes | n (%) | 104 | (53.1) | 48 | (24.1) | 131 | (70.4) | 52 | (27.2) | 140 | (75.7) | 70 | (36.8) | 151 | (72.2) | 73 | (36.3) |
| No | n (%) | 92 | (46.9) | 151 | (75.9) | 55 | (29.6) | 139 | (72.8) | 45 | (24.3) | 120 | (63.2) | 58 | (27.8) | 128 | (63.7) |

| | | | | | | | | | |
|---|---|---|---|---|---|---|---|---|---|
| Miss- | n (%) | 13 | 2 | 23 | 10 | 24 | 11 | — | — |
| ing | | | | | | | | | |

| | | | | |
|---|---|---|---|---|
| p-value | <0.001 | <0.001 | <0.001 | <0.001 |

Both buttocks 1-level responder

| | | | | | | | | | | | | | | | | | |
|---|---|---|---|---|---|---|---|---|---|---|---|---|---|---|---|---|---|
| Yes | n (%) | 51 | (26.0) | 17 | (8.5) | 96 | (51.6) | 23 | (12.0) | 106 | (57.3) | 36 | (18.9) | 111 | (53.1) | 38 | (18.9) |
| No | n (%) | 145 | (74.0) | 182 | (91.5) | 90 | (48.4) | 168 | (88.0) | 79 | (42.7) | 154 | (81.1) | 98 | (46.9) | 163 | (81.1) |

| | | | | | | | | | |
|---|---|---|---|---|---|---|---|---|---|
| Miss- | n (%) | 13 | 2 | 23 | 10 | 24 | 11 | — | — |
| ing | | | | | | | | | |

| | | | | |
|---|---|---|---|---|
| p-value | <0.001 | <0.001 | <0.001 | <0.001 |

Target buttock 2-level responder

| | | | | | | | | | | | | | | | | | |
|---|---|---|---|---|---|---|---|---|---|---|---|---|---|---|---|---|---|
| Yes | n (%) | 6 | (3.1) | 1 | (0.5) | 20 | (10.8) | 3 | (1.6) | 32 | (17.3) | 3 | (1.6) | 32 | (15.3) | 3 | (1.5) |
| No | n (%) | 190 | (96.9) | 198 | (99.5) | 166 | (89.2) | 188 | (98.4) | 153 | (82.7) | 187 | (98.4) | 177 | (84.7) | 198 | (98.5) |

| | | | | | | | | | |
|---|---|---|---|---|---|---|---|---|---|
| Miss- | n (%) | 13 | 2 | 23 | 10 | 24 | 11 | — | — |
| ing | | | | | | | | | |

| | | | | |
|---|---|---|---|---|
| p-value | 0.062 | <0.001 | <0.001 | <0.001 |

Non-target buttock 2-level responder

| Yes | n (%) | 4 | (2.0) | 0 | | 24 | (12.9) | 4 | (2.1) | 28 | (15.1) | 5 | (2.6) | 29 | (13.9) | 5 | (2.5) |
|---|---|---|---|---|---|---|---|---|---|---|---|---|---|---|---|---|---|
| No | n (%) | 192 | (98.0) | 199 | (100.0) | 162 | (87.1) | 187 | (97.9) | 157 | (84.9) | 185 | (97.4) | 180 | (86.1) | 196 | (97.5) |

| | | | | | | | | | |
|---|---|---|---|---|---|---|---|---|---|
| Miss- | n (%) | 13 | 2 | 23 | 10 | 24 | 11 | — | — |
| ing | | | | | | | | | |

| | | | | |
|---|---|---|---|---|
| p-value | 0.038 | <0.001 | <0.001 | <0.001 |

At least 1 buttock 2-level responder

| | | | | | | | | | | | | | | | | | |
|---|---|---|---|---|---|---|---|---|---|---|---|---|---|---|---|---|---|
| Yes | n (%) | 7 | (3.6) | 1 | (0.5) | 30 | (16.1) | 5 | (2.6) | 44 | (23.8) | 6 | (3.2) | 45 | (21.5) | 6 | (3.0) |
| No | n (%) | 189 | (96.4) | 198 | (99.5) | 156 | (83.9) | 186 | (97.4) | 141 | (76.2) | 184 | (96.8) | 164 | (78.5) | 195 | (97.0) |

| | | | | | | | | | |
|---|---|---|---|---|---|---|---|---|---|
| Miss- | n (%) | 13 | 2 | 23 | 10 | 24 | 11 | — | — |
| ing | | | | | | | | | |

| | | | | |
|---|---|---|---|---|
| p-value | 0.033 | <0.001 | <0.001 | <0.001 |

Both buttocks 2-level responder

| Yes | n (%) | 3 | (1.5) | 0 | | 14 | (7.5) | 2 | (1.0) | 16 | (8.6) | 2 | (1.1) | 16 | (7.7) | 2 | (1.0) |
|---|---|---|---|---|---|---|---|---|---|---|---|---|---|---|---|---|---|
| No | n (%) | 193 | (98.5) | 199 | (100.0) | 172 | (92.5) | 189 | (99.0) | 169 | (91.4) | 188 | (98.9) | 193 | (92.3) | 199 | (99.0) |

| | | | | | | | | | |
|---|---|---|---|---|---|---|---|---|---|
| Miss- | n (%) | 13 | 2 | 23 | 10 | 24 | 11 | — | — |
| ing | | | | | | | | | |

| | | | | |
|---|---|---|---|---|
| p-value | 0.080 | 0.001 | <0.001 | 0.001 |

TABLE 42
Composite CR-PCSS and PR-PCSS 1-level and 2-level responders for target buttock,
non-target buttock, at least 1 buttock, and both buttocks (mITT Population).

| Composite | | Day 22 | | Day 43 | |
|---|---|---|---|---|---|
| CR-PCSS/ | | CCH | Placebo | CCH | Placebo |
| PR-PCSS | Statistic | (N = 209) | (N = 201) | (N = 209) | (N = 201) |

Target buttock 1-level responder

| | | | | | | | | | |
|---|---|---|---|---|---|---|---|---|---|
| Yes | n (%) | 36 | (18.4) | 4 | (2.0) | 77 | (41.4) | 16 | (8.4) |
| No | n (%) | 160 | (81.6) | 195 | (98.0) | 109 | (58.6) | 175 | (91.6) |

| | | | | | |
|---|---|---|---|---|---|
| Missing | n (%) | 13 | 2 | 23 | 10 |

| | | |
|---|---|---|
| p-value | <0.001 | <0.001 |

Non-target buttock 1-level responder

| | | | | | | | | | |
|---|---|---|---|---|---|---|---|---|---|
| Yes | n (%) | 36 | (18.4) | 7 | (3.5) | 77 | (41.4) | 16 | (8.4) |
| No | n (%) | 160 | (81.6) | 192 | (96.5) | 109 | (58.6) | 175 | (91.6) |

| | | | | | |
|---|---|---|---|---|---|
| Missing | n (%) | 13 | 2 | 23 | 10 |

| | | |
|---|---|---|
| p-value | <0.001 | <0.001 |

At least buttock 1-level responder

| | | | | | | | | | |
|---|---|---|---|---|---|---|---|---|---|
| Yes | n (%) | 52 | (26.5) | 10 | (5.0) | 97 | (52.2) | 23 | (12.0) |
| No | n (%) | 144 | (73.5) | 189 | (95.0) | 89 | (47.8) | 168 | (88.0) |

| | | | | | |
|---|---|---|---|---|---|
| Missing | n (%) | 13 | 2 | 23 | 10 |

| | | |
|---|---|---|
| p-value | <0.001 | <0.001 |

Both buttocks 1-level responder

| | | | | | | | | | |
|---|---|---|---|---|---|---|---|---|---|
| Yes | n (%) | 20 | (10.2) | 1 | (0.5) | 57 | (30.6) | 9 | (4.7) |
| No | n (%) | 176 | (89.8) | 198 | (99.5) | 129 | (69.4) | 182 | (95.3) |

| | | | | | |
|---|---|---|---|---|---|
| Missing | n (%) | 13 | 2 | 23 | 10 |

| | | |
|---|---|---|
| p-value | <0.001 | <0.001 |

Target buttock 2-level responder

| Yes | n (%) | 1 | (0.5) | 0 | | 5 | (2.7) | 2 | (1.0) |
|---|---|---|---|---|---|---|---|---|---|
| No | n (%) | 195 | (99.5) | 199 | (100.0) | 181 | (97.3) | 189 | (99.0) |

| | | | | | |
|---|---|---|---|---|---|
| Missing | n (%) | 13 | 2 | 23 | 10 |

| | | |
|---|---|---|
| p-value | 0.330 | 0.245 |

Non-target buttock 2-level responder

| Yes | n (%) | 1 | (0.5) | 0 | | 9 | (4.8) | 1 | (0.5) |
|---|---|---|---|---|---|---|---|---|---|
| No | n (%) | 195 | (99.5) | 199 | (100.0) | 177 | (95.2) | 190 | (99.5) |

| | | | | | |
|---|---|---|---|---|---|
| Missing | n (%) | 13 | 2 | 23 | 10 |

| | | |
|---|---|---|
| p-value | 0.292 | 0.009 |

At least 1 buttock 2-level responder

| Yes | n (%) | 2 | (1.0) | 0 | | 12 | (6.5) | 2 | (1.0) |
|---|---|---|---|---|---|---|---|---|---|
| No | n (%) | 194 | (99.0) | 199 | (100.0) | 174 | (93.5) | 189 | (99.0) |

| | | | | | |
|---|---|---|---|---|---|
| Missing | n (%) | 13 | 2 | 23 | 10 |

| | | |
|---|---|---|
| p-value | 0.152 | 0.005 |

Both buttocks 2-level responder

| Yes | n (%) | 0 | | 0 | | 2 | (1.1) | 1 | (0.5) |
|---|---|---|---|---|---|---|---|---|---|
| No | n (%) | 196 | (100.0) | 199 | (100.0) | 184 | (98.9) | 190 | (99.5) |

| Missing | n (%) | 13 | 2 | 23 | 10 |
|---|---|---|---|---|---|
| | p-value | — | | 0.598 | |
| | | | | Day 71 | |
| Composite | | Day 71 | | (LOCF) | |
| CR-PCSS/ | | CCH | Placebo | CCH | Placebo |
| PR-PCSS | Statistic | (N = 209) | (N = 201) | (N = 209) | (N = 201) |

Target buttock 1-level responder

| | | | | | | | | | |
|---|---|---|---|---|---|---|---|---|---|
| Yes | n (%) | 89 | (48.1) | 23 | (12.1) | 93 | (44.5) | 23 | (11.4) |
| No | n (%) | 96 | (51.9) | 167 | (87.9) | 116 | (55.5) | 178 | (88.6) |

| | | | | | |
|---|---|---|---|---|---|
| Missing | n (%) | 24 | 11 | — | — |

| | | |
|---|---|---|
| p-value | <0.001 | <0.001 |

Non-target buttock 1-level responder

| | | | | | | | | | |
|---|---|---|---|---|---|---|---|---|---|
| Yes | n (%) | 92 | (49.7) | 29 | (15.3) | 97 | (46.4) | 30 | (14.9) |
| No | n (%) | 93 | (50.3) | 161 | (84.7) | 112 | (53.6) | 171 | (85.1) |

| | | | | | |
|---|---|---|---|---|---|
| Missing | n (%) | 24 | 11 | — | — |

| | | |
|---|---|---|
| p-value | <0.001 | <0.001 |

At least buttock 1-level responder

| | | | | | | | | | |
|---|---|---|---|---|---|---|---|---|---|
| Yes | n (%) | 108 | (58.4) | 36 | (18.9) | 115 | (55.0) | 37 | (18.4) |
| No | n (%) | 77 | (41.6) | 154 | (81.1) | 94 | (45.0) | 164 | (81.6) |

| | | | | | |
|---|---|---|---|---|---|
| Missing | n (%) | 24 | 11 | — | — |

| | | |
|---|---|---|
| p-value | <0.001 | <0.001 |

Both buttocks 1-level responder

| | | | | | | | | | |
|---|---|---|---|---|---|---|---|---|---|
| Yes | n (%) | 73 | (39.5) | 16 | (8.4) | 75 | (35.9) | 16 | (8.0) |
| No | n (%) | 112 | (60.5) | 174 | (91.6) | 134 | (64.1) | 185 | (92.0) |

| | | | | | |
|---|---|---|---|---|---|
| Missing | n (%) | 24 | 11 | — | — |

| | | |
|---|---|---|
| p-value | <0.001 | <0.001 |

Target buttock 2-level responder

| | | | | | | | | | |
|---|---|---|---|---|---|---|---|---|---|
| Yes | n (%) | 12 | (6.5) | 1 | (0.5) | 12 | (5.7) | 1 | (0.5) |
| No | n (%) | 173 | (93.5) | 189 | (99.5) | 197 | (94.3) | 200 | (99.5) |

| | | | | | |
|---|---|---|---|---|---|
| Missing | n (%) | 24 | 11 | — | — |

| | | |
|---|---|---|
| p-value | 0.001 | 0.003 |

Non-target buttock 2-level responder

| | | | | | | | | | |
|---|---|---|---|---|---|---|---|---|---|
| Yes | n (%) | 13 | (7.0) | 4 | (2.1) | 13 | (6.2) | 4 | (2.0) |
| No | n (%) | 172 | (93.0) | 186 | (97.9) | 196 | (93.8) | 197 | (98.0) |

| | | | | | |
|---|---|---|---|---|---|
| Missing | n (%) | 24 | 11 | — | — |

| | | |
|---|---|---|
| p-value | 0.021 | 0.032 |

At least 1 buttock 2-level responder

| | | | | | | | | | |
|---|---|---|---|---|---|---|---|---|---|
| Yes | n (%) | 20 | (10.8) | 4 | (2.1) | 20 | (9.6) | 4 | (2.0) |
| No | n (%) | 165 | (89.2) | 186 | (97.9) | 189 | (90.4) | 197 | (98.0) |

| | | | | | |
|---|---|---|---|---|---|
| Missing | n (%) | 24 | 11 | — | — |

| | | |
|---|---|---|
| p-value | <0.001 | 0.001 |

Both buttocks 2-level responder

| | | | | | | | | | |
|---|---|---|---|---|---|---|---|---|---|
| Yes | n (%) | 5 | (2.7) | 1 | (0.5) | 5 | (2.4) | 1 | (0.5) |
| No | n (%) | 180 | (97.3) | 189 | (99.5) | 204 | (97.6) | 200 | (99.5) |

| | | | | | |
|---|---|---|---|---|---|
| Missing | n (%) | 24 | 11 | — | — |

| | | |
|---|---|---|
| p-value | 0.084 | 0.105 |

TABLE 43
S-GAIS of target and non-target buttock by visit (mITT Population).

| | | Day 22 | | Day 43 | | Day 71 | | Day 71 (LOCF) | |
|---|---|---|---|---|---|---|---|---|---|
| | | CCH | Placebo | CCH | Placebo | CCH | Placebo | CCH | Placebo |
| S-GAIS | Statistic | (N = 209) | (N = 201) | (N = 209) | (N = 201) | (N = 209) | (N = 201) | (N = 209) | (N = 201) |

Target buttock

| Very much | n (%) | 5 | (2.6) | 1 | (0.5) | 4 | (2.2) | 0 | (0.0) | 10 | (5.4) | 0 | (0.0) | 10 | (4.8) | 0 | (0.0) |
|---|---|---|---|---|---|---|---|---|---|---|---|---|---|---|---|---|---|
| improved (3) | | | | | | | | | | | | | | | | | |
| Much | n (%) | 5 | (2.6) | 4 | (2.0) | 30 | (16.2) | 8 | (4.2) | 28 | (15.1) | 9 | (4.7) | 29 | (13.9) | 9 | (4.5) |
| improved (2) | | | | | | | | | | | | | | | | | |
| Improved (1) | n (%) | 89 | (45.4) | 31 | (15.7) | 101 | (54.6) | 47 | (24.7) | 88 | (47.3) | 37 | (19.4) | 96 | (45.9) | 38 | (18.9) |
| No | n (%) | 91 | (46.4) | 153 | (77.7) | 44 | (23.8) | 128 | (67.4) | 54 | (29.0) | 130 | (68.1) | 68 | (32.5) | 139 | (69.2) |
| change (0) | | | | | | | | | | | | | | | | | |
| Worse (−1) | n (%) | 5 | (2.6) | 8 | (4.1) | 5 | (2.7) | 7 | (3.7) | 5 | (2.7) | 14 | (7.3) | 5 | (2.4) | 14 | (7.0) |
| Much | n (%) | 1 | (0.5) | 0 | (0.0) | 0 | (0.0) | 0 | (0.0) | 0 | (0.0) | 0 | (0.0) | 0 | (0.0) | 0 | (0.0) |
| worse (−2) | | | | | | | | | | | | | | | | | |
| Very much | n (%) | 0 | (0.0) | 0 | (0.0) | 1 | (0.5) | 0 | (0.0) | 1 | (0.5) | 1 | (0.5) | 1 | (0.5) | 1 | (0.5) |
| worse (−3) | | | | | | | | | | | | | | | | | |
| Missing | N | 13 | | 4 | | 24 | | 11 | | 23 | | 10 | | — | | — | |
| | Mean | 0.5 | (0.73) | 0.2 | (0.54) | 0.9 | (0.82) | 0.3 | (0.61) | 0.9 | (0.92) | 0.2 | (0.68) | 0.8 | (0.90) | 0.2 | (0.67) |
| | (SD) | | | | | | | | | | | | | | | | |

| | | | | |
|---|---|---|---|---|
| p = | <0.001 | <0.001 | <0.001 | <0.001 |
| value | | | | |

Non-target buttock

| Very much | n (%) | 3 | (1.5) | 2 | (1.0) | 6 | (3.2) | 0 | (0.0) | 10 | (5.4) | 1 | (0.5) | 10 | (4.8) | 1 | (0.5) |
|---|---|---|---|---|---|---|---|---|---|---|---|---|---|---|---|---|---|
| improved (3) | | | | | | | | | | | | | | | | | |
| Much | n (%) | 6 | (3.1) | 5 | (2.6) | 29 | (15.7) | 8 | (4.2) | 29 | (15.6) | 6 | (3.1) | 30 | (14.4) | 6 | (3.0) |
| improved (2) | | | | | | | | | | | | | | | | | |
| Improved (1) | n (%) | 94 | (48.0) | 25 | (12.8) | 92 | (49.7) | 46 | (24.2) | 85 | (45.7) | 43 | (22.5) | 95 | (45.5) | 44 | (21.9) |
| No | n (%) | 86 | (43.9) | 151 | (77.0) | 49 | (26.5) | 129 | (67.9) | 54 | (29.0) | 130 | (68.1) | 66 | (31.6) | 138 | (68.7) |
| change (0) | | | | | | | | | | | | | | | | | |
| Worse (−1) | n (%) | 6 | (3.1) | 12 | (6.1) | 8 | (4.3) | 6 | (3.2) | 7 | (3.8) | 10 | (5.2) | 7 | (3.3) | 11 | (5.5) |
| Much | n (%) | 1 | (0.5) | 1 | (0.5) | 1 | (0.5) | 1 | (0.5) | 0 | (0.0) | 0 | (0.0) | 0 | (0.0) | 0 | (0.0) |
| worse (−2) | | | | | | | | | | | | | | | | | |
| Very much | n (%) | 0 | (0.0) | 0 | (0.0) | 0 | (0.0) | 0 | (0.0) | 1 | (0.5) | 1 | (0.5) | 1 | (0.5) | 1 | (0.5) |
| worse (−3) | | | | | | | | | | | | | | | | | |
| Missing | N | 13 | | 5 | | 24 | | 11 | | 23 | | 10 | | — | | — | |
| | Mean | 0.5 | (0.70) | 0.1 | (0.62) | 0.9 | (0.87) | 0.3 | (0.62) | 0.9 | (0.94) | 0.2 | (0.67) | 0.8 | (0.92) | 0.2 | (0.66) |
| | (SD) | | | | | | | | | | | | | | | | |

| | | | | |
|---|---|---|---|---|
| p = | <0.001 | <0.001 | <0.001 | <0.001 |
| value | | | | |

TABLE 44
S-GAIS 1-level and 2-level responders for target buttock, non-target buttock, at least 1 buttock, and both buttocks (mITT Population).

| | | Day 22 | | Day 43 | | Day 71 | | Day 71 (LOCF) | |
|---|---|---|---|---|---|---|---|---|---|
| | | CCH | Placebo | CCH | Placebo | CCH | Placebo | CCH | Placebo |
| S-GAIS | Statistic | (N = 209) | (N = 201) | (N = 209) | (N = 201) | (N = 209) | (N = 201) | (N = 209) | (N = 201) |

Target buttock 1-level responder

| | | | | | | | | | | | | | | | | | |
|---|---|---|---|---|---|---|---|---|---|---|---|---|---|---|---|---|---|
| Yes | n (%) | 99 | (50.5) | 36 | (18.3) | 135 | (73.0) | 55 | (28.9) | 126 | (67.7) | 46 | (24.1) | 135 | (64.6) | 47 | (23.4) |

| | | | | | | | | | |
|---|---|---|---|---|---|---|---|---|---|
| Missing | n | 13 | 4 | 24 | 11 | 23 | 10 | — | — |

| | | | | |
|---|---|---|---|---|
| p-value | <0.001 | <0.001 | <0.001 | <0.001 |

Non-target buttock 1-level responder

| | | | | | | | | | | | | | | | | | |
|---|---|---|---|---|---|---|---|---|---|---|---|---|---|---|---|---|---|
| Yes | n (%) | 103 | (52.6) | 32 | (16.3) | 127 | (68.6) | 54 | (28.4) | 124 | (66.7) | 50 | (26.2) | 135 | (64.6) | 51 | (25.4) |

| | | | | | | | | | |
|---|---|---|---|---|---|---|---|---|---|
| Missing | n | 13 | 5 | 24 | 11 | 23 | 10 | — | — |

| | | | | |
|---|---|---|---|---|
| p-value | <0.001 | <0.001 | <0.001 | <0.001 |

At least buttock 1-level responder

| | | | | | | | | | | | | | | | | | |
|---|---|---|---|---|---|---|---|---|---|---|---|---|---|---|---|---|---|
| Yes | n (%) | 119 | (60.7) | 45 | (23.0) | 144 | (77.8) | 67 | (35.3) | 137 | (73.7) | 60 | (31.4) | 148 | (70.8) | 62 | (30.8) |

| | | | | | | | | | |
|---|---|---|---|---|---|---|---|---|---|
| Missing | n | 13 | 5 | 24 | 11 | 23 | 10 | — | — |

| | | | | |
|---|---|---|---|---|
| p-value | <0.001 | <0.001 | <0.001 | <0.001 |

Both buttocks 1-level responder

| | | | | | | | | | | | | | | | | | |
|---|---|---|---|---|---|---|---|---|---|---|---|---|---|---|---|---|---|
| Yes | n (%) | 83 | (42.3) | 23 | (11.7) | 118 | (63.8) | 42 | (22.1) | 113 | (60.8) | 36 | (18.8) | 122 | (58.4) | 36 | (17.9) |

| | | | | | | | | | |
|---|---|---|---|---|---|---|---|---|---|
| Missing | n | 13 | 4 | 24 | 11 | 23 | 10 | — | — |

| | | | | |
|---|---|---|---|---|
| p-value | <0.001 | <0.001 | <0.001 | <0.001 |

Target buttock 2-level responder

| | | | | | | | | | | | | | | | | | |
|---|---|---|---|---|---|---|---|---|---|---|---|---|---|---|---|---|---|
| Yes | n (%) | 10 | (5.1) | 5 | (2.5) | 34 | (18.4) | 8 | (4.2) | 38 | (20.4) | 9 | (4.7) | 39 | (18.7) | 9 | (4.5) |

| | | | | | | | | | |
|---|---|---|---|---|---|---|---|---|---|
| Missing | n | 13 | 4 | 24 | 11 | 23 | 10 | — | — |

| | | | | |
|---|---|---|---|---|
| p-value | 0.192 | <0.001 | <0.001 | <0.001 |

Non-target buttock 2-level responder

| | | | | | | | | | | | | | | | | | |
|---|---|---|---|---|---|---|---|---|---|---|---|---|---|---|---|---|---|
| Yes | n (%) | 9 | (4.6) | 7 | (3.6) | 35 | (18.9) | 8 | (4.2) | 39 | (21.0) | 7 | (3.7) | 40 | (19.1) | 7 | (3.5) |

| | | | | | | | | | |
|---|---|---|---|---|---|---|---|---|---|
| Missing | n | 13 | 5 | 24 | 11 | 23 | 10 | — | — |

| | | | | |
|---|---|---|---|---|
| p-value | 0.661 | <0.001 | <0.001 | <0.001 |

At least 1 buttock 2-level responder

| | | | | | | | | | | | | | | | | | |
|---|---|---|---|---|---|---|---|---|---|---|---|---|---|---|---|---|---|
| Yes | n (%) | 13 | (6.6) | 8 | (4.1) | 42 | (22.7) | 10 | (5.3) | 50 | (26.9) | 10 | (5.2) | 51 | (24.4) | 10 | (5.0) |

| | | | | | | | | | |
|---|---|---|---|---|---|---|---|---|---|
| Missing | n | 13 | 5 | 24 | 11 | 23 | 10 | — | — |

| | | | | |
|---|---|---|---|---|
| p-value | 0.305 | <0.001 | <0.001 | <0.001 |

Both buttocks 2-level responder

| | | | | | | | | | | | | | | | | | |
|---|---|---|---|---|---|---|---|---|---|---|---|---|---|---|---|---|---|
| Yes | n (%) | 6 | (3.1) | 4 | (2.0) | 27 | (14.6) | 6 | (3.2) | 27 | (14.5) | 6 | (3.1) | 28 | (13.4) | 6 | (3.0) |

| | | | | | | | | | |
|---|---|---|---|---|---|---|---|---|---|
| Missing | n | 13 | 4 | 24 | 11 | 23 | 10 | — | — |

| | | | | |
|---|---|---|---|---|
| p-value | 0.523 | <0.001 | <0.001 | <0.001 |

TABLE 45
I-GAIS ratings of the target and non-target buttocks by visit (mITT Population).

| | | Day 22 | | Day 43 | | Day 71 | | Day 71 (LOCF) | |
|---|---|---|---|---|---|---|---|---|---|
| | | CCH | Placebo | CCH | Placebo | CCH | Placebo | CCH | Placebo |
| I-GAIS | Statistic | (N = 209) | (N = 201) | (N = 209) | (N = 201) | (N = 209) | (N = 201) | (N = 209) | (N = 201) |

Target buttock

| Very much | n (%) | | | | | | | | | | | | | | | | |
|---|---|---|---|---|---|---|---|---|---|---|---|---|---|---|---|---|---|
| improved (3) | | | | | | | | | | | | | | | | | |
| Much | n (%) | 0 | (0.0) | 0 | (0.0) | 1 | (0.5) | 0 | (0.0) | 2 | (1.1) | 1 | (0.5) | 3 | (1.4) | 1 | (0.5) |
| improved (2) | | | | | | | | | | | | | | | | | |
| Improved (1) | n (%) | 20 | (10.3) | 1 | (0.5) | 36 | (19.5) | 3 | (1.6) | 45 | (24.3) | 3 | (1.6) | 46 | (22.0) | 3 | (1.5) |
| No | n (%) | 88 | (45.1) | 18 | (9.2) | 93 | (50.3) | 33 | (17.4) | 84 | (45.4) | 42 | (22.1) | 94 | (45.0) | 42 | (20.9) |
| change (0) | | | | | | | | | | | | | | | | | |
| Worse (−1) | n (%) | 85 | (43.6) | 175 | (89.3) | 52 | (28.1) | 152 | (80.0) | 49 | (26.5) | 143 | (75.3) | 61 | (29.2) | 154 | (76.6) |
| Much | n (%) | 2 | (1.0) | 2 | (1.0) | 3 | (1.6) | 2 | (1.1) | 5 | (2.7) | 1 | (0.5) | 5 | (2.4) | 1 | (0.5) |
| worse (−2) | | | | | | | | | | | | | | | | | |
| Very much | n (%) | 0 | (0.0) | 0 | (0.0) | 0 | (0.0) | 0 | (0.0) | 0 | (0.0) | 0 | (0.0) | 0 | (0.0) | 0 | (0.0) |
| worse (−3) | | | | | | | | | | | | | | | | | |
| Missing | N | 0 | (0.0) | 0 | (0.0) | 0 | (0.0) | 0 | (0.0) | 0 | (0.0) | 0 | (0.0) | 0 | (0.0) | 0 | (0.0) |

| | | | | | | | | |
|---|---|---|---|---|---|---|---|---|
| Mean | 14 | 5 | 24 | 11 | 24 | 11 | — | — |
| (SD) | | | | | | | | |

| | | | | |
|---|---|---|---|---|
| P = | 0.6 (0.68) | 0.1 (0.34) | 0.9 (0.74) | 0.2 (0.46) |
| value | | | | |

Non-target buttock

| Very much | n (%) | 0 | (0.0) | 0 | (0.0) | 2 | (1.1) | 1 | (0.5) | 1 | (0.5) | 1 | (0.5) | 2 | (1.0) | 1 | (0.5) |
|---|---|---|---|---|---|---|---|---|---|---|---|---|---|---|---|---|---|
| improved (3) | | | | | | | | | | | | | | | | | |
| Much | n (%) | 11 | (5.6) | 3 | (1.5) | 33 | (17.8) | 2 | (1.1) | 43 | (23.2) | 6 | (3.2) | 44 | (21.1) | 6 | (3.0) |
| improved (2) | | | | | | | | | | | | | | | | | |
| Improved (1) | n (%) | 96 | (49.2) | 25 | (12.8) | 99 | (53.5) | 31 | (16.3) | 86 | (46.5) | 34 | (17.9) | 98 | (46.9) | 34 | (16.9) |
| No | n (%) | 87 | (44.6) | 167 | (85.2) | 49 | (26.5) | 155 | (81.6) | 50 | (27.0) | 149 | (78.4) | 60 | (28.7) | 160 | (79.6) |
| change (0) | | | | | | | | | | | | | | | | | |
| Worse (−1) | n (%) | 1 | (0.5) | 1 | (0.5) | 2 | (1.1) | 1 | (0.5) | 5 | (2.7) | 0 | (0.0) | 5 | (2.4) | 0 | (0.0) |
| Much | n (%) | 0 | (0.0) | 0 | (0.0) | 0 | (0.0) | 0 | (0.0) | 0 | (0.0) | 0 | (0.0) | 0 | (0.0) | 0 | (0.0) |
| worse (−2) | | | | | | | | | | | | | | | | | |
| Very much | n (%) | 0 | (0.0) | 0 | (0.0) | 0 | (0.0) | 0 | (0.0) | 0 | (0.0) | 0 | (0.0) | 0 | (0.0) | 0 | (0.0) |
| worse (−3) | | | | | | | | | | | | | | | | | |
| Missing | N | 14 | | 5 | | 24 | | 11 | | 24 | | 11 | | 0 | | 0 | |
| | Mean | 0.6 | (0.60) | 0.2 | (0.41) | 0.9 | (0.72) | 0.2 | (0.47) | 0.9 | (0.79) | 0.3 | (0.54) | 0.9 | (0.79) | 0.2 | (0.52) |
| | (SD) | | | | | | | | | | | | | | | | |

| | | | | |
|---|---|---|---|---|
| P = | <0.001 | <0.001 | <0.001 | <0.001 |
| value | | | | |

TABLE 46
I-GAIS 1-level and 2-level responders for target buttock, non-target
buttock, at least 1 buttock, and both buttocks (mITT Population).

| | | Day 22 | | Day 43 | | Day 71 | | Day 71 (LOCF) | |
|---|---|---|---|---|---|---|---|---|---|
| | | CCH | Placebo | CCH | Placebo | CCH | Placebo | CCH | Placebo |
| I-GAIS | Statistic | (N = 209) | (N = 201) | (N = 209) | (N = 201) | (N = 209) | (N = 201) | (N = 209) | (N = 201) |

Target buttock 1-level responder

| | | | | | | | | | | | | | | |
|---|---|---|---|---|---|---|---|---|---|---|---|---|---|---|
| Yes | n (%) | 108 | (55.4) | 19 | (9.7) | 130 | (70.3) | 36 (18.9) | 131 | (70.8) | 46 (24.2) | 143 | (68.4) | 46 (22.9) |

| | | | | | | | | | |
|---|---|---|---|---|---|---|---|---|---|
| Missing | n | 14 | 5 | 24 | 11 | 24 | 11 | — | — |

| | | | | |
|---|---|---|---|---|
| p-value | <0.001 | <0.001 | <0.001 | <0.001 |

Non-target buttock 1-level responder

| | | | | | | | | | | | | | | |
|---|---|---|---|---|---|---|---|---|---|---|---|---|---|---|
| Yes | n (%) | 107 | (54.9) | 28 | (14.3) | 134 | (72.4) | 34 (17.9) | 130 | (70.3) | 41 (21.6) | 144 | (68.9) | 41 (20.4) |

| | | | | | | | | | |
|---|---|---|---|---|---|---|---|---|---|
| Missing | n | 14 | 5 | 24 | 11 | 24 | 11 | — | — |

| | | | | |
|---|---|---|---|---|
| p-value | <0.001 | <0.001 | <0.001 | <0.001 |

At least 1 buttock 1-level responder

| | | | | | | | | | | | | | | |
|---|---|---|---|---|---|---|---|---|---|---|---|---|---|---|
| Yes | n (%) | 125 | (64.1) | 30 | (15.3) | 139 | (75.1) | 40 (21.1) | 141 | (76.2) | 52 (27.4) | 157 ( | 75.1) | 52 (25.9) |

| | | | | | | | | | |
|---|---|---|---|---|---|---|---|---|---|
| Missing | n | 14 | 5 | 24 | 11 | 24 | 11 | — | — |

| | | | | |
|---|---|---|---|---|
| p-value | <0.001 | <0.001 | <0.001 | <0.001 |

Both buttocks 1-level responder

| | | | | | | | | | | | | | | |
|---|---|---|---|---|---|---|---|---|---|---|---|---|---|---|
| Yes | n (%) | 90 | (46.2) | 17 | (8.7) | 125 | (67.6) | 30 (15.8) | 120 | (64.9) | 35 (18.4) | 130 | (62.2) | 35 (17.4) |

| | | | | | | | | | |
|---|---|---|---|---|---|---|---|---|---|
| Missing | n | 14 | 5 | 24 | 11 | 24 | 11 | — | — |

| | | | | |
|---|---|---|---|---|
| p-value | <0.001 | <0.001 | <0.001 | <0.001 |

Target buttock 2-level responder

| | | | | | | | | | | | | | | |
|---|---|---|---|---|---|---|---|---|---|---|---|---|---|---|
| Yes | n (%) | 20 | (10.3) | 1 | (0.5) | 37 | (20.0) | 3 (1.6) | 47 | (25.4) | 4 (2.1) | 49 | (23.4) | 4 (2.0) |

| | | | | | | | | | |
|---|---|---|---|---|---|---|---|---|---|
| Missing | n | 14 | 5 | 24 | 11 | 24 | 11 | — | — |

| | | | | |
|---|---|---|---|---|
| p-value | <0.001 | <0.001 | <0.001 | <0.001 |

Non-target buttock 2-level responder

| | | | | | | | | | | | | | | |
|---|---|---|---|---|---|---|---|---|---|---|---|---|---|---|
| Yes | n (%) | 11 | (5.6) | 3 | (1.5) | 35 | (18.9) | 3 (1.6) | 44 | (23.8) | 7 (3.7) | 46 | (22.0) | 7 (3.5) |

| | | | | | | | | | |
|---|---|---|---|---|---|---|---|---|---|
| Missing | n | 14 | 5 | 24 | 11 | 24 | 11 | — | — |

| | | | | |
|---|---|---|---|---|
| p-value | 0.034 | <0.001 | <0.001 | <0.001 |

At least 1 buttock 2-level responder

| | | | | | | | | | | | | | | |
|---|---|---|---|---|---|---|---|---|---|---|---|---|---|---|
| Yes | n (%) | 22 | (11.3) | 3 | (1.5) | 46 | (24.9) | 5 (2.6) | 59 | (31.9) | 7 (3.7) | 62 | (29.7) | 7 (3.5) |

| | | | | | | | | | |
|---|---|---|---|---|---|---|---|---|---|
| Missing | n | 14 | 5 | 24 | 11 | 24 | 11 | — | — |

| | | | | |
|---|---|---|---|---|
| p-value | <0.001 | <0.001 | <0.001 | <0.001 |

Both buttocks 2-level responder

| | | | | | | | | | | | | | | |
|---|---|---|---|---|---|---|---|---|---|---|---|---|---|---|
| Yes | n (%) | 9 | (4.6) | 1 | (0.5) | 26 | (14.1) | 1 (0.5) | 32 | (17.3) | 4 (2.1) | 33 | (15.8) | 4 (2.0) |

| | | | | | | | | | |
|---|---|---|---|---|---|---|---|---|---|
| Missing | n | 14 | 5 | 24 | 11 | 24 | 11 | — | — |

| | | | | |
|---|---|---|---|---|
| p-value | 0.010 | <0.001 | <0.001 | <0.001 |

TABLE 47
PR-CIS change from baseline (mITT Population).

| | | Baseline | | Day 71 | | Day 71 (LOCF) | |
|---|---|---|---|---|---|---|---|
| | | CCH | Placebo | CCH | Placebo | CCH | Placebo |
| PR-CIS | Statistic | (N = 209) | (N = 201) | (N = 209) | (N = 201) | (N = 209) | (N = 201) |

Total score

| | | | | | | | | | | | | |
|---|---|---|---|---|---|---|---|---|---|---|---|---|
| Mean (SD) | 52.4 | (8.00) | 51.5 | (9.49) | 39.1 | (13.39) | 44.6 | (12.71) | 39.7 | (13.34) | 44.7 | (12.72) |

| | | | | | | | | | | |
|---|---|---|---|---|---|---|---|---|---|---|
| Mean (SD) | — | — | −13.2 | (12.80) | −7.0 | (12.05) | −12.8 | (12.62) | −6.9 | (11.97) |

change
from
baseline

| | | | |
|---|---|---|---|
| p-value | — | <0.001 | <0.001 |

Abbreviated score

| | | | | | | | | | | | | |
|---|---|---|---|---|---|---|---|---|---|---|---|---|
| Mean (SD) | 44.4 | (6.68) | 43.9 | (8.00) | 33.1 | (11.33) | 37.9 | (10.55) | 33.6 | (11.27) | 38.1 | (10.55) |

| | | | | | | | | | | |
|---|---|---|---|---|---|---|---|---|---|---|
| Mean (SD) | — | — | −11.3 | (10.80) | −6.1 | (10.25) | −10.9 | (10.65) | −5.9 | (10.19) |

change
from
baseline

| | | | |
|---|---|---|---|
| p-value | — | <0.001 | <0.001 |

#1 impact of happiness

| | | | | | | | | | | | | |
|---|---|---|---|---|---|---|---|---|---|---|---|---|
| Mean (SD) | 0.8 | (1.89) | 0.6 | (1.29) | 4.1 | (2.88) | 2.1 | (2.64) | 3.9 | (2.89) | 2.1 | (2.65) |

| | | | | | | | | | | |
|---|---|---|---|---|---|---|---|---|---|---|
| Mean (SD) | — | — | 3.3 | (3.34) | 1.5 | (2.68) | 3.2 | (3.30) | 1.5 | (2.67) |

change
from
baseline

| | | | |
|---|---|---|---|
| p-value | — | <0.001 | <0.001 |

#2 impact of bothersome

| | | | | | | | | | | | | |
|---|---|---|---|---|---|---|---|---|---|---|---|---|
| Mean (SD) | 8.6 | (2.52) | 8.3 | (2.81) | 6.5 | (2.85) | 7.0 | (3.18) | 6.6 | (2.84) | 7.1 | (3.16) |

| | | | | | | | | | | |
|---|---|---|---|---|---|---|---|---|---|---|
| Mean (SD) | — | — | −2.0 | (3.37) | −1.3 | (3.72) | −1.9 | (3.31) | −1.3 | (3.71) |

change
from
baseline

| | | | |
|---|---|---|---|
| p-value | — | 0.052 | 0.063 |

#3 impact of self-consciousness

| | | | | | | | | | | | | |
|---|---|---|---|---|---|---|---|---|---|---|---|---|
| Mean (SD) | 8.8 | (2.13) | 8.6 | (2.44) | 6.9 | (2.67) | 7.5 | (2.78) | 7.0 | (2.64) | 7.5 | (2.77) |

| | | | | | | | | | | |
|---|---|---|---|---|---|---|---|---|---|---|
| Mean (SD) | — | — | −2.0 | (2.84) | −1.2 | (2.83) | −1.8 | (2.90) | −1.1 | (2.82) |

change
from
baseline

| | | | |
|---|---|---|---|
| p-value | — | 0.012 | 0.022 |

#4 impact of embarrassment

| | | | | | | | | | | | | |
|---|---|---|---|---|---|---|---|---|---|---|---|---|
| Mean (SD) | 9.0 | (1.64) | 8.8 | (2.16) | 6.7 | (2.82) | 7.6 | (2.61) | 6.8 | (2.79) | 7.7 | (2.60) |

| | | | | | | | | | | |
|---|---|---|---|---|---|---|---|---|---|---|
| Mean (SD) | — | — | −2.3 | (2.71) | −1.2 | (2.63) | −2.2 | (2.67) | −1.2 | (2.62) |

change
from
baseline

| | | | |
|---|---|---|---|
| p-value | — | <0.001 | <0.001 |

#5 impact of old appearance

| | | | | | | | | | | | | |
|---|---|---|---|---|---|---|---|---|---|---|---|---|
| Mean (SD) | 8.0 | (2.23) | 7.6 | (2.45) | 6.0 | (2.86) | 6.6 | (3.06) | 6.1 | (2.86) | 6.7 | (3.05) |

| | | | | | | | | | | |
|---|---|---|---|---|---|---|---|---|---|---|
| Mean (SD) | — | — | −1.9 | (3.04) | −1.0 | (2.99) | −1.8 | (2.98) | −0.9 | (2.96) |

change
from
baseline

| | | | |
|---|---|---|---|
| p-value | — | 0.009 | 0.009 |

#6 impact of body shape concern

| | | | | | | | | | | | | |
|---|---|---|---|---|---|---|---|---|---|---|---|---|
| Mean (SD) | 8.9 | (1.54) | 8.8 | (1.69) | 7.0 | (2.66) | 7.8 | (2.38) | 7.1 | (2.64) | 7.9 | (2.37) |

| | | | | | | | | | | |
|---|---|---|---|---|---|---|---|---|---|---|
| Mean (SD) | — | — | −1.8 | (2.52) | −0.9 | (2.31) | −1.8 | (2.50) | −0.9 | (2.29) |

change
from
baseline

| | | | |
|---|---|---|---|
| p-value | — | <0.001 | <0.001 |

TABLE 48
Subject self-rating score (SSRS) by visit (mITT Population).

| | | Baseline | | | | Day 71 | | | | Day 71 (LOCF) | | | |
|---|---|---|---|---|---|---|---|---|---|---|---|---|---|
| SSRS | | CCH | | Placebo | | CCH | | Placebo | | CCH | | Placebo | |
| Score | Statistic | (N = 209) | | (N = 201) | | (N = 209) | | (N = 201) | | (N = 209) | | (N = 201) | |
| Extremely | n (%) | 0 | (0.0) | 1 | (0.5) | 6 | (3.2) | 1 | (0.5) | 6 | (3.0) | 1 | (0.5) |
| satisfied (6) | | | | | | | | | | | | | |
| Satisfied (5) | n (%) | 2 | (1.0) | 1 | (0.5) | 38 | (20.4) | 12 | (6.3) | 39 | (19.7) | 12 | (6.2) |
| Slightly | n (%) | 3 | (1.4) | 6 | (3.0) | 46 | (24.7) | 18 | (9.4) | 46 | (23.2) | 18 | (9.2) |
| satisfied (4) | | | | | | | | | | | | | |
| Neither | n (%) | 6 | (2.9) | 4 | (2.0) | 34 | (18.3) | 38 | (19.9) | 36 | (18.2) | 39 | (20.0) |
| satisfied nor | | | | | | | | | | | | | |
| dissatisfied (3) | | | | | | | | | | | | | |
| Slightly | n (%) | 26 | (12.4) | 15 | (7.5) | 17 | (9.1) | 34 | (17.8) | 19 | (9.6) | 34 | (17.4) |
| dissatisfied (2) | | | | | | | | | | | | | |
| Dissatisfied (1) | n (%) | 61 | (29.2) | 79 | (39.5) | 30 | (16.1) | 42 | (22.0) | 34 | (17.2) | 43 | (22.1) |
| Extremely | n (%) | 111 | (53.1) | 94 | (47.0) | 15 | (8.1) | 46 | (24.1) | 18 | (9.1) | 48 | (24.6) |
| dissatisfied (0) | | | | | | | | | | | | | |
| Missing | N | 0 | | 1 | | 23 | | 10 | | 11 | | 6 | |
| | Mean (SD) | 0.7 | (0.99) | 0.8 | (1.03) | 3.1 | (1.68) | 1.9 | (1.55) | 3.0 | (1.71) | 1.9 | (1.55) |

| | | | |
|---|---|---|---|
| p-value | 0 (0.0) | 1 (0.5) | 6 (3.2) |

One-level SSRS responder

| | | | | | | | | |
|---|---|---|---|---|---|---|---|---|
| n (%) | 90 | (48.4) | 31 | (16.2) | 91 | (46.0) | 31 | (15.9) |

| | |
|---|---|
| p-value | <0.001 |

With respect to Subject Satisfaction with Cellulite Treatment, a greater proportion of subjects treated with CCH were satisfied or very satisfied with their cellulite treatment than those treated with placebo (46.8% vs 13.6%, respectively; p<0.001). There was a statistically significant (p<0.001) difference in mean (SD) subject satisfaction scores at Day 71 between the CCH (0.3 [1.05]) and the placebo (−0.6 [1.04]) treatment groups. This is summarized in Table 49 below.

TABLE 49
Subject Satisfaction with Cellulite Treatment at Day 71 (mITT Population)

| | | Day 71 | | | | Day 71 (LOCF)^{a} | | | |
|---|---|---|---|---|---|---|---|---|---|
| | | CCH | | Placebo | | CCH | | Placebo | |
| Satisfaction Score | Statistic | (N = 209) | | (N = 201) | | (N = 209) | | (N = 201) | |
| Very Satisfied (+2) | n (%) | 15 | (8.1) | 4 | (2.1) | 15 | (7.6) | 4 | (2.1) |
| Satisfied (+1) | n (%) | 72 | (38.7) | 22 | (11.5) | 74 | (37.4) | 22 | (11.3) |
| Neither Satisfied Nor Dissatisfied (0) | n (%) | 60 | (32.3) | 73 | (38.2) | 64 | (32.3) | 75 | (38.5) |
| Dissatisfied (−1) | n (%) | 24 | (12.9) | 47 | (24.6) | 29 | (14.6) | 48 | (24.6) |
| Very Dissatisfied (−2) | n (%) | 15 | (8.1) | 45 | (23.6) | 16 | (8.1) | 46 | (23.6) |
| Missing | n | 23 | | 10 | | 11 | | 6 | |
| | Mean (SD) | 0.3 | (1.05) | −0.6 | (1.04) | 0.2 | (1.05) | −0.6 | (1.04) |
| | p-value^{b} | <0.001 | | | | <0.001 | | | |
| Subject Satisfaction Responder (Score ≥1) | n (%) | 87 | (46.8) | 26 | (13.6) | 89 | (44.9) | 26 | (13.3) |

| | | |
|---|---|---|
| p-value^{c} | <0.001 | <0.001 |

^{a}If Day 71 Subject Satisfaction with Cellulite Treatment assessment was missing, the last assessment post the initial injection was carried forward to Day 71.
^{b}p-value for Subject Satisfaction with Cellulite Treatment was based on Wilcoxon rank sum test.
^{c}p-value for 1-level subjects satisfaction responder was based on a CMH test adjusted for analysis center.

### Efficacy Conclusions

The primary endpoint was the proportion of 2-level CR-PCSS/PR-PCSS composite responders in the target buttock at Day 71 in the ITT Population. A 2-level CR PCSS/PR-PCSS composite responder was defined as a subject with an improvement from baseline of at least 2 levels of severity in the CR-PCSS and an improvement from baseline in at least 2 levels of severity in the PR-PCSS.

In this study, a statistically significant difference (p=0.002) was seen in the proportion of 2 level CR-PCSS/PR-PCSS composite responders in CCH treated subjects (12 [5.6%]) compared to placebo treated subjects (1 [0.5%]).

In order to confirm the drug effect using orthogonal scales, a series of secondary endpoints were evaluated. There were statistically significant differences in response that favored subjects treated with CCH over subjects treated with placebo in 7 of the 8 key secondary endpoints using the ITT Population. In severity assessments, a negative change from baseline indicates improvement in cellulite. Results for the target and nontarget buttock are outlined below:
Proportion of 1-level PR-PCSS responders in the target buttock at Day 71 (124 [57.9%] in CCH treated subjects vs 61 [29.6%] in placebo treated subjects, p<0.001).
Proportion of 2-level PR-PCSS responders in the target buttock at Day 71 (45 [21.0%] in CCH treated subjects vs 12 [5.8%] in placebo treated subjects, p<0.001).
Proportion of 1-level CR-PCSS/PR-PCSS composite responders in the target buttock at Day 71 (89 [41.6%] in CCH treated subjects vs 23 [11.2%] in placebo treated subjects, p<0.001).
Proportion of 1-level SSRS responders at Day 71 (90 [42.1%] in CCH treated subjects vs 31 [15.0%] in placebo treated subjects, p<0.001).
Mean (SD) change from baseline of PR-CIS Total Score at Day 71 (−11.5 [12.74] in CCH treated subjects vs −6.5 [11.74] in placebo treated subjects, p<0.001).
Proportion of 1-level S-GAIS responders in the target buttock at Day 71 (126 [58.9%] in CCH treated subjects vs 46 [22.3%] in placebo treated subjects, p<0.001).
Proportion of 2-level S-GAIS responders in the target buttock at Day 71 (38 [17.8%] in CCH treated subjects vs 9 [4.4%] in placebo treated subjects, p<0.001).

For 2-level CR-PCSS/PR-PCSS composite responders in the nontarget buttock, the proportion of responders was greater in the CCH treated subjects than in the placebo treated subjects (13 [6.1%] vs 4 [1.9%], respectively, p=0.033), but this difference did not reach statistical significance through the multiplicity test with the family type I error rate of 5%. However, this result with the analyses of secondary endpoints of the nontarget buttock strongly support a trend of a difference in the nontarget buttock response that favors CCH treated subjects compared to placebo treated subjects All sensitivity analyses supported the results for the primary and key secondary endpoints.

There were also statistically significant differences between CCH treated subjects and placebo treated subjects in all supportive endpoints by Day 71 of the study in the mITT Population (and in many cases statistically significant differences were seen as early as after a single treatment with CCH). In severity assessments, a negative change from baseline indicates an improvement in cellulite. Results for the target and nontarget buttock are outlined below:
At Day 71, the mean (SD) change from baseline in PR-PCSS was greater in subjects treated with CCH than in those treated with placebo in the target buttock (0.9 [0.89] vs 0.4 [0.64], respectively; p<0.001) and the nontarget buttock (0.9 [0.84] vs 0.4 [0.67], respectively; p<0.001).
At Day 71, the proportion of 1-level PR-PCSS responders was greater in subjects treated with CCH than in placebo treated subjects in the target buttock (66.7% vs 31.9%, respectively, p<0.001) and in the nontarget buttock (64.5% vs 35.6%, respectively; p<0.001).
At Day 71, the mean (SD) change from baseline in CR-PCSS was greater in subjects treated with CCH than in those treated with placebo in the target buttock (0.8 [0.72] vs 0.2 [0.55], respectively; p<0.001) and the nontarget buttock (0.8 [0.89] vs 0.2 [0.61], respectively; p<0.001).
At Day 71, the proportion of 1-level CR-PCSS responders was greater in subjects treated with CCH than in placebo treated subjects in the target buttock (65.4% vs 27.4%, respectively; p<0.001) and in the nontarget buttock (67.6% vs 28.4%, respectively; p<0.001).
At Day 71, the proportion of 1-level CR-PCSS/PR-PCSS composite responders was greater in subjects treated with CCH than in placebo treated subjects in the target buttock (48.1% vs 12.1%, respectively; p<0.001) and in the nontarget buttock (49.7% vs 15.3%, respectively, p<0.001).
At Day 71, the mean (SD) S-GAIS was greater in subjects treated with CCH in the target buttock when compared to subjects treated with placebo (0.9 [0.92] vs 0.2 [0.68], respectively, p<0.001). The results were similar for the nontarget buttock (0.9 [0.94] in CCH treated subjects vs 0.2 [0.67] in placebo treated subjects, p<0.001).
At Day 71, the proportion of 1-level S-GAIS responders was greater in subjects treated with CCH than in placebo treated subjects in the target buttock (67.7% vs 24.1%, respectively; p<0.001) and the nontarget buttock (66.7% vs 26.2%, respectively; p<0.001).
At Day 71, the mean (SD) I-GAIS was statistically significantly (p<0.001) greater in subjects treated with CCH in the target buttock when compared to subjects treated with placebo (0.9 [0.81] vs 0.3 [0.52] respectively). The results were similar for the nontarget buttock (0.9 [0.79] in CCH treated subjects vs 0.3 [0.54] in placebo treated subjects; p<0.001).
At Day 71, the proportion of 1-level I-GAIS responders was statistically significantly greater in subjects treated with CCH than in placebo treated subjects in the target buttock (70.8% vs 24.2%, respectively; p<0.001) and the nontarget buttock (70.3% vs 21.6%, respectively; p<0.001).
The mean (SD) PR-CIS change from baseline at Day 71 was statistically significantly favorable for CCH treated subjects vs placebo treatment subjects in total score (13.2 [12.80] vs −7.0 [12.05], respectively, p<0.001) and abbreviated score (12.8 [12.62] vs 6.1 [110.25], respectively, p<0.001). All individual impact scores also had statistically significant differences at Day 71 (happiness with the appearance of cellulite [p<0.001], self-consciousness [p=0.012], embarrassment [p<0.001], older appearance [p=0.009], body shape concern [p<0.001]) except for bothersome where the trend favored CCH treated subjects but did not reach statistical significance (p=0.052).
At Day 71 the proportion of PR-CIS responders was greater in subjects treated with CCH than in those treated with placebo for total score (50.0% vs 26.2%, respectively) and for the abbreviated score (52.2% vs 29.3%, respectively). These differences were statistically significant (p<0.001). In addition, the proportion of responders for each individual impact score was greater in CCH treated subjects than in placebo treated subjects. These differences were also statistically significant.

Additional supportive results included:
Day 71, the mean (SD) SSRS score was greater in subjects treated with CCH (3.1 [1.68]) than in those treated with placebo (1.9 [1.55]). This difference was statistically significant (p<0.001). In addition, a greater proportion of subjects treated with CCH were 1-level SSRS responders than those treated with placebo: 48.4% vs 16.2%, respectively. This difference was also statistically significant (p<0.001).
A greater proportion of subjects treated with CCH were satisfied or very satisfied with their cellulite treatment than those treated with placebo (46.8% vs 13.6%, respectively; p<0.001). There was a statistically significant (p<0.001) difference in mean (SD) subject satisfaction scores at Day 71 between the CCH (0.3 [1.05]) and the placebo (0.6 [1.04]) treatment groups.
A series of cross-tabulations show consistency between PR-PCSS and S-GAIS. A 1 level change in the PR-PCSS was associated with similar changes in S-GAIS.
By site analyses of the primary and secondary endpoints indicated that response was seen across multiple sites and that no single site markedly impacted the results of any efficacy analysis.

### Conclusion

This study met its primary endpoint with statistically significant difference in the proportion of 2-level CR-PCSS/PR-PCSS composite responders in CCH treated subjects compared to placebo treated subjects when given as 0.84 mg in each buttock (1.68 mg total) for up to 3 treatment sessions 21 days apart in adult women with EFP of the bilateral buttocks. There were statistically significant differences in response that favored subjects treated with CCH over subjects treated with placebo in 7 of 8 key secondary endpoints and all supportive endpoints.

There were no significant or unexpected safety concerns following administration of CCH. The majority of AEs occurred at the site of injection and resolved within 14 days. No clinically meaningful or concerning trends were observed with regard to hematology or chemistry laboratory parameters, or vital signs. Immunogenicity results did not reveal any potential differences from those seen in other clinical studies with this drug.

Based on the results of this study, CCH is a safe and effective treatment for EFP in the buttocks of adult females at a dose of 0.84 mg per buttock (total dose 1.68 mg/per treatment session) given at 3 treatment sessions at least 21 days apart.

### Example 4—Release-1/Release-2

Examples 2 and 3 above report on the two identically designed, multicenter, double-blind, randomized, placebo-controlled phase 3 studies (RELEASE-1 and RELEASE-2) of adult women with moderate to severe cellulite (rating 3-4 on PR-PCSS and CR-PCSS) on both buttocks who received up to 3 treatment sessions of subcutaneous CCH 0.84 mg or placebo per treatment area.

The Phase 3 study had no cap (enrollment criteria) on BMI or Hexsel severity scale. The lack of a enrollment criteria of Hexsel was a difference from the Phase 2 study design. Despite this difference, the study achieved the stringent primary endpoint and passed 15 of 16 secondary endpoints. The removal of the Hexsel cap in cellulite severity may have contributed to the enrollment of subjects with higher BMIs, which increased the average BMI in the Phase 3 trials. In Phase 3, 210 out of 423 patients were obese while 32 out of 94 were obese in Phase 2b. An effect of the drug was seen in all BMI groups, and none of the patient-centric outcome measures were affected by BMI or other factors. Accordingly, collagenase represents a safe and effective treatment for cellulite.

Responders from the completed Phase 2b trial are currently being followed in a rollover extension study that is looking at 5-year safety and durability. In addition, subjects from RELEASE-1 and RELEASE-2 are currently being followed in a rollover extension study. Other analyses of the data revealed the following results.

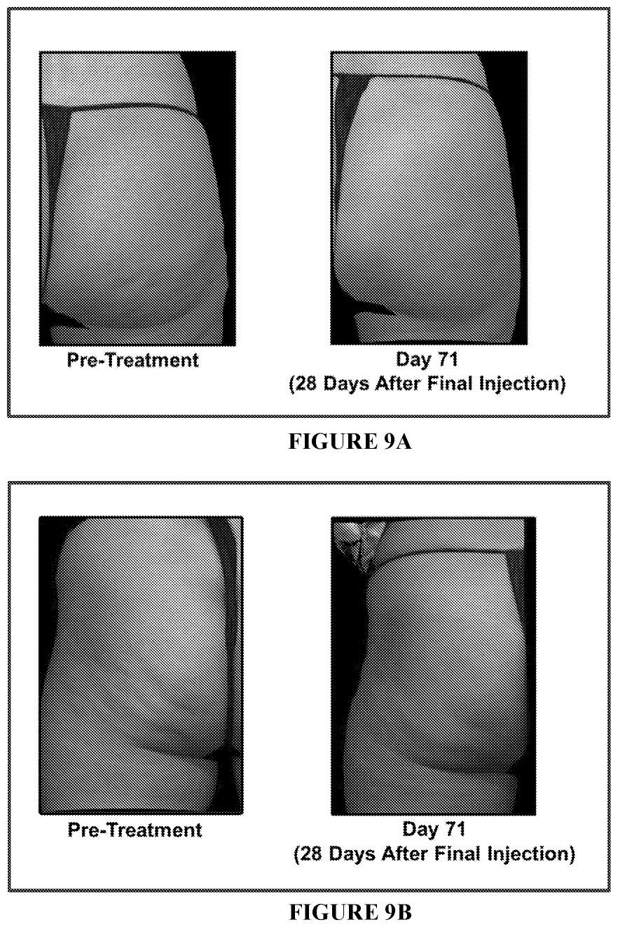

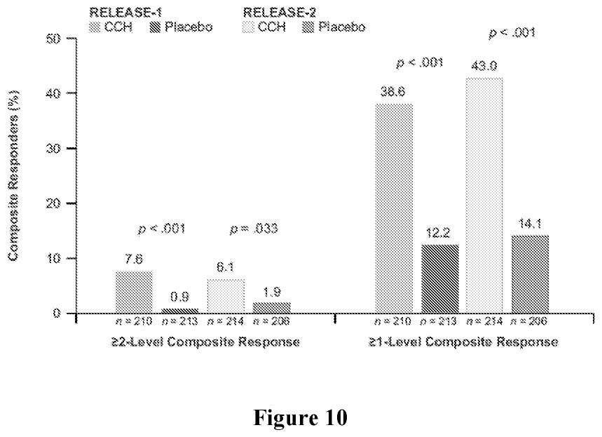

843 women received ≥1 injection (CCH vs placebo: RELEASE-1, n=210 vs n=213; RELEASE-2, n=214 vs n=206). A statistically significant greater percentage of CCH-treated women were greater than or equal to 2-level composite responders versus placebo in RELEASE-1 (about 7.6% vs. about 1.9%; p=0.006) and RELEASE-2 (about 5.6% vs. about 0.5%; p=0.002), and greater than or equal to 1-level composite responders in RELEASE-1 (about 37.1% vs. about 17.8%; p=<0.001) and RELEASE-2 (about 41.6% vs. about 11.2%; p<0.001). The results showing efficacy as measured by PR-PCSS and CR-PCSS are illustrated in FIGS. 8 and 9.

The following subject populations were among the populations that were analyzed:

The Intent-to-treat (ITT) Population included all randomized subjects who had at least 1 injection of study drug. All demographic and baseline characteristic summaries were based on this population. The primary and key secondary efficacy parameters were based on this population.

The Modified Intent-to-treat (mITT) Population included all ITT Population subjects with a baseline and at least 1 post injection evaluation of both the investigator CR-PCSS and subject PR-PCSS for both the target and nontarget buttocks. All secondary and supportive efficacy evaluations were based on the mITT Population.

For each of the identically designed trials, sample size was estimated based on the assumption that the percentage of ≥2-level composite responders for the target buttock would be ≥12% in the CCH group and ≤3% in the placebo group (odds ratio≥4.4), with a participant discontinuation rate of ˜10%. With these assumptions, each trial needed a sample size of 420 women randomly assigned in a 1:1 ratio to the 2 treatment groups. This distribution of women was intended to provide 90% power with a type 1 error of 0.05 to detect statistically significant changes in the primary and key secondary end points. All women randomly assigned to treatment who received ≥1 injection of study medication were included in the ITT and safety populations. Data for the primary and key secondary efficacy end points were analyzed using the Cochran-Mantel-Haenszel test, with adjustment for study center. The change from baseline to Day 71 in the PR-CIS total score was evaluated by analysis of covariance with treatment group and analysis center as factors after adjustment for the baseline value. Women with missing efficacy data at Day 71 were considered nonresponders. Demographics, safety, and immunogenicity data were summarized using descriptive statistics. Statistical analysis was performed using SAS® statistical software package Version 9.3 or higher (SAS Institute Inc., Cary, NC).

Clinically meaningful change was estimated using anchor-based methods, based on guidance from the US Food and Drug Administration, (US Department of Health and Human Services, Food and Drug Administration. Guidance for industry: patient-reported outcome measures: use in medical product development to support labeling claims. Published December 2009. Available from: https://www.fda.gov/downloads/drugs/guidances/ucm193282.pdf. Accessed Apr. 23, 2019). The S-GAIS served as the anchor for the PR-PCSS. The ability of the PR-PCSS to detect change in cellulite severity was analyzed using pooled data from the 2 studies. Outcomes were determined using PR-PCSS (dependent variable) and the S-GAIS (independent variable) data analyzed with a Kruskal-Wallis 1-way ANOVA model to determine the clinically meaningful change thresholds.

Consistent with earlier studies of CCH for the treatment of cellulite, CCH was well-tolerated in the Phase 3 studies by all dose groups with most adverse events (AEs) being mild to moderate and primarily limited to the local injection area. The most common AEs in the trial were injection site bruising, injection site pain, injection site discoloration, injection site nodule and injection site pruritus. Furthermore, there was a very low discontinuation rate in CCH treated groups in both RELEASE 1 and 2 due to adverse events of approximately 4.3% and 3.7%, respectively.

In RELEASE-1/RELEASE-2, on average more than 7 out of 10 subjects saw a 1-level improvement in the PR-PCSS score after the first treatment. Those subjects also experienced a substantial increase (statistically significant improvement compared to placebo, p<0.01) in PR-CIS Happiness scores (i.e., happiness with the appearance of cellulite). In other aspects of the clinical trial, more than 50% of the subjects had a 1-level increase in the PR-PCSS score, and more than 65% of the subjects scored “Improved” or “Very Improved” or “Very Much Improved” using the Subject Global Aesthetic Improvement Scale (S-GAIS). The CR-PCSS, PR-PCSS, I-GAIS, S-GAIS and/or PR-CIS results were independent of age, BMI or skin color. And, significantly, within the subpopulation of women with normal body weight/BMI, their improvement in the appearance of cellulite was higher than the overall study population. For example, about 11% normal weight patients, about 8% overweight patients, and about 2.5% obese patients were 2-level composite responders by PR- and CR-PCSS.

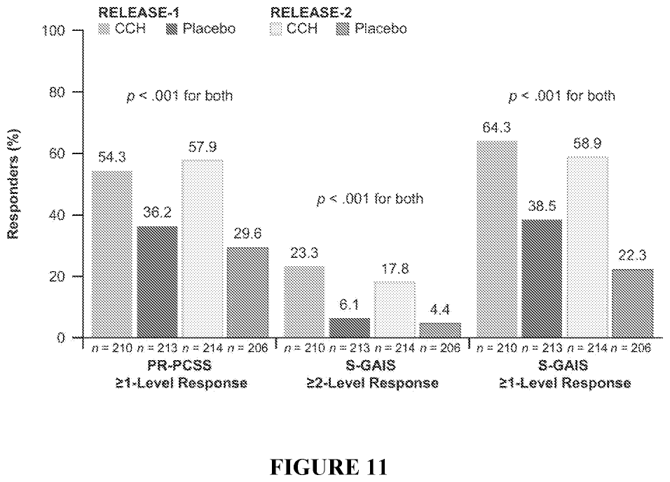

FIG. 10 is a bar chart of the primary endpoint and key secondary endpoint of composite responders in the non-targeted buttock (for purposes of data analysis), defined as patients with greater than equal to 2-level or greater than or equal to 1-level severity improvement from baseline in both CR-PCSS and PR-PCSS at Day 71.

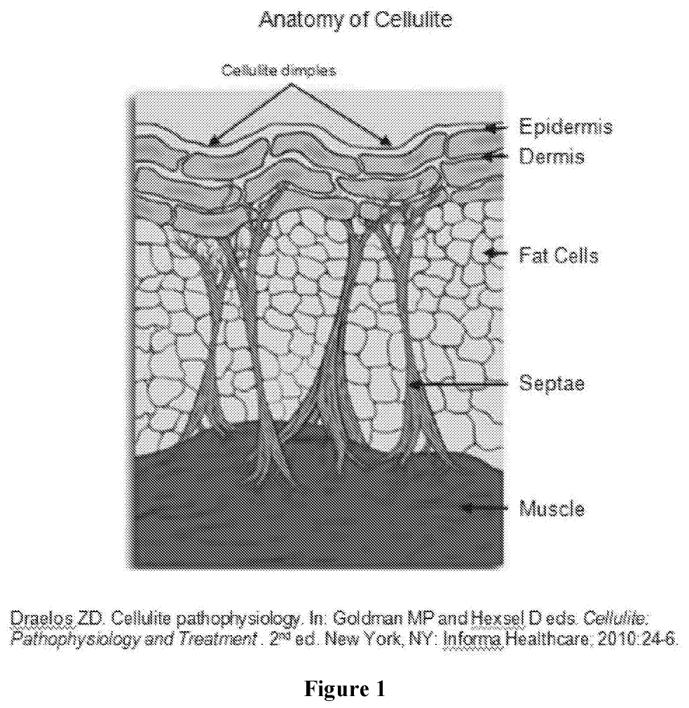

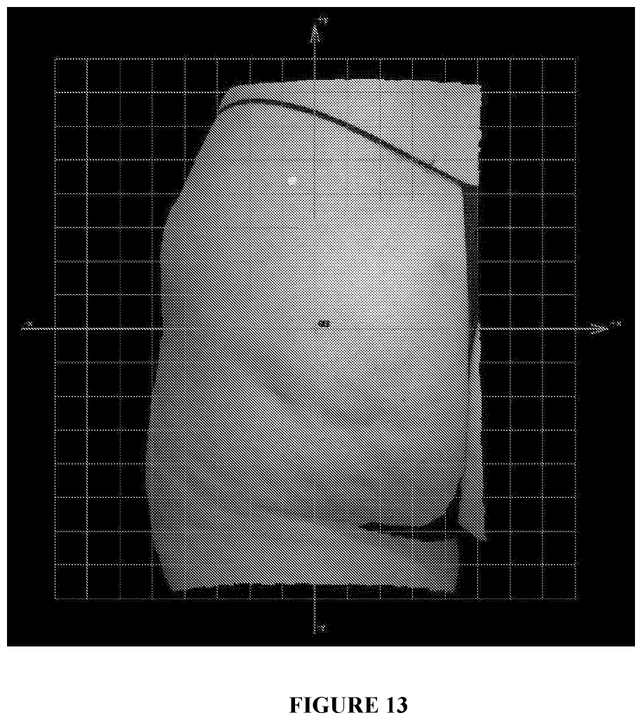

Significantly greater percentages of women treated with CCH were ≥2-level composite responders in both the CR-PCSS and PR-PCSS for the target buttock at Day 71 (primary efficacy end point) compared with placebo in both studies (RELEASE-1, p=0.006; RELEASE-2, p=0.002; FIG. 8). In addition, a significantly greater percentage of women were ≥1-level composite responders for the target buttock at Day 71 compared with placebo (RELEASE-1 and -2, p<0.001 for both; FIG. 8). The percentages of 2-level and 1-level composite responders at Day 71 in the nontarget buttock (FIG. 10) were similar to the percentages for the target buttock in both studies. Photographic images of a 2-level (FIG. 9A) and 1-level (FIG. 9B) composite response show improvement in skin topography at Day 71 after treatment with CCH compared with baseline. In addition, both studies showed significant improvements (p<0.001 for all) from baseline to Day 71 for women treated with CCH compared with those receiving placebo for PR-PCSS (1-level improvement) and S-GAIS ratings (>1- and 2-level improvements; FIG. 11) and for PR-CIS total score (FIG. 12). Ability to detect change for the PR-PCSS demonstrated that the 1-level improvement change threshold was indicative of clinically meaningful change for women, as this level of change was associated with ratings of improvement on the S-GAIS as an external anchor variable. Unless otherwise specified in this example, “Days” as used in studies 302 and 303 were relative to the initial dose (Day 1) in studies 302 and 303.

Both studies showed significant improvements (p<0.001 for all) from baseline to Day 71 for women treated with CCH compared with those receiving placebo for PR-PCSS (≥1-level improvement) and S-GAIS ratings (≥1- and >2-level improvements; FIG. 11) and for PR-CIS total score (FIG. 12). Ability to detect change for the PR-PCSS demonstrated that the 1-level improvement change threshold was indicative of clinically meaningful change for women, as this level of change was associated with ratings of improvement on the S-GAIS as an external anchor variable.

The collagenase injections had a rapid effect. It provided mean subject PR-PCSS scores separated from placebo 21 days after the first treatment and showed continuous and significant improvement after subsequent treatments. Likewise, it provided mean subject CR-PCSS scores separated from placebo 21 days after the first treatment and showed continuous and significant improvement after subsequent treatments.

Over 11% of patients studied in phase 3 had a 2-level composite response in at least one buttock as measured by the CR-PCSS and PR-PCSS scales.

Both patients and physicians exhibited response in photonumeric scales as follows:
Over two-thirds of patients saw at least a 1-level PR-PCSS response in either buttock by day 71 from the first treatment
Over two-thirds of physicians saw at least 1-level CR-PCSS response in either buttock by day 71 from the first treatment
Over three-fourths of patients had 1-level CR-PCSS and/or 1-level PR-PCSS response in either buttock by day 71 from the first treatment

Both patients and physicians witnessed global improvement as follows:
Over three-fourths of patients saw improvement using the S-GAIS in either buttock by day 71 from the first treatment
Over three-fourths of physicians saw improvement using the I-GAIS in either buttock by day 71 from the first treatment

Patient response across photonumeric and other patient-centric scales were as follows:
Over two-thirds of patients were 1-level PR-PCSS responders and/or 1-level SSRS responders in either buttock by day 71 from the first treatment
Over two-thirds of patients were 1-level PR-PCSS responders and/or 1-level SSCT responders in either buttock by day 71 from the first treatment
Over three-fourths of patients were 1-level PR-PCSS responders and/or 1-level S-GAIS responders in either buttock by day 71 from the first treatment
Over three-fourths of patients were 1-level PR-PCSS responders and/or 1-level PR-CIS: Happy responders in either buttock by day 71 from the first treatment
Over three-fourths of patients were 1-level PR-PCSS responders and/or 1-level PR-CIS: Bother responders in either buttock by day 71 from the first treatment
Over three-fourths of patients were 1-level PR-PCSS responders and/or 1-level PR-CIS: Self Conscious responders in either buttock by day 71 from the first treatment

The Patient Reported Cellulite Impact Scale (PR-CIS) showed statistical improvement across all domains:
Unhappiness with the appearance of cellulite
Bother
Self-consciousness
Embarrassment
Looking older
Looking overweight/out of shape

The CR-PCSS, PR-PCSS, I-GAIS, S-GAIS and/or PR-CIS results were independent of age, BMI or skin color. And, significantly, within the subpopulation of women with normal body weight/BMI, their improvement in the appearance of cellulite was higher than the overall study population.

The subjects (on CCH) who saw a 1-level improvement in the PR-PCSS saw a substantial increase (statistically significant against placebo) in PR-CIS Happy scores. Additionally, Respondents in the study saw a significant separation from placebo after the 1st injection.

Subjects receiving CCH showed statistically significant levels of improvement in the appearance of cellulite with treatment, as measured by the trial's primary endpoint (RELEASE-1, p=0.006 & RELEASE-2, p=0.002), which was at least a 2-level composite improvement in cellulite severity in the target buttock at Day 71 as compared to subjects receiving placebo (FIG. 8). In addition, RELEASE-1 passed 8 out of 8 key secondary endpoints and RELEASE-2 passed 7 out of 8 key secondary endpoints. CCH was well-tolerated in the actively-treated subjects with most adverse events (AEs) being mild to moderate in severity and primarily limited to the local injection area.

The PR-CIS Happy scores in subjects who were at least 1-level PR-PCSS responders on target or non-target buttocks or both are presented in Table 50. As shown, CCH treatment was fast-acting to improve subject happiness with the appearance of cellulite.

TABLE 50
PR-CIS Happy scores in subjects who were at least 1-level PR-
PCSS responders on target or non-target buttocks or both.

| Treatment | Mean Baseline | Mean change from Baseline |
|---|---|---|
| Arm, n | PR-CIS Score (SD) | at Day 71 PR-CIS Score (SD) |
| Active, 277 | 0.9 (2.10) | 4.4 (3.31) |
| Placebo, 171 | 0.9 (2.05) | 2.6 (3.59) |

PR-CIS Happy Score 0 = Not at all, 10 = Extremely

### Primary Endpoint for RELEASE-1:

7.6 percent of subjects receiving CCH demonstrated a highly significant (p=0.006) improvement in the composite investigators' and patients' assessments of the appearance of cellulite, as measured by a two-level response in both the Clinician Reported-Photonumeric Cellulite Severity Scale (CR-PCSS) and Patient Reported-Photonumeric Cellulite Severity Scale (PR-PCSS) scores, for the target buttock at Day 71, compared to only 1.9 percent of placebo subjects.

### Primary Endpoint for RELEASE-2:

5.6 percent of subjects receiving CCH demonstrated a highly significant (0.002) improvement in the composite investigators' and patients' assessments of the appearance of cellulite, as measured by a two-level response in both the CR-PCSS and PR-PCSS scores, for the target buttock at Day 71, compared to only 0.5 percent of placebo subjects.

Secondary Endpoints for both RELEASE-1 and RELEASE-2:
37.1 percent of subjects in RELEASE-1, and 41.6 percent of subjects in RELEASE-2 receiving CCH demonstrated a highly significant 1-level response in the composite investigators' and patients' assessments of the appearance of cellulite, as measured by both the CR-PCSS and PR-PCSS scores, for the target buttock at Day 71, compared to only 17.8 percent and 11.2 percent of placebo subjects respectively.
24.3 percent of subjects in RELEASE-1, and 21.0 percent of subjects in RELEASE-2 receiving CCH demonstrated a highly statistically significant 2-level improvement on the patients' assessment of the appearance of cellulite in the target buttock at Day 71, as measured by the PR-PCSS scores compared to only 12.2 percent and 5.8 percent of placebo subjects respectively.
54.3 percent of subjects in RELEASE-1, and 57.9 percent of subjects in RELEASE-2 receiving CCH demonstrated a highly statistically significant 1-level improvement on the patients' assessment of the appearance of cellulite in the target buttock at Day 71, as measured by the PR-PCSS scores compared to only 36.2 percent and 29.6 percent of placebo subjects respectively.
48.6 percent of subjects in RELEASE-1, and 42.1 percent of subjects in RELEASE-2 receiving CCH demonstrated a highly statistically significant 1-level improvement on the patients' assessment of the appearance of cellulite in the target buttock at Day 71, as measured by the SSRS (Subject Self Rating Scale) compared to only 22.5 percent and 15.0 percent of placebo subjects respectively.
54.3 percent of subjects in RELEASE-1, and 46.8 percent of subjects in RELEASE-2 receiving CCH reported being “Satisfied” or “Very Satisfied” with their cellulite treatment as assessed by the Subject Satisfaction with Cellulite Treatment Assessment at Day 71, compared to only 25.8 percent and 13.6 percent of placebo subjects respectively.
73.3 percent of subjects in RELEASE-1, and 67.8 percent of subjects in RELEASE-2 receiving CCH were reported as “Improved” or “Very Improved” or “Very Much Improved” in global appearance of their cellulite area as assessed by the Subject-Global Aesthetic Improvement Scale in the target buttock at Day 71, compared to only 43.2 percent and 24.1 percent of placebo subjects respectively.
Subjects receiving CCH demonstrated a statistically significant improvement in the composite investigators' and patients' assessments of the appearance of cellulite, as measured by a 2-level improvement in both the CR-PCSS and PR-PCSS scores, for the non-target buttock at Day 71 for RELEASE-1 study but failed to show statistical significance in RELEASE-2 study.

Patients receiving collagenase treatment had a ≥2-point improvement from baseline for both the CR-PCSS and PR-PCSS score at about 71 days after treatment, or had a ≥1-point improvement from baseline for both the CR-PCSS and PR-PCSS score at about 71 days post-treatment. Such patients had a 2-point improvement from baseline for both the CR-PCSS and PR-PCSS score at about 6 months after treatment, or had a 1-point improvement from baseline for both the CR-PCSS and PR-PCSS score at about 6 months post-treatment, or had a 2-point improvement from baseline for both the CR-PCSS and PR-PCSS score at about 12 months after treatment, or had a ≥1-point improvement from baseline for both the CR-PCSS and PR-PCSS score at about 12 months post-treatment. Further, such patients had a ≥2-point or ≥1-point improvement from baseline for both the CR-PCSS and PR-PCSS score at about 22 days, 43 days, 90 days, or 180 days after treatment.

Patients exhibited a 3-point improvement from baseline for both the CR-PCSS and PR-PCSS score at about 6 months post-treatment, or had a 3-point improvement from baseline for both the CR-PCSS and PR-PCSS score at about 12 months after treatment, or had a 3-point improvement from baseline for both the CR-PCSS and PR-PCSS score at about 12 months post-treatment. Further, such patients had a 3-point improvement from baseline for both the CR-PCSS and PR-PCSS score at about 22 days, 43 days, 90 days, or 180 days after treatment. In another aspect, the collagenase treatment exhibited durability (as defined herein).

The Patient Reported Cellulite Impact Scale (PR-CIS) showed statistical improvement across at least one domain selected from the group consisting of unhappiness with the appearance of cellulite, bother, self-consciousness, embarrassment, looking older, and looking overweight/out of shape. The percentage of responders (of the non-placebo group) responding “Yes” in the two-level composite improvement at day 71 post-treatment of the target buttock of the treated population was about 15.4%.

Additional conclusions include:
1. Over one-third of the patients saw at least a 1-level PR-PCSS response in either buttock. Over two-thirds of the patients saw at least a 1-level PR-PCSS response in either buttock. All the patients saw at least a 1-level PR-PCSS response in either buttock.
2. Over one-third of the patients saw at least a 1-level PR-PCSS response in either buttock by day 22. Over two-thirds of the patients saw at least a 1-level PR-PCSS response in either buttock by day 22. All the patients saw at least a 1-level PR-PCSS response in either buttock by day 22.
3. Over one-third of the patients saw at least a 1-level PR-PCSS response in either buttock by day 43. Over two-thirds of the patients saw at least a 1-level PR-PCSS response in either buttock by day 43. All the patients saw at least a 1-level PR-PCSS response in either buttock by day 43.
4. Over one-third of the patients saw at least a 1-level PR-PCSS response in either buttock by day 71. Over two-thirds of the patients saw at least a 1-level PR-PCSS response in either buttock by day 71. All the patients saw at least a 1-level PR-PCSS response in either buttock by day 71.
5. Over one-third of the physicians saw at least a 1-level CR-PCSS response in either buttock Over two-thirds of the physicians saw at least a 1-level CR-PCSS response in either buttock. All the physicians saw at least a 1-level CR-PCSS response in either buttock.
6. Over one-third of the physicians saw at least a 1-level CR-PCSS response in either buttock by day 22. Over two-thirds of the physicians saw at least a 1-level CR-PCSS response in either buttock by day 22. All the physicians saw at least a 1-level CR-PCSS response in either buttock by day 22.
7. Over one-third of the physicians saw at least a 1-level CR-PCSS response in either buttock by day 43. Over two-thirds of the physicians saw at least a 1-level CR-PCSS response in either buttock by day 43. All the physicians saw at least a 1-level CR-PCSS response in either buttock by day 43.
8. Over one-third of the physicians saw at least a 1-level CR-PCSS response in either buttock by day 71. Over two-thirds of the physicians saw at least a 1-level CR-PCSS response in either buttock by day 71. All the physicians saw at least a 1-level CR-PCSS response in either buttock by day 71.
9. Over three-fourths of the patients have 1-level CR-PCSS and/or 1-level PR-PCSS response in either buttock by day 71.
10. Over three-fourths of physicians saw improvement using the I-GAIS in either buttock by day 43.
11. Over one-third of patients were 1-level PR-PCSS responders and/or 1-level SSRS responders in either buttock. Over two-thirds of patients were 1-level PR-PCSS responders and/or 1-level SSRS responders in either buttock. All the patients were 1-level PR-PCSS responders and/or 1-level SSRS responders in either buttock.
12. Over one-third of patients were 1-level PR-PCSS responders and/or 1-level SSRS responders in either buttocks by day 22. Over two-thirds of patients were 1-level PR-PCSS responders and/or 1-level SSRS responders in either buttock by day 22. All the patients were 1-level PR-PCSS responders and/or 1-level SSRS responders in either buttock by day 22.
13. Over one-third of patients were 1-level PR-PCSS responders and/or 1-level SSRS responders in either buttock by day 43. Over two-thirds of patients were 1-level PR-PCSS responders and/or 1-level SSRS responders in either buttock by day 43. All the patients were 1-level PR-PCSS responders and/or 1-level SSRS responders in either buttock by day 43.
14. Over one-third of patients were 1-level PR-PCSS responders and/or 1-level SSRS responders in either buttock by day 71. Over two-thirds of patients were 1-level PR-PCSS responders and/or 1-level SSRS responders in either buttocks by day 71. All the patients were 1-level PR-PCSS responders and/or 1-level SSRS responders in either buttock by day 71.
15. Over one-third of patients were 1-level PR-PCSS responders and/or 1-level SSCT responders in either buttock. Over two-thirds of patients were 1-level PR-PCSS responders and/or 1-level SSCT responders in either buttock. All the patients were 1-level PR-PCSS responders and/or 1-level SSCT responders in either buttock.
16. Over one-third of patients were 1-level PR-PCSS responders and/or 1-level SSCT responders in either buttock by day 22. Over two-thirds of patients were 1-level PR-PCSS responders and/or 1-level SSCT responders in either buttock by day 22. All the patients were 1-level PR-PCSS responders and/or 1-level SSCT responders in either buttock by day 22.
17. Over one-third of patients were 1-level PR-PCSS responders and/or 1-level SSCT responders in either buttock by day 43. Over two-thirds of patients were 1-level PR-PCSS responders and/or 1-level SSCT responders in either buttock by day 43. All the patients were 1-level PR-PCSS responders and/or 1-level SSCT responders in either buttock by day 43.
18. Over one-third of patients were 1-level PR-PCSS responders and/or 1-level SSCT responders in either buttock by day 71. Over two-thirds of patients were 1-level PR-PCSS responders and/or 1-level SSCT responders in either buttock by day 71. All the patients were 1-level PR-PCSS responders and/or 1-level SSCT responders in either buttock by day 71.
19. Over one-fourth of patients were 1-level PR-PCSS responders and/or 1-level S-GAIS responders in either buttock. Over one-half of patients were 1-level PR-PCSS responders and/or 1-level S-GAIS responders in either buttock. Over three-fourths of patients were 1-level PR-PCSS responders and/or 1-level S-GAIS responders in either buttocks. All the patients were 1-level PR-PCSS responders and/or 1-level S-GAIS responders in either buttock.
20. Over one-fourth of patients were 1-level PR-PCSS responders and/or 1-level S-GAIS responders in either buttock by day 22. Over one-half of patients were 1-level PR-PCSS responders and/or 1-level S-GAIS responders in either buttock by day 22. Over three-fourths of patients were 1-level PR-PCSS responders and/or 1-level S-GAIS responders in either buttock by day 22. All the patients were 1-level PR-PCSS responders and/or 1-level S-GAIS responders in either buttock by day 22.
21. Over one-fourth of patients were 1-level PR-PCSS responders and/or 1-level S-GAIS responders in either buttock by day 43. Over one-half of patients were 1-level PR-PCSS responder and/or 1-level S-GAIS responder in either buttock by day 43. Over three-fourths of patients were 1-level PR-PCSS responders and/or 1-level S-GAIS responders in either buttocks by day 43. All the patients were 1-level PR-PCSS responders and/or 1-level S-GAIS responders in either buttock by day 43.
22. Over one-fourth of patients were 1-level PR-PCSS responders and/or 1-level S-GAIS responder in either buttocks by day 71. Over one-half of patients were 1-level PR-PCSS responders and/or 1-level S-GAIS responders in either buttock by day 71. Over three-fourths of patients were 1-level PR-PCSS responders and/or 1-level S-GAIS responders in either buttock by day 71. All the patients were 1-level PR-PCSS responders and/or 1-level S-GAIS responders in either buttock by day 71.
23. Over one-fourth of patients were 1-level PR-PCSS responders and/or 1-level PR-CIS: Happy responders in either buttock. Over one-half of patients were 1-level PR-PCSS responders and/or 1-level PR-CIS: Happy responders in either buttock. Over three-fourths of patients were 1-level PR-PCSS responders and/or 1-level PR-CIS: Happy responders in either buttock. All the patients were 1-level PR-PCSS responders and/or 1-level PR-CIS: Happy responders in either buttock.
24. Over one-fourth of patients were 1-level PR-PCSS responders and/or 1-level PR-CIS: Happy responders in either buttock by day 22. Over one-half of patients were 1-level PR-PCSS responders and/or 1-level PR-CIS: Happy responders in either buttock by day 22. Over three-fourths of patients were 1-level PR-PCSS responders and/or 1-level PR-CIS: Happy responders in either buttock by day 22. All the patients were 1-level PR-PCSS responders and/or 1-level PR-CIS: Happy responders in either buttock by day 22.
25. Over one-fourth of patients were 1-level PR-PCSS responders and/or 1-level PR-CIS: Happy responders in either buttock by day 43. Over one-half of patients were 1-level PR-PCSS responders and/or 1-level PR-CIS: Happy responders in either buttock by day 43. Over three-fourths of patients were 1-level PR-PCSS responders and/or 1-level PR-CIS: Happy responders in either buttock by day 43. All the patients were 1-level PR-PCSS responders and/or 1-level PR-CIS: Happy responders in either buttock by day 43.
26. Over one-fourth of patients were 1-level PR-PCSS responders and/or 1-level PR-CIS: Happy responders in either buttock by day 71. Over one-half of patients were 1-level PR-PCSS responders and/or 1-level PR-CIS: Happy responders in either buttock by day 71. Over three-fourths of patients were 1-level PR-PCSS responders and/or 1-level PR-CIS: Happy responders in either buttock by day 71. All the patients were 1-level PR-PCSS responders and/or 1-level PR-CIS: Happy responders in either buttock by day 71.
27. Over one-fourth of patients were 1-level PR-PCSS responders and/or 1-level PR-CIS: Bother responders in either buttock. Over one-half of patients were 1-level PR-PCSS responders and/or 1-level PR-CIS: Bother responders in either buttock. Over three-fourths of patients were 1-level PR-PCSS responders and/or 1-level PR-CIS: Bother responders in either buttock. All the patients were 1-level PR-PCSS responders and/or 1-level PR-CIS: Bother responders in either buttock.
28. Over one-fourth of patients were 1-level PR-PCSS responders and/or 1-level PR-CIS: Bother responders in either buttock by day 22. Over one-half of patients were 1-level PR-PCSS responders and/or 1-level PR-CIS: Bother responders in either buttock by day 22. In yet another embodiment, over three-fourths of patients were 1-level PR-PCSS responders and/or 1-level PR-CIS: Bother responders in either buttock by day 22. All the patients were 1-level PR-PCSS responders and/or 1-level PR-CIS: Bother responders in either buttock by day 22.
29. Over one-fourth of patients were 1-level PR-PCSS responders and/or 1-level PR-CIS: Bother responders in either buttocks by day 43. Over one-half of patients were 1-level PR-PCSS responders and/or 1-level PR-CIS: Bother responders in either buttock by day 43. Over three-fourths of patients were 1-level PR-PCSS responders and/or 1-level PR-CIS: Bother responders in either buttocks by day 43. All the patients were 1-level PR-PCSS responders and/or 1-level PR-CIS: Bother responders in either buttock by day 43.
30. Over one-fourth of patients were 1-level PR-PCSS responders and/or 1-level PR-CIS: Bother responders in either buttock by day 71. Over one-half of patients were 1-level PR-PCSS responders and/or 1-level PR-CIS: Bother responders in either buttock by day 71. Over three-fourths of patients were 1-level PR-PCSS responders and/or 1-level PR-CIS: Bother responders in either buttock by day 71. All the patients were 1-level PR-PCSS responders and/or 1-level PR-CIS: Bother responders in either buttock by day 71.
31. Over one-fourth of patients were 1-level PR-PCSS responders and/or 1-level PR-CIS: Self Conscious responders in either buttocks. In another embodiment, over one-half of patients were 1-level PR-PCSS responders and/or 1-level PR-CIS: Self Conscious responders in either buttocks. Over three-fourths of patients were 1-level PR-PCSS responders and/or 1-level PR-CIS: Self Conscious responders in either buttock. All the patients were 1-level PR-PCSS responders and/or 1-level PR-CIS: Self Conscious responders in either buttock.
32. Over one-fourth of patients were 1-level PR-PCSS responders and/or 1-level PR-CIS: Self Conscious responders in either buttock by day 22. Over one-half of patients were 1-level PR-PCSS responders and/or 1-level PR-CIS: Self Conscious responders in either buttocks by day 22. Over three-fourths of patients were 1-level PR-PCSS responders and/or 1-level PR-CIS: Self Conscious responders in either buttocks by day 22. All the patients were 1-level PR-PCSS responders and/or 1-level PR-CIS: Self Conscious responders in either buttock by day 22.
33. Over one-fourth of patients were 1-level PR-PCSS responders and/or 1-level PR-CIS: Self Conscious responders in either buttocks by day 43. Over one-half of patients were 1-level PR-PCSS responders and/or 1-level PR-CIS: Self Conscious responders in either buttock by day 43. In yet another embodiment, over three-fourths of patients were 1-level PR-PCSS responders and/or 1-level PR-CIS: Self Conscious responders in either buttock by day 43. All the patients were 1-level PR-PCSS responders and/or 1-level PR-CIS: Self Conscious responders in either buttock by day 43.
34. Over one-fourth of patients were 1-level PR-PCSS responders and/or 1-level PR-CIS: Self Conscious responders in either buttock by day 71. In another embodiment, over one-half of patients were 1-level PR-PCSS responders and/or 1-level PR-CIS: Self Conscious responders in either buttocks by day 71. Over three-fourths of patients were 1-level PR-PCSS responders and/or 1-level PR-CIS: Self Conscious responders in either buttock by day 71. All the patients were 1-level PR-PCSS responders and/or 1-level PR-CIS: Self Conscious responders in either buttocks by day 71
35. Anchor-based analyses in the current studies indicated a PR-PCSS score change≥1 was clinically meaningful. Over half of the women treated with CCH in both studies had a PR-PCSS score change≥1 (54.3% and 57.9%). A composite≥1-level response in both PR-PCSS and CR-PCSS was observed in >35% of women, and a 1-level S-GAIS response was observed in >55% of women.
36. Based on the change from baseline in PR-CIS total scores, women treated with CCH reported a lower overall visual and emotional impact of cellulite post-treatment compared with placebo-treated women, i.e., improved quality of life and self-esteem.
37. AEs resolved quickly, were mild to moderate in intensity, and were infrequent causes of study discontinuation (<5% of women in either study).
38. The current studies include the largest combined patient population in a cellulite study to date (N=843) and the multicenter, randomized, double-blind, placebo-controlled study design of each.
39. The broad study entry criteria, including women with severe cellulite. Among women with severe cellulite at baseline, >15% and >35% of CCH-treated women assessed by the CR-PCSS and PR-PCSS, respectively, had improvement in the target buttock at Day 71. As noted previously, the primary end point, a ≥2-level composite improvement from baseline, is considered a stringent criterion; this is a limitation, given that this outcome is difficult to achieve in clinical practice. Despite this stringent criterion, however, a significantly larger percentage of women receiving CCH met the primary end point compared with placebo.
40. Based on 2 phase 3 randomized, placebo-controlled studies, CCH is a safe and efficacious treatment for women with moderate to severe cellulite of the buttocks.

### Side by Side Results of Examples 2 and 3 (Studies 302 and 303):

Since the analyses conducted in Examples 2 and 3 were identical, a side-by-side table of results of the primary and key secondary endpoints in Examples 2 and 3 is presented in Table 51.

TABLE 51
Side by Side Results for Primary and Key Secondary Endpoints in Examples 2 and 3 (ITT Population)

| | Example 2 (Study 302) | | | | | | Example 3 (Study 303) | | | | | |
|---|---|---|---|---|---|---|---|---|---|---|---|---|
| | Frequency of Responders | | | | Treatment Difference | | Frequency of Responders | | | | Treatment Difference | |
| | CCH | | Placebo | | | Statistical | CCH | | Placebo | | | Statistical |
| Endpoint | (N = 210) | | (N = 213) | | p-value^{a} | Significance^{b} | (N = 214) | | (N = 206) | | p value^{a} | Significance^{b} |
| Two-level Composite | 16 | (7.6) | 4 | (1.9) | 0.006^{c} | * | 12 | (5.6) | 1 | (0.5) | 0.002^{c} | * |
| Responder of the Target | | | | | | | | | | | | |
| Buttock | | | | | | | | | | | | |
| Day 71, n (%) | | | | | | | | | | | | |
| Subject 1-level PR- | 114 | (54.3) | 77 | (36.2) | <0.001 | * | 124 | (57.9) | 61 | (29.6) | <0.001 | * |
| PCSS Responders of the | | | | | | | | | | | | |
| Target Buttock at Day | | | | | | | | | | | | |
| 71, n (%) | | | | | | | | | | | | |
| Subject 2-level PR- | 51 | (24.3) | 26 | (12.2) | 0.001 | * | 45 | (21.0) | 12 | (5.8) | <0.001 | * |
| PCSS Responders of the | | | | | | | | | | | | |
| Target Buttock at Day | | | | | | | | | | | | |
| 71, n (%) | | | | | | | | | | | | |
| Subject/Investigator 1- | 78 | (37.1) | 38 | (17.8) | <0.001 | | 89 | (41.6) | 23 | (11.2) | <0.001 | * |
| level Composite | | | | | | | | | | | | |
| Responders of the Target | | | | | | | | | | | | |
| Buttock at Day 71, n (%) | | | | | | | | | | | | |
| Subject/Investigator 2- | 16 | (7.6) | 2 | (0.9) | <0.001 | * | 13 | (6.1) | 4 | (1.9) | 0.033 | |
| level Composite | | | | | | | | | | | | |
| Responders of the | | | | | | | | | | | | |
| Nontarget Buttock at | | | | | | | | | | | | |
| Day 71 Based on CR- | | | | | | | | | | | | |
| PCSS and PR-PCSS, n | | | | | | | | | | | | |
| (%) | | | | | | | | | | | | |
| Subject 1-level SSRS | 102 | (48.6) | 48 | (22.5) | <0.001 | * | 90 | (42.1) | 31 | (15.0) | <0.001 | * |
| Responders at Day 71, n | | | | | | | | | | | | |
| (%) | | | | | | | | | | | | |
| Change from baseline | −10.9 | (12.51) | −5.9 | (11.62) | <0.001 | * | −11.5 | (12.74) | −6.5 | (11.74) | <0.001 | * |
| (Day 1) of the PR-CIS | | | | | | | | | | | | |
| Total rating at Day 71, | | | | | | | | | | | | |
| mean (SD) | | | | | | | | | | | | |
| Subject 1-level S-GAIS | 135 | (64.3) | 82 | (38.5) | <0.001 | * | 126 | (58.9) | 46 | (22.3) | <0.001 | * |
| Responders of Target | | | | | | | | | | | | |
| Buttock at Day 71, n (%) | | | | | | | | | | | | |
| Subject 2-level S-GAIS | 49 | (23.3) | 13 | (6.1) | <0.001 | * | 38 | (17.8) | 9 | (4.4) | <0.001 | * |
| Responders of Target | | | | | | | | | | | | |
| Buttock at Day 71, n (%) | | | | | | | | | | | | |

^{a}P-value from individual analysis without multiplicity adjustment
^{b}Asterisk (*) represents a family-wise statistical significance at significance level of 0.05 after multiplicity adjustment.
^{c}P-value was based on CMH test adjusted for analysis center.

Results from the pooled analyses of the pivotal Phase 3 studies (Studies −302 and −303; “Pool 1”), and the randomized, double-blind placebo controlled studies (Studies −201, −301, and −302; “Pool 2”) compare the results across studies. Results from Study −202, −205, and −304 support the persistence of effect, and results of Study −202 support the lack of tolerance (the loss of an ability to respond to therapeutic dose(s) over time) upon re-exposure to CCH. The results of the primary efficacy analysis of the individual studies, (Studies −201, −302 and −303), and the integrated results in Pools 1 and 2 demonstrated that reductions in cellulite severity were observed more frequently in the CCH treatment group compared to the placebo treatment group as measured by the composite of the clinician (investigator) and patient (subject) scales.

Comparison results for the primary and key secondary efficacy endpoints (Pool 1-ITT population) are presented in Table 52.

TABLE 52
Multiple Comparison Results for the Primary and Key Secondary Efficacy Endpoints (Pool 1- ITT Population)

| | | | Frequency of | | | | Treatment | |
|---|---|---|---|---|---|---|---|---|
| | | | Responders | | | | Difference | |
| | | | CCH 0.84 | | | | | Statistical |
| | Efficacy | | mg/buttock | | Placebo | | Raw | Significance |
| Order | Group | Endpoint | (N = 424) | | (N = 419) | | p-Value ^{a} | at 0.05 ^{b} |
| 1 | Primary | Two-level composite responders | 28 | (6.6) | 5 | (1.2) | <0.001 | * |
| | | of the target buttock at Day 71 | | | | | | |
| | | based on CR-PCSS and PR- | | | | | | |
| | | PCSS, n (%) | | | | | | |
| 2 | Secondary | One-level PR-PCSS responders | 238 | (56.1) | 138 | (32.9) | <0.001 | * |
| | Family #1 | of the target buttock at Day 71, n (%) | | | | | | |
| 2 | Secondary | Two-level PR-PCSS responders | 96 | (22.6) | 38 | (9.1) | <0.001 | * |
| | Family #1 | of the target buttock at Day 71, n (%) | | | | | | |
| 2 | Secondary | One-level composite responders | 167 | (39.4) | 61 | (14.6) | <0.001 | * |
| | Family #1 | of the target buttock at Day 71, n (%) | | | | | | |
| 2 | Secondary | Two-level composite responders | 29 | (6.8) | 6 | (1.4) | <0.001 | * |
| | Family #1 | of the nontarget buttock at Day | | | | | | |
| | | 71 based on CR-PCSS and PR- | | | | | | |
| | | PCSS, n (%) | | | | | | |
| 3 | Secondary | Subject 1-level SSRS responders | 192 | (45.3) | 79 | (18.9) | <0.001 | * |
| | Family #2 | at Day 71, n (%) | | | | | | |
| 3 | Secondary | Change from baseline (Day 1) of | −11.7 | (12.58) | −6.2 | (11.67) | <0.001 | * |
| | Family #2 | the PR-CIS total rating at Day | | | | | | |
| | | 71, mean (SD) | | | | | | |
| 4 | Secondary | Subject 1-level S-GAIS | 261 | (61.6) | 128 | (30.5) | <0.001 | * |
| | Family #3 | responders of target buttock at | | | | | | |
| | | Day 71, n (%) | | | | | | |
| 4 | Secondary | Subject 2-level S-GAIS | 87 | (20.5) | 22 | (5.3) | <0.001 | * |
| | Family #3 | responders of target buttock at | | | | | | |
| | | Day 71, n (%) | | | | | | |

^{a} P-value from individual analysis without multiplicity adjustment
^{b} * represents a family-wise statistical significance at significance level of 0.05 after multiplicity adjustment.
Note:
Percentages are based on the number of N for each column.

Relevant clinical data are provided in Tables 53-61 below.

TABLE 53
Subject/Investigator Composite Responders for the
Target Buttock at Day 71 (Pool 1-ITT Population)

| | | Study Drug | | CCH 0.84 mg/buttock | |
|---|---|---|---|---|---|
| | | CCH 0.84 | | vs Placebo | |
| End- | Sta- | mg/buttock | Placebo | Odds Ratio | |
| point^{a} | tistic^{b} | (N = 424) | (N = 419) | (95% CI) | p-value ^{c} |

Two-level Composite Responder^{d}

| | | | | | | |
|---|---|---|---|---|---|---|
| Yes | n (%) | 28 (6.6) | 5 | (1.2) | 5.88 (2.25, | <0.001 |
| | | | | | 15.38) | |
| No | n (%) | 396 (93.4) | 414 | (98.8) | | |

One-level Composite Responder^{e}

| | | | | | | |
|---|---|---|---|---|---|---|
| Yes | n (%) | 167 (39.4) | 61 | (14.6) | 3.84 (2.73, | <0.001 |
| | | | | | 5.40) | |
| No | n (%) | 257 (60.6) | 358 | (85.4) | | |

^{a}Subjects without Day 71 PR-PCSS and/or CR-PCSS assessments of the target buttock were considered non-responders.
^{b}Percentages were based on the number of N for each column
^{c} P-values were based on the CMH test adjusted for analysis center.
^{d}A 2-level composite responder for the target buttock was a subject with a reduction in cellulite severity of at least 2 levels from baseline on the target buttock in both PR-PCSS and CR-PCSS at the Day 71 visit.
^{e}A 1-level composite responder for the target buttock is a subject with a reduction in cellulite severity of at least 1- level from baseline on the target buttock in both PR-PCSS and CR-PCSS at the Day 71 visit.

TABLE 54
Subject PR-PCSS Responders of the Target Buttock
at Day 71 (Pool 1- ITT Population)

| | | Study Drug | | CCH 0.84 mg/buttock | |
|---|---|---|---|---|---|
| | | CCH 0.84 | | vs Placebo | |
| End- | | mg/buttock | Placebo | Odds Ratio | |
| point^{a} | Statistic^{b} | (N = 424) | (N = 419) | (95% CI) | p-Value^{c} |

Two-level PR-PCSS Responder ^{d}

| | | | | | | |
|---|---|---|---|---|---|---|
| Yes | n (%) | 96 | (22.6) | 38 (9.1) | 3.04 (2.01, | <0.001 |
| | | | | | 4.60) | |
| No | n (%) | 328 | (77.4) | 381 (90.9) | | |

One-level PR-PCSS Responder ^{e}

| | | | | | | |
|---|---|---|---|---|---|---|
| Yes | n (%) | 238 | (56.1) | 138 (32.9) | 2.62 (1.98, | <0.001 |
| | | | | | 3.47) | |
| No | n (%) | 186 | (43.9) | 281 (67.1) | | |

^{a}Any subject without Day 71 PR-PCSS assessments on the target buttock was considered a non-responder.
^{b}Percentages were based on the number of N for each column.
^{c}P-values were based on the CMH test adjusted for analysis center.
^{d} A 2-level PR-PCSS responder for the target buttock was a subject with a reduction in cellulite severity of at least 2 levels from baseline on the target buttock in PR-PCSS at the Day 71 visit.
^{e} A 1-level PR-PCSS responder for the target buttock is a subject with a reduction in cellulite severity of at least 1 level from baseline on the target buttock in PR-PCSS at the Day 71 visit.

TABLE 55
PR-PCSS Rating and Change from Baseline for the
Target Buttock by Visit (Pool 1- ITT Population)

| | Study Drug | |
|---|---|---|
| | CCH 0.84 mg/buttock | Placebo |
| Statistic^{a} | (N = 424) | (N = 419) |

Baseline

| | | | | | |
|---|---|---|---|---|---|
| None (0) | n (%) | 0 | (0.0) | 0 | (0.0) |
| Almost None (1) | n (%) | 0 | (0.0) | 0 | (0.0) |
| Mild (2) | n (%) | 0 | (0.0) | 0 | (0.0) |
| Moderate (3) | n (%) | 179 | (42.2) | 168 | (40.1) |
| Severe (4) | n (%) | 245 | (57.8) | 251 | (59.9) |

| | | | |
|---|---|---|---|
| Not Done | N | 0 | 0 |
| | Mean | 3.6 | 3.6 |
| | SD | 0.49 | 0.49 |

Change from Baseline at Day 22 ^{b}

| | | | | | |
|---|---|---|---|---|---|
| −4 | n (%) | 0 | (0.0) | 0 | (0.0) |
| −3 | n (%) | 2 | (0.5) | 1 | (0.2) |
| −2 | n (%) | 17 | (4.4) | 6 | (1.5) |
| −1 | n (%) | 124 | (32.3) | 65 | (16.2) |
| 0 | n (%) | 228 | (59.4) | 315 | (78.6) |
| +1 | n (%) | 13 | (3.4) | 14 | (3.5) |

| | | | |
|---|---|---|---|
| Not Done | N | 40 | 18 |
| | Mean | −0.4 | −0.2 |
| | SD | 0.65 | 0.50 |

Change from Baseline at Day 43 ^{b}

| | | | | | |
|---|---|---|---|---|---|
| −4 | n (%) | 0 | (0.0) | 1 | (0.3) |
| −3 | n (%) | 11 | (2.9) | 1 | (0.3) |
| −2 | n (%) | 64 | (17.1) | 25 | (6.5) |
| −1 | n (%) | 150 | (40.1) | 98 | (25.4) |
| 0 | n (%) | 137 | (36.6) | 252 | (65.3) |
| +1 | n (%) | 12 | (3.2) | 9 | (2.3) |

| | | | |
|---|---|---|---|
| Not Done | N | 50 | 33 |
| | Mean | −0.8 | −0.4 |
| | SD | 0.86 | 0.68 |

Change from Baseline at Day 73 ^{b}

| | | | | | |
|---|---|---|---|---|---|
| −4 | n (%) | 1 | (0.3) | 2 | (0.5) |
| −3 | n (%) | 17 | (4.6) | 2 | (0.5) |
| −2 | n (%) | 78 | (21.1) | 34 | (8.9) |
| −1 | n (%) | 142 | (38.4) | 100 | (26.3) |
| 0 | n (%) | 125 | (33.8) | 227 | (59.7) |
| +1 | n (%) | 7 | (1.9) | 15 | (3.9) |

| | | | |
|---|---|---|---|
| Not Done | N | 54 | 39 |
| | Mean | −0.9 | −0.4 |
| | SD | 0.91 | 0.77 |

^{a}Percentages were based on the number of evaluable subjects at each time point for each column
^{b} Change from baseline was visit value minus baseline. Negative change reflects an improvement in cellulite severity; positive change reflects a worsening in cellulite severity.
Note:
Missing PR-PCSS ratings at any time point are not imputed and therefore excluded in the summary.

TABLE 56
One-level SSRS Responders at Day 71 (Pool 1- ITT Population)

| | | Study Drug | | | | |
|---|---|---|---|---|---|---|
| | | CCH 0.84 | | | CCH 0.84 mg/buttock vs Placebo | |
| One-level SSRS | | mg/buttock | Placebo | | Odds Ratio | |
| Responder ^{a} | Statistic ^{b} | (N = 424) | (N = 419) | | (95% CI) | p-Value^{c} |
| Yes | n (%) | 192 (45.3) | 79 | (18.9) | 3.61 (2.64, 4.95) | <0.001 |
| No | n (%) | 232 (54.7) | 340 | (81.1) | | |

^{a} A 1-level SSRS responder was a subject with at least ‘slightly satisfied’ (SSRS rating ≥4; ie, ‘slightly satisfied’ (4), ‘very satisfied’ (5) or ‘extremely satisfied’ (6) with her appearance of the cellulite on her buttocks at Day 71 visit. Any subjects without SSRS assessments at Day 71 were considered non-responders
^{b} Percentages were based on the number of evaluable subjects at each time point for each column.
^{c} The p-value was based on the CMH test adjusted for analysis center.

TABLE 57
Change from Baseline in the PR-CIS Total Rating

| Study Drug | | | | CCH 0.84 mg/buttock | |
|---|---|---|---|---|---|
| | | CCH 0.84 | | vs Placebo | |
| | | mg/buttock | Placebo | Difference | |
| | Statistic | (N = 424) | (N = 419) | (95% CI) | p-Value ^{a} |

Baseline

| Value | N | 423 | | 415 | |
|---|---|---|---|---|---|
| | Mean (SD) | 51.8 | (8.45) | 51.6 | (9.58) |

| | | |
|---|---|---|
| Median | 54.0 | 55.0 |
| Min, Max | 14, 60 | 10, 60 |

Day 71

| Value^{b} | N | 424 | | 419 | | | |
|---|---|---|---|---|---|---|---|
| | Mean (SD) | 40.1 | (13.30) | 45.4 | (12.77) | | |
| | Median | 41.0 | | 48.0 | | | |
| | Min, Max | 0, 60 | | 0, 60 | | | |
| Change from | N | 423 | | 415 | | | |
| Baseline ^{c} | | | | | | | |
| | Mean (SD) | −11.7 | (12.58) | −6.2 | (11.67) | | |
| | Median | −10.0 | | −3.0 | | | |
| | LSMean (SE) | −11.5 | (0.57) | −6.0 | (0.58) | −5.4 (−7.0, −3.9) | <0.001 |

| | | |
|---|---|---|
| Min, Max | −60, 27 | −58, 41 |

^{a} The p-value was based on an ANCOVA model with factors of treatment, study, analysis center within study, and a covariate of baseline value.
^{b}The baseline value was imputed to Day 71 for any subjects without a Day 71 PR-CIS Total Score.
^{c} Change from baseline was the visit value minus baseline value.

TABLE 58
S-GAIS Responders for the Target Buttock
at Day 71 (Pool 1-ITT Population)

| | | Study Drug | | CCH 0.84 mg/buttock | |
|---|---|---|---|---|---|
| | | CCH 0.84 | | vs Placebo | |
| S-GAIS | Statis- | mg/buttock | Placebo | Odds Ratio | p- |
| Response^{a} | tic ^{b} | (N = 424) | (N = 419) | (95% CI) | Value ^{c} |

One-level responder (≥+1)^{d}

| | | | | | | |
|---|---|---|---|---|---|---|
| Yes | n (%) | 261 | (61.6) | 128 (30.5) | 3.68 (2.75, | <0.001 |
| | | | | | 4.92) | |
| No | n (%) | 163 | (38.4) | 291 (69.5) | | |

Two-level responder (≥+2) ^{e}

| | | | | | | |
|---|---|---|---|---|---|---|
| Yes | n (%) | 87 | (20.5) | 22 (5.3) | 4.72 (2.89, | <0.001 |
| | | | | | 7.72) | |
| No | n (%) | 337 | (79.5) | 397 (94.7) | | |

^{a}Any subjects without S-GAIS assessments at Day 71 were considered as non-responders.
^{b} Percentages were based on the number of evaluable subjects at each time point for each column.
^{c} -values were based on CMH test adjusted for analysis center.
^{d}A 1-level S-GAIS responder for the target buttock was defined as a subject with S-GAIS rating of at least 1 (ie, 1, 2 or 3) on the target buttock
^{e} A 2-level S-GAIS responder for the target buttock was defined as a subject with S-GAIS rating of at least 2 (ie, 2 or 3) on the target buttock.

TABLE 59
CR-PCSS Responders of the Target Buttock
at Day 71 (Pool 1- ITT Population)

| | | Study Drug | | CCH 0.84 mg/buttock | |
|---|---|---|---|---|---|
| | | CCH 0.84 | | vs Placebo | |
| | Statis- | mg/buttock | Placebo | Odds Ratio | p- |
| Endpoint ^{a} | tic^{b} | (N = 424) | (N = 419) | (95% CI) | Value ^{c} |

Two-level CR-PCSS Responder ^{d}

| | | | | | | |
|---|---|---|---|---|---|---|
| Yes | n (%) | 67 | (15.8) | 15 (3.6) | 5.12 (2.87, | <0.001 |
| | | | | | 9.14) | |
| No | n (%) | 357 | (84.2) | 404 (96.4) | | |

One-level CR-PCSS Responder^{e}

| | | | | | | |
|---|---|---|---|---|---|---|
| Yes | n (%) | 228 | (53.8) | 114 (27.2) | 3.15 (2.35, | <0.001 |
| | | | | | 4.22) | |
| No | n (%) | 196 | (46.2) | 305 (72.8) | | |

^{a} Any subjects without Day 71 CR-PCSS assessments of the target buttock were considered non-responders.
^{b}Percentages were based on the number of N for each column
^{c} P-values were based on the CMH test adjusted for analysis center.
^{d} A 2-level CR-PCSS responder for the target buttock was a subject with a reduction in cellulite severity of at least 2 levels from baseline on the target buttock in CR-PCSS at the Day 71 visit.
^{e}A 1-level CR-PCSS responder for the target buttock was a subject with a reduction in cellulite severity of at least 1-level from baseline on the target buttock in CR-PCSS at the Day 71 visit.

TABLE 60
CR-PCSS Rating and Change from Baseline for the
Target Buttock by Visit (Pool 1-ITT Population)

| | Study Drug | |
|---|---|---|
| | CCH 0.84 mg/buttock | Placebo |
| Statistic ^{a} | (N = 424) | (N = 419) |

Baseline

| | | | | | |
|---|---|---|---|---|---|
| None (0) | n (%) | 0 | (0.0) | 0 | (0.0) |
| Almost None (1) | n (%) | 0 | (0.0) | 0 | (0.0) |
| Mild (2) | n (%) | 0 | (0.0) | 0 | (0.0) |
| Moderate (3) | n (%) | 257 | (60.6) | 259 | (61.8) |
| Severe (4) | n (%) | 167 | (39.4) | 160 | (38.2) |

| | | | |
|---|---|---|---|
| Not Done | n | 0 | 0 |
| | Mean | 3.4 | 3.4 |
| | SD | 0.49 | 0.49 |

Change from Baseline at Day 22^{b}

| | | | | | |
|---|---|---|---|---|---|
| −4 | n (%) | 0 | (0.0) | 0 | (0.0) |
| −3 | n (%) | 0 | (0.0) | 1 | (0.2) |
| −2 | n (%) | 10 | (2.6) | 2 | (0.5) |
| −1 | n (%) | 129 | (33.7) | 61 | (15.2) |
| 0 | n (%) | 237 | (61.9) | 314 | (78.3) |
| +1 | n (%) | 7 | (1.8) | 23 | (5.7) |

| | | | |
|---|---|---|---|
| Not Done | n | 41 | 18 |
| | Mean | −0.4 | −0.1 |
| | SD | 0.57 | 0.49 |

Change from Baseline at Day 43^{b}

| | | | | | |
|---|---|---|---|---|---|
| −4 | n (%) | 0 | (0.0) | 0 | (0.0) |
| −3 | n (%) | 0 | (0.0) | 0 | (0.0) |
| −2 | n (%) | 48 | (12.8) | 10 | (2.6) |
| −1 | n (%) | 159 | (42.5) | 64 | (16.6) |
| 0 | n (%) | 156 | (41.7) | 291 | (75.4) |
| +1 | n (%) | 11 | (2.9) | 21 | (5.4) |

| | | | |
|---|---|---|---|
| Not Done | N | 50 | 33 |
| | Mean | −0.7 | −0.2 |
| | SD | 0.74 | 0.55 |

Change from Baseline at Day 71^{b}

| | | | | | |
|---|---|---|---|---|---|
| −4 | n (%) | 0 | (0.0) | 0 | (0.0) |
| −3 | n (%) | 4 | (1.1) | 3 | (0.8) |
| −2 | n (%) | 63 | (17.1) | 12 | (3.1) |
| −1 | n (%) | 161 | (43.8) | 99 | (26.0) |
| 0 | n (%) | 129 | (35.1) | 249 | (65.4) |
| +1 | n (%) | 11 | (3.0) | 18 | (4.7) |

| | | | |
|---|---|---|---|
| Not Done | n | 56 | 38 |
| | Mean | −0.8 | −0.3 |
| | SD | 0.80 | 0.64 |

^{a} Percentages were based on the number of evaluable subjects at each time point for each column.
^{b}Change from baseline was visit value minus baseline. Negative change reflects an improvement in cellulite severity; positive change reflects a worsening in cellulite severity.

TABLE 61
SSCTA at Day 71 (Pool 1- ITT Population)

| | | Study Drug | | | |
|---|---|---|---|---|---|
| | | CCH 0.84 | | | |
| | | mg/buttock | | Placebo | |
| Day 71 | Statistic^{a} | (N = 424) | | (N = 419) | |
| Very Satisfied (+2) | n (%) | 39 | (9.9) | 11 | (2.8) |
| Satisfied (+1) | n (%) | 154 | (39.2) | 64 | (16.6) |
| Neither Satisfied nor | n (%) | 116 | (29.5) | 131 | (33.9) |
| Dissatisfied (0) | | | | | |
| Dissatisfied (−1) | n (%) | 52 | (13.2) | 90 | (23.3) |
| Very Dissatisfied (−2) | n (%) | 32 | (8.1) | 90 | (23.3) |

| | | | |
|---|---|---|---|
| Not Done | N | 31 | 33 |
| | Mean | 0.3 | −0.5 |
| | SD | 1.08 | 1.11 |

^{a}Percentages are based on the number of evaluable subjects for each column.
Note:
Missing Subject Satisfaction with Cellulite Treatment ratings at any time point were not imputed and therefore excluded in the summary.

Results of 2 multicenter, randomized, double-blind, placebo-controlled pivotal studies demonstrated that CCH significantly reduced the severity of cellulite as assessed by the proportion of 2-level composite responders on Day 71 compared to placebo. During the pivotal Phase 3 studies, a clear separation of CCH treatment from placebo on the CR-PCSS and the PR-PCSS ratings was observed as early as 21 days after the first treatment session.

Subgroup analyses demonstrated that there was no apparent impact of age, race, ethnicity, BMI, Fitzpatrick Skin Type, family history of cellulite, baseline cellulite severity, or immunogenicity status, on efficacy. To further evaluate the effect of CCH in the ≥65 year age group and in the ≥30 kg/m² BMI category, supplemental post-hoc analyses were performed. The post-hoc analyses selected were key secondary endpoints in the pivotal studies and included the 1-level composite response in the target buttock, the 2-level composite response in the non-target buttock, and 2-level and 1-level PR PCSS responder analyses. Based, on this analysis, the totality of the evidence supports an efficacy trend favoring CCH compared to placebo in all subgroups.

Statistically significant differences between CCH and placebo treatment groups were also observed in both pooled analyses for all key secondary endpoints. These included: the proportion of 1-level PR PCSS responders of the target buttock, the proportion of 2-level PR-PCSS responders of the target buttock, the proportion of 1-level composite responders of the target buttock, the proportion of 2 level composite responders of the non-target buttock, the proportion of 1-level SSRS responders, the change from baseline (Day 1) of the PR-CIS Total Score, the 1-level S-GAIS responders of the target buttock, and the 2 level S-GAIS responders of the target buttock.

These key secondary endpoints included scales that evaluated patient-reported impact measures of treatment benefit that are relevant and clinically meaningful to subjects. The PR-CIS assessed the visual and emotional impact of cellulite, the SSRS assessed satisfaction with the appearance of their cellulite, and the S-GAIS assessed visual appearance. Overall, the patient-reported satisfaction and emotional impact measures showed a greater and statistically significant improvement in the CCH treatment group as compared to the placebo treatment group. The reduction in the severity of cellulite was evident as early as 21 days after administration of the first treatment session of CCH 0.84 mg per treatment area and was sustained throughout the 71-day double-blind study period. The results of Study −304, up to Day 180, demonstrated durability (the persistence of effect) on the PR-PCSS and CR-PCSS for up to 6 months after completing the parent studies.

### Example 5—Phase 3B, Open-Label, Long-Term Study to Evaluate the Safety and Temporal Pattern of Response of Cch in the Treatment of Cellulite (Study 304)

This was a multicenter, open-label, long term follow-up study from Examples 2 and 3 above. The study evaluated subjects who previously received CCH 0.84 mg per buttock in the concurrent treatment (total dose of up to 1.68 mg×3 treatment sessions) of 2 bilateral buttocks with EFP in adult women in a 71-day double-blind, placebo-controlled study (Example 2 (Study 302) or Example 3 (Study 303), hereafter “parent studies” or “parent study”). After completion in one of the aforementioned studies, subjects were (and will be) followed for up to 5 years from Day 71 (i.e., 70 days after first treatment) of the parent studies, which was approximately 28 days after the last exposure to CCH in those studies.

Unless otherwise specified in this example, “Days” as used in study 304 were relative to Day 71 from initial dose (Day 1) in either study 302 or 303. Cellulite assessments were performed at Day 180 of this study, which was approximately 180 days after the reported Day 71 in Examples 2 and 3, and approximately 251 days after the subjects' first treatment with CCH. Thus, “Day 180” for purposes of this Example 5 means approximately 180 days after the Day 71 of Examples 2 and 3. The Day 180 assessments for this Example 5 were conducted in a blinded fashion in that the subject, investigator and site personnel were blinded to the treatment received in the Phase 3 study until assessments were complete.

The primary objectives of this study were to assess (a) the long-term safety of CCH for cellulite in the treatment of EFP in adult women; (b) the safety of CCH when used for retreatment in the treatment of EFP in adult women; and (c) the long-term immunogenicity profile of CCH following treatment of EFP in adult women. The secondary objective of this study was to assess the time to reduction of response (TRR) in adult women with EFP treated with CCH, e.g., the durability of the treatment. This study is currently ongoing. This Example represents data collected up to and including Apr. 1, 2019. It includes 479 subjects who completed at least the Day 180 assessment visit by that date.

Of the 755 subjects (CCH treated and placebo treated) who completed the studies of Examples 2 and 3, 617 subjects (CCH treated and placebo treated) entered this open-label extension study. Once these subjects were unblinded at Day 180 of this study, only subjects who received active CCH thereafter remained in this study.

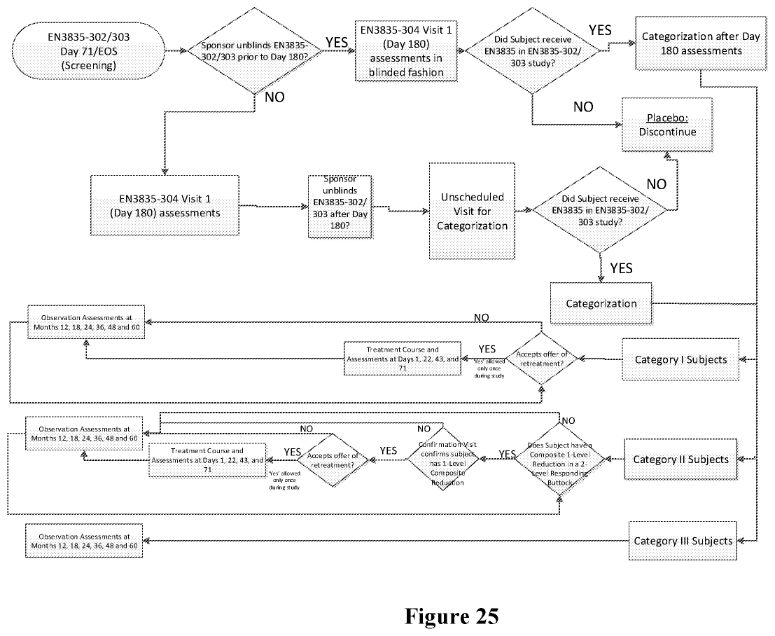

The study design is outlined in FIG. 25. After Day 180 assessments were completed (i.e., 180 days after Day 71 of Examples 2 and 3), the site was unblinded to each subject's study treatment in the parent studies (approximately 6 months after Day 71 of the parent studies, and approximately 251 days after the first dose of CCH in the parent studies), subjects who received placebo during those studies were withdrawn and did not continue in the study. Subjects who received CCH were thereafter classified into 1 of the following 3 categories:
Category I (1-level composite responders) includes subjects who received CCH in the parent studies and in which the maximum composite response in either (or both) buttock(s) at Day 71 in the parent study was a maximum of a 1-level composite improvement in cellulite severity as assessed by the PR-PCSS and the CR-PCSS. In cases where there was an improvement in both buttocks, the composite improvement of 1 level must have occurred in the same buttock to be eligible (i.e., the PR-PCSS and CR-PCSS improvement must have been in the same buttock). Subjects showing a 2-level improvement in 1 scale, but only a 1-level improvement in the other scale were also considered Category I subjects, provided the composite improvement was within the same buttock. Category I subjects are eligible for 1 additional retreatment course (i.e., 3 treatment sessions at approximately 21 days apart) in up to 2 buttocks in this open-label study. Retreatment can begin after assessments on Day 180 and after unblinding of the subjects' treatment during the parent study. Screening for retreatment may have begun at the Day 180 Visit if the study drug blind from the parent studies had been broken. Only buttocks with a composite 1-level improvement or less will be retreated in Category I subjects. Assessment of open-label efficacy following initiation of retreatment (Treatment Visit 1, Treatment Day 1) will be determined up to Treatment Visit 4 (Day 71+5 days) of this open-label study, after which subjects will be observed, depending when during the 5 year period the subject received retreatment, every 6 months for 2 years and then annually for up to 5 years (Day 1800) after Day 71 of the parent studies 302 and 303. Eligible subjects who qualified for Category I status who choose not to receive additional retreatment will have observation visits (Visits 2 through 7, i.e., months 12, 18, 24, 36, 48 and 60).
Category II (2-level composite responders) includes subjects who received CCH in the parent studies and who had a composite improvement in cellulite severity at Day 71 of the parent studies of at least 2 levels in at least 1 buttock in both the PR-PCSS and the CR-PCSS (i.e., 2-level composite responder). These subjects will be observed for TRR (defined below) and safety approximately every 6 months for 2 years and then annually for up to 5 years (Day 1800) from Day 71 of the parent studies. Category II subjects must have at least a 1-level composite response reduction compared to Day 71 of the parent studies and no longer maintain a 2-level composite response compared to Day 1 in the parent study on either or both buttocks to be eligible for retreatment after which they will participate in observation visits.
Category III (non-composite responders) includes all other subjects who received active CCH in the parent studies but did not meet criteria to be included in Category I or II. These subjects will be observed for safety every 6 months for 2 years and annually for up to 5 years (Day 1800), and are not eligible for retreatment during the study.

The Category I subjects and Category II subjects eligible for retreatment will receive study drug in 3 retreatment visits unless the buttock has no treatable EFP dimples and the investigator rates a buttock with a score of 0 on the CR-PCSS at Treatment Visit 1, Treatment Visit 2 and/or Treatment Visit 3. During each retreatment visit, the buttock(s) to be treated will be photographed before and after marking dimples and injection sites while the subject is standing in a consistent, standardized relaxed pose as described elsewhere herein.

The treatment area is defined as eligible buttock(s). A subject will be eligible for treatment after Day 180 in either or both buttocks (i.e., left and/or right buttock). All cellulite assessments will be performed on each individual treatment area. Treatment areas will be evaluated separately. The dimples selected within each buttock should be well-defined, evident when the subject is standing in a consistent relaxed pose, and suitable for treatment. The same dimples within a buttock or different dimples within a buttock can be retreated at the designated treatment visits, but injections must be all within the buttocks (e.g., 12 injections per buttock) for all the treatment visits.

If no injections in a particular buttock (right/left) are given at a treatment visit (for instance, a treatment visit e.g., Treatment Visit 2), subjects will still be assessed for retreatment in the contralateral buttock at that visit (Treatment Visit 2) if the buttock was treated during Treatment Visit 1. When the subject returns for Treatment Visit 3, each of the buttocks will again be evaluated by the subject (using PR-PCSS) and Investigator (using CR-PCSS). If the investigator rates cellulite severity greater than 0 using the CR-PCSS in the eligible buttock(s), then injections will be given.

Category I and Category II subjects who participate in the observational phase of the study will have cellulite assessments completed during the observational visits at Months 12, 18, 24, 36, 48, and 60. At these observation visits, if the ratings indicate that the buttock(s) is eligible for retreatment and the subject has not received CCH previously in this study 304 (Example 5), the subject will be offered 1 course of retreatment for the eligible buttock(s).

The retreatment course for eligible Category I and Category II subjects consists of 3 retreatment sessions separated by 21 days unless the buttock has no treatable EFP dimples i.e., the investigator rates a score of 0 on the CR-PCSS at Treatment Visit 1, 2 and/or Treatment Visit 3. Each retreatment session consists of 12 injections (0.07 mg/0.3 mL per injection) of CCH for a dose of 0.84 mg and volume of 3.6 mL (identical to the treatment course administered in the double-blind study), in each qualifying buttock, with up to 2 buttocks treated concurrently. One and only 1 retreatment course in up to 2 qualifying buttocks concurrently is offered to eligible Category I and Category II subjects during this 5 year study. After initiation of retreatment (on Treatment Visit 1; Treatment Day 1), subjects are assessed for safety on each day of injection during retreatment, and assessed for safety and cellulite severity on Treatment Visit 2 (Day 22), Treatment Visit 3 (Day 43) and Treatment Visit 4 (Day 71). After retreatment, these subjects will be observed, depending when during the 5 year period the subject received retreatment, every 6 months for 2 years and then annually for up to 5 years (Day 1800) after Day 71 of the parent studies (Examples 2 and 3).

For study purposes, reduction of response (i.e., lessening of response or loss of response) is defined as a 1-level composite worsening of the cellulite severity improvement observed at Day 71 of the parent studies on both the PR-PCSS and CR-PCSS. TRR was defined as the time from assessing efficacy (Day 71 of the parent studies) to the study visit at which a composite 1-level loss of response in both PR-PCSS and CR-PCSS was observed in this study. Confirmation of reduction of response and/or complete loss of response is established during a follow-up study visit approximately 2 weeks after a composite reduction of response is first detected.

The digital photographs are not direct efficacy measurements; however, digital photographs are utilized in the assessment of certain efficacy measurements. Each buttock is photographed, using a digital camera in a standardized manner as described above, at the following time points for each of the two buttocks:
Screening, Day 180, and Confirmation Visits (no markings of dimples or injection sites) for all subjects
Before and after marking dimples and injection sites (prior to injections) during Treatment Visits (Day 1, 22, and 43) for Category I and/or eligible Category II subjects
During the Day 71 visit (End of Treatment/Early Termination), Observation Assessments Visits (Day 360, 540, 720, 1080, 1440, and 1800/End of Study (“EOS”)), and Confirmation Visits (no dimple or injection site markings)

After the Day 180 Visit and unblinding of the double-blind parent studies, photography is limited to Category I and Category II subjects.

Up to 843 subjects that received study drug in the parent studies could have participated in this study. A total of 617 subjects completed the parent studies and consented to participate in this study. Of those, 130 did not meet entry criteria, withdrew consent or were lost to follow-up prior to the Day 180 Visit. In addition, 4 subjects attended the Day 180 Visit, but withdrew from the study before unblinding and 4 subjects had not been unblinded at the time of the interim data cut-off (Apr. 1, 2019). Therefore, a total of 479 subjects completed the Day 180 Visit and were unblinded at the time of the data cut-off.

Of these 479 subjects, 238 received placebo in the parent studies, completed Day 180 visit, and then were withdrawn from further participation in this study. Therefore, 241 subjects who had received CCH in the parent studies and who completed the Day 180 Visit in this study were eligible to continue. Of the 241 eligible subjects, 198 had consented to participate in the open-label phase of the study at the time of the data cut-off (Apr. 1, 2019). Of those, 30 subjects were eligible for, and opted to receive CCH retreatment prior to the data cut-off, Tables 62 and 63.

TABLE 62
Summary of subject participation in this study.

| | Number of | Number of | Total |
|---|---|---|---|
| | Subjects | Subjects | Number of |
| | (CCH) | (Placebo) | Subjects |
| Signed Screening A ICF | | | 617 |
| Completed Day 180 | 241 | 238 | 479 |
| Subjects Entered Open | 198 | — | 198 |
| Label Study | | | |
| Category I | 84 | — | 84 |
| Category II | 19 | — | 19 |
| Category III | 95 | — | 95 |
| Time to Reduction of | 103 | — | 103 |
| Response (TRR) Population | | | |

TABLE 63
Summary of the “time to reduction of response (TRR)
population” participation in this study.

| Time to Reduction of | | | |
|---|---|---|---|
| Response (TRR) | Category I | Category II | Total TRR |
| Population | n = 84 | N = 19 | n = 103 |
| Open Label Retreated | 27 | 3 | 30 |
| (OLR) Population | | | |
| Completed 3 Treatment | 13 | — | 13 |
| Sessions (up to and | | | |
| including Day 43) | | | |
| Completed Re-Treatment | 4 | — | 4 |
| Phase (Day 71) | | | |

### Dose and Mode of Administration

CCH for cellulite is a sterile lyophilized powder comprising 0.92 mg of collagenase clostridium histolyticum, as described above. Subjects who qualified for the study were given a maximum dose of 0.84 mg of CCH or placebo per buttock per treatment session (total dose of 1.68 mg per treatment session), administered as 12 subcutaneous injections per buttock at 3 treatment sessions 21 days apart (Table 64).

TABLE 64
Study Retreatment (Eligible Category I and Category II Subjects Only)

| | | Number of | | Injection | |
|---|---|---|---|---|---|
| | Injection | Injections | | Volume (mL) | |
| Dose per | Volume | at Each | | per Each | |
| Each | per Each | Treatment | Dose (mg) at Each | Treatment | Cumulative |
| Injection^{a} | Injection | Visit | Treatment Visit | Visit | EFP Dose |
| CCH 0.07 mg | 0.3 mL | 12 per | 0.84 mg per | 3.6 mL per | 5.04 mg |
| | | buttock × | buttock × | buttock × | (3 treatment |
| | | up to 2 | up to 2 | up to 2 | visits × |
| | | buttocks = | buttocks = 1.68 mg | buttocks = up | 0.84 mg per |
| | | up to 24 | (12 injections | to 7.2 mL | buttock × |
| | | injections | per buttock × | (24 injections × | up to 2 |
| | | | 0.07 mg/injection × | 0.3 mL) | buttocks) |
| | | | up to 2 buttocks) | | |

^{a}Each injection of study drug is 0.3 mL administered as three 0.1 mL aliquots.

Study drug is injected subcutaneously while the subject is in a prone position using a syringe with a 30-gauge ½-inch needle. Each injection site will receive a single skin injection of study drug administered as three 0.1-mL aliquots to Positions A, B, and C (for a total injection volume of 0.3 mL) as shown in FIG. 7. The depth of injection corresponds to the length of the treatment needle (0.5 inches) from the tip of the needle to the hub or base of the needle without downward pressure.

During each retreatment visit, 4 syringes per buttock are prepared for dosing. Each syringe contains 0.9 mL of study drug (i.e., 3 injections in each syringe). Twelve (12) skin injections of 0.3 mL per injection are administered within each eligible buttock getting retreated during each treatment visit. Subjects who qualify for retreatment in this study are given a maximum dose of 0.84 mg of CCH per buttock per treatment day, administered as up to 12 subcutaneous injections per buttock at 3 treatment visits 21 days apart.

### Criteria for Evaluation

Efficacy was evaluated using the CR-PCSS and PR-PCSS, Patient Reported Cellulite Impact Scale (PR-CIS), Subject Global Aesthetic Improvement Scale (S-GAIS), Subject Self-rating Scale (SSRS), and Subject Satisfaction with Cellulite Treatment Assessment.

The safety endpoints included adverse events (AEs), vital signs, physical examination, laboratory tests, and immunogenicity.

### Statistical Methods

The following analysis populations were used:
Day 180 Observational Population includes all rollover subjects from the parent studies.
Open-label Observation Population includes all subjects who entered the open-label study and received CCH in the parent studies. This population includes all Category I, II, and III subjects.
TRR before Retreatment Population includes all subjects who had at least a 1-level or 2-level improvement in both CR-PCSS and PR-PCSS ratings at Day 71 of the parent study for either/both treated buttocks. This population includes Category I and II subjects. Reduction of response is evaluated separately for subjects who had at least a 1-level and 2-level improvement in both CR-PCSS and PR-PCSS ratings during the parent study in each treated buttock. The reduction of response prior to retreatment is analyzed using this population.
Open-label Retreated (OLR) Population includes all subjects in the TRR Population who are retreated in this open-label study.
TRR after Retreatment Population includes all subjects in the OLR Population who have at least a 1-level or 2-level composite improvement in both CR-PCSS and PR-PCSS ratings at Day 71/End of Treatment Clinic Visit in the open-label treatment phase. Reduction of response is evaluated separately for subjects who have at least a 1-level and 2-level composite improvement in both CR-PCSS and PR-PCSS ratings during the open-label treatment phase in each treated buttock. The reduction of response after retreatment is analyzed using this population.

### Efficacy Analyses

The response definitions used in the study were as follows.

Reduction of Response in Cellulite Severity Scale (PR-PCSS and CR-PCSS): Reduction of response is assessed separately for prior to retreatment and after retreatment. Table 65 outlines the definitions for reduction of response in this study.

TABLE 65
Reduction of Response

| Variables | Prior to Retreatment | After Retreatment |
|---|---|---|
| Reduction of | Worsening of cellulite severity | Worsening of cellulite severity |
| Response | improvement on PR-PCSS or CR- | improvement on PR-PCSS or CR- |
| | PCSS in this study prior to | PCSS during the observation phase |
| | retreatment compared to the score | after retreatment compared to the |
| | at Day 71/EOS in the parent study | score at Day 71/End of Treatment |
| | (Studies 302/303). This reduction is | Clinic Visit in the open-label |
| | also referred as a Partial Loss of | treatment phase. |
| | Response. | |
| 2-level Reduction | Worsening of cellulite severity | Worsening of cellulite severity |
| | improvement by at least 2 levels on | improvement by at least 2 levels on |
| | PR-PCSS or CR-PCSS in this study | PR-PCSS or CR-PCSS during the |
| | prior to retreatment compared to the | observation phase after retreatment |
| | score at Day 71/EOS in the parent | compared to the score at Day |
| | study (i.e., a change from baseline | 71/End of Treatment Clinic Visit in |
| | PR-PCSS and/or CR-PCSS rating | the open-label treatment phase (i.e., |
| | of 2, 3, or 4) | a change from baseline PR-PCSS |
| | | and/or CR-PCSS rating of 2, 3, or 4) |
| 1-level Reduction | Worsening of cellulite severity | Worsening of cellulite severity |
| | improvement by at least 1 level on | improvement by at least 1 level on |
| | PR-PCSS or CR-PCSS in this study | PR-PCSS or CR-PCSS during the |
| | after retreatment compared to the | observation phase after retreatment |
| | score at Day 71/EOS in the parent | compared to the score at Day |
| | study (i.e., a change from baseline | 71/End of Treatment Clinic Visit in |
| | PR-PCSS and/or CR-PCSS rating | the open-label treatment phase (i.e., |
| | of 1, 2, 3, or 4) | a change from baseline PR-PCSS |
| | | and/or CR-PCSS rating of 1, 2, 3, or 4) |
| Composite | Composite Reduction was assessed using both the subject PR-PCSS | |
| Reduction | responder classification and investigator CR-PCSS responder, where both | |
| | the scales had same level of reduction. If the classification was missing for | |
| | 1 or both components (i.e., the PR-PCSS component or the CR-PCSS | |
| | component), then the composite responder classification was missing for | |
| | that visit. | |
| 2-level Composite | Worsening of cellulite severity | Worsening of cellulite severity |
| Reduction | improvement by at least 2 levels on | improvement by at least 2 levels on |
| | both PR-PCSS and CR-PCSS | both PR-PCSS and CR-PCSS |
| | compared to the score at Day | compared to the score at Day |
| | 71/EOS in the parent study. | 71/End of Treatment Clinic Visit in |
| | | the open-label treatment phase. |
| 1-level Composite | Worsening of cellulite severity | Worsening of cellulite severity |
| Reduction | improvement by only 1 level on | improvement by only 1 level on |
| | both PR-PCSS and CR-PCSS | both PR-PCSS and CR-PCSS |
| | compared to the score at Day | compared to the score at Day |
| | 71/EOS in the parent study. | 71/End of Treatment Clinic Visit in |
| | | the open-label treatment phase. |
| Complete Loss of | Worsening of cellulite severity | Worsening of cellulite severity |
| Response | improvement on PR-PCSS and CR- | improvement on PR-PCSS and CR- |
| | PCSS ratings in this study prior to | PCSS ratings compared to the score |
| | retreatment compared to the score | at Day 71/End of Treatment Clinic |
| | at Day 71/EOS in the parent study, | Visit in the open-label treatment |
| | where cellulite severity returns to | phase, where cellulite severity |
| | baseline levels of the double-blind | returns to baseline levels of the |
| | studies (i.e., Day 1 of the parent | retreatment phase (i.e., Day 1 of the |
| | study) or worse. | open-label retreatment phase) or |
| | | worse. |

Time to Reduction of Response: Table 66 outlines how the TRR was calculated.

TABLE 66
Time to Reduction of Response

| Variables | Prior to Retreatment | After Retreatment |
|---|---|---|
| Time to | Number of days from the time of | Number of days from the time of |
| Composite | assessing composite improvement | assessing composite improvement |
| Reduction of | in cellulite severity on PR-PCSS | in cellulite severity on PR-PCSS |
| Response by 2 | and CR-PCSS at Day 71/EOS in the | and CR-PCSS at Day 71/End of |
| levels | parent study to the study visit prior | Treatment Clinic Visit in the open- |
| | to retreatment at which at least a 2- | label treatment phase to the study |
| | level worsening of response in both | visit during the observation phase |
| | PR-PCSS and CR-PCSS ratings is | after retreatment at which at least a |
| | observed for the first time. | 2-level worsening of response in |
| | | both PR-PCSS and CR-PCSS |
| | | ratings is observed for the first time. |
| Time to | Number of days from the time of | Number of days from the time of |
| Composite | assessing composite improvement | assessing composite improvement |
| Reduction of | in cellulite severity on PR-PCSS | in cellulite severity on PR-PCSS |
| Response by 1 | and CR-PCSS at Day 71/EOS in the | and CR-PCSS at Day 71/End of |
| level | parent study to the study visit prior | Treatment Clinic Visit in the open- |
| | to retreatment at which only a 1- | label treatment phase to the study |
| | level of worsening of response in | visit during the observation phase |
| | both PR-PCSS and CR-PCSS | after retreatment at which only a |
| | ratings is observed for the first time. | 1-level of worsening of response in |
| | | both PR-PCSS and CR-PCSS |
| | | ratings is observed for the first time. |
| Time to Complete | Number of days from the time of | Number of days from the time of |
| Loss of Response | assessing composite improvement | assessing composite improvement |
| | in cellulite severity on PR-PCSS | in cellulite severity on PR-PCSS |
| | and CR-PCSS at Day 71/EOS in the | and CR-PCSS at Day 71/End of |
| | parent study to the study visit prior | Treatment Clinic Visit in the open- |
| | to retreatment at which the | label treatment phase to the study |
| | complete loss of response in both | visit during the observation phase |
| | PR-PCSS and CR-PCSS is | after retreatment at which the |
| | observed. | complete loss of response in both |
| | | PR-PCSS and CR-PCSS is |
| | | observed. |

Improvement of Response in Cellulite Severity Scale (PR-PCSS and CR-PCSS): For subjects who received retreatment in this study, the improvement of response in cellulite severity during retreatment phase (i.e., at Day 22, 43, and 71) compared to Day 1 of the retreatment phase is assessed as:
2-level Improvement: defined as improvement in cellulite severity at least by 2 levels on PR-PCSS or CR-PCSS ratings after receiving the retreatment compared to the corresponding rating at Day 1 of the Treatment Course (Treatment Session 1) of this study (i.e., change from baseline PR-PCSS and/or CR-PCSS rating of −2, or −3).
1-level Improvement: defined as improvement in cellulite severity at least by 1 level on PR-PCSS or CR-PCSS ratings after receiving the retreatment compared to the corresponding rating at Day 1 of the Treatment Course (Treatment Session 1) of this study (i.e., change from baseline PR-PCSS and/or CR-PCSS rating of −1,−2, or −3).
2-level Responder: defined as a subject with an improvement in the PR-PCSS or CR-PCSS ratings of at least 2 levels compared to the ratings at Day 1 of the Treatment Course (Treatment Session 1) of this study.
1-level Responder: defined as a subject with an improvement in the PR-PCSS or CR PCSS ratings of at least 1 level compared to the ratings at Day 1 of the Treatment Course (Treatment Session 1) of this study.
1- or 2-level Composite Improvement is assessed using both the PR-PCSS and CR-PCSS (e.g., for 1 level composite improvement—the improvement in response of at least 1-level is observed in both PR-PCSS and CR-PCSS for the same buttock. For the 2 level composite improvement—the improvement of at least 2 levels is observed in both PR-PCSS and CR-PCSS for the same buttock). If the classification is missing for 1 or both components (i.e., the PR-PCSS component or the CR-PCSS component), then the composite responder classification is missing for that visit.
2-level Composite Responder: defined as a subject who is at least a 2-level PR-PCSS responder and at least a 2-level CR-PCSS responder.
1-level Composite Responder: defined as a subject who is at least a 1-level PR-PCSS responder and at least a 1-level CR-PCSS responder.

### PR-CIS Responder:

For PR-CIS total score, a PR-CIS responder is defined as a subject with a reduction in the PR-CIS total score of at least 12 from baseline (i.e., average of at least 2 for each item) at an evaluation time point.
For PR-CIS abbreviated total score, a PR-CIS responder is defined as a subject with a reduction in the PR-CIS total score of at least 10 from baseline (i.e., average of at least 2 for each item) at an evaluation time point.
A PR-CIS responder for each item is defined as a subject with a reduction in the PR-CIS item score of at least 2 from the score at Day 71 of the open-label phase at an evaluation time point.
Item #1 on the PR-CIS asking—“how happy the subject is about their appearance of cellulite” is reversed by subtracting the subject's reported assessment from 10 (i.e., for purposes of the composite, scoring for the “happy” question is reversed (reflected) to make it directionally consistent with the other questions). In this manner, a higher number on 6 of the PR-CIS questions will reflect a more negative impact.

Subject Global Aesthetic Improvement Scale: A 2-level S-GAIS responder is defined as a subject with S-GAIS rating of at least 2 (i.e., 2, or 3) at an evaluation time point. A 1-level S-GAIS responder is defined as a subject with S-GAIS rating of at least 1 (i.e., 1, 2, or 3) at an evaluation time point.

Subject Satisfaction with Cellulite Treatment Assessment: A responder is defined as a subject with a response of “Satisfied” or “Very Satisfied” in the subject satisfaction with cellulite treatment assessment during an evaluation.

Subject Self-Rating Scale: A 1-Level SSRS responder is defined as a subject who is at least slightly satisfied (i.e., slightly satisfied [4], very satisfied [5] or extremely satisfied [6]) with the appearance of the cellulite on her buttocks during an evaluation. A 2-Level SSRS responder is defined as a subject who is at least very satisfied (i.e., very satisfied [5] or extremely satisfied [6]) with the appearance of the cellulite on her buttocks during an evaluation.

### Efficacy Results

### A. Analysis of Efficacy—Day 180 Observational Population

### 1. Cellulite Severity

a. PR-PCSS and CR-PCSS

Table 67 outlines the changes in PR-PCSS and CR-PCSS from Day 1 of the parent studies to Day 71 of the parent studies, and to Day 180 of this study (180 days after Day 71 of the parent studies, or approximately 251 days after the first dose of study drug in the parent studies) in the Day 180 Observational Population.

TABLE 67
Change from Day 1 of the parent studies in Cellulite Severity Ratings Up to the
Day 180 Visit (Day 180 Observational Population, as defined in this study 304)

| | | Study Treatment in Studies 302/303 | | | | | | | |
|---|---|---|---|---|---|---|---|---|---|
| Cellulite Severity Scale | | CCH | | | | Placebo | | | |
| Cellulite Severity Rating | Statistic^{a} | Left Buttock | | Right Buttock | | Left Buttock | | Right Buttock | |
| PR-PCSS | | | | | | | | | |
| Change from Day 1 at Day | N | 241 | | 241 | | 238 | | 238 | |
| 71/EOS in Studies 302/303 | | | | | | | | | |
| −4 | n (%) | 0 | | 1 | (0.4) | 1 | (0.4) | 1 | (0.4) |
| −3 | n (%) | 4 | (1.7) | 11 | (4.6) | 0 | | 2 | (0.8) |
| −2 | n (%) | 50 | (20.7) | 42 | (17.4) | 25 | (10.5) | 25 | (10.5) |
| −1 | n (%) | 95 | (39.4) | 93 | (38.6) | 61 | (25.6) | 56 | (23.5) |
| 0 | n (%) | 84 | (34.9) | 88 | (36.5) | 144 | (60.5) | 146 | (61.3) |
| +1 | n (%) | 8 | (3.3) | 6 | (2.5) | 7 | (2.9) | 8 | (3.4) |
| +2 | n (%) | 0 | | 0 | | 0 | | 0 | |
| +3 | n (%) | 0 | | 0 | | 0 | | 0 | |
| | Mean | −0.8 | | −0.9 | | −0.5 | | −0.5 | |
| | SD | 0.85 | | 0.92 | | 0.75 | | 0.79 | |
| PR-PCSS | | | | | | | | | |
| Change from Day 1 of Studies | N | 241 | | 241 | | 237 | | 237 | |
| 302/303 at Day 180/Early | | | | | | | | | |
| Termination | | | | | | | | | |
| −4 | n (%) | 1 | (0.4) | 0 | | 0 | | 1 | (0.4) |
| −3 | n (%) | 5 | (2.1) | 10 | (4.1) | 1 | (0.4) | 2 | (0.8) |
| −2 | n (%) | 30 | (12.4) | 34 | (14.1) | 15 | (6.3) | 22 | (9.3) |
| −1 | n (%) | 92 | (38.2) | 86 | (35.7) | 62 | (26.2) | 65 | (27.4) |
| 0 | n (%) | 101 | (41.9) | 98 | (40.7) | 145 | (61.2) | 134 | (56.5) |
| +1 | n (%) | 12 | (5.0) | 13 | (5.4) | 14 | (5.9) | 13 | (5.5) |
| +2 | n (%) | 0 | | 0 | | 0 | | 0 | |
| +3 | n (%) | 0 | | 0 | | 0 | | 0 | |
| | Mean | −0.7 | | −0.7 | | −0.3 | | −0.4 | |
| | SD | 0.87 | | 0.92 | | 0.71 | | 0.80 | |
| CR-PCSS | | | | | | | | | |
| Change from Day 1 at Day | N | 241 | | 241 | | 238 | | 238 | |
| 71/EOS in Studies 302/303 | | | | | | | | | |
| −4 | n (%) | 0 | | 0 | | 0 | | 0 | |
| −3 | n (%) | 2 | (0.8) | 1 | (0.4) | 0 | | 3 | (1.3) |
| −2 | n (%) | 39 | (16.2) | 37 | (15.4) | 10 | (4.2) | 11 | (4.6) |
| −1 | n (%) | 110 | (45.6) | 103 | (42.7) | 65 | (27.3) | 55 | (23.1) |
| 0 | n (%) | 82 | (34.0) | 94 | (39.0) | 149 | (62.6) | 156 | (65.5) |
| +1 | n (%) | 8 | (3.3) | 6 | (2.5) | 14 | (5.9) | 13 | (5.5) |
| +2 | n (%) | 0 | | 0 | | 0 | | 0 | |
| +3 | n (%) | 0 | | 0 | | 0 | | 0 | |
| | Mean | −0.8 | | −0.7 | | −0.3 | | −0.3 | |
| | SD | 0.79 | | 0.76 | | 0.64 | | 0.70 | |
| CR-PCSS | | | | | | | | | |
| Change from Day 1 of Studies | N | 241 | | 241 | | 236 | | 236 | |
| 302/303 at Day 180/Early | | | | | | | | | |
| Termination | | | | | | | | | |
| −4 | n (%) | 0 | | 0 | | 0 | | 0 | |
| −3 | n (%) | 1 | (0.4) | 1 | (0.4) | 0 | | 1 | (0.4) |
| −2 | n (%) | 31 | (12.9) | 35 | (14.5) | 15 | (6.4) | 12 | (5.1) |
| −1 | n (%) | 89 | (36.9) | 88 | (36.5) | 80 | (33.9) | 76 | (32.2) |
| 0 | n (%) | 101 | (41.9) | 101 | (41.9) | 124 | (52.5) | 129 | (54.7) |
| +1 | n (%) | 19 | (7.9) | 16 | (6.6) | 17 | (7.2) | 18 | (7.6) |

| | | | | | |
|---|---|---|---|---|---|
| +2 | n (%) | 0 | 0 | 0 | 0 |
| +3 | n (%) | 0 | 0 | 0 | 0 |
| | Mean | −0.6 | −0.6 | −0.4 | −0.4 |
| | SD | 0.83 | 0.83 | 0.72 | 0.72 |

^{a}Percentages are based on the number of subjects (N) with each buttock evaluated at each visit.

In the 241 subjects who were treated with CCH in the parent studies and completed the Day 180 assessments of cellulite severity in this study, the mean (SD) change from Day 1 to Day 71 in the parent studies in PR-PCSS was −0.8 (0.85) for the left buttock and −0.9 (0.92) for the right buttock. In these same subjects, the mean (SD) change in PR-PCSS from Day 1 in the parent studies to Day 180 in this study was −0.7 (0.87) for the left buttock and −0.7 (0.92) for the right buttock.

The mean (SD) CR-PCSS change from Day 1 to Day 71 in the parent studies was −0.8 (0.79) for the left buttock and −0.7 (0.76) for the right buttock. The mean change (SD) in CR-PCSS from Day 1 of the parent studies to Day 180 of this study was −0.6 (0.83) for the left buttock and −0.6 (0.83) for the right buttock.

In the 238 subjects treated with placebo in the parent studies, the mean (SD) PR-PCSS change from Day 1 to Day 71 was −0.5 (0.75) and −0.5 (0.79) in the left and right buttock, respectively. The mean change from Day 1 in the parent study to Day 180 in this study in PR-PCSS was −0.3 (0.71) in the left buttock and −0.4 (0.80) in the right buttock.

In placebo treated subjects, the mean (SD) CR-PCSS change from Day 1 to Day 71 was −0.3 (0.64) and −0.3 (0.70) in the left and right buttock, respectively. The mean change from Day 1 in the parent study to Day 180 in this study in CR-PCSS was −0.4 (0.72) in the left buttock and −0.4 (0.72) in the right buttock.

The mean changes in CR-PCSS and PR-PCSS from Day 1 to Day 71 (in the parent study) and from Day 1 in the parent study to Day 180 in this study indicate that subjects in the Day 180 Observational Population are representative of the population of the parent studies. The difference between CCH treated subjects and placebo treated subjects at Day 180 is consistent with the results seen in the parent studies.

b. PR-CIS

Table 68 outlines the changes in PR-CIS from Day 1 of the parent studies to Day 71 of the parent studies, and to Day 180 of this study in the Day 180 Observational Population.

TABLE 68
Change from Day 1 of the parent studies in PR-CIS Total, Abbreviated,
and Item Scores up to Day 180/Early Termination Visit (Day 180
Observational Population, as defined in this study 304)

| | | Study Treatment in | |
|---|---|---|---|
| | | Studies 302/303 | |
| | | CCH | Placebo |
| PR-CIS^{a} Score | Sta- | (N = | (N = |
| Visit | tistic | 241) | 238) |
| Total Score | | | |
| Change from Day 1 to Day 71/EOS in | n | 239 | 233 |
| Studies 302/303 | | | |
| | Mean | −11.8 | −6.5 |
| | SD | 11.77 | 12.12 |
| | Median | −10.0 | −4.0 |
| | min | −53 | −49 |
| | max | 27 | 41 |
| Change from Day 1 of in Studies 302/302 | n | 241 | 234 |
| to Day 180/Early Termination | | | |
| | Mean | −10.5 | −5.4 |
| | SD | 11.76 | 11.30 |
| | Median | −10.0 | −3.0 |
| | min | −50 | −50 |
| | max | 25 | 47 |
| Abbreviated Score | | | |
| Change from Day 1 to Day 71/EOS in | n | 239 | 233 |
| Studies 302/303 | | | |
| | Mean | −10.3 | −5.7 |
| | SD | 9.95 | 10.30 |
| | Median | −10.0 | −4.0 |
| | min | −44 | −43 |
| | max | 19 | 34 |
| Change from Day 1 of in Studies 302/302 | n | 241 | 234 |
| to Day 180/Early Termination | | | |
| | Mean | −9.3 | −5.2 |
| | SD | 9.88 | 9.90 |
| | Median | −9.0 | −3.0 |
| | min | −40 | −44 |
| | max | 22 | 37 |
| Question 1: Impact of Happiness | | | |
| Change from Day 1 to Day 71/EOS in | n | 239 | 233 |
| Studies 302/303 | | | |
| | Mean | −3.3 | −1.6 |
| | SD | 3.50 | 3.20 |
| | Median | −3.0 | 0.0 |
| | min | −10 | −10 |
| | max | 10 | 10 |
| Change from Day 1 of in Studies 302/302 | n | 241 | 234 |
| to Day 180/Early Termination | | | |
| | Mean | −2.8 | −1.5 |
| | SD | 3.25 | 3.00 |
| | Median | −3.0 | 0.0 |
| | min | −10 | −10 |
| | max | 10 | 10 |
| Question 2: Impact of Bothersome | | | |
| Change from Day 1 to Day 71/EOS in | n | 239 | 233 |
| Studies 302/303 | | | |
| | Mean | −1.8 | −1.3 |
| | SD | 3.50 | 3.92 |
| | Median | −2.0 | 0.0 |
| | min | −10 | −10 |
| | max | 10 | 10 |
| Change from Day 1 of in Studies 302/302 | n | 241 | 234 |
| to Day 180/Early Termination | | | |
| | Mean | −1.8 | −1.3 |
| | SD | 3.67 | 3.84 |
| | Median | −2.0 | −1.0 |
| | min | −10 | −10 |
| | max | 10 | 10 |
| Question 3: Impact of Self-consciousness | | | |
| Change from Day 1 to Day 71/EOS in | n | 239 | 233 |
| Studies 302/303 | | | |
| | Mean | −1.8 | −1.0 |
| | SD | 2.61 | 2.51 |
| | Median | −1.0 | 0.0 |
| | min | −10 | −10 |
| | max | 9 | 8 |
| Change from Day 1 of in Studies 302/302 | n | 241 | 234 |
| to Day 180/Early Termination | | | |
| | Mean | −1.7 | −1.1 |
| | SD | 2.81 | 2.90 |
| | Median | −2.0 | 0.0 |
| | min | −10 | −10 |
| | max | 10 | 10 |
| Question 4: Impact of Embarrassment | | | |
| Change from Day 1 to Day 71/EOS in | n | 239 | 233 |
| Studies 302/303 | | | |
| | Mean | −2.0 | −1.0 |
| | SD | 2.75 | 2.56 |
| | Median | −2.0 | 0.0 |
| | min | −10 | −10 |
| | max | 8 | 9 |
| Change from Day 1 of in Studies 302/302 | n | 241 | 234 |
| to Day 180/Early Termination | | | |
| | Mean | −1.9 | −0.9 |
| | SD | 2.56 | 2.58 |
| | Median | −2.0 | 0.0 |
| | min | −10 | −10 |
| | max | 10 | 10 |
| Question 5: Impact of Older Appearance | | | |
| Change from Day 1 to Day 71/EOS in | n | 239 | 233 |
| Studies 302/303 | | | |
| | Mean | −1.5 | −0.8 |
| | SD | 2.72 | 2.92 |
| | Median | −1.0 | 0.0 |
| | min | −10 | −10 |
| | max | 8 | 10 |
| Change from Day 1 of in Studies 302/302 | n | 241 | 234 |
| to Day 180/Early Termination | | | |
| | Mean | −1.2 | −0.2 |
| | SD | 2.88 | 2.61 |
| | Median | −1.0 | 0.0 |
| | min | −10 | −10 |
| | max | 8 | 10 |
| Question 6: Impact of Body Shape | | | |
| Concern | | | |
| Change from Day 1 to Day 71/EOS in | n | 239 | 233 |
| Studies 302/303 | | | |
| | Mean | −1.4 | −0.7 |
| | SD | 2.50 | 2.37 |
| | Median | −1.0 | 0.0 |
| | min | −10 | −10 |
| | max | 8 | 9 |
| Change from Day 1 of in Studies 302/302 | n | 241 | 234 |
| to Day 180/Early Termination | | | |
| | Mean | −1.1 | −0.4 |
| | SD | 2.52 | 2.47 |
| | Median | 0.0 | 0.0 |
| | min | −10 | −10 |
| | max | 8 | 7 |

^{a}A more negative change in score indicates a greater reduction of the impact of cellulite on the subject’s life.

In subjects who were treated with CCH in the parent studies and completed the Day 180 assessments of PR-CIS in this study, the mean (SD) change from Day 1 to Day 71 in the parent studies in PR-CIS Total Score was −11.8 (11.77). In these same subjects, the mean (SD) change in PR-CIS Total Score from Day 1 in the parent studies to Day 180 in this study was −10.5 (11.76).

In placebo treated subjects in the parent studies, the mean (SD) change from Day 1 to Day 71 in the parent studies in PR-CIS Total Score was −6.5 (12.12). In these same subjects, the mean (SD) change in PR-CIS Total Score from Day 1 in the parent studies to Day 180 in this study was −5.4 (11.30).

For the PR-CIS Abbreviated Score, the mean (SD) change from Day 1 to Day 71 in the parent studies was −10.3 (9.95) and the mean (SD) change in PR-CIS Abbreviated Score from Day 1 in the parent studies to Day 180 in this study was −9.3 (9.88) in CCH treated subjects. In placebo treated subjects, the mean (SD) change from Day 1 to Day 71 in the parent studies was −5.7 (10.30) and the mean (SD) change in PR-CIS Abbreviated Score from Day 1 in the parent studies to Day 180 in this study was −5.2 (9.90).

Similar results were seen when examining changes in response for each individual item in the PR-CIS.

The mean changes in PR-CIS total, abbreviated and item scores from Day 1 to Day 71 (in the parent studies) and from Day 1 in the parent studies to Day 180 in this study also indicate that subjects in the Day 180 Observational Population are representative of the population of the parent studies. The difference between CCH treated subjects and placebo treated subjects at Day 180 is consistent with the results seen in the parent studies.

### 2. Persistence of Treatment Effect

a. PR-PCSS and CR-PCSS Day 180 Observational Population

Table 69 outlines the change from Day 71 of the parent studies to Day 180 of this study in PR-PCSS and CR-PCSS in the Day 180 Observational Population.

TABLE 69
Change from Day 71/EOS in the parent studies in PR-PCSS and CR-PCSS at Day 180/Early
Termination Visit (Day 180 Observational Population, as defined in this study 304)

| | | Study Treatment in Studies 302/303 | | | | | | | |
|---|---|---|---|---|---|---|---|---|---|
| | | CCH | | | | Placebo | | | |
| Cellulite Severity Scale | | Left | | Right | | Left | | Right | |
| Cellulite Severity Rating | Statistic^{a} | Buttock | | Buttock | | Buttock | | Buttock | |
| PR-PCSS | | | | | | | | | |
| Change from Day 71/EOS in | N | 241 | | 241 | | 237 | | 237 | |
| Studies 302/303 to Day | | | | | | | | | |
| 180/Early Termination | | | | | | | | | |
| −4 | n (%) | 1 | (0.4) | 0 | | 0 | | 1 | (0.4) |
| −3 | n (%) | 0 | | 0 | | 0 | | 1 | (0.4) |
| −2 | n (%) | 3 | (1.2) | 7 | (2.9) | 6 | (2.5) | 5 | (2.1) |
| −1 | n (%) | 33 | (13.7) | 35 | (14.5) | 25 | (10.5) | 40 | (16.9) |
| 0 | n (%) | 130 | (53.9) | 127 | (52.7) | 152 | (64.1) | 139 | (58.6) |
| +1 | n (%) | 66 | (27.4) | 60 | (24.9) | 46 | (19.4) | 45 | (19.0) |
| +2 | n (%) | 7 | (2.9) | 11 | (4.6) | 7 | (3.0) | 5 | (2.1) |
| +3 | n (%) | 1 | (0.4) | 0 | | 0 | | 0 | |
| +4 | n (%) | 0 | | 1 | (0.4) | 1 | (0.4) | 1 | (0.4) |
| | Mean | 0.2 | | 0.2 | | 0.1 | | 0.0 | |
| | SD | 0.81 | | 0.86 | | 0.76 | | 0.84 | |
| CR-PCSS | | | | | | | | | |
| Change from Day 71/EOS in | N | 241 | | 241 | | 236 | | 236 | |
| Studies 302/303 to Day | | | | | | | | | |
| 180/Early Termination | | | | | | | | | |
| −4 | n (%) | 0 | | 0 | | 0 | | 0 | |
| −3 | n (%) | 0 | | 0 | | 0 | | 0 | |
| −2 | n (%) | 3 | (1.2) | 2 | (0.8) | 7 | (3.0) | 5 | (2.1) |
| −1 | n (%) | 33 | (13.7) | 40 | (16.6) | 50 | (21.2) | 53 | (22.5) |
| 0 | n (%) | 125 | (51.9) | 134 | (55.6) | 142 | (60.2) | 134 | (56.8) |
| +1 | n (%) | 71 | (29.5) | 59 | (24.5) | 32 | (13.6) | 39 | (16.5) |
| +2 | n (%) | 8 | (3.3) | 4 | (1.7) | 5 | (2.1) | 4 | (1.7) |

| | | | | | | | | |
|---|---|---|---|---|---|---|---|---|
| +3 | n (%) | 1 | (0.4) | 2 | (0.8) | 0 | 1 | (0.4) |

| | | | | | |
|---|---|---|---|---|---|
| +4 | n (%) | 0 | 0 | 0 | 0 |
| | Mean | 0.2 | 0.1 | −0.1 | −0.1 |
| | SD | 0.78 | 0.76 | 0.74 | 0.76 |

^{a}Percentages are based on the number of subjects with each buttock evaluated at each visit.

For PR-PCSS, the mean (SD) change from Day 71 in the parent studies to Day 180 in this study in CCH treated subjects was 0.2 (0.81) in the left buttock and 0.2 (0.86) in the right buttock. In placebo treated subjects, the mean (SD) change on PR-PCSS was 0.1 (0.76) and 0.0 (0.84) in the left and right buttock, respectively.

For CR-PCSS, the mean (SD) change from Day 71 in the parent studies to Day 180 in this study in CCH treated subjects was 0.2 (0.78) in the left buttock and 0.1 (0.76) in the right buttock. In placebo treated subjects, the mean (SD) change on CR-PCSS was −0.1 (0.74) and −0.1 (0.76) in the left and right buttock respectively.

The small differences in PR-PCSS and CR-PCSS between Day 71 and Day 180 indicate a persistence of treatment effect up to 6 months after Day 71 in the parent studies in subjects treated with CCH. These results also indicate that the mean treatment effect was virtually identical between the left and right buttock.

b. PR-CIS Day 180 Observational Population

Table 70 outlines the change from Day 71 of the parent studies to Day 180 of this study in PR-CIS total, abbreviated, and item scores in the Day 180 Observational Population.

TABLE 70
Change from Day 71/EOS of Studies 302/303 in PR-CIS Total Score, Abbreviated
Score and Item Scores at Day 180/Early Termination Visit (Day 180
Observational Population, as defined in this study 304)

| | | Study Treatment in Studies 302/303 | |
|---|---|---|---|
| | | CCH | Placebo |
| PR-CIS^{a} | Statistic | (N = 241) | (N = 238) |
| Total Score | | | |
| Day 71/EOS - Study Studies 302/303 | n | 239 | 236 |
| | Mean | 40.1 | 44.9 |
| | SD | 12.72 | 13.00 |
| | Median | 40.0 | 47.5 |
| | min | 6 | 6 |
| | max | 60 | 60 |
| Day 180/Early Termination | n | 241 | 237 |
| | Mean | 41.3 | 45.9 |
| | SD | 12.13 | 11.76 |
| | Median | 43.0 | 49.0 |
| | min | 8 | 5 |
| | max | 60 | 60 |
| Change from Day 71/EOS of the Studies 302/303 | n | 239 | 235 |
| Studies to Day 180/Early Termination | | | |
| | Mean | 1.2 | 1.0 |
| | SD | 11.32 | 10.14 |
| | Median | 1.0 | 0.0 |
| | min | −50 | −30 |
| | max | 43 | 39 |
| Abbreviated Score | | | |
| Day 71/-EOS - Studies 302/303 | n | 239 | 236 |
| | Mean | 33.7 | 38.2 |
| | SD | 10.63 | 10.76 |
| | Median | 34.0 | 40.0 |
| | min | 5 | 5 |
| | max | 50 | 50 |
| Day 180/Early Termination | n | 241 | 237 |
| | Mean | 34.7 | 38.6 |
| | SD | 10.21 | 9.97 |
| | Median | 35.0 | 40.0 |
| | min | 7 | 5 |
| | max | 50 | 50 |
| Change from Day 71/EOS of the 302/303 | n | 239 | 235 |
| Studies to Day 180/Early Termination | | | |
| | Mean | 1.0 | 0.4 |
| | SD | 9.45 | 8.98 |
| | Median | 1.0 | 0.0 |
| | min | −40 | −30 |
| | max | 33 | 36 |
| Question 1: Impact of Happiness | | | |
| Change from Day 71/EOS of the 302/303 | n | 239 | 235 |
| Studies to Day 180/Early Termination | | | |
| | Mean | 0.5 | 0.1 |
| | SD | 2.68 | 2.88 |
| | Median | 0.0 | 0.0 |
| | min | −9 | −10 |
| | max | 8 | 10 |
| Question 2: Impact of Bothersome | | | |
| Change from Day 71/EOS of the 302/303 | n | 239 | 235 |
| Studies to Day 180/Early Termination | | | |
| | Mean | 0.0 | −0.0 |
| | SD | 3.55 | 3.78 |
| | Median | 0.0 | 0.0 |
| | min | −10 | −10 |
| | max | 10 | 10 |
| Question 3: Impact of Self-consciousness | | | |
| Change from Day 71/EOS of the 302/303 | n | 239 | 235 |
| Studies to Day 180/Early Termination | | | |
| | Mean | 0.1 | −0.1 |
| | SD | 2.63 | 2.92 |
| | Median | 0.0 | 0.0 |
| | min | −10 | −10 |
| | max | 10 | 10 |
| Question 4: Impact of Embarrassment | | | |
| Change from Day 71/EOS of the 302/303 | n | 239 | 235 |
| Studies to Day 180/Early Termination | | | |
| | Mean | 0.1 | 0.1 |
| | SD | 2.62 | 2.27 |
| | Median | 0.0 | 0.0 |
| | min | −10 | −10 |
| | max | 10 | 10 |
| Question 5: Impact of Older Appearance | | | |
| Change from Day 71/EOS of the 302/303 | n | 239 | 235 |
| Studies to Day 180/Early Termination | | | |
| | Mean | 0.3 | 0.6 |
| | SD | 2.92 | 2.42 |
| | Median | 0.0 | 0.0 |
| | min | −10 | −6 |
| | max | 10 | 7 |
| Question 6: Impact of Body Shape Concern | | | |
| Change from Day 71/EOS of the 302/303 | n | 239 | 235 |
| Studies to Day 180/Early Termination | | | |
| | Mean | 0.3 | 0.4 |
| | SD | 2.59 | 2.56 |
| | Median | 0.0 | 0.0 |
| | min | −10 | −10 |
| | max | 10 | 10 |

^{a}A more negative change in PR-CIS score indicates a greater reduction of impact of cellulite on the subject's life.

In the Day 180 Observational Population, the mean (SD) change from Day 71 of the parent studies to Day 180 of this study in Total PR-CIS score was 1.2 (11.32) for the CCH treated subjects and 1.0 (10.14) for the placebo treated subjects. For PR-CIS Abbreviated score, the mean change from Day 71 of the parent studies to Day 180 of this study was 1.0 (9.45) and 0.4 (8.98) for the CCH and placebo treated subjects, respectively. Similar small mean (SD) changes were seen in the individual item scores (Happiness with appearance of cellulite: CCH 0.5 [2.68], placebo 0.1 [2.88]; Bothersome: CCH 0.0 [3.55], placebo 0.0 [3.78]; Self-consciousness: CCH 0.1 [2.63], placebo −0.1 [2.92]; Embarrassment: CCH 0.1 [2.62], placebo 0.1 [2.27]; Older Appearance: CCH 0.3 [2.92], placebo 0.6 [2.42]; Body Shape Concern: CCH 0.3 [2.59], placebo 0.4 [2.56]).

The small differences in PR-CIS total, abbreviated and item scores between Day 71 and Day 180 indicate a persistence of treatment effect up to 6 months after Day 71 in the parent studies in subjects treated with CCH. In addition, the proportion of PR-CIS responders remained consistent from Day 71 to Day 180.

c. Subject Self-rating Scale Day 180 Observational Population

At Day 71 in the parent studies, 24.7% of subjects treated with CCH in the parent studies were 2-level SSRS responders compared to 11.4% of placebo treated subjects in the Day 180 Observational Population. At Day 180, 14.9% of subjects treated with CCH in the parent studies were 2-level SSRS responders compared to 8.9% of placebo treated subjects.

At Day 71 in the parent studies, 51.5% of subjects treated with CCH in the parent studies were 1-level SSRS responders compared to 21.2% of placebo treated subjects in the Day 180 Observational Population. At Day 180, 36.9% of subjects treated with CCH in the parent studies were 2-level SSRS responders compared to 17.3% of placebo treated subjects (Table 71).

These results indicate that the proportion of SSRS responders was reduced over time for both the CCH treated and placebo treated subjects. However, the difference in SSRS responders between CCH treated subjects and placebo treated subjects was similar at Day 180 to the difference that was observed at Day 71 of the parent studies.

TABLE 71
Number of Subjects with Subject Self-rating Scale Responders and
Individual Ratings up to Day 180/Early Termination Visit (Day
180 Observational Population, as defined in this study 304)

| | Responders | | | |
|---|---|---|---|---|
| | Day 71^{a} SSRS | | Day 180 SSRS | |
| | CCH^{b} | Placebo^{b} | CCH^{b} | Placebo^{b} |
| | (N = 239) | (N = 236) | (N = 241) | (N = 237) |
| SSRS Ratings | n (%)^{c} | n (%)^{c} | n (%)^{c} | n (%)^{c} |
| Ratings | | | | |
| 6 (Extremely satisfied) | 12 (5.0) | 3 (1.3) | 4 (1.7) | 2 (0.8) |
| 5 (Satisfied) | 47 (19.7) | 24 (10.2) | 32 (13.3) | 19 (8.0) |
| 4 (Slightly satisfied) | 64 (26.8) | 23 (9.7) | 53 (22.0) | 20 (8.4) |
| 3 (Neither satisfied nor dissatisfied) | 31 (13.0) | 42 (17.8) | 47 (19.5) | 44 (18.6) |
| 2 (Slightly dissatisfied) | 24 (10.0) | 33 (14.0) | 23 (9.5) | 17 (7.2) |
| 1 (Dissatisfied) | 40 (16.7) | 51 (21.6) | 55 (22.8) | 65 (27.4) |
| 0 (Extremely dissatisfied) | 21 (8.8) | 60 (25.4) | 27 (11.2) | 70 (29.5) |
| 2-Level Responder^{d} | 59 (24.7) | 27 (11.4) | 36 (14.9) | 21 (8.9) |
| 1-Level Responder^{e} | 123 (51.5) | 50 (21.2) | 89 (36.9) | 41 (17.3) |

^{a}Day 71 in the 302/302 studies.
^{b}Treatment received in the 302/303 studies.
^{c}Percentages were based on the number of subjects observed at that visit.
^{d}A 2-level responder was defined as subject who was Satisfied or Extremely Satisfied (5 or 6).
^{e}A 1-level responder was defined as subject who was Slightly Satisfied, Satisfied, or Extremely Satisfied (4, 5, or 6).

### B. Analysis of Efficacy—TRR Before Retreatment Population

The TRR before Retreatment Population consists of 103 subjects who were treated with CCH and had a 1-level or 2-level composite responses in CR-PCSS and PR-PCSS in the parent studies and who consented to continue in the Open-label Phase of the current study. Nineteen of those subjects were classified as Category II and 84 were classified as Category I.

### 1. Two-Level Reduction in Response—CR-PCSS and PR-PCSS

There were 19 subjects in this study who had at least a 2-level composite improvement in cellulite severity in at least 1 buttock in the parent study (Category II). None of those subjects had a 2-level composite reduction in cellulite severity at Day 180. When considering the CR-PCSS and PR-PCSS individually in Category II subjects, 1 (5.3%) subject had at least a 2-level reduction in response in CR-PCSS, and 4 (21.1%) subjects had at least a 2-level reduction of response in PR-PCSS at Day 180, as defined in this study 304 (Table 72).

TABLE 72
Number of Subjects with a Reduction of Response in Cellulite Severity Rating
by at Least 2 Levels Prior to Retreatment (TRR before Retreatment Population)

| | Category II (N = 19) | |
|---|---|---|
| | Number of Subjects | Number of Subjects |
| | Not Retreated | with Reduction |
| | Prior to Visit | of Response |
| Visit | n | n (%)^{a} |
| Total Subjects^{b} | | |
| At least 2-Level Composite Reduction | 19 | 0 |
| Day 180/ET | | |
| At least 2-Level Reduction in CR-PCSS | 19 | 1 (5.3) |
| Day 180/ET | | |
| At least 2-Level Reduction in PR-PCSS | 19 | 4 (21.1) |
| Day 180/Early Termination | | |
| Subjects Responded in Left Buttock Only | | |
| At least 2-Level Composite Reduction | 6 | 0 |
| Day 180/Early Termination | | |
| At least 2-Level Reduction in CR-PCSS | 6 | 0 |
| Day 180/Early Termination | | |
| At least 2-Level Reduction in PR-PCSS | 6 | 2 (33.3) |
| Day 180/Early Termination | | |
| Subjects Responded in Right Buttock Only | | |
| At least 2-Level Composite Reduction | 6 | 0 |
| Day 180/Early Termination | | |
| At least 2-Level Reduction in CR-PCSS | 6 | 0 |
| Day 180/Early Termination | | |
| At least 2-Level Reduction in PR-PCSS | 6 | 2 (33.3) |
| Day 180/Early Termination | | |
| Subjects Responded in Both Buttocks | | |
| Left Buttock | | |
| At least 2-Level Composite Reduction | 7 | 0 |
| Day 180/Early Termination | | |
| At least 2-Level Reduction in CR-PCSS | 7 | 3 (42.9) |
| Day 180/Early Termination | | |
| At least 2-Level Reduction in PR-PCSS | 7 | 0 |
| Day 180/Early Termination | | |
| Right Buttock | | |
| At least 2-Level Composite Reduction | 7 | 1 (14.3) |
| Day 180/Early Termination | | |
| At least 2-Level Reduction in CR-PCSS | 7 | 1 (14.3) |
| Day 180/Early Termination | | |
| At least 2-Level Reduction in PR-PCSS | 7 | 1 (14.3) |
| Day 180/Early Termination | | |

^{a}Percentages at subject level are based on the number of responders (i.e., subjects who had composite improvement at Day 71/EOS in the parent study) with buttock not treated prior to the respective visit.
^{b}If a subject had a 2-level response in both buttocks, then the subject was counted only once.

Similar results were observed when considering the buttocks individually. In the 19 Category II subjects described above, there were 26 total buttocks with at least a 2-level composite improvement in cellulite in the parent studies. There was at least a 2-level composite reduction in response in 1 (3.8%) buttock at Day 180. In addition, a reduction in CR-PCSS of at least 2-levels was seen in 4 (15.4%) buttocks and a reduction in PR-PCSS response of at least 2 levels was seen in 5 (19.2%) buttocks at Day 180.

### 2. One-Level Reduction in Response—CR-PCSS and PR-PCSS

There were 19 Category II subjects in the TRR before Retreatment Population. Of these, 3 (15.8%) had a 1-level composite reduction in response prior to retreatment. Among 84 Category I subjects in the TRR before Retreatment Population, 10 (11.9%) had a 1-level composite reduction in response prior to retreatment. Overall (Category I and Category II combined, 103 subjects), 13 (12.6%) subjects had a 1-level composite reduction in response at Day 180 (Table 73).

Table 74 presents the number of buttocks with a reduction of response in cellulite severity rating by 1 level prior to retreatment (TRR before Retreatment Population).

TABLE 73
Number of Subjects with a Reduction of Response in Cellulite Severity Rating
by 1 Level Prior to Retreatment (TRR before Retreatment Population)

| | Category I | | | Category II | | | Overall | | |
|---|---|---|---|---|---|---|---|---|---|
| | Number of | Number of | | Number of | Number of | | Number of | Number of | |
| | Subjects Not | Subjects with | | Subjects Not | Subjects with | | Subjects Not | Subjects with | |
| | Retreated Prior | Reduction in | | Retreated Prior | Reduction in | | Retreated Prior | Reduction in | |
| | to the Visit | Response | | to the Visit | Response | | to the Visit | Response | |
| Visits | n | n (%)^{a} | | n | n (%) | | n | n (%) | |
| Total Subjects^{b} | | | | | | | | | |
| 1-Level Composite Reduction | 84 | 10 | (11.9) | 19 | 3 | (15.8) | 103 | 13 | (12.6) |
| Day 180/Early Termination | | | | | | | | | |
| 1-Level Reduction in CR-PCSS | 84 | 25 | (29.8) | 19 | 11 | (57.9) | 103 | 36 | (35.0) |
| Day 180/Early Termination | | | | | | | | | |
| 1-Level Reduction in PR-PCSS | 84 | 28 | (33.3) | 19 | 11 | (57.9) | 103 | 39 | (37.9) |
| Day 180/Early Termination | | | | | | | | | |
| Responded with Left Buttock Only | | | | | | | | | |
| 1-Level Composite Reduction | 23 | 7 | (30.4) | 6 | 1 | (16.7) | 29 | 8 | (27.6) |
| Day 180/Early Termination | | | | | | | | | |
| 1-Level Reduction in CR-PCSS | 23 | 13 | (56.5) | 6 | 4 | (66.7) | 29 | 17 | (58.6) |
| Day 180/Early Termination | | | | | | | | | |
| 1-Level Reduction in PR-PCSS | 23 | 12 | (52.2) | 6 | 4 | (66.7) | 29 | 16 | (55.2) |
| Day 180/Early Termination | | | | | | | | | |
| Responded with Right Buttock Only | | | | | | | | | |
| 1-Level Composite Reduction | 13 | 1 | (7.7) | 6 | 1 | (16.7) | 19 | 2 | (10.5) |
| Day 180/Early Termination | | | | | | | | | |
| 1-Level Reduction in CR-PCSS | 13 | 2 | (15.4) | 6 | 4 | (66.7) | 19 | 6 | (31.6) |
| Day 180/Early Termination | | | | | | | | | |
| 1-Level Reduction in PR-PCSS | 13 | 6 | (46.2) | 6 | 4 | (66.7) | 19 | 10 | (52.6) |
| Day 180/Early Termination | | | | | | | | | |

TABLE 74
Number of Buttocks with a Reduction of Response in Cellulite Severity Rating
by 1 Level Prior to Retreatment (TRR before Retreatment Population)

| | Category I | | | Category II | | | Category III | | |
|---|---|---|---|---|---|---|---|---|---|
| | Number of | Number of | | Number of | Number of | | Number of | Number of | |
| | Buttocks Not | Buttocks with | | Buttocks Not | Buttocks with | | Buttocks Not | Buttocks with | |
| | Retreated Prior | Reduction in | | Retreated Prior | Reduction in | | Retreated Prior | Reduction in | |
| | to the Visit | Response | | to the Visit | Response | | to the Visit | Response | |
| Visits | n | n (%)^{a} | | n | n (%) | | n | n (%) | |
| Total Buttocks^{b} | | | | | | | | | |
| 1-Level Composite Reduction | 132 | 19 | (14.4) | 26 | 6 | (23.1) | 158 | 25 | (15.8) |
| Day 180/Early Termination | | | | | | | | | |
| 1-Level Reduction in CR-PCSS | 132 | 45 | (34.1) | 26 | 15 | (57.7) | 158 | 60 | (38.0) |
| Day 180/Early Termination | | | | | | | | | |
| 1-Level Reduction in PR-PCSS | 132 | 48 | (36.4) | 26 | 15 | (57.7) | 158 | 63 | (39.9) |
| Day 180/Early Termination | | | | | | | | | |
| Responded with Left Buttock Only | | | | | | | | | |
| 1-Level Composite Reduction | 23 | 7 | (30.4) | 6 | 1 | (16.7) | 29 | 8 | (27.6) |
| Day 180/Early Termination | | | | | | | | | |
| 1-Level Reduction in CR-PCSS | 23 | 13 | (56.5) | 6 | 4 | (66.7) | 29 | 17 | (58.6) |
| Day 180/Early Termination | | | | | | | | | |
| 1-Level Reduction in PR-PCSS | 23 | 12 | (52.2) | 6 | 4 | (66.7) | 29 | 16 | (55.2) |
| Day 180/Early Termination | | | | | | | | | |
| Responded with Right Buttock Only | | | | | | | | | |
| 1-Level Composite Reduction | 13 | 1 | (7.7) | 6 | 1 | (16.7) | 19 | 2 | (10.5) |
| Day 180/Early Termination | | | | | | | | | |
| 1-Level Reduction in CR-PCSS | 13 | 2 | (15.4) | 6 | 4 | (66.7) | 19 | 6 | (31.6) |
| Day 180/Early Termination | | | | | | | | | |
| 1-Level Reduction in PR-PCSS | 13 | 6 | (46.2) | 6 | 4 | (66.7) | 19 | 10 | (52.6) |
| Day 180/Early Termination | | | | | | | | | |
| Responded with Both Buttocks | | | | | | | | | |
| Left Buttock | | | | | | | | | |
| 1-Level Composite Reduction | 48 | 5 | (10.4) | 7 | 2 | (28.6) | 55 | 7 | (12.7) |
| Day 180/Early Termination | | | | | | | | | |
| 1-Level Reduction in CR-PCSS | 48 | 16 | (33.3) | 7 | 3 | (42.9) | 55 | 19 | (34.5) |
| Day 180/Early Termination | | | | | | | | | |
| 1-Level Reduction in PR-PCSS | 48 | 15 | (31.3) | 7 | 3 | (42.9) | 55 | 18 | (32.7) |
| Day 180/Early Termination | | | | | | | | | |
| Right Buttock | | | | | | | | | |
| 1-Level Composite Reduction | 48 | 6 | (12.5) | 7 | 2 | (28.6) | 55 | 8 | (14.5) |
| Day 180/Early Termination | | | | | | | | | |
| 1-Level Reduction in CR-PCSS | 48 | 14 | (29.2) | 7 | 4 | (57.1) | 55 | 18 | (32.7) |
| Day 180/Early Termination | | | | | | | | | |
| 1-Level Reduction in PR-PCSS | 48 | 15 | (31.3) | 7 | 4 | (57.1) | 55 | 19 | (34.5) |
| Day 180/Early Termination | | | | | | | | | |

^{a}Percentages are based on the number of responders (subjects who had composite improvement at Day 71/EOS of the parent study) with buttocks not retreated prior to the respective visit
^{b}If a subject had a 1-level response in both buttocks, the subject was counted only once.

Similar results were observed when considering the buttocks individually. In the 19 Category II subjects described above, there were 26 total buttocks that had at least a 2-level composite response in the parent studies. Of these, a 1-level composite reduction in response was seen in 6 (23.1%) buttocks. In Category I subjects, there was a 1-level reduction of response in 19 (14.4%) of 132 buttocks. Overall (Category I and Category II, 158 buttocks), 15.8% of buttocks had 1-level composite reduction of response at Day 180.

### 3. Time to Reduction of Response—CR-PCSS and PR-PCSS

TRR was defined as the number of days from Day 71/EOS in the parent study to the study visit in the current trial when a composite worsening of response in CR-PCSS and PR-PCSS was first observed. No subjects had a 2-level reduction in response so it was not possible to calculate a time to a 2-level reduction in response. The mean (SD) time to a 1-level reduction of response was 187.5 (10.87) days in Category I subjects and 182.7 (17.47) days in Category II subjects.

### 4. Complete Loss of Response—CR-PCSS and PR-PCSS

Complete loss of response was defined as the return to baseline (of the parent study) in both CR-PCSS and PR-PCSS ratings. No Category II subjects (subjects with at least a 2-level composite response in the parent study) had a complete loss of response at Day 180. Of the 84 Category I subjects, 4 (4.8%) had a complete loss of response (Table 75).

TABLE 75
Number of Subjects with Complete Loss of Response Prior
to Retreatment (TRR before Retreatment Population)

| | Buttocks Not | Number of Subjects |
|---|---|---|
| | Retreated Prior | with Complete |
| | to Day 180 | Loss of Response |
| Subject Category | n | n (%) |
| Category II | 19 | 0 (0.0) |
| Category I | 84 | 4 (4.8) |
| Overall | 103 | 4 (3.9) |

### 5. Time to Complete Loss of Response Prior to Retreatment—CR-PCSS and PR-PCSS

Time to complete loss of response was defined as the number of days from Day 71/EOS in the parent study to the study visit in the current trial when complete loss of response was first observed. No subjects in Category II had a complete loss of response. The mean (SD) time to complete loss of response in Category I was 183.0 (2.83) days.

### 6. Cellulite Severity—TRR Before Retreatment Population

a. PR-PCSS and CR-PCSS

Overall (in Category I and Category II subjects combined), the mean (SD) PR-PCSS change from Day 1 to Day 71/EOS in the parent studies was −1.3 (0.64) in the left buttock and −1.4 (0.83) in the right buttock. The overall mean (SD) change in PR-PCSS from Day 1 in the parent studies to Day 180 was −1.0 (0.80) for the left buttock and −1.1 (0.93) for the right buttock in the TRR before Retreatment Population.

For Category I subjects the mean (SD) PR-PCSS change from Day 1 to Day 71 in the parent studies was −1.2 (0.60) and −1.2 (0.80) in the left and right buttocks, respectively. The mean (SD) PR-PCSS change from Day 1 in the parent study to Day 180 of this study was −1.0 (0.77) and −1.1 (0.96) in the left and right buttocks, respectively.

For Category II subjects the mean (SD) PR-PCSS change from Day 1 to Day 71 in the parent studies was −1.7 (0.65) and −2.1 (0.62) in the left and right buttocks, respectively. The mean (SD) PR-PCSS change from Day 1 in the parent study to Day 180 of this study was −1.1 (0.94) and −1.2 (0.79) in the left and right buttocks, respectively.

The overall (Category I and Category II subjects combined) mean (SD) change in CR-PCSS from Day 1 to Day 71/EOS in the parent studies to was −1.2 (0.57) for the left buttock and −1.1 (0.63) for the right buttock. The overall mean (SD) change in CR-PCSS from Day 1 in the parent studies to Day 180 was −0.8 (0.78) for the left buttock and −0.9 (0.78) for the right buttock.

For Category I subjects the mean (SD) CR-PCSS change from Day 1 to Day 71 in the parent studies was −1.1 (0.46) and −0.9 (0.54) in the left and right buttocks, respectively. The mean (SD) CR-PCSS change from Day 1 in the parent study to Day 180 of this study was −0.7 (0.74) and −0.9 (0.78) in the left and right buttocks, respectively.

For Category II subjects the mean (SD) PR-PCSS change from Day 1 to Day 71 in the parent studies was −1.8 (0.63) and −1.8 (0.37) in the left and right buttocks, respectively. The mean (SD) PR-PCSS change from Day 1 in the parent study to Day 180 of this study was −1.2 (0.85) and −1.2 (0.76) in the left and right buttocks, respectively.

These results support a persistence of response in this population.

b. PR-CIS

Similar results were seen for PR-CIS total, abbreviated, and item scores and support a persistence of response in this population.

### 7. Persistence of Treatment Effect

a. Persistence of Treatment Effect—CR-PCSS and PR-PCSS-TRR Before Retreatment Population

Table 76 displays the change in CR-PCSS and PR-PCSS for the TRR before Retreatment Population from Day 71 in the parent studies (Studies 302/303) to Day 180 in this study.

TABLE 76
Change from Day 71/EOS in Studies 302/303 in PR-PCSS and CR-PCSS at
Day 180/Early Termination Visit (TRR before Retreatment Population)

| | | Category I (N = 84) | | Category II (N = 19) | | | | Overall (N = 103) | |
|---|---|---|---|---|---|---|---|---|---|
| Cellulite Severity Scale | | Left | Right | Left | | Right | | Left | Right |
| Cellulite Severity Rating | Statistic^{a} | Buttock | Buttock | Buttock | | Buttock | | Buttock | Buttock |
| PR-PCSS | | | | | | | | | |
| Change from Day 71/EOS in Studies | N | 84 | 84 | 19 | | 19 | | 103 | 103 |
| 302/303 to Day 180/Early Termination | | | | | | | | | |
| −4 | n (%) | 0 | 0 | 0 | | 0 | | 0 | 0 |
| −3 | n (%) | 0 | 0 | 0 | | 0 | | 0 | 0 |
| −2 | n (%) | 0 | 1 (1.2) | 0 | | 0 | | 0 | 1 (1.0) |
| −1 | n (%) | 12 (14.3) | 17 (20.2) | 1 | (5.3) | 0 | | 13 (12.6) | 17 (16.5) |
| 0 | n (%) | 42 (50.0) | 38 (45.2) | 8 | (42.1) | 7 | (36.8) | 50 (48.5) | 45 (43.7) |
| +1 | n (%) | 30 (35.7) | 24 (28.6) | 7 | (36.8) | 8 | (42.1) | 37 (35.9) | 32 (31.1) |
| +2 | n (%) | 0 | 4 (4.8) | 3 | (15.8) | 4 | (21.1) | 3 (2.9) | 8 (7.8) |
| +3 | n (%) | 0 | 0 | 0 | | 0 | | 0 | 0 |
| +4 | n (%) | 0 | 0 | 0 | | 0 | | 0 | 0 |
| | Mean | 0.2 | 0.2 | 0.6 | | 0.8 | | 0.3 | 0.3 |
| | SD | 0.68 | 0.84 | 0.83 | | 0.76 | | 0.72 | 0.87 |
| CR-PCSS | | | | | | | | | |
| Change from Day 71/EOS in Studies | N | 84 | 84 | 19 | | 19 | | 103 | 103 |
| 302/303 to Day 180/Early Termination | | | | | | | | | |
| −4 | n (%) | 0 | 0 | 0 | | 0 | | 0 | 0 |
| −3 | n (%) | 0 | 0 | 0 | | 0 | | 0 | 0 |
| −2 | n (%) | 0 | 0 | 0 | | 0 | | 0 | 0 |
| −1 | n (%) | 7 (8.3) | 18 (21.4) | 2 | (10.5) | 0 | | 9 (8.7) | 18 (17.5) |
| 0 | n (%) | 44 (52.4) | 46 (54.8) | 8 | (42.1) | 8 | (42.1) | 52 (50.5) | 54 (52.4) |
| +1 | n (%) | 28 (33.3) | 18 (21.4) | 6 | (31.6) | 10 | (52.6) | 34 (33.0) | 28 (27.2) |
| +2 | n (%) | 5 (6.0) | 1 (1.2) | 2 | (10.5) | 0 | | 7 (6.8) | 1 (1.0) |
| +3 | n (%) | 0 | 1 (1.2) | 1 | (5.3) | 1 | (5.3) | 1 (1.0) | 2 (1.9) |

| | | | | | | | |
|---|---|---|---|---|---|---|---|
| +4 | n (%) | 0 | 0 | 0 | 0 | 0 | 0 |
| | Mean | 0.4 | 0.1 | 0.6 | 0.7 | 0.4 | 0.2 |
| | SD | 0.72 | 0.77 | 1.02 | 0.75 | 0.79 | 0.80 |

^{a}Percentages are based on the number of subjects with buttock not retreated prior to the respective visit.

In Category I subjects, the mean (SD) PR-PCSS change from Day 71 in the parent studies to Day 180 in this study was 0.2 (0.68) in the left buttock and 0.2 (0.84) in the right buttock. For CR-PCSS, the mean (SD) change from Day 71 in the parent studies to Day 180 in this study in was 0.4 (0.72) in the left buttock and 0.1 (0.77) in the right buttock.

For Category II subjects, the mean (SD) PR-PCSS change from Day 71 in the parent studies to Day 180 in this study in was 0.6 (0.83) in the left buttock and 0.8 (0.76) in the right buttock. For CR-PCSS, the mean (SD) change from Day 71 in the parent studies to Day 180 in this study in CCH treated subjects was 0.6 (1.02) in the left buttock and 0.7 (0.75) in the right buttock.

In the overall TRR Population, the mean (SD) PR-PCSS change from Day 71 in the parent studies to Day 180 in this study was 0.3 (0.72) in the left buttock and 0.3 (0.87) in the right buttock. For CR-PCSS, the mean (SD) change from Day 71 in the parent studies to Day 180 in this study was 0.4 (0.79) in the left buttock and 0.2 (0.80) in the right buttock.

The small differences in the overall TRR Population PR-PCSS and CR-PCSS scores between Day 71 and Day 180 indicate a persistence of treatment effect up to 6 months after Day 71 in the parent studies in subjects treated with CCH. These results also indicate that the mean treatment effect was virtually identical between the left and right buttocks.

b. Persistence of Treatment Effect—PR-CIS—TRR Before Retreatment Population

Table 77 displays the change in PR-CIS total, abbreviated, and item scores from Day 71/EOS for the Studies 302/303 studies to Day 180/Early Termination in this study in the TRR before Retreatment Population.

TABLE 77
Change from Day 71/End of Study in the Studies 302/303
Studies in PR-CIS Total, Abbreviated, and Item Scores
Prior to Retreatment (TRR before Retreatment Population)

| | | Category I | Category II | Overall |
|---|---|---|---|---|
| PR-CIS^{a} | Statistic | (N = 84) | (N = 19) | (N = 103) |
| Total Score | | | | |
| Change from Day 71/EOS of the 302/303 | n | 84 | 19 | 103 |
| Studies to Day 180/Early Termination | | | | |
| | Mean | −0.6 | 2.5 | 0.0 |
| | SD | 11.20 | 16.04 | 12.21 |
| | Median | 0.0 | −1.0 | 0.0 |
| | min | −50 | −21 | −50 |
| | max | 22 | 43 | 43 |
| Abbreviated Score | | | | |
| Change from Day 71/EOS of the 302/303 | n | 84 | 19 | 103 |
| Studies to Day 180/Early Termination | | | | |
| | Mean | −0.5 | 2.8 | 0.1 |
| | SD | 9.12 | 12.32 | 9.80 |
| | Median | 0.0 | 2.0 | 0.0 |
| | min | −40 | −14 | −40 |
| | max | 18 | 33 | 33 |
| Question 1: Impact of Happiness | | | | |
| Change from Day 71 of the 302/303 | n | 84 | 19 | 103 |
| Studies to Day 180/Early Termination | | | | |
| | Mean | 0.6 | 1.4 | 0.7 |
| | SD | 2.31 | 2.39 | 2.34 |
| | Median | 0.0 | 1.0 | 1.0 |
| | min | −6 | −4 | −6 |
| | max | 8 | 5 | 8 |
| Question 2: Impact of Bothersome | | | | |
| Change from Day 71/EOS of the 302/303 | n | 84 | 19 | 103 |
| Studies to Day 180/Early Termination | | | | |
| | Mean | −0.4 | 0.6 | −0.2 |
| | SD | 2.75 | 3.90 | 3.00 |
| | Median | 0.0 | 2.0 | 0.0 |
| | min | −10 | −6 | −10 |
| | max | 8 | 8 | 8 |
| Question 3: Impact of Self-consciousness | | | | |
| Change from Day 71/EOS of the 302/303 | n | 84 | 19 | 103 |
| Studies to Day 180/Early Termination | | | | |
| | Mean | −0.4 | 0.6 | −0.2 |
| | SD | 2.67 | 3.37 | 2.82 |
| | Median | 0.0 | 0.0 | 0.0 |
| | min | −10 | −4 | −10 |
| | max | 8 | 8 | 8 |
| Question 4: Impact of Embarrassment | | | | |
| Change from Day 71/EOS of the 302/303 | n | 84 | 19 | 103 |
| Studies to Day 180/Early Termination | | | | |
| | Mean | −0.2 | 0.6 | −0.1 |
| | SD | 2.71 | 3.44 | 2.86 |
| | Median | 0.0 | 1.0 | 0.0 |
| | min | −10 | −6 | −10 |
| | max | 5 | 8 | 8 |
| Question 5: Impact of Older Appearance | | | | |
| Change from Day 71/EOS of the 302/303 | n | 84 | 19 | 103 |
| Studies to Day 180/Early Termination | | | | |
| | Mean | −0.1 | −0.3 | −0.1 |
| | SD | 2.80 | 4.12 | 3.06 |
| | Median | 0.0 | 0.0 | 0.0 |
| | min | −10 | −9 | −10 |
| | max | 6 | 10 | 10 |
| Question 6: Impact of Body Shape Concern | | | | |
| Change from Day 71/EOS of the 302/303 | n | 84 | 19 | 103 |
| Studies to Day 180/Early Termination | | | | |
| | Mean | −0.0 | −0.4 | −0.1 |
| | SD | 2.79 | 2.78 | 2.78 |
| | Median | 0.0 | −1.0 | 0.0 |
| | min | −10 | −5 | −10 |
| | max | 9 | 4 | 9 |

^{a}A more negative change in score indicates a greater reduction of impact of cellulite on the subject's life.

In the TRR before Retreatment Population overall (Category I and Category II combined), the mean (SD) change from Day 71 of the parent studies to Day 180 of this study in Total PR-CIS score was 0.0 (12.21). For Category I subjects the mean (SD) change from Day 71 to Day 180 was −0.6 (11.20) and the mean change from Day 71 to Day 180 for Category II subjects was 2.5 (16.04).

The mean (SD) change from Day 71 of the parent studies to Day 180 of this study in Abbreviated PR-CIS score was 0.1 (9.80). For Category I subjects the mean (SD) change from Day 71 to Day 180 was −0.5 (9.12) and the mean change from Day 71 to Day 180 for Category II subjects was 2.8 (12.32).

Similar small mean (SD) changes were seen in the overall individual item scores (Happiness with appearance of cellulite: 0.7; Bothersome: −0.2; Self-consciousness: −0.2; Embarrassment: −0.1; Older Appearance: −0.1; Body Shape Concern: −0.1).

The small differences in the overall TRR Population PR-CIS scores between Day 71 and Day 180 support the PR-PCSS and CR-PCSS results and indicate a persistence of treatment effect up to 6 months after Day 71 of the parent studies in subjects treated with CCH.

PR-CIS, S-GAIS, Subjects Satisfaction with Cellulite Treatment, and SSRS Responders—TRR before Retreatment Population

At Day 180, subjects in Category I and Category II in the TRR before Retreatment Population displayed PR-CIS (total, abbreviated, and item scores), S-GAIS, Subject Satisfaction with Cellulite Treatment, and SSRS responses. A greater proportion of Category II subjects were responders than Category I subjects.

### Efficacy Conclusions

### A. Day 180 Observational Population

The Day 180 Observational Population consisted of 479 subjects that completed the Day 180 Visit (including cellulite assessments) and were unblinded at the time of the data cut-off (Apr. 1, 2019). The Day 180 Visit occurred approximately 180 days after the completion of the parent studies and approximately 251 days after the first dose of study drug in the parent studies.

The mean changes in CR-PCSS, PR-PCSS, and PR-CIS from Day 1 to Day 71 (in the parent study) and from Day 1 in the parent study to Day 180 in this study indicate that subjects in the Day 180 Observational Population are representative of the population of the parent studies. The difference between CCH treated subjects and placebo treated subjects at Day 180 is consistent with the results seen in the parent studies.
In the 241 subjects who were treated with CCH in the parent studies, the mean (SD) change from Day 1 to Day 71 in the parent studies in PR-PCSS was −0.8 (0.85) and −0.9 (0.92) in the left and right buttock, respectively, and the mean (SD) changes in PR-PCSS from Day 1 in the parent studies to Day 180 in this study was −0.7 (0.87) and −0.7 (0.92) in the left and right buttock, respectively. In subjects treated with placebo in the parent studies, the mean (SD) PR-PCSS change from Day 1 to Day 71 was −0.5 (0.75) and −0.5 (0.79) in the left and right buttock, respectively. The mean change from Day 1 in the parent studies to Day 180 in this study in PR-PCSS was −0.3 (0.71) and −0.4 (0.80) in the left and right buttock respectively.
In subjects who were treated with CCH in the parent studies, the mean (SD) CR-PCSS change from Day 1 to Day 71 in the parent studies was −0.8 (0.79) and −0.7 (0.76) for the left and right buttock, respectively. The mean change (SD) in CR-PCSS from Day 1 of the parent studies to Day 180 of this study was −0.6 (0.82) for the left buttock and −0.6 (0.83) for the right buttock. In placebo treated subjects, the mean (SD) CR-PCSS change from Day 1 to Day 71 was −0.3 (0.64) and −0.3 (0.70) in the left and right buttock, respectively. The mean change from Day 1 in the parent study to Day 180 in this study in CR-PCSS was −0.4 (0.72) and −0.4 (0.72) in the left and right buttock, respectively.
In subjects who were treated with CCH in the parent studies and completed the Day 180 assessments of PR-CIS in this study, the mean (SD) change from Day 1 to Day 71 in the parent studies in PR-CIS Total Score was −11.8 (11.77). In these same subjects, the mean (SD) change in PR-CIS Total Score from Day 1 in the parent studies to Day 180 in this study was −10.5 (11.76). In placebo treated subjects in the parent studies, the mean (SD) change from Day 1 to Day 71 in the parent studies in PR-CIS Total Score was −6.5 (12.12). In these same subjects, the mean (SD) change in PR-CIS Total Score from Day 1 in the parent studies to Day 180 in this study was −5.4 (11.30). Similar changes were seen for the PR-CIS abbreviated and item scores.

The small differences in PR-PCSS, CR-PCSS, and PR-CIS between Day 71 of the parent study and Day 180 in this study indicate a persistence of treatment effect up to 6 months after Day 71 of the parent studies in subjects treated with CCH. In addition, the proportion of PR-CIS responders remained consistent from Day 71 to Day 180. The results for PR-PCSS and CR-PCSS also indicate that the mean treatment effect was virtually identical between the left and right buttock.
For PR-PCSS, the mean (SD) change from Day 71 in the parent studies to Day 180 in this study in CCH treated subjects was 0.2 (0.81) and 0.2 (0.86) in the left and right buttock, respectively. In placebo treated subjects, the mean (SD) change on PR-PCSS was 0.1 (0.76) and 0.0 (0.84) in the left and right buttock, respectively.
For CR-PCSS, the mean (SD) change from Day 71 in the parent studies to Day 180 in this study in CCH treated subjects was 0.2 (0.78) and 0.1 (0.76) in the left and right buttock, respectively. In placebo treated subjects, the mean (SD) change on CR-PCSS was −0.1 (0.74) and −0.1 (0.76) in the left and right buttock, respectively.
In the Day 180 Observational Population, the mean (SD) change from Day 71 of the parent studies to Day 180 of this study in Total PR-CIS score was 1.2 (11.32) for the CCH treated subjects and 1.0 (10.14) for the placebo treated subjects. For PR-CIS Abbreviated score, the mean change from Day 71 of the parent studies to Day 180 of this study was 1.0 (9.45) and 0.4 (8.98) for the CCH and placebo treated subjects, respectively. Similar small mean (SD) changes were seen in the individual item scores

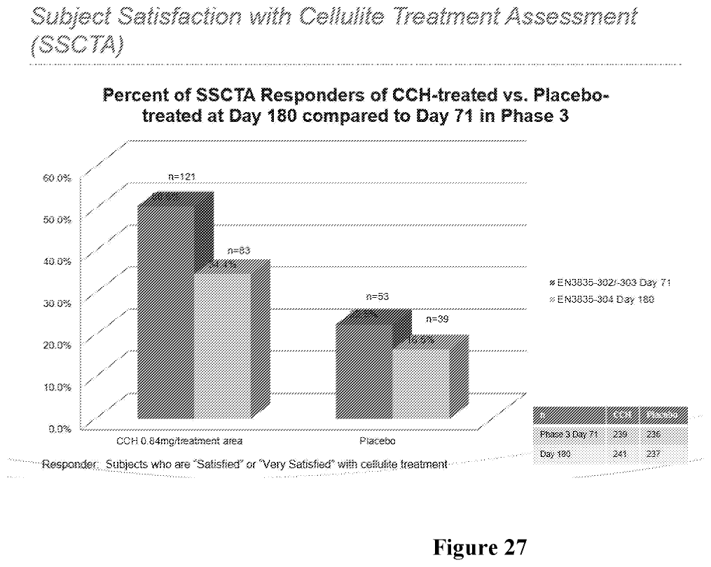

The proportion of S-GAIS, Subject Satisfaction with Cellulite Treatment, and SSRS responders at Day 180 decreased in both the CCH treated and placebo treated subjects in the parent studies. However, the response rate in CCH treated subjects remained higher than that of placebo treated subjects at Day 180 in this study.
At Day 71 in the parent studies, 19.1% of subjects treated with CCH were S-GAIS 2-level responders in the left buttock and 21.6% were 2-level responders in the right buttock compared to 6.3% (left buttock) and 7.1% (right buttock) of placebo treated subjects. At Day 180 of this study 304, 12.4% and 11.6% of CCH treated subjects were 2-level S-GAIS responders in the left and right buttock, respectively. For placebo treated subjects, the percentage of subjects who were 2-level S-GAIS responders at Day 180 was 3.8% for each of the right and left buttocks.
At Day 71 in the parent studies, 7.5% of subjects treated with CCH were S-GAIS 1-level responders in each of the left and right buttocks compared to 37.4% (left buttock) and 37.0% (right buttock) of placebo treated subjects. At Day 180, 53.5% and 51.0% of CCH treated subjects were 1-level S-GAIS responders in the left and right buttock, respectively. For placebo treated subjects, the percentage of subjects who were 1-level S-GAIS responders at Day 180 was 27.8% and 28.3% in the left and right buttock, respectively.
At Day 71, 50.6% of subjects treated with CCH in the parent studies were Satisfied or Very Satisfied with cellulite treatment compared to 22.5% of placebo treated subjects in the Day 180 Observational Population. At Day 180, 34.4% of subjects treated with CCH in the parent studies were Satisfied or Very Satisfied with cellulite treatment compared to 16.5% of placebo treated subjects (FIG. 27).
At Day 71 in the parent studies, 24.7% of subjects treated with CCH were 2-level SSRS responders compared to 11.4% of placebo treated subjects in the Day 180 Observational Population. At Day 180, 14.9% of subjects treated with CCH in the parent studies were 2-level SSRS responders compared to 8.9% of placebo treated subjects.
At Day 71 in the parent studies, 51.5% of subjects treated with CCH were 1-level SSRS responders compared to 21.2% of placebo treated subjects in the Day 180 Observational Population. At Day 180, 36.9% of subjects treated with CCH in the parent studies were 2-level SSRS responders compared to 17.3% of placebo treated subjects.
   B. TRR before Retreatment Population

The TRR before Retreatment Population consists of 103 subjects who were treated with CCH and had 1-level or 2-level composite responses in CR-PCSS and PR-PCSS in the parent studies and who consented to continue in the Open-label Phase of the current study. Nineteen of those subjects were classified as Category II and 84 were classified as Category I.

No subjects in Category II and few subjects in Category I had a reduction in composite response or complete loss of response as measured by PR-PCSS and CR-PCSS. Time to reduction of response and time to complete reduction of response were reflective of the duration of the study as of the data cut-off. Time to reduction of response and time to complete reduction of response are provided below but additional longer term data are needed to draw conclusions from these data about duration of response.
There were 19 subjects in this study who had at least a 2-level composite improvement in cellulite severity in at least 1 buttock in the parent study (Category II). None of those subjects had a 2-level composite reduction in cellulite severity at Day 180 in this study. When considering the CR-PCSS and PR-PCSS individually in Category II subjects, 1 (5.3%) subject had at least a 2-level reduction in response in CR-PCSS, and 4 (21.1%) subjects had at least a 2-level reduction of response in PR-PCSS at Day 180. Similar results were observed when considering the buttocks individually.
There were 19 Category II subjects in the TRR before Retreatment Population. Of these, 3 (15.8%) had a 1-level composite reduction in response prior to retreatment. Among 84 Category I subjects in the TRR before Retreatment Population, 10 (11.9%) had a 1-level composite reduction in response prior to retreatment. Overall (Category I and Category II combined, 103 subjects), 13 (12.6%) subjects had a 1-level composite reduction in response at Day 180.
Complete loss of response was defined as the return to baseline (in the parent study) in both CR-PCSS and PR-PCSS ratings. No Category II subjects (subjects with at least a 2-level composite response in the parent study) had a complete loss of response at Day 180. Of the 84 Category I subjects, 4 (4.8%) had a complete loss of response.
TRR was defined as the number of days from Day 71/EOS in the parent study to the study visit in the current trial when a composite worsening of response in CR-PCSS and PR-PCSS was first observed. No subjects had a 2-level reduction in response so it was not possible to calculate a time to a 2-level reduction in response. The mean (SD) time to a 1-level reduction of response was 187.5 (10.87) days in Category I subjects and 182.7 (17.47) days in Category II subjects.
Time to complete loss of response was defined as the number of days from Day 71/EOS in the parent study to the study visit in the current trial when complete loss of response was first observed. No subjects in Category II had a complete loss of response. The mean (SD) time to complete loss of response in Category I was 183.0 (2.83) days.

The small overall differences in PR-PCSS, CR-PCSS, and PR-CIS in the TRR before Retreatment Population between Day 71 and Day 180 indicate a persistence of treatment effect up to 6 months after Day 71 in the parent studies in subjects treated with CCH. These results also indicate that the mean treatment effect was virtually identical between the left and right buttock.
In Category I subjects, the mean (SD) PR-PCSS change from Day 71 in the parent studies to Day 180 in this study was 0.2 (0.68) in the left buttock and 0.2 (0.84) in the right buttock. For CR-PCSS the mean (SD) change from Day 71 in the parent studies to Day 180 in this study in was 0.4 (0.72) in the left buttock and 0.1 (0.77) in the right buttock.
For Category II subjects, the mean (SD) PR-PCSS change from Day 71 in the parent studies to Day 180 in this study in was 0.6 (0.83) in the left buttock and 0.8 (0.76) in the right buttock. For CR-PCSS the mean (SD) change from Day 71 in the parent studies to Day 180 in this study in CCH treated subjects was 0.6 (1.02) in the left buttock and 0.7 (0.75) in the right buttock.
The mean (SD) change from Day 71 of the parent studies to Day 180 of this study in Total PR-CIS score was 0.0 (12.21). For Category I subjects the mean (SD) change from Day 71 to Day 180 was −0.6 (11.20) and the mean change from Day 71 to Day 180 for Category II subjects was 2.5 (16.04).
The mean (SD) change from Day 71 of the parent studies to Day 180 of this study in Abbreviated PR-CIS score was 0.1 (9.80). For Category I subjects the mean (SD) change from Day 71 to Day 180 was −0.5 (9.12) and the mean change from Day 71 to Day 180 for Category subjects was 2.8 (12.32).
Similar small mean (SD) changes were seen in the overall individual item scores (Happiness with appearance of cellulite: 0.7; Bothersome: −0.2; Self-consciousness: −0.2; Embarrassment: −0.1; Older Appearance: −0.1; Body Shape Concern: −0.1)

At Day 180, the proportion of subjects in Category I and Category II in the TRR before Retreatment Population who were PR-CIS (total, abbreviated, and item scores), S-GAIS, Subject Satisfaction with Cellulite Treatment, and SSRS responders was similar to that seen in the Day 180 Observational Population, and in the parent studies.

### C. OLR Population

An insufficient number of subjects had completed retreatment at the time of the data cut-off to evaluate efficacy in the Open-label Retreatment Population.

### Safety

The safety profile of CCH was consistent with the safety results of the previous studies. The AE profile during the Day 180 Observational Period for CCH treated subjects (in the parent studies) is very similar to that of subjects treated with placebo in the parent studies, suggesting that there are no long term safety concerns for CCH as of the data cut-off. There was little difference in the frequency or type of AEs between the different subject categories (I, II, or III). Nothing significant was found with respect to immunogenicity, which was consistent with previous studies.

### Conclusion

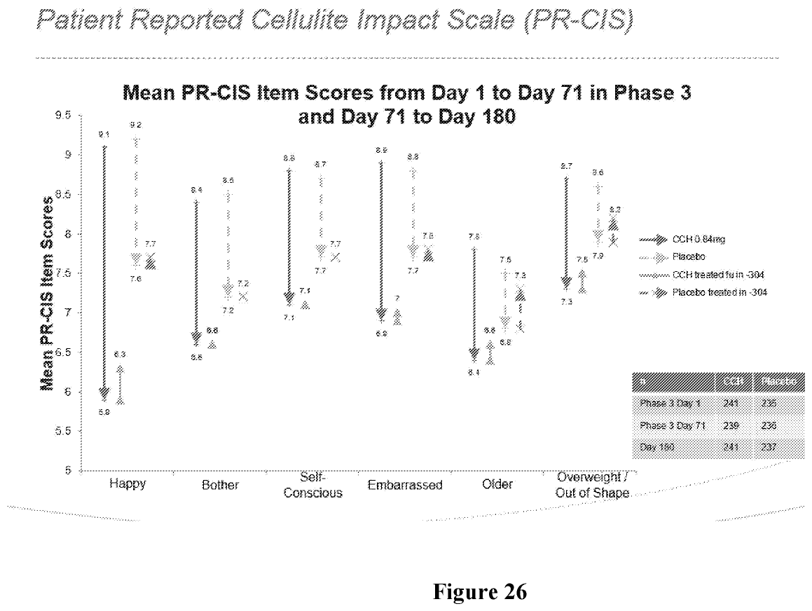

The results of this study as of the data cut-off (Apr. 1, 2019) indicate a persistence of treatment effect for subjects treated with CCH for buttock EFP of at least 180 days (6 months) after Day 71 of the Phase 3 double-blind parent studies when measured by PR-PCSS, CR-PCSS, and PR-CIS (FIG. 26). No subjects had a 2-level reduction in composite response in this study, and no subjects who were 2-level composite responders in the parent study had a complete loss of response by Day 180. There was no indication of long term safety concerns for subjects treated with CCH in the parent studies. For the 30 subjects who have been retreated with CCH in this study as of the data cut-off (Apr. 1, 2019), the AE profile is similar to that seen in other clinical trials of CCH in cellulite.

### Example 6—Phase 2 Open-Label Extension Study of Cch in the Treatment of Cellulite (Study 202)

The current Example (Study 202) is a Phase 2, open-label extension study of a previous study (Study 201), which assessed safety and effectiveness of CCH in adult women. In the current example, the long-term safety was evaluated in eligible subjects treated in Study 201. In addition, the safety, effectiveness, and immunogenicity upon re-exposure to CCH 0.84 mg per treatment area was evaluated in subjects that enrolled and who received their second treatment course. In this study, the safety of re-exposure, either in a previously treated treatment area (termed retreatment) or in a naive treatment area (termed redosing) in subjects that previously received CCH was assessed. Finally, persistence of effect was evaluated in CCH-treated subjects from the previous study (Study 201, double-blind/open-label assessments) and from this current study (Study 202, open-label). For subjects treated with CCH in the previous study (Study 201), the treatment area treated was assessed for up to approximately 2 years for long-term persistence of effect. For subjects reexposed (retreated or redosed), the treatment areas were assessed for up to approximately 1 year. Unless otherwise specified in this example, “Days” as used in this study 202 were relative to the initial dose (Day 1) in study 201. In study 201, 0.84 mg of CCH or placebo was administered to one treatment area (1 buttock or 1 thigh) per patient.

More specifically, the current study assessed the long-term safety of CCH at scheduled intervals over 1 year (up to 2 years) in all subjects with cellulite who elected to enroll in this study regardless of their decision to receive treatment (re-treatment or re-dosing) in this study. Following the unblinding of the treatment subjects received in the previous study (Study 201), eligible subjects could elect to receive CCH treatment. If the subject received CCH in Study 201 and the cellulite severity ratings of the treated area had returned to or were higher (indicating worse cellulite severity) than Study 201 baseline ratings, the subject could elect to have the previously treated area retreated with CCH (termed retreatment) in one Treatment Course. If the subject received CCH in Study 201, the subject could elect to treat a qualifying area other than the area treated with CCH in Study 201 (termed redosing). Subjects that received placebo in Study 201 had the option to receive 2 treatment courses (a course consisted of 3 treatment sessions 21 days apart) of CCH in this study. Each treatment course comprised CCH 0.84 mg administered as 12 subcutaneous injections per treatment area for up to 3 sessions in the same or different qualifying treatment area of the buttocks or thighs. Treatment Course 2 comprised subjects who were retreated or redosed.

Subjects from Study 201 who opted to receive no treatment (observation-only subjects, Observation Phase) were followed for safety and cellulite severity assessments at 3-month intervals from Day 1 in study 201 for up to 2 years. Persistence of response was assessed for 2 years in subjects who received CCH in Study 201 and showed at least a 1-level composite PR-PCSS/CR-PCSS improvement in cellulite severity (Long-term Durability Phase in double-blind treated subjects). Durability of response was defined as subjects who were CR-PCSS and PR-PCSS responders and maintained this response (i.e., lack of complete loss, did not return to baseline or worse) in a CCH-treated area. Subjects that received treatment in this study continued to be observed at Day 71 after first exposure to CCH in this study and then at 3-month intervals after the first exposure to CCH in each treated area in this study for 1 year.

Prior to retreatment or redosing, independent assessments by subjects and investigators were conducted on the each of the subject's 4 treatment areas (i.e., left buttock, right buttock, left posterolateral thigh, and right posterolateral thigh) using the PR-PCSS, CR-PCSS, and Hexsel CSS to assess the severity of cellulite at screening.

### A. Overall Study Design and Plan

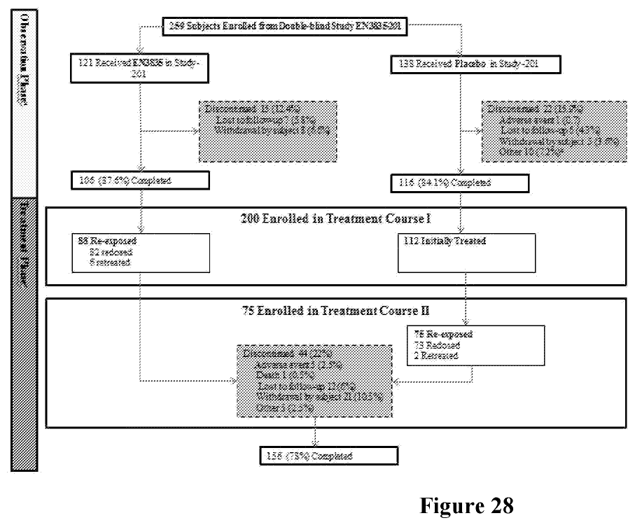

Following completion of safety and cellulite assessments at Day 71 of Study 201, subjects were asked if they wished to continue in the open-label extension to the double-blind study (Screening A). At the time of entry into the current study, subjects and investigators were blinded to the identity of the study drug that the subject received in Study 201. Until the Study-201 study drug blind was broken, subjects underwent observation-only visits at 3-month intervals±7 days (relative to the initial dose in Study 201) where both safety and cellulite severity assessments of the treated treatment area were conducted in a double-blinded manner (FIG. 28).

Following the unblinding of the treatment that they received in Study 201, eligible subjects could elect to receive CCH treatment. If subjects opted not to receive treatment, they were placed in Observation Population and observed for up to 2 years. Up to 14 days before initiating treatment with CCH on open-label treatment visit Day 1, a screening evaluation (Screening B) was conducted to determine if specified inclusion and exclusion criteria were met and to determine the treatment areas, if any, that qualified for treatment either by retreatment, redosing, or a first treatment. Subjects that received CCH in Study 201 had the option to receive a treatment course in the same or a different qualifying treatment area. Subjects that received placebo in study 201 had the option to receive 2 treatment courses of CCH in this study. Each treatment course comprised up to 3 sessions each in the same or different qualifying treatment area. After at least 28 days following the end of the first treatment course (e.g., the screening B visit of second treatment course could be performed on Day 71 after treating the first treatment area), the selected treatment area could be retreated or redosed.

Subjects electing not to receive further CCH treatments (observation-only subjects) continued to be followed for safety and cellulite severity assessments at 3-month intervals through month 12 or beyond. Treatment durability was assessed for up to 2 years.

During Screening B, photographs were taken of each of the subject's 4 treatment areas (quadrants) (i.e., left buttock, right buttock, left posterolateral thigh, and right posterolateral thigh). Subjects received instructions for use of the PR-PCSS and subsequently used the scale to rate the severity of their cellulite in each of the 4 treatment areas (quadrants) by comparing digital images of each of their treatment areas (quadrants) displayed on standardized computer monitors with the PR-PCSS instrument. This independent self-assessment occurred in a private setting to minimize any potential bias from site personnel. The investigator then assessed the subject's 4 treatment areas (quadrants) live in real-time using the CR-PCSS. The investigator rated the 4 treatment areas (quadrants) using the Hexsel Cellulite Severity Scale (CSS). Subjects were required to have at least 1 quadrant that met the following criteria for inclusion into the Treatment Phase of the study:
a score of 3 or 4 (moderate or severe) as reported by the subject (PR-PCSS), and
a score of 3 or 4 (moderate or severe) as reported by the investigator (CR-PCSS), and
a Hexsel CSS score no greater than 13.

Subjects were grouped for analysis as the following populations:
The Observation Population included all subjects who rolled over from Study 201. Upon unblinding, those who opt not to be reexposed remain in the Observation Population.
The Safety Population included all subjects who received at least 1 dose of CCH in this study.
The Effectiveness Population included all safety subjects who had a baseline and at least 1 post-baseline assessment on both the CR-PCSS and PR-PCSS on the treatment area selected for treatment in the current study.
The Overall Durability Population was defined as all active responders who had both CR-PCSS and PR-PCSS assessments at Day 1 and Day 71 of a treatment course and 180 days or beyond after Day 1. The active responders were subjects treated with CCH with improvement of at least 1 level at Day 71 of a treatment course on each scale (CR-PCSS and PR-PCSS) from the baseline.
The Durability Population for Double-blind Treated Subjects was defined as all subjects in the Overall Durability Population who showed an improvement on Day 71 of at least 1 level on each scale (CR-PCSS and PR-PCSS) from the baseline for the treatment area treated with CCH in the double-blind study (Study 201).
The Durability Population for Open-label Subjects was defined as all subjects in the Overall Durability Population who showed an improvement on Day 71 of at least 1 level on both the CR-PCSS and the PR-PCSS from the baseline for the treatment area treated with CCH in the current open-label study (Study 202).

The following 2 phases were summarized in the statistical analysis of the study:
Observation Phase: defined as the time period from Screening A to the first treatment date of the same treatment area in the current study (Study 202), or the end of Study 202 if there was no treatment received for the same treatment area in Study 202. The Observation Phase was defined within each treated treatment area, unless the summary was for subject level only and could not be differentiated by the treated treatment area, (i.e., disposition, AE). For subject-level summaries, the Observation Phase was defined as the time period from Screening A to the first treatment date in Study 202 or the end of Study 202 if there was no treatment received in Study 202.
Treatment Phase: defined as the time period from the first treatment date of a selected treatment area in Study 202 to the end of Study 202. The Treatment Phase was defined within each treated treatment area, unless the summary was for subject level only and could not be differentiated by the treated treatment area (i.e., disposition, AEs). For subject-level summaries, Treatment Phase was defined as the time period from the first treatment date in Study 202 to the end of Study 202. All data after Day 1 of the first CCH treatment in Study 202 was included. Therefore, data from the Observation Phase was included in the Treatment Phase.

A subject was excluded from treatment in the study (but not from the observation assessments) if she used any of the following for the treatment of EFP on the legs or buttock within the timelines identified below or intended to use any of the following at any time during the course of the study: (a) liposuction on the side of the body selected for treatment during the 12-month period before injection of CCH, (b) injections (e.g., mesotherapy); radio frequency device treatments; laser treatment; or surgery (including subcision and/or powered subcision) within the selected treatment area during the 12-month period before injection of CCH, (c) Endermologie® or similar treatments within the selected treatment area during the 6-month period before injection of CCH, (d) massage therapy within the selected treatment area during the 3-month period before injection of CCH, and (e) creams (e.g., Celluvera™, TriLastin®) to prevent or mitigate EFP within the selected treatment area during the 2-week period before injection of CCH.

The investigator reviewed his/her assessments and the subject's assessment to determine which treatment areas (quadrants), if any, were eligible. The eligible quadrant selected to receive treatment in the open-label Study 202 was at the discretion of the subject. If the subject received CCH in study 201 and the cellulite severity scores of the treated area had returned to or were higher than Study 201 baseline scores, the subject could elect to have the previously treated quadrant retreated with CCH (termed retreatment). If the subject received CCH in Study 201, the subject could elect to treat a qualifying quadrant other than the one treated with CCH in Study 201 (termed redosing).

For subjects who received active drug in the assigned treatment area in the double-blind study, the treatment area must have had a cellulite severity score (or greater) than the 201 baseline scores of PR-PCSS and CR-PCSS to qualify for retreatment.

Upon completion of treatment (Treatment Phase Day 71), the subject was followed at 3-month intervals per the study schedule up to Day 360. If the subject was administered placebo in Study 201, up to 2 treatment courses of CCH could be administered across 2 treatment areas (buttocks or posterolateral thighs), separately, in Study 202, if eligible. For subjects treated with CCH in Study 201, the treatment area treated was assessed for Long-term Durability, up to Day 720.

The use of the open-label extension design allowed for the following:
Collection of safety data over a 12-month period to assist in further defining the safety profile of CCH in this population.
Evaluation of safety data and immunogenicity over a 12-month period after repeat exposure (retreatment/redosing), as well as monitoring subjects previously treated with CCH over a 12-month period.
Previously placebo-treated subjects to have exposure to CCH.
Assessment of durability of the response to CCH (cellulite severity assessments).

The primary objective of this study was to assess long-term safety of CCH 0.84 mg at scheduled intervals over 1 year (12 months) or more in all subjects with EFP who elected to enroll in this open-label trial regardless of their decision to receive treatment (retreatment or redosing) with open-label CCH or opt to receive no treatment.

The secondary objectives of this study were:
To evaluate safety and immunogenicity of retreating or redosing a subject that had previously received treatment with CCH.
To evaluate the durability of response to CCH in EFP severity over the 12-month post initial dosing of CCH in subjects previously receiving active treatment in Study CCH-201 using the PR-PCSS and the CR-PCSS.
To evaluate the durability of response to CCH in EFP severity beyond 12 months post-initial dosing of CCH in subjects previously receiving active treatment in Study 201 using the PR-PCSS and the CR-PCSS.
To evaluate the long-term response to CCH in assessments of EFP including subject satisfaction, the Investigator Global Aesthetic Improvement Scale (I-GAIS), and the Subject Global Aesthetic Improvement Scale (S-GAIS).
To assess cellulite severity assessments in quadrants treated in this study with CCH.
To evaluate immunogenicity after exposure to CCH.

The persistence of the effect of CCH 0.84 mg per treatment area was assessed for up to 2 years. This included persistence of effect for subjects treated in Study 202 (up to 1 year) and longer-term persistence of effect for subjects treated with CCH in Study 201 who were rolled over into Study 202 and were observed for up to 2 years.

The impact of re-exposure to 2 treatment courses (each treatment course was comprised of 3 treatment sessions) was assessed in 162 subjects. Subjects who received their first treatment course in Study 201 or in the current study, could receive, if eligible, a second treatment course in the current study (termed retreatment or redosing depending if the area had been previously treated with CCH or was naïve, respectively). For subjects who received CCH in the assigned treatment area in Study 201, the treatment area must have had a cellulite severity score equal to (or greater) than the Study 201 baseline to be retreated.

Table 78 represents a description of the current study.

TABLE 78
Description of Study

| | | | | Median | |
|---|---|---|---|---|---|
| | | | | (range) | |
| Number of subjects | | Study | | Age of | |
| by treatment group | | Duration | | Female | |
| Planned/Enrolled/ | | (from | | Subjects | Efficacy |
| Completed | Study design | Day 1) | Dose and regimen | Enrolled | Assessments |
| 350/259/222 | 201 extension | Up to 2 | For treatment naïve: | 49 (19-71) | CR-PCSS, PR- |
| Re-exposed: 163 | with | years | 0.84 mg × 3 sessions | years) | PCSS, I-GAIS, |
| Retreated: 8/8 | retreatment | | (as 12 subcutaneous | | S-GAIS, |
| Redosed: 155 | option | | injections). | | SSCTA, |
| | | | Total dose: 2.52 mg | | Hexsel CSS. |
| | | | For re-exposure : 0.84 | | |
| | | | mg × 6 sessions (as 12 | | |
| | | | subcutaneous | | |
| | | | injections) | | |
| | | | Total dose: 5.04 mg | | |

Subjects who qualified for, and elected to receive treatment were administered a maximum dose of 0.84 mg of CCH administered as up to 12 subcutaneous injections per treatment area (see Table 79). A subject was limited to receive a maximum of 2 treatment courses of CCH in total. If the subject received the first course of CCH in Study 201, the second course of CCH was administered in the current study. If the subject was administered placebo in Study 201, up to 2 treatment courses of CCH could be administered across 2 treatment areas (both buttocks or posterolateral thighs), separately, in the current study if eligible.

TABLE 79
CCH Dose and Volume per Treatment Course

| | | | | Maximum | |
|---|---|---|---|---|---|
| | | Maximum | | Injection | |
| | | Number of | Maximum | Volume | |
| | Injection | Injections | Dose (mg) | (mL) per | |
| | Volume | per Each | per Each | Each | Maximum |
| Dose per | per Each | Treatment | Treatment | Treatment | Cumulative |
| Each Injection^{a} | Injection | Session | Session | Session | EFP Dose |
| CCH 0.07 mg | 0.3 mL | 12 injections | 0.84 mg | 3.6 mL | 2.52 mg |
| | | | (12 injections × | (12 injections × | (3 treatment |
| | | | 0.07 mg) | 0.3 mL) | sessions × |
| | | | | | 0.84 mg) |

^{a}Each injection of CCH was 0.3 mL administered as three 0.1-mL aliquots.

Before injection, the investigator or qualified designee selected dimples within the chosen treatment area that were well defined, evident when the subject was standing, and suitable for treatment. Because the goal of treatment was to improve the aesthetic appearance of the entire treatment area, the investigator was instructed to select dimples that in his or her opinion would most improve the aesthetic appearance of the entire treatment area. The same dimples within a treatment area or different dimples within a treatment area may have been treated at each treatment session, but injections must all have been within the selected treatment area for all 3 sessions.

Injection sites within a dimple were spaced approximately 2 cm apart, if a dimple required more than 1 injection. Each injection site was marked with a “dot” using a surgical marker. For round dimples, the “dot” was placed in the center of the dimple; for elongated dimples, “dots” were spaced out approximately 2 cm along the longer axis of the dimple. The investigator or qualified designee then used a surgical marker to circle each of the dimples selected for treatment. Circles in the selected treatment area could not overlap.

Each injection site received a single skin injection of CCH for cellulite described above and administered as three 0.1-mL aliquots to Positions A, B, and C (for a total injection volume of 0.3 mL) as shown in FIG. 7. The depth of injection corresponded to the length of the treatment needle (0.5 inches) from the tip of the needle to the hub or base of the needle without downward pressure. During each treatment session, the investigator was supplied with 4 syringes. Each syringe contained 0.9 mL of CCH (i.e., up to 3 injections in each syringe). Up to 12 skin injections of 0.3 mL per injection were administered within the selected treatment area during each treatment session.

### B. Dosing and Time of Dosing

The dose selected for this study is the same dose as the dose used in the parent study, Study 201. Subjects who qualified for, and elected to receive treatment were administered CCH 3 times, 21 days apart (Study Days 1, 22, and 43). Subjects who received placebo in Study 201 could have elected to receive 2 courses of treatment in this study to a qualifying treatment area(s). Treatment Courses I and II administered within this study (Study 202) were separated by no less than 28 days after the final dose of CCH in the first course of this study.

The current study was an open-label study. At the time of entry into this study, subjects and investigators remained blinded to the identity of the study drug administered in Study 201. Until the 201 study drug blind was broken by the sponsor, subjects underwent observation-only visits at 3-month intervals±7 days (relative to the initial dose in 201) where both safety and cellulite severity assessments of the treated treatment area were conducted in a double-blinded manner.

### C. Effectiveness Methodology

Digital photography was utilized to assess certain cellulite severity parameters at specific time points, for subjects in the observation-only group as well as those electing to be retreated or redosed with CCH. At the Screening B visit for subjects who elected to receive redosing or retreatment, the investigator or qualified designee photographed each treatment area using a sponsor-supplied standardized digital camera. The subject was in the standing position for each photography session and wore a standardized garment for photographs.

Cellulite assessments conducted by the investigator were independent of assessments conducted by the subjects. Therefore, all subject cellulite assessments were completed before the investigator's cellulite assessments were conducted. At Screening B, after both the subject's and investigator's assessments were completed, the subject's assessments were compared to the clinician's assessments to determine treatment area eligibility. If more than 1 treatment area was eligible, the subject selected 1 treatment area for treatment.

Subjects with a treatment area originally treated with CCH in the double-blind study, and assessed for treatment durability beyond Day 360 completed the PR-PCSS at specific time points. Subjects electing retreatment or redosing after the study drug blind was broken in Study 201, were rescreened (Screening B visit [Baseline]) within 14 days before dosing Day 1.

Digital photographs were taken of all 4 treatment areas (quadrants) for qualifying purposes at Screening B. Subjects then performed the PR-PCSS for both buttocks and thighs. At the beginning of visits on Days 22, 43, and 71 digital photographs of the selected treatment area was taken and the PR-PCSS for the selected treatment area was completed.

Subjects in the observation-only group completed the S-GAIS at specific time points using the pretreatment Day 1 image (baseline) of the assigned treatment area in the double-blind study for comparison. For subjects electing to receive CCH treatment, the S-GAIS assessment was conducted at specific time point and compared back to the pre-dosing Screening B image (Baseline for treatment subjects) of the selected treatment area. Subjects who elected to receive CCH through retreatment or redosing completed Subject Satisfaction with Cellulite Treatment Scale assessment. For subjects in the observation-only group, the CR-PCSS was completed at specific time points.

For subjects who elected to receive CCH as retreatment or redosing, the investigator determined the severity of cellulite of the 4 treatment areas (quadrants) by live assessments using the CR-PCSS for buttock and thigh after the subject completed their self-assessment using the PR-PCSS at the Screening B visit (Baseline). The eligible treatment area selected was at the discretion of the subject. Before injections on treatment visit Days 1, 22 and 43 and on visit Day 71, investigators evaluated the selected treatment area by live assessments using the CR-PCSS. For subjects originally treated with CCH in the double-blind study who were assessed for treatment durability beyond Day 360, the CR-PCSS was completed at specific time points.

The I-GAIS was completed for subjects in the observation only group at the final study visit (month 12 or early termination) using the pretreatment Day 1 image of the assigned treatment area of the double-blind study. The I-GAIS was completed for subjects who elected to receive CCH treatment on Day 71 of the treatment course and the 12-month or EOS visit. To conduct this assessment, the investigator determined the degree of improvement from the Screening B digital image of the selected treatment area by comparing the cellulite in a live assessment on Day 71 and at Month 12 or EOS visit to the Screening B pretreatment (Baseline) image of the subject's selected treatment area.

For subjects in the observation-only group, the Hexsel CSS was conducted at specific time points. For subjects that elected retreatment, the Hexsel CSS was conducted at specific time points.

The composite endpoints for cellulite severity were the proportions of composite responders. A composite responder was defined as a subject with a treatment area with an improvement from baseline of at least 2 (or 1) levels of severity in the CR-PCSS and an improvement from baseline of at least 2 (or 1) levels of severity in the PR-PCSS. These endpoints were summarized by treated treatment area and overall (buttocks and thighs) and by study day using appropriate descriptive statistics.

Other endpoints for treated treatment areas (quadrants) included: Change from baseline for PR-PCSS.
Proportion at each level of improvement in the PR-PCSS:
   Proportion of responders defined as subjects with an improvement from baseline of at least 2 levels of severity in the PR-PCSS (subject rated).
   Proportion of responders defined as subjects with an improvement from baseline of at least 1 level of severity in the PR-PCSS (subject rated).
Change from baseline for CR-PCSS
Proportion at each level of improvement in the CR-PCSS:
   Proportion of investigator responders defined as subjects with an improvement from baseline of at least 2 levels of severity in the CR-PCSS (investigator rated).
   Proportion of investigator responders defined as subjects with an improvement in from baseline of at least 1 level of severity in the CR-PCSS (investigator rated).
Proportion of responders at each level of the I-GAIS:
   Proportion of subjects at each level of the I-GAIS on Day 71 and Day 360.
Proportion of responders at each level of the S-GAIS:
   Proportion of subjects at each level of the S-GAIS on Day 71 and Day 360.
Proportion of responders at each level of the subject satisfaction with cellulite treatment scale.
Change in the Hexsel CSS total score from the Screening Visit.

Observation endpoints for each treated treatment area included:
Proportion of composite responders defined as subjects with an improvement in severity from baseline of at least 2 (or 1) in the CR-PCSS and PR-PCSS.
Change from baseline in PR-PCSS.
Proportion of each level of improvement in the PR-PCSS.
   Proportion of responders defined as subjects with an improvement from baseline of at least 2 levels of severity in the PR-PCSS.
   Proportion of responders defined as subjects with an improvement from baseline of at least 1 level of severity in the PR-PCSS.
Change from baseline in CR-PCSS.
Proportion at each level of improvement in the CR-PCSS:
   Proportion of investigator responders defined as subjects with an improvement from baseline of at least 2 levels of severity in the CR-PCSS (investigator rated).
   Proportion of investigator responders defined as subjects with an improvement in from baseline of at least 1 level of severity in the CR-PCSS (investigator rated).
Proportion of responders at each level of the subject satisfaction with cellulite treatment scale at Day 360.
Change in the Hexsel CSS total score from Day 71 of Study 201 and Day 360 or long-term durability visits beyond Day 360.

### D. Durability Methodology

An assessment of treatment durability included observations of up to 2 years in subjects who received active treatment in Study 201 and scored an improvement of at least 1 level on both the CR-PCSS and the PR-PCSS. For subjects who received CCH in Study 201 and also received CCH in the current study, the original treatment area treated in Study 201 was assessed until Day 720 of the current study, in addition to any treatment areas (quadrants) treated during the open-label study. The Durability Population also included subjects who received CCH in Study 201 but opted not to receive additional treatments in this study and subjects who received placebo in Study 201 and received CCH in the current study.

For subjects assessed for durability beyond Day 360, subjects must: (a) have participated in and completed the double-blind Study 201, (b) have received active CCH in the double-blind Study 201, (c) have achieved an improvement of at least 1 level on both the CR-PCSS and the PR-PCSS at the same visit on or before Day 71 in the double-blind Study 201, and (d) have been willing to apply sunscreen to the CCH-201 treated treatment area before each exposure to the sun while participating in the study (i.e., screening through EOS).

The durability of treatment effect was based on the longitudinal responses of the cellulite assessments of CR-PCSS and PR-PCSS. The number and percentage of responders at each level (i.e., 1-level or 2-level improvement) for CR-PCSS, PR-PCSS and their combinations (i.e., composite score, 1-level or 2-level improvement for both scores) associated with treated treatment areas (quadrants) were summarized by study day.

Treatment failure was defined as active responders where the CR-PCSS and PR-PCSS returned to baseline or worse in an CCH-treated treatment area during a certain follow-up period. The number and percentage were summarized by study day, (i.e., Days 180, 360, 540, and 720).

Responders were from the following 2 populations:
Durability Population for Double-blind Treated Subjects: All subjects in the Durability Population who showed an improvement on Day 71 (of Study 201) of at least 1 level on each scale (CR-PCSS and PR-PCSS) from the baseline for the treatment area treated with CCH in double-blind study (Study 201) and followed for up to 2 years.
Durability Population for Open-label Subjects: All subjects in the Durability Population who showed an improvement on Day 71 (of Study 202) of at least 1 level on both the CR-PCSS and the PR-PCSS from the baseline for the treatment area treated with CCH in Study 202 who were then followed up to 1 year.

### E. Safety Assessments

Safety variables included: AEs, injection site reactions/local tolerability, vital signs, clinical laboratory parameters (including hematology, blood chemistry, and urinalysis), and immunogenicity.

### F. Study Subjects

Two hundred fifty-nine subjects rolled over from Study 201, participated in the current study, and were included in the Observation Population (Table 80). The Observation Population comprised 121 subjects that had been treated previously with CCH and 138 subjects that had been treated previously with placebo in Study 201. Fifty-three (20.5%) subjects in the Observation Population were included in the Durability Population for Double-blind treated subjects. These subjects had shown at least a 1-level improvement on both the CR-PCSS and PR-PCSS at the same visit on or before the Day 71 Visit in Study 201.

TABLE 80
Analysis Populations for the Observation Phase

| | | Study Treatment in Study 201 | | |
|---|---|---|---|---|
| | | CCH | | |
| | Statistic | 0.84 mg | Placebo | Overall |
| Observation Population | n (%) | 121 (100.0) | 138 (100.0) | 259 (100.0) |
| Durability Population for | n (%) | 53 (43.8) | 0 (0.0) | 53 (20.5) |
| Double-blind Treated Subjects | | | | |

During the Treatment Phase, 200 subjects received at least 1 dose of CCH in this study and were included in the Safety Population (Table 81). Of the subjects included in the Safety Population, 193 subjects had baseline and at least 1 post-baseline assessment on both the CR-PCSS and PR-PCSS on the treatment area selected for treatment in the current study, and were included in the Effectiveness Population. Three subjects switched selection of the treatment region from buttock to thigh and received treatment in buttock and then in thigh in Study 202 treatment courses, 8 subjects switched the treatment region from thigh to buttock in Study 202 treatment courses. These subjects were counted for both treatment regions.

During the Treatment Phase, subjects received up to 2 treatment courses. Subjects who received placebo in Study 201 could have received up to 2 treatment courses in this study. Subjects who received CCH in Study 201 received 1 treatment course in this study.

During the study, 163 subjects received a second treatment course in this study and were included in the Safety Population. This retreated/redosed treatment group comprised 88 subjects that received their second treatment course of CCH in this study (having received their first treatment course in Study 201) and 75 subjects that received both their first and second treatment courses in this study.

The Effectiveness Population included 162 subjects that received a second treatment course in this study. This retreated/redosed treatment group comprised 87 subjects that received their second treatment course of CCH in this study having received their first treatment course in Study 201 and 75 subjects that received both their first and second treatment courses in this study.

Forty-two subjects in the Durability Population entered the Treatment Phase in this study and were included in the Durability Population for Double-blind Treated Subjects (Table 81).

The Durability Population for Open-label Treated Subjects comprised 124 subjects. All subjects in the Durability Population who showed an improvement on Day 71 of at least 1 level on both the CR-PCSS and the PR-PCSS from the baseline for the treatment area were treated with CCH in the open-label study (Study 202). This population comprised the following groups:
1. Subjects who received CCH in Study 201 who responded in Treatment Course 1 in Study CCH-201 that were either redosed or retreated.
   Subjects retreated that received CCH in study 201 and the cellulite severity scores of the treated area had returned to or were worse than Study 201 baseline scores. These subjects could elect to have the previously treated area retreated with CCH
   Subjects redosed received CCH in Study 201
2. Subjects who received placebo in Study 201 who were initially treated in Study 202 and were responders in Treatment Course I.
3. Subjects who received placebo in Study 201 who received 2 treatment courses in Study 202 and were responders to Treatment Course 2.

TABLE 81
Analysis Populations for the Treatment Phase

| | | Treatment Region with CCH 0.84 mg | | |
|---|---|---|---|---|
| | Statistic | Buttock | Thigh | Overall |
| Safety Population | N | 112 | 99 | 200 |
| Effectiveness Population | n (%) | 111 (99.1) | 93 (93.9) | 193 (96.5) |
| Durability Population | n (%) | 93 (83.0) | 65 (65.7) | 152 (76.0) |
| Durability Population for | n (%) | 26 (23.2) | 16 (16.2) | 42 (21.0) |
| Double-blind Treated Subjects | | | | |
| Durability Population for | n (%) | 72 (64.3) | 54 (54.5) | 124 (62.0) |
| Open-label Treated Subjects | | | | |

### G. Disposition of Subjects:

FIG. 28 summarizes how the subjects were disposed.

(i) Disposition of Subjects (Observation Phase): During the Observation Phase, 259 subjects were evaluated with 222 (85.7%) subjects completing this phase (Table 82). Overall, 37 subjects discontinued for the following reasons: an AE of a mild increase in eosinophils without exposure to CCH (1 subject; 0.4%), lost to follow-up (13 subjects; 5%), withdrawal by subject (13 subjects, 5%), and other (10 subjects, 3.9%). The other category included screen failures, subjects declining to participate in the Treatment Phase, site closure on Study Day 272 of subject enrollment, or subjects not compliant with study visits.

TABLE 82
Subject Disposition During the Observation Phase (All Subjects)

| | | Study Treatment in Study-201 | | |
|---|---|---|---|---|
| | | CCH | | |
| | Statistic | 0.84 mg | Placebo | Overall |
| Participated | N | 121 | 138 | 259 |
| Completed | n (%) | 106 (87.6) | 116 (84.1) | 222 (85.7) |
| Discontinued | n (%) | 15 (12.4) | 22 (15.9) | 37 (14.3) |

Reason for Discontinuation

| | | | | |
|---|---|---|---|---|
| Adverse Event | n (%) | 0 (0.0) | 1 (0.7) | 1 (0.4) |
| Lost to Follow-up | n (%) | 7 (5.8) | 6 (4.3) | 13 (5.0) |
| Withdrawal by Subject | n (%) | 8 (6.6) | 5 (3.6) | 13 (5.0) |
| Other | n (%) | 0 (0.0) | 10 (7.2) | 10 (3.9) |

(ii) Disposition of Subjects (Treatment Phase): Two hundred sixteen subjects were screened for the Treatment Phase, 200 subjects were enrolled, and 156 (78%) subjects completed the Treatment Phase (Table 83). Overall, there were more buttock treated regions than thigh treated regions. One hundred four subjects were treated in the buttock and 96 were treated in the thigh regions.

During the Treatment Phase, 163 subjects were re-exposed to CCH. The group of 163 subjects re-exposed subjects comprised 88 subjects re-exposed to CCH in Treatment Course I, having received their first treatment course in Study 201 and 75 subjects re-exposed in Treatment Course 2.

During the Treatment Phase, 112 subjects were initially treated in Treatment Course 1 and 88 subjects were re-exposed in Treatment Course 1 (82 subjects that were redosed and only 6 subjects were retreated). The 82 redosed subjects received CCH in Study 201, and elected to have treatment in a qualifying treatment area other than the one treated with in study 201. The 6 subjects that were retreated received CCH in Study 201 and the cellulite severity scores of their treated area had returned to the baseline score or were higher than Study 201 baseline scores. These subjects elected to have the previously treated treatment area retreated with CCH.

Seventy-five subjects received both their first and second treatment courses in Study 202. All subjects, by definition, in Treatment Course 2 were re-exposed to CCH. The majority (73/75 subjects; 97.3%) of subjects were redosed and only 2 subjects were retreated.

One hundred fifty-six subjects completed the study. The distribution of subjects discontinuing CCH was similar in subjects treated in the buttocks vs. thigh (22 [21.2%] vs 22 [22.9%] subjects).

TABLE 83
Subject Disposition During the Treatment Phase (All Subjects)

| | Treatment Region with CCH 0.84 mg | | |
|---|---|---|---|
| Statistic | Buttock | Thigh | Overall |

Treatment Course I

| | | | | |
|---|---|---|---|---|
| Screened | N | | | 216 |
| Enrolled in Treatment Course I | N | 104 | 96 | 200 |
| Redosing | n (%) | 48 (46.2) | 34 (35.4) | 82 (41.0) |
| Retreatment | n (%) | 4 (3.8) | 2 (2.1) | 6 (3.0) |
| Initially treated | n (%) | 52 (50.0) | 60 (62.5) | 112 (56.0) |

Treatment Course 2

| | | | | |
|---|---|---|---|---|
| Screened | N | | | 78 |
| Enrolled in Treatment Course 2 | n (%) | 43 (41.3) | 32 (33.3) | 75 (37.5) |
| Redosing | n (%) | 41 (39.4) | 32 (33.3) | 73 (36.5) |
| Retreatment | n (%) | 2 (1.9) | 0 (0.0) | 2 (1.0) |
| Completion Status | | | | |
| Completed | n (%) | 87 (83.7) | 69 (71.9) | 156 (78.0) |
| Discontinued | n (%) | 22 (21.2) | 22 (22.9) | 44 (22.0) |
| Reason for Discontinuation | | | | |
| Adverse Event | n (%) | 0 (0.0) | 5 (5.2) | 5 (2.5) |
| Death | n (%) | 0 (0.0) | 1 (1.0) | 1 (0.5) |
| Lost to Follow-up | n (%) | 9 (8.7) | 3 (3.1) | 12 (6.0) |
| Withdrawal by Subject | n (%) | 8 (7.7) | 13 (13.5) | 21 (10.5) |
| Other | n (%) | 5 (4.8) | 0 (0.0) | 5 (2.5) |

### H. Efficacy Results

### 1. Clinical Endpoints

The response on the composite CR-PCSS/PR-PCSS endpoint was assessed during the Treatment Phase and during the Observation Phase in active responders from Study 201. The composite endpoint for cellulite severity was defined as the proportion of composite responders. Responders were defined as subjects with an improvement from baseline (in Study 201) of at least 1 level of severity in the CR-PCSS and an improvement from baseline of at least 1-level of severity in the PR-PCSS. Active responders were subjects treated with CCH in Study 201 with an improvement of at least 1-level on both the CR-PCSS and the PR-PCSS from the baseline for the treatment area in Study 201.

During the Observation Phase of Study 202, subjects that rolled over from Study 201 and were active responders in the area treated, were observed for up to 2 years. Subjects treated during the Treatment Phase of Study 202, and were 2-level composite responders were observed for up to 1 year. Treatment failure was defined as active responders who had received CCH and whose CR-PCSS and PR-PCSS return to the baseline in an CCH-treated area. Durability of treatment effect was calculated as the inverse of treatment failure. Analyses were conducted in subjects that were 2-level composite responders and subjects with at least a 1-level composite response.

The composite endpoints for cellulite severity were defined as the proportions of composite responders. This was defined as subjects with an improvement from baseline (in Study 201) of at least 2 (or 1) levels of severity in the CR-PCSS and an improvement from baseline of at least 2 (or 1) levels of severity in the PR-PCSS. This analysis was conducted for subjects enrolled in the Observation and Treatment Phases. The investigator's assessments of the durability of response in 1-level and 2-level composite responders during the Observation Phase were conducted in a double-blinded manner.

Assessments were conducted by treatment area and by subject. Thus, response was assessed separately by treatment area for a subject that may have been treated with CCH in different treatment areas during different treatment courses. Subjects that responded in 1 treatment area were considered 1-level or 2-level composite responders, as applicable, since they may not have responded equally or at all in another treatment area, if treated. Durability of response was assessed in subjects with at least a 1-level or 2-level composite response at Day 71 in Study 201.

There were 26 subjects, (including 19 buttock- and 7 thigh-treated regions) that were 2-level composite responders at Day 180 or beyond. These subjects received either 1 or 2 treatment courses during Study 202. All evaluable subjects that were observed in Study 202, Days 180 (26 subjects; 100%) and 360 (21 subjects; 100%) maintained a 2-level response. There were no subjects who were 2-level composite responders in whose CR-PCSS and PR-PCSS ratings for a newly treated area returned to their baseline (in Study 202) ratings or worse. For subjects treated in this study, a 2-level composite response persisted and was similar at Days 180 and 360, and was similar in buttock and thigh-treated regions.

There were 124 subjects (including 72 buttock- and 54 thigh-treated regions) that were 1-level composite responders at Day 180 and 114 subjects that maintained a 1-level composite response at Day 360 during Study 202. There were no subjects assessed as treatment failures whose CR-PCSS and PR-PCSS ratings returned to their baseline (in Study 202) ratings or worse. The 1-level composite response persisted and was similar at Days 180 and 360 and was similar in the buttock- and thigh-treated regions.

### 2. Persistence of Efficacy

This study assessed the long-term safety of CCH at scheduled intervals over 2 years (24 months) in all subjects with cellulite who elected to enroll. Eligible subjects were treated in either 1 buttock or 1 thigh and depending on eligibility, could be treated with a second course of treatment in 1 buttock or 1 thigh. The persistence of response was assessed for up to 2 years in subjects who were treated with CCH in Study 201 and up to 12 months in subjects who received their initial treatment of CCH in Study 202. The data available in subjects evaluated at Day 720 in Study 202, who were treated in Study 201, was supportive of the persistence of the CR-PCSS and PR-PCSS response for up to 2 years.

The results of this study provide supportive data for up to 2-years on the long-term safety and persistence of the effect of CCH 0.84 mg per treatment area×3 treatment sessions 21 days apart. This included persistence of effect for subjects treated in Study 202 (up to 1 year), and longer-term persistence of effect of subjects treated with CCH in Study 201 and enrolled in the extension study in Study 202 that were observed for up to 2 years.

Subjects enrolled in the current open-label study (Study 202), experienced approximately 1-level reductions (i.e., improvement) in the severity of cellulite on the CR-PCSS and the PR-PCSS from baseline. This magnitude of reduction was similar to the reductions in the CR-PCSS and PR-PCSS experienced by subjects in the double-blind studies.

(a) Persistence of the PR-PCSS Response for Up to One Year: The persistence in the change from baseline in the PR-PCSS was consistent between the groups of subjects treated with CCH in Study 201, treated in Study 202, and re-exposed in Study 202. Of the 259 subjects that enrolled in Study 202 from Study 201, mean (SD) change from baseline at Day 71 in Study 201, was −1.2 (0.89) in the CCH-treated group (n=121) and −0.6 (0.74) in the placebo-treated group (n=138). Upon observation for up to 1 year, the CCH-treated subjects continued to show improvement in the PR-PCSS rating. The mean (SD) change from baseline in the CCH-treated group was −0.7 (0.88), −0.7 (0.96), and −1.0 (0.91), on Days 180 (n=119), 270 (n=105), and 360 (n=96).

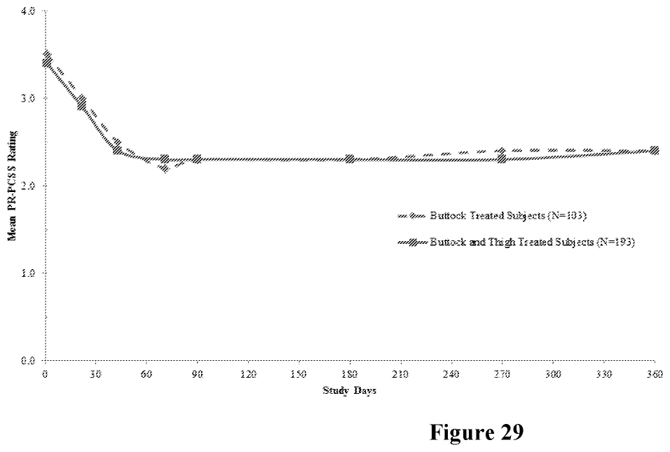

Of the subjects enrolled and treated in Study 202, 193 subjects (103 buttock- and 90 thigh-treated regions) were assessed for effectiveness during Treatment Course 1. Subjects that received Treatment Course 1 included subjects initially treated with CCH and subjects either retreated or redosed. Upon observation for up to 1-year, the CCH-treated subjects continued to show an improvement from baseline in the PR-PCSS rating. The overall mean (SD) change from baseline was −1.1 (0.85), −1.1 (0.87), and −1.0 (0.83) on Days 180, 270, and 360, respectively. The response was similar for both buttock (n=103) and thigh-treated regions (n=90) (FIG. 29).

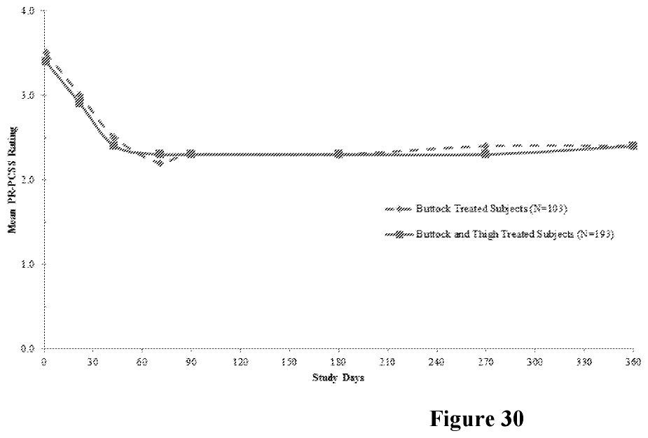

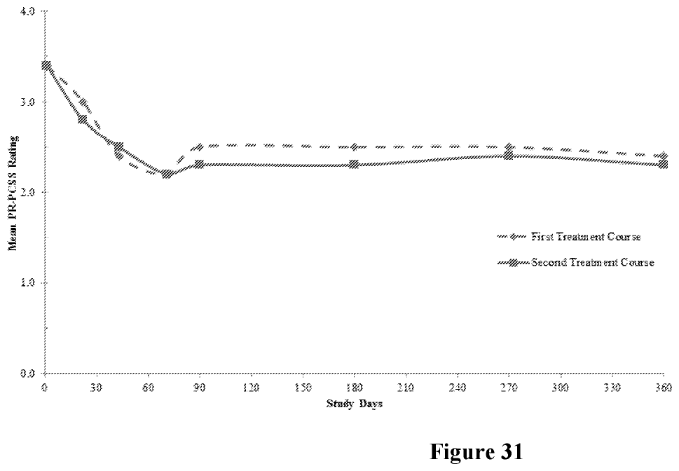

In this study, 162 subjects that were re-exposed and evaluated for effectiveness upon observation for up to 1-year, the CCH-treated subjects continued to show an improvement from baseline in the PR-PCSS at Days 180 (−1.0 [0.83]) and 360 (−1.0 [0.83]). The response was similar in the buttock and thigh-treated regions after the first treatment course as after the second treatment course (FIG. 30 and FIG. 31).

(b) Persistence of the CR-PCSS Response for up to One Year: The persistence in the change from baseline in the CR-PCSS was consistent between the groups of subjects treated with CCH in Study 201, treated in Study 202, and re-exposed in Study 202.

Of the 121 CCH-treated subjects that enrolled in Study 202 from Study −201, the mean (SD) change from baseline on Day 71 in Study −201, was −0.8 (0.88) in the CCH-treated group and −0.3 (0.6) in the placebo-treated group. Upon observation for up to 1-year, the CCH-treated subjects continued to show improvement in CR-PCSS ratings. The overall mean (SD) change from baseline was −0.6 (0.80), −0.6 (0.76) and −0.9 (0.86) on Days 180, 270 and 360, respectively.

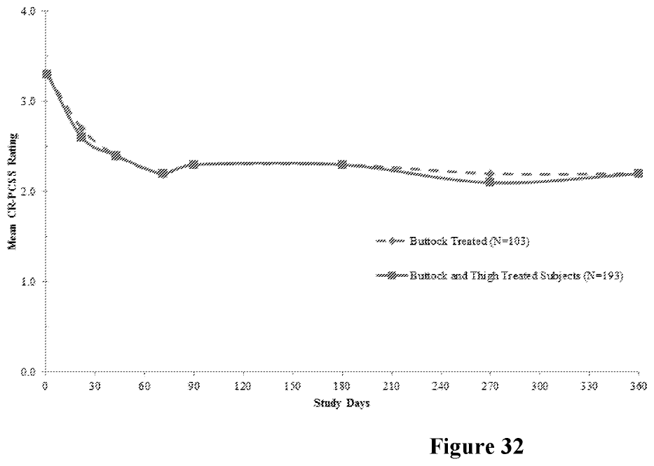

Of the subjects enrolled and treated in Study 202, 193 CCH-treated subjects (103 buttock-treated and 90 thigh-treated regions) were assessed for effectiveness during Treatment Course 1 of this study. Subjects that received Treatment Course 1 included subjects initially treated and subjects either retreated or redosed. Upon observation for up to 1-year, the CCH-treated subjects continued to show improvement in the CR-PCSS ratings. The overall mean (SD) change from baseline was −1.0 (0.80), −1.1 (0.83), and −1.1 (0.82) on Days 180, 270, and 360, respectively (FIG. 32). The response was similar for both buttock (n=103) and thigh-treated regions (n=90).

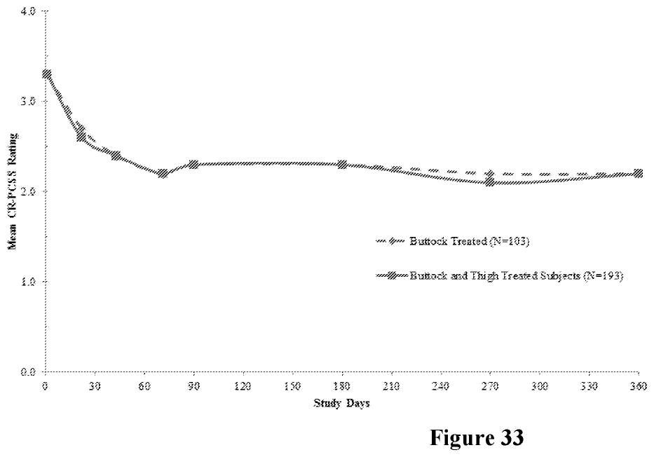

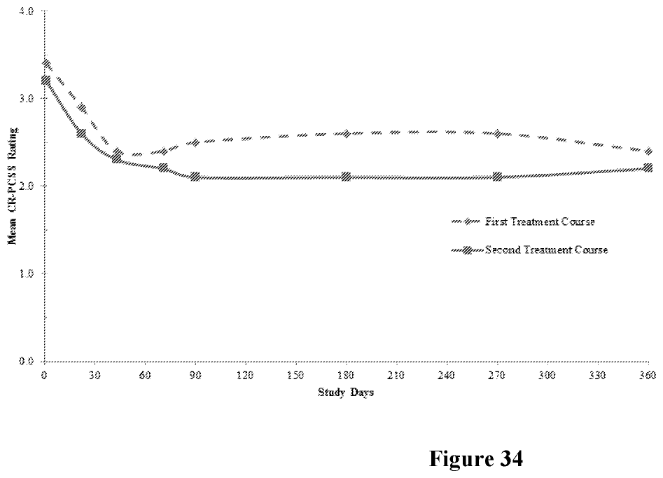

Of the 162 subjects that were re-exposed in Study 202 and evaluated for effectiveness upon observation for up to 1-year, the CCH-treated subjects continued to show improvement in the CR-PCSS at Days 180 and 360. The overall mean (SD) change from baseline was −1.0 (0.80) at Day 180 and −0.9 (0.77) at Day 360. The response was similar in the buttock and thigh-treated regions after the first treatment course as after the second treatment course (FIG. 33 and FIG. 34).

(c) Persistence of the Two-level Composite Response for up to Two Years: Seven subjects who were 2-level composite responders (5 buttock and 2 thigh CCH-treated regions), were evaluated for up to 720 days. At Day 720, none of the 7 subjects had their CR-PCSS and PR-PCSS ratings return to their baseline scores (in Study 201) or worse.

(d) Persistence of the One-Level Composite Response for Up to One Year: Twenty-three subjects that were ≥1-level composite responders (13 buttock and 10 CCH-treated thigh regions) were assessed for up to 720 days. At Day 720, in 21 (91.3%) subjects, none had their CR-PCSS and PR-PCSS ratings return to their baseline scores (in Study 201) or worse.

### 3. Tolerance Effects

Tolerance (the loss of an ability to respond to therapeutic dose(s) over time upon re-exposure to CCH) to a second treatment course was assessed in this study. Data from the current study supported the lack of tolerance (the loss of an ability to respond to therapeutic dose(s) over time) upon re-exposure to CCH. 163 subjects were re-exposed to treatment. This group comprised 88 subjects re-exposed in Treatment Course 1, having received their first treatment course in Study 201, and 75 subjects re-exposed in Treatment Course 2.

Seventy-five subjects received both their first and second treatment courses in Study 202. All subjects, by definition, in Treatment Course 2 were re-exposed to CCH. The majority (73/75 subjects; 97.3%) of subjects were redosed and only 2 subjects were retreated. There are no signals suggestive of any safety concerns in subjects re-exposed (redosed and/or retreated) to CCH treatment.

(a) PR-PCSS Rating Change from Baseline (Retreated/Redosed): In this study, 162 subjects (which included 91 buttock- and 71 thigh-treated regions) were assessed for effectiveness on the PR-PCSS after their first treatment course and after their second treatment course of CCH (1 subject was not included in the effectiveness population due to a missing assessment) (Table 84).

When assessed at Day 71 and Day 360 after initial treatment, the change in the PR-PCSS observed after the second treatment course was similar and superimposable to the changes observed at the same time points after the first treatment course. At Day 360, the overall mean (SD) change in the PR-PCSS rating after the first treatment course was −1.0 (0.82) (Table 84) and after the second treatment course was −1.0 (0.83) (Table 85). Similar decreases were observed in buttock- and thigh-treated regions. Changes in the buttock-treated area (FIG. 31) were similar to changes for the combined analysis of thigh- and buttock-treated regions (FIG. 30). Subjects re-exposed in Study 202, experienced a reduction in the severity of cellulite on the PR-PCSS of a magnitude similar to the response observed in subjects in double-blind studies (Studies 201, 302, and 303) after their first and only treatment course.

TABLE 84
PR-PCSS Rating and Change from Baseline by Visit After the First
Treatment Course for Subjects Who Received Their First and Second
Treatment Courses of CCH (Effectiveness Population)

| | | Treatment Region with CCH 0.84 mg | | |
|---|---|---|---|---|
| | | Buttock | Thigh | Overall |
| First Treatment Course^{a} | Statistic | (N = 91) | (N = 71) | (N = 162)^{b} |
| Baseline | | | | |
| None (0) | n (%) | 0 (0.0) | 0 (0.0) | 0 (0.0) |
| Almost None (1) | n (%) | 0 (0.0) | 0 (0.0) | 0 (0.0) |
| Mild (2) | n (%) | 0 (0.0) | 0 (0.0) | 0 (0.0) |
| Moderate (3) | n (%) | 56 (61.5) | 47 (66.2) | 103 (63.6) |
| Severe (4) | n (%) | 35 (38.5) | 24 (33.8) | 59 (36.4) |
| | N | 91 | 71 | 162 |
| | Mean (SD) | 3.4 (0.49) | 3.3 (0.48) | 3.4 (0.48) |
| | Median | 3.0 | 3.0 | 3.0 |
| | Min, Max | 3, 4 | 3, 4 | 3, 4 |

Change from Baseline to Treatment Day 71

| | | | |
|---|---|---|---|
| N | 91 | 71 | 162 |
| Mean (SD) | −1.2 (0.92) | −1.1 (0.75) | −1.2 (0.85) |
| Median | −1.0 | −1.0 | −1.0 |
| Min, Max | −3, 0 | −3, 0 | −3, 0 |

Change from Baseline to Treatment Observation Day 180

| | | | |
|---|---|---|---|
| N | 88 | 71 | 159 |
| Mean (SD) | −0.9 (1.01) | −0.9 (0.77) | −0.9 (0.91) |
| Median | −1.0 | −1.0 | −1.0 |
| Min, Max | −4, 1 | −3, 0 | −4, 1 |

Change from Baseline to Treatment Observation Day 360

| | | | |
|---|---|---|---|
| N | 78 | 69 | 147 |
| Mean (SD) | −1.0 (0.87) | −1.1 (0.77) | −1.0 (0.82) |
| Median | −1.0 | −1.0 | −1.0 |
| Min, Max | −3, 0 | −2, 1 | −3, 1 |

^{a}During Study 202, the first treatment course was named the first treatment visit.

TABLE 85
PR-PCSS Rating and Change from Baseline by Visit After the Second
Treatment Course for Subjects Received Their First and Second
Treatment Courses of CCH (Effectiveness Population)

| | | Treatment Region with CCH 0.84 mg | | |
|---|---|---|---|---|
| | | Buttock | Thigh | Overall |
| Second Treatment Course^{a} | Statistic | (N = 95) | (N = 67) | (N = 162)^{b} |
| Baseline | | | | |
| None (0) | n (%) | 0 (0.0) | 0 (0.0) | 0 (0.0) |
| Almost None (1) | n (%) | 0 (0.0) | 0 (0.0) | 0 (0.0) |
| Mild (2) | n (%) | 0 (0.0) | 0 (0.0) | 0 (0.0) |
| Moderate (3) | n (%) | 57 (60.0) | 51 (76.1) | 108 (66.7) |
| Severe (4) | n (%) | 38 (40.0) | 16 (23.9) | 54 (33.3) |
| | N | 95 | 67 | 162 |
| | Mean (SD) | 3.4 (0.49) | 3.2 (0.43) | 3.3 (0.47) |
| | Median | 3.0 | 3.0 | 3.0 |
| | Min, Max | 3, 4 | 3, 4 | 3, 4 |

Change from Baseline to Treatment Day 71

| | | | |
|---|---|---|---|
| N | 92 | 67 | 159 |
| Mean (SD) | −1.2 (0.85) | −0.9 (0.74) | −1.1 (0.82) |
| Median | −1.0 | −1.0 | −1.0 |
| Min, Max | −3, 1 | −3, 1 | −3, 1 |

Change from Baseline to Treatment Observation Day 180

| | | | |
|---|---|---|---|
| N | 90 | 65 | 155 |
| Mean (SD) | −1.1 (0.82) | −0.8 (0.80) | −1.0 (0.83) |
| Median | −1.0 | −1.0 | −1.0 |
| Min, Max | −3, 1 | −2, 1 | −3, 1 |

Change from Baseline to Treatment Observation Day 360

| | | | |
|---|---|---|---|
| N | 81 | 62 | 143 |
| Mean (SD) | −1.1 (0.82) | −0.8 (0.81) | −1.0 (0.83) |
| Median | −1.0 | −1.0 | −1.0 |
| Min, Max | −3, 1 | −3, 1 | −3, 1 |

^{a}During Study 202, the second treatment course was named the second treatment visit.
^{b}One hundred sixty-two subjects received a second treatment course in this study and were included in the Effectiveness Population. This retreated/redosed treatment group comprised 87 subjects that received their second treatment course in Study 202, after having received their first treatment course in Study 201, and 75 subjects that received both their first and second treatment courses in Study 202.

(b) CR-PCSS Rating Change from Baseline (Retreated/Redosed): In this study, 162 subjects (including 91 buttock- and 71 thigh-treated regions) received their first and second treatment course of CCH and were assessed for effectiveness on the CR-PCSS (1 subject was not included in the effectiveness population due to a missing assessment) (Table 86).

When assessed at Day 71 and Day 360 after initial treatment, the change in the CR-PCSS observed after the second treatment course was similar and superimposable to the changes observed at the same time points after the first treatment course. At Day 360, the overall mean (SD) change in the CR-PCSS rating after the first treatment course was −1.1 (0.88) (Table 86) and after the second treatment course was −0.9 (0.77) (Table 87). Similar decreases were observed in buttock and thigh-treated regions. Changes in the buttock-treated area (FIG. 34) were similar to changes for the combined analysis of thigh- and buttock-treated regions, FIG. 33. Subjects re-exposed in Study −202, experienced a reduction in the severity of cellulite on the CR-PCSS of a magnitude similar to the response observed in subjects in the double-blind studies (Studies 201, 302, and 303) after their first and only treatment course.

TABLE 86
CR-PCSS Rating and Change from Baseline by Visit After the First
Treatment Course for Subjects Who Received Their First and Second
Treatment Courses of CCH (Effectiveness Population)

| | | Treatment Region with CCH 0.84 mg | | |
|---|---|---|---|---|
| First CCH Treatment | | Buttock | Thigh | Overall |
| Course^{a} | Statistic | (N = 91) | (N = 71) | (N = 162) |
| Baseline | | | | |
| None (0) | n (%) | 0 (0.0) | 0 (0.0) | 0 (0.0) |
| Almost None (1) | n (%) | 0 (0.0) | 0 (0.0) | 0 (0.0) |
| Mild (2) | n (%) | 0 (0.0) | 0 (0.0) | 0 (0.0) |
| Moderate (3) | n (%) | 59 (64.8) | 53 (74.6) | 112 (69.1) |
| Severe (4) | n (%) | 32 (35.2) | 18 (25.4) | 50 (30.9) |
| | N | 91 | 71 | 162 |
| | Mean (SD) | 3.4 (0.48) | 3.3 (0.44) | 3.3 (0.46) |
| | Median | 3.0 | 3.0 | 3.0 |
| | Min, Max | 3, 4 | 3, 4 | 3, 4 |
| Change from Baseline to | N | 91 | 71 | 162 |
| Treatment Day 71 | | | | |
| | Mean (SD) | −0.9 (0.90) | −0.9 (0.74) | −0.9 (0.83) |
| | Median | −1.0 | −1.0 | −1.0 |
| | Min, Max | −3, 1 | −2, 0 | −3, 1 |
| Change from Baseline to | N | 89 | 71 | 160 |
| Treatment Day 180 | | | | |
| | Mean (SD) | −0.8 (0.82) | −0.9 (0.79) | −0.9 (0.81) |
| | Median | −1.0 | −1.0 | −1.0 |
| | Min, Max | −3, 1 | −3, 1 | −3, 1 |
| Change from Baseline to | N | 80 | 69 | 149 |
| Treatment Day 360 | | | | |
| | Mean (SD) | −1.0 (0.80) | −1.2 (0.95) | −1.1 (0.88) |
| | Median | −1.0 | −1.0 | −1.0 |
| | Min, Max | −3, 0 | −3, 1 | −3, 1 |

^{a}During Study -202, the first treatment course was named the first treatment visit.

TABLE 87
CR-PCSS Rating and Change from Baseline by Visit After the Second
Treatment Course for Subjects Who Received Their First and
Second Treatment Courses of CCH (Effectiveness Population)

| | | Treatment Region with 0.84 mg | | |
|---|---|---|---|---|
| | | Buttock | Thigh | Overall |
| Second Treatment Course^{a} | Statistic | (N = 95) | (N = 67) | (N = 162)^{b} |
| Baseline | | | | |
| None (0) | n (%) | 0 (0.0) | 0 (0.0) | 0 (0.0) |
| Almost None (1) | n (%) | 0 (0.0) | 0 (0.0) | 0 (0.0) |
| Mild (2) | n (%) | 0 (0.0) | 0 (0.0) | 0 (0.0) |
| Moderate (3) | n (%) | 76 (80.0) | 52 (77.6) | 128 (79.0) |
| Severe (4) | n (%) | 19 (20.0) | 15 (22.4) | 34 (21.0) |
| | N | 95 | 67 | 162 |
| | Mean (SD) | 3.2 (0.40) | 3.2 (0.42) | 3.2 (0.41) |
| | Median | 3.0 | 3.0 | 3.0 |
| | Min, Max | 3, 4 | 3, 4 | 3, 4 |

Change from Baseline to Treatment Day 71

| | | | |
|---|---|---|---|
| N | 92 | 67 | 159 |
| Mean (SD) | −1.1 (0.82) | −0.9 (0.74) | −1.0 (0.79) |
| Median | −1.0 | −1.0 | 1.0 |
| Min, Max | −4, 0 | −2, 0 | −4, 0 |

Change from Baseline to Treatment Observation Day 180

| | | | |
|---|---|---|---|
| N | 90 | 64 | 154 |
| Mean (SD) | −1.1 (0.72) | −1.0 (0.91) | −1.0 (0.80) |
| Median | −1.0 | −1.0 | −1.0 |
| Min, Max | −3, 0 | −4, 0 | −4, 0 |

Change from Baseline to Treatment Observation Day 360

| | | | |
|---|---|---|---|
| N | 81 | 61 | 142 |
| Mean (SD) | −1.0 (0.81) | −0.9 (0.72) | −0.9 (0.77) |
| Median | −1.0 | −1.0 | −1.0 |
| Min, Max | −3, 1 | −3, 0 | −3, 1 |

^{a}During Study 202, the second treatment course was named the second treatment visit.
^{b}One hundred sixty-two subjects received a second treatment course in this study and were included in the Effectiveness Population. This retreated/redosed treatment group comprised 87 subjects that received their second treatment course in Study 202, after having received their first treatment course in Study 201, and 75 subjects that received both their first and second treatment courses in Study 202.

(c) Changes in the Hexsel CSS Total Rating, I-GAIS, S-GAIS, and Subject Satisfaction with EFP Treatment Scale in Subjects Retreated/Redosed: In re-exposed subjects, the effectiveness of CCH in reducing the severity of cellulite was assessed by the Hexsel CSS, improving aesthetic appearance was assessed by the investigator (I-GAIS) and by the subject (5-GAIS), and on the Subject Satisfaction with EFP Treatment Scale. The effectiveness after treatment as assessed by these scales was similar in treatment-naïve subjects and after treatment in subjects re-exposed supporting a lack of tolerance or tachyphylaxis to CCH when retreated or redosed.

### I. Safety Evaluation

The safety profile of CCH was consistent with the safety results of the previous studies. There were no clinically meaningful changes in concomitant medications during the study. There were no clinically meaningful trends observed in the mean changes from baseline in serum chemistry parameters, hematology parameters, urinalysis results, or vital signs during the study. There were no clinically relevant findings in subjects with anti-AUX-I and anti-AUX-II antibodies or in subjects with neutralizing antibodies.

### J. Summary of Results and Conclusions

The durability of response (lack of complete loss) was observed in evaluable 2-level composite responders from Study 201. No subjects returned to baseline CR-PCSS and PR-PCSS levels or worse in a CCH-treated area. A durable response was observed during the:
Observation Phase in 19 (100%) and 16 (100%) evaluable double-blind treated subjects at Days 180 and 360, respectively.
Long-term Durability Phase in 7 (100%) evaluable double-blind treated subjects at Days 540 and 720.
Treatment Phase in 26 (100%) and 21 (100%) evaluable open-label treated subjects at Days 180 and 360, respectively.

The durability of response (lack of complete loss) was demonstrated in evaluable 1-level composite responders from Study −201. A durable response was observed during the:
Observation Phase in 50 (94.3%) and 43 (95.6%) evaluable double-blind treated subjects at Days 180 and 360, respectively.
Long-term Durability Phase in 22 (95.6%) and in 21 (91.3%) evaluable double-blind treated subjects at Days 540 and 720, respectively.
Treatment Phase in 124 (100%) and 114 (100%) evaluable open-label treated subjects at Days 180 and 360, respectively.

Among 2-level CR-PCSS active responders from Study 201, a 2-level response was maintained during the:
Observation Phase in 10 (47.6%) evaluable double-blind treated subjects at Day 360.
Long-term Durability Phase in 3 (30%) and 4 (40%) evaluable double-blind treated subjects at Days 540 and 720, respectively.
Treatment Phase in 32 (62.7%) and 26 (56.5%) evaluable open-label treated subjects at Days 180 and 360.

Among 1-level CR-PCSS active responders, a 1-level response was maintained during the:
Observation Phase in 38 (84.4%) double-blind treated subjects at Day 360.
Long-term Durability Phase in 19 (82.6%) double-blind treated subjects at Day 540 and 16 (69.6%) double-blind treated subjects at Day 720.
Treatment Phase in 109 (87.9%) evaluable open-label treated subjects and 96 (84.2%) open-label treated subjects at Days 180 and 360.

Among-2-level PR-PCSS active responders from Study 201, a 2-level response was maintained during the
Observation Phase in 10 (31.3%) and 14 (48.3%) evaluable double-blind treated subjects at Days 180 and 360, respectively.
Long-term Durability Phase in 7 (53.8%) evaluable double-blind treated subjects at Days 540 and 720.
Treatment Phase in 33 (66.7%) and 24 (54.5%) evaluable open-label treated subjects at Days 180 and 360, respectively.

Among 1-level PR-PCSS active responders from Study 201, a 1-level response was maintained during the:
Observation Phase in 40 (75.5%) and 35 (77.8%) evaluable double-blind treated subjects at Days 180 and 360, respectively.
Long-term Durability Phase in 19 (82.6%) and 20 (87.0%) evaluable double-blind treated subjects at Days 180 and 360, respectively.
Treatment Phase in 103 (83.1%) and 92 (80.7%) evaluable open-label treated subjects at Days 180 and 360, respectively.

The durability of response was also observed in 2-level and 1-level CR-PCSS and 2-level and 1-level PR-PCSS responders and in responders from Study 201 during the Observation Phase, Long-term Durability Phase and Treatment Phase.

A decrease of approximately 1-level in the CR-PCSS rating at Day 360 was observed during the Observation Phase and in double-blind and open-label treated subjects during the Treatment Phase receiving Treatment Courses 1 and 2. Similar effectiveness was observed in subjects redosed and retreated; at treatment Observation Day 360 after a second treatment of CCH (subjects re-exposed vs. treatment-naïve), the mean (SD) change in the CR-PCSS rating was −0.9 (0.77).

A decrease of approximately 1-level in the PR-PCSS rating at Day 360 was observed during the Observation Phase and in double-blind and open-label treated subjects during the Treatment Phase receiving Treatment Course 1 and Treatment Course 2. Similar effectiveness was observed in subjects redosed and retreated at Observation Day 360, after a second treatment of CCH, CCH (subjects re-exposed vs. treatment-naïve) the mean (SD) change in the PR-PCSS rating was −1.0 (0.83).

The effectiveness of CCH in reducing the severity of cellulite was observed during the Observation, Long-term Durability, and Treatment Phases (Treatment Courses 1 and 2) on the Hexsel CSS, I-GAIS, S-GAIS, and on the Subject Satisfaction with EFP Treatment Scale. The magnitude of change on these scales after CCH treatment was comparable between retreated and redosed subjects and treatment-naïve subjects.
On the Hexsel CSS Total Score during the:
   Observation Phase, in CCH-treated subjects, at Observation Visit Day 360, there was a mean (SD) change in the Hexsel CSS total score of −2.2 (2.34) from the Day 71 baseline as compared to a mean (SD) change of −1.2 (1.47) in placebo-treated subjects.
   Long-term Durability Phase, the change from baseline (Study 201) at Day 540 was −1.0 (0.83) and at Day 720 was −2.9 (1.74).
   Treatment Phase, the change from baseline (Study 202) at Day 360 in retreated and redosed subjects was −2.4 (2.37).
By investigators on the I-GAIS during the:
   Observation Phase, the cellulite of more than half (64.9%) of CCH-treated subjects, was assessed by investigators on the I-GAIS as very much improved (4.1%), much improved (23.7%), or improved (37.1%).
   Treatment Phase, at Day 360 for Treatment Course 1, the cellulite of approximately three-quarters (73.2%) of subjects in total, was assessed as improved (81 subjects; 49.4%), much improved (32 subject; 19.5%), or very much improved (7 subjects; 4.3%).
   Treatment Phase, at Day 360 for Treatment Course 2 more than half (64.2%) of subjects overall, was assessed as improved (24 subjects; 35.8%), much improved (16 subjects; 23.9%), or very much improved (3 subjects; 4.5%).
   Treatment Phase, at Day 360, in subjects receiving a second CCH treatment, the cellulite of more than two-thirds (71.8%) of retreated and redosed subjects was assessed as improved 64 (45.1%), much improved 33 (23.2%), or very much improved 5 (3.5%).
By subjects on the S-GAIS during the:
   Observation Phase, more than two-thirds (69.1%) of the CCH-treated subjects in total, assessed their cellulite as either improved (46 subjects; 47.4%), much improved (18 subjects; 18.6%), or very much improved (3 subjects; 3.1%).
   During the Treatment Phase, at Day 360 for Treatment Course 1, more than two-thirds (66.2%) of subjects overall, assessed their cellulite as either improved (73 subjects; 44.8%), much improved (18 subjects; 11%), or very much improved 17 (10.4%).
   During the Treatment Phase, at Day 360, for Treatment Course 2, approximately half (51.5%) of subjects assessed their cellulite as either improved (24 subjects; 35.3%), much improved (6 subjects; 8.8%), or very much improved (5 subjects; 7.4%).
   During the Treatment Phase at Day 360 subjects receiving a second CCH treatment more than half (64.4%) of subjects overall, assessed their cellulite as either improved (64 subjects; 44.8%), much improved (14 subjects; 9.8%), or very much improved 16 (11.2%).
By subjects on the Subject Satisfaction with EFP Treatment Scale, during the:
   Observation Phase, at Day 360, 41(42.3%) and 14 (14.4%) CCH-treated subjects responded that they were either satisfied or were very satisfied with treatment.
   During the Treatment Phase, at Day 360 for Treatment Course 1, Observation Day 360, 55 (33.7%) subjects were satisfied and 26 (16.0%) subjects were very satisfied with treatment.
   During the Treatment Phase, at Day 360 for Treatment Course 2, Observation Day 360, 21 (30.9%) subjects were satisfied and 10 (14.7%) subjects were very satisfied with treatment.
   During the Treatment Phase, in subjects receiving a second CCH treatment, 50 (35%), subjects were satisfied and 25 (17.5%) subjects were very satisfied with treatment.

Data from this study, demonstrated the persistence of effect of CCH 0.84 mg per treatment area×3 treatment sessions for up to 1 year in 193 subjects. The persistence of effect was also demonstrated in for up to 2 years after CCH treatment in subjects sampled for the 2-year evaluation. The results also demonstrated that that administration of CCH at doses of 0.84 mg subcutaneously per treatment area per treatment session×3 treatment sessions 21 days apart, to the buttock or thigh was effective for both treatment of naïve subjects and in subjects retreated and redosed. A reduction in the severity of cellulite was shown on multiple instruments including investigator and subject rating scales. Results of effectiveness assessments were similar for buttock and thigh-treated regions.

In 162 subjects re-exposed to a second treatment course of CCH 0.84 mg per treatment area×3 treatment sessions, and evaluated for effectiveness, no tolerance to treatment was observed (i.e., no loss of treatment effectiveness). This was supported by the results of Study 202, where the reduction observed on the CR-PCSS and PR-PCSS were similar for both the 162 subject's response after their second treatment course compared to their first treatment course as well as to the response observed in the pivotal Phase 3 studies.

Results of this study demonstrate that the persistence of effect was sustained for up to 1-year as evidenced by the change from baseline of the PR-PCSS and CR-PCSS ratings and sustained response on the PR-PCSS and CR-PCSS persisted for 1 year. Study results were also supportive of the persistence of 1-level and 2-level composite response for up to 2 years

Subjects re-exposed (i.e., retreated or redosed) to CCH in Study 202, experienced a reduction in the severity of cellulite on the CR-PCSS and the PR-PCSS after their second treatment course of a magnitude similar to the response observed in those subjects after their first treatment course and to subjects in the double-blind studies (Studies 201, 302, and 303).

The durability of response to CCH was demonstrated in evaluable subjects during Observation, Long-term Durability, and Treatment Phases of the study. Among 2-level composite responders in Study 201, all evaluable subjects maintained durability of response in that none returned to baseline CR-PCSS and PR-PCSS levels at Days 180 and 360. Among 1-level composite responders in Study 201, almost all subjects maintained a durable treatment response at Days 180 and 360. Similar results were observed in subjects treated in this study; all evaluable subjects maintained a durable response at Days 180 and 360.

Long-term durability from baseline scores in Study 201 of up to 2 years was demonstrated in evaluable 2-level and 1-level composite responders, in CR-PCSS responders, and PR-PCSS responders. The effects of CCH on the severity of cellulite from the baseline in Study 201 were maintained up to at least 1 year from the baseline in Study 201 as measured by the Hexsel CSS Total Score, I-GAIS, S-GAIS, and Subject Satisfaction with EFP Treatment Scores.

The effectiveness of CCH 0.84 mg per treatment area was assessed in both treatment naïve and in subjects re-exposed (retreated/redosed) in this study. The result of effectiveness measurements assessed by the CR-PCSS, PR-PCSS, Hexsel CSS total score, I-GAIS, S-GAIS and the Subject Satisfaction with EFP Treatment scores were consistent with the results in the Observation Populations and demonstrated improvement in cellulite severity, whether assessed by the investigator or subject. Similar results were observed in the subjects retreated and redosed. It is important to note that of the subjects that opted to receive a second dose, few subjects were retreated with the majority redosed.

The overall results of this Phase 2 long-term, open-label, multicenter study of CCH provide further evidence of the safety, tolerability, effectiveness, and long-term durability of CCH in the treatment of EFP.

The embodiments described herein are intended to be merely exemplary. Persons skilled in the art will understand that variations and modifications may be made without departing from the scope of the invention encompassed by the claims below.

### Embodiments

The following list of embodiments is intended to complement, rather than displace or supersede, the previous descriptions.

Embodiment 1. A method of reducing the severity of cellulite in both buttocks of a human patient, comprising the steps of:
a. providing a collagenase composition having at least two of the following characteristics:
   i. Vₘₐₓ(min⁻¹) of about 0.08 to 7.70 (SRC assay), or about 0.3 to 30.5 (GPA assay);
   ii. K_{M}, of about 4.1 to 410 nanoMolar (SRC assay), or about 0.03 to 3.1 mM (GPA assay);
   iii. K_{cat} (sec⁻¹) of about 1.1 to 107 (SRC assay), or about 93 to 9,179 (GPA assay);
   iv. 1/K_{cat}, microseconds of about 376 to 37,222 (SRC assay), or about 4 to 428 (GPA assay);
   v. K_{cat}/K_{M}, mM⁻¹sec⁻¹ of about 5,140 to 508,814 (SRC assay), or about 60 to 5,934 (GPA assay);
   vi. A molecular mass from about 60 kDa to about 130 kDa, or about 70 to about 130 kDa, or about 80 to about 120 kDa, or about 90 to about 120 kDa, or about 100 to about 110 kDa;
   vii. A purity by area of at least 80% as measured by reverse phase HPLC (high pressure liquid chromatography);
   viii. A potency of about 5,000 to about 30,000 f-SRC units/mg;
   ix. A potency of about 175,000 to about 500,000 f-GPA units/mg;
   x. A potency of about 5,000 to about 25,000 ABC units/mg;
   xi. Less than or equal to 1% by area of an impurity selected from the group consisting of clostripain, gelatinase, and leupeptin;
   xii. Less than or equal to 1 cfu/mL bioburden; and
b. injecting a therapeutically effective amount of the collagenase composition into dimples in both buttocks according to Treatment I, wherein an improvement in an appearance of the cellulite is established by a scale or other measurement tools selected from the group consisting of Hexsel Cellulite Severity Scale (Hexsel CSS), Hexsel Depression Depth Score, Likert Scale, Dimple Analysis, Clinician Reported Photonumeric Cellulite Severity Scale (CR-PCSS), Patient Reported Photonumeric Cellulite Severity Scale (PR-PCSS), Investigator Global Aesthetic Improvement Scale (I-GAIS), Subject Global Aesthetic Improvement Scale (S-GAIS), Patient Reported Cellulite Impact Scale (PR-CIS), PR-CIS Abbreviated, Subject Self-Rating Scale (SSRS), Subject Satisfaction with Cellulite Treatment (SSCT), Clinician assessment of cellulite severity (photography or other imagery), Body-Q, and a validated photonumeric or other scale used by clinicians and/or patients to assess cellulite severity, improvement, and/or patient satisfaction.
   Embodiment 2. The method of embodiment 1 wherein the dimples treated are not dependent on their size or distance from each other.
   Embodiment 3. The method of embodiment 1 wherein the dimples treated are devoid of skin laxity, flaccidity or sagging skin.
   Embodiment 4. The method of embodiment 1 wherein the patient has a plurality of treatment visits and different dimples are treated at different treatment visits.
   Embodiment 5. The method of embodiment 1 wherein the injections are made with a 12 inch needle.
   Embodiment 6. The method of embodiment 1 wherein the injections are administered by a clinician who does not rely on a spacer, ruler, paper or other device to limit the location of the injections.
   Embodiment 7. The method of embodiment 1 wherein at least one injection occurs at a nadir of the dimple.
   Embodiment 8. The method of embodiment 1 wherein a plurality of injections occur within 2 cm of each other.
   Embodiment 9. The method of embodiment 1 wherein the dimples treated are less than 1 cm long or are more than 2 cm long.
   Embodiment 10. The method of embodiment 1 wherein the patient experiences a rapid rate of response to therapy.
   Embodiment 11. The method of embodiment 1 wherein when the treatment is administered to a population of patients who all have CR-PCSS baseline ratings of moderate or severe, the treatment results in an outcome selected from the group consisting of:
a. At least 50% of the patients show improvement in severity at Day 22, 43, or 71 from baseline of at least 1 level of severity in the CR-PCSS as assessed live by the clinician of the buttocks;
b. at least 50% of the patients show improvement in severity at Day 22, 43, or 71 from baseline of at least 1 level of severity in the PR-PCSS as assessed by the subject while viewing a digital image of the buttocks;
c. at least 5% of the patients show improvement in severity at Day 22, 43, or 71 from baseline of at least 2 levels of severity in the CR-PCSS as assessed live by the clinician of the buttocks;
d. at least 5% of the patients show improvement in severity at Day 22, 43, or 71 from baseline of at least 2 levels of severity in the PR-PCSS as assessed by the subject while viewing a digital image of the buttocks; and
e. at least 5% of the patients experience a decrease in dimple size.
   Embodiment 12. The method of embodiment 11 wherein the dimple size reduction parameter is selected from the group consisting of volume, length, width, and depth.
   Embodiment 13. The method of embodiment 11 wherein the dimple size reduction is at least a 10% decrease at Day 22, 43, or 71 from baseline.
   Embodiment 14. The method of embodiment 1 wherein the cumulative collagenase dose injected is about 5.04 mg.
   Embodiment 15. The method of embodiment 1 wherein the collagenase composition comprises AUX-I and AUX-II having the following characteristics:
a. AUX-I (SRC assay):
   i. Vmax, min⁻¹: About 0.08 to 7.70
   ii. K_{M}: About 4.1 to 410 nanoMolar
   iii. K_{cat}, sec⁻¹: About 1.1 to 107
   iv. 1/K_{cat}, microseconds: About 376 to 37,222
   v. K_{cat}/K_{M}, mM⁻¹sec⁻¹: About 5,140 to 508,814
b. AUX-II (GPA assay)
   i. Vₘₐₓ, min⁻¹: About 0.3 to 30.5
   ii. K_{M}, mM: About 0.03 to 3.1
   iii. K_{cat}, sec⁻¹: About 93 to 9,179
   iv. 1/K_{cat}, microseconds: About 4 to 428
   v. K_{cat}/K_{M}, mM⁻¹sec⁻¹: About 60 to 5,934
      Embodiment 16. The method of embodiment 1 wherein the collagenase composition comprises AUX-I and AUX-II having the following characteristics:
a. AUX-I (SRC assay):
   i. Vmax, min⁻¹: About 3.8
   ii. K_{M}, mM: About 2.07×10⁻⁴
   iii. K_{cat}, sec⁻¹: About 53
   iv. 1/K_{cat}, microseconds: About 18,799
   v. k_{cat}/K_{M}, mM⁻¹sec⁻¹: About 256,977
b. AUX II (GPA assay):
   i. Vmax, min⁻¹: About 15.4
   ii. K_{M}, mM: About 1.6
   iii. Kcat, sec⁻¹: About 4,636
   iv. 1/K_{cat}, microseconds: About 216
   v. k_{cat}/K_{M}, mM⁻¹sec⁻¹: About 2,997
      Embodiment 17. The method of embodiment 1 wherein the composition comprises at least 3 of the characteristics.
      Embodiment 18. The method of embodiment 1 wherein the composition comprises at least 4 of the characteristics.
      Embodiment 19. The method of embodiment 1 wherein the composition comprises at least 5 of the characteristics.
      Embodiment 20. The method of embodiment 1 wherein the composition comprises about 1 mg to 20 mg of one or more collagenases.
      Embodiment 21. The method of embodiment 1 wherein the composition comprises CCH.
      Embodiment 22. The method of embodiment 1 wherein the composition has a potency of about 10,000 ABC units/0.58 mg and the therapeutically effective amount is about 1 mg to 20 mg.
      Embodiment 23. The method of embodiment 1 wherein the composition has a potency of about 15,000 ABC units/mg to 20,000 ABC units/mg and the therapeutically effective amount is about 1 mg to 20 mg.
      Embodiment 24. The method of embodiment 1 wherein the therapeutically effective amount is about 1 mg to 10 mg and the composition has a potency of about 20,000 to about 30,000 f-SRC units/mg or about 175,000 to about 300,000 f-GPA units/mg.
      Embodiment 25. The method of embodiment 1 wherein when the treatment is administered to a population of patients, the treatment results in at least 5% of patients maintaining their level of improvement versus pretreatment baseline for at least 71 days after the initial dose.
      Embodiment 26. The method of embodiment 25 wherein at least 10% of patients maintain their level of improvement versus pretreatment baseline for at least 71 days after the initial dose.
      Embodiment 27. The method of embodiment 25 wherein at least 20% of patients maintain their level of improvement versus pretreatment baseline for at least 71 days after the initial dose.
      Embodiment 28. The method of embodiment 1 wherein when the treatment is administered to a population of patients, the treatment results in at least 5% of patients demonstrating improvement versus pretreatment baseline and showing an additional increase in improvement over time.
      Embodiment 29. The method of embodiment 1 wherein the treatment results in at least one of the following efficacy endpoints as measured by CR-PCSS and/or PR-PCSS:
a. An improvement in severity at Day 22, 43, 71, 90, 180 or 365 from baseline (pretreatment “Day 1”) of at least 2 levels of severity in the CR-PCSS as assessed live by the clinician of the buttocks;
b. An improvement in severity at Day 22, 43, 71, 90, 180 or 365 from baseline (Day 1) of at least 2 levels of severity in the PR-PCSS as assessed by the patient while viewing a digital image of the buttocks;
c. An improvement demonstrated by a 2-level composite response at Day 22, 43, 71, 90, 180 or 365 defined as a patient with an improvement from baseline of at least 2 levels of severity in the CR-PCSS and an improvement from baseline of at least 2 levels of severity in the PR-PCSS;
d. An improvement in severity at Day 22, 43, 71, 90, 180 or 365 from baseline (Day 1) of at least 1 level of severity in the CR-PCSS as assessed live by the clinician of the buttocks;
e. An improvement in severity at Day 22, 43, 71, 90, 180 or 365 from baseline (Day 1) of at least 1 level of severity in the PR-PCSS as assessed by the patient while viewing a digital image of the buttocks;
f. An improvement demonstrated by a 1-level composite response at Day 22, 43, 71, 90, 180 or 365 defined as a patient with an improvement from baseline of at least 1 level of severity in the CR-PCSS and an improvement from baseline of at least 1 level of severity in the PR-PCSS; and
g. In a population of patients who all had CR-PCSS ratings of moderate or severe at baseline, the improvement in at least one treatment area was statistically significant compared to placebo wherein the improvement is one or more of a. to f. above.
   Embodiment 30. The method of embodiment 1 wherein the treatment results in at least one of the following efficacy endpoints as measured by dimple analysis wherein:
a. depth decreases by at least 5%;
b. width decreases by at least 5%;
c. length decreases by at least 5%;
d. overall volume decreases by at least 5%; and
e. surface area decreases by at least 5%.
   Embodiment 31. A method of reducing the severity of cellulite in both buttocks of a human patient, comprising the steps of:
a. providing a collagenase composition having at least two of the following characteristics:
   i. Vₘₐₓ(min⁻¹) of about 0.08 to 7.70 (SRC assay), or about 0.3 to 30.5 (GPA assay);
   ii. K_{M}, of about 4.1 to 410 nanoMolar (SRC assay), or about 0.03 to 3.1 mM (GPA assay);
   iii. K_{cat} (sec⁻¹) of about 1.1 to 107 (SRC assay), or about 93 to 9,179 (GPA assay);
   iv. 1/K_{cat}, microseconds of about 376 to 37,222 (SRC assay), or about 4 to 428 (GPA assay);
   v. K_{cat}/K_{M}, mM⁻¹sec⁻¹ of about 5,140 to 508,814 (SRC assay), or about 60 to 5,934 (GPA assay);
   vi. A molecular mass from about 60 kDa to about 130 kDa, or about 70 to about 130 kDa, or about 80 to about 120 kDa, or about 90 to about 120 kDa, or about 100 to about 110 kDa;
   vii. A purity by area of at least 80% as measured by reverse phase HPLC (high pressure liquid chromatography);
   viii. A potency of about 5,000 to about 30,000 f-SRC units/mg;
   ix. A potency of about 175,000 to about 500,000 f-GPA units/mg;
   x. A potency of about 5,000 to about 25,000 ABC units/mg;
   xi. Less than or equal to 1% by area of an impurity selected from the group consisting of clostripain, gelatinase, and leupeptin;
   xii. Less than or equal to 1 cfu/mL bioburden; and
b. injecting a therapeutically effective amount of the collagenase composition into dimples in both buttocks according to Treatment I, wherein bruising significantly decreases or resolves in color intensity at between about 3 days and 20 days after a treatment visit.
   Embodiment 32. The method of embodiment 31 wherein the cumulative collagenase dose injected is about 5.04 mg.
   Embodiment 33. The method of embodiment 31 wherein the collagenase composition comprises AUX-I and AUX-II having the following characteristics:
a. AUX-I (SRC assay):
   i. Vₘₐₓ, min⁻¹: About 0.08 to 7.70
   ii. K_{M}: About 4.1 to 410 nanoMolar
   iii. K_{cat}, sec⁻¹: About 1.1 to 107
   iv. 1/K_{cat}, microseconds: About 376 to 37,222
   v. K_{cat}/K_{M}, mM⁻¹sec⁻¹: About 5,140 to 508,814
b. AUX-II (GPA assay)
   i. Vₘₐₓ, min⁻¹: About 0.3 to 30.5
   ii. K_{M}, mM: About 0.03 to 3.1
   iii. K_{cat}, sec⁻¹: About 93 to 9,179
   iv. 1/K_{cat}, microseconds: About 4 to 428
   v. K_{cat}/K_{M}, mM⁻¹sec⁻¹: About 60 to 5,934
      Embodiment 34. The method of embodiment 31 wherein the collagenase composition comprises AUX-I and AUX-II having the following characteristics:
a. AUX-I (SRC assay):
   i. Vₘₐₓ, min⁻¹: About 3.8
   ii. K_{M}, mM: About 2.07×10⁻⁴
   iii. K_{cat}, sec⁻¹: About 53
   iv. 1/K_{cat}, microseconds: About 18,799
   v. k_{cat}/K_{M}, mM⁻¹sec⁻¹: About 256,977
b. AUX II (GPA assay):
   i. Vₘₐₓ, min⁻¹: About 15.4
   ii. K_{M}, mM: About 1.6
   iii. K_{cat}, sec⁻¹: About 4,636
   iv. 1/K_{cat}, microseconds: About 216
   v. k_{cat}/K_{M}, mM⁻¹sec⁻¹: About 2,997
      Embodiment 35. The method of embodiment 31 wherein the composition comprises at least 3 of the characteristics.
      Embodiment 36. The method of embodiment 31 wherein the composition comprises at least 4 of the characteristics.
      Embodiment 37. The method of embodiment 31 wherein the composition comprises at least 5 of the characteristics.
      Embodiment 38. The method of embodiment 31 wherein the composition comprises about 1 mg to 20 mg of one or more collagenases.
      Embodiment 39. The method of embodiment 31 wherein the composition comprises CCH.
      Embodiment 40. The method of embodiment 31 wherein the composition has a potency of about 10,000 ABC units/0.58 mg and the therapeutically effective amount is about 1 mg to 20 mg.
      Embodiment 41. The method of embodiment 31 wherein the composition has a potency of about 15,000 ABC units/mg to 20,000 ABC units/mg and the therapeutically effective amount is about 1 mg to 20 mg.
      Embodiment 42. The method of embodiment 31 wherein the therapeutically effective amount is about 1 mg to 10 mg and the composition has a potency of about 20,000 to about 30,000 f-SRC units/mg or about 175,000 to about 300,000 f-GPA units/mg.
      Embodiment 43. The method of embodiment 31 wherein when the treatment is administered to a population of patients, the treatment results in at least 5% of patients maintaining their level of improvement versus pretreatment baseline for at least 71 days after the initial dose.
      Embodiment 44. The method of embodiment 43 wherein at least 10% of patients maintain their level of improvement versus pretreatment baseline for at least 71 days after the initial dose.
      Embodiment 45. The method of embodiment 43 wherein at least 20% of patients maintain their level of improvement versus pretreatment baseline for at least 71 days after the initial dose.
      Embodiment 46. The method of embodiment 31 wherein when the treatment is administered to a population of patients, the treatment results in at least 5% of patients demonstrating improvement versus pretreatment baseline and showing an additional increase in improvement over time.
      Embodiment 47. A method of reducing the severity of cellulite in both buttocks of a human patient, comprising the steps of:
a. providing a collagenase composition having at least two of the following characteristics:
   i. Vₘₐₓ(min⁻¹) of about 0.08 to 7.70 (SRC assay), or about 0.3 to 30.5 (GPA assay);
   ii. K_{M}, of about 4.1 to 410 nanoMolar (SRC assay), or about 0.03 to 3.1 mM (GPA assay);
   iii. K_{cat} (sec⁻¹) of about 1.1 to 107 (SRC assay), or about 93 to 9,179 (GPA assay);
   iv. 1/K_{cat}, microseconds of about 376 to 37,222 (SRC assay), or about 4 to 428 (GPA assay);
   v. K_{cat}/K_{M}, mM⁻¹ sec⁻¹ of about 5,140 to 508,814 (SRC assay), or about 60 to 5,934 (GPA assay);
   vi. A molecular mass from about 60 kDa to about 130 kDa, or about 70 to about 130 kDa, or about 80 to about 120 kDa, or about 90 to about 120 kDa, or about 100 to about 110 kDa:
   vii. A purity by area of at least 80% as measured by reverse phase HPLC (high pressure liquid chromatography);
   viii. A potency of about 5,000 to about 30,000 f-SRC units/mg;
   ix. A potency of about 175,000 to about 500,000 f-GPA units/mg;
   x. A potency of about 5,000 to about 25,000 ABC units/mg;
   xi. Less than or equal to 1% by area of an impurity selected from the group consisting of clostripain, gelatinase, and leupeptin;
   xii. Less than or equal to 1 cfu/mL bioburden; and
b. injecting a therapeutically effective amount of the collagenase composition into dimples of both buttocks according to Treatment I, wherein an improvement in an appearance of the cellulite is established by CR-PCSS.
   Embodiment 48. The method of embodiment 47 wherein the cumulative collagenase dose injected is about 5.04 mg.
   Embodiment 49. The method of embodiment 47 wherein the collagenase composition comprises AUX-I and AUX-II having the following characteristics:
a. AUX-I (SRC assay):
   i. Vₘₐₓ, min⁻¹: About 0.08 to 7.70
   ii. K_{M}: About 4.1 to 410 nanoMolar
   iii. K_{cat}, sec⁻¹: About 1.1 to 107
   iv. 1/K_{cat}, microseconds: About 376 to 37,222
   v. K_{cat}/K_{M}, mM⁻¹sec⁻¹: About 5,140 to 508,814
b. AUX-II (GPA assay)
   i. Vₘₐₓ, min⁻¹: About 0.3 to 30.5
   ii. K_{M}, mM: About 0.03 to 3.1
   iii. K_{cat}, sec⁻¹: About 93 to 9,179
   iv. 1/K_{cat}, microseconds: About 4 to 428
   v. K_{cat}/K_{M}, mM⁻¹sec⁻¹: About 60 to 5,934
      Embodiment 50. The method of embodiment 47 wherein the collagenase composition comprises AUX-I and AUX-II having the following characteristics:
a. AUX-I (SRC assay):
   i. Vₘₐₓ, min⁻¹: About 3.8
   ii. K_{M}, mM: About 2.07×10⁻⁴
   iii. K_{cat}, sec⁻¹: About 53
   iv. 1/K_{cat}, microseconds: About 18,799
   v. k_{cat}/K_{M}, mM⁻¹sec⁻¹: About 256,977
b. AUX II (GPA assay):
   i. Vₘₐₓ, min⁻¹: About 15.4
   ii. K_{M}, mM: About 1.6
   iii. K_{cat}, sec⁻¹: About 4,636
   iv. 1/K_{cat}, microseconds: About 216
   v. k_{cat}/K_{M}, mM⁻¹sec⁻¹: About 2,997
      Embodiment 51. The method of embodiment 47 wherein the composition comprises at least 3 of the characteristics.
      Embodiment 52. The method of embodiment 47 wherein the composition comprises at least 4 of the characteristics.
      Embodiment 53. The method of embodiment 47 wherein the composition comprises at least 5 of the characteristics.
      Embodiment 54. The method of embodiment 47 wherein the composition comprises about 1 mg to 20 mg of one or more collagenases.
      Embodiment 55. The method of embodiment 47 wherein the composition comprises CCH.
      Embodiment 56. The method of embodiment 47 wherein the composition has a potency of about 10,000 ABC units/0.58 mg and the therapeutically effective amount is about 1 mg to 20 mg.
      Embodiment 57. The method of embodiment 47 wherein the composition has a potency of about 15,000 ABC units/mg to 20,000 ABC units/mg and the therapeutically effective amount is about 1 mg to 20 mg.
      Embodiment 58. The method of embodiment 47 wherein the therapeutically effective amount is about 1 mg to 10 mg and the composition has a potency of about 20,000 to about 30,000 f-SRC units/mg or about 175,000 to about 300,000 f-GPA units/mg.
      Embodiment 59. The method of embodiment 47 wherein when the treatment is administered to a population of patients, the treatment results in at least 5% of patients maintaining their level of improvement versus pretreatment baseline for at least 71 days after the initial dose.
      Embodiment 60. The method of embodiment 59 wherein at least 10% of patients maintain their level of improvement versus pretreatment baseline for at least 71 days after the initial dose.
      Embodiment 61. The method of embodiment 59 wherein at least 20% of patients maintain their level of improvement versus pretreatment baseline for at least 71 days after the initial dose.
      Embodiment 62. The method of embodiment 47 wherein when the treatment is administered to a population of patients, the treatment results in at least 5% of patients demonstrating improvement versus pretreatment baseline and showing an additional increase in improvement over time.
      Embodiment 63. The method of embodiment 47 wherein the treatment results in at least one of the following efficacy endpoints as measured by CR-PCSS:
a. An improvement in severity at Day 22, 43, 71, 90, 180 or 365 days from baseline (pretreatment “Day 1”) of at least 2 levels of severity in the CR-PCSS as assessed live by the clinician of the buttocks;
b. An improvement in severity at Day 22, 43, 71, 90, 180 or 365 from baseline (Day 1) of at least 2 levels of severity in the PR-PCSS as assessed by the patient while viewing a digital image of the buttocks;
c. An improvement demonstrated by a 2-level composite response at Day 22, 43, 71, 90, 180 or 365 defined as a patient with an improvement from baseline of at least 2 levels of severity in the CR-PCSS and an improvement from baseline of at least 2 levels of severity in the PR-PCSS;
d. An improvement in severity at Day 22, 43, 71, 90, 180 or 365 from baseline (Day 1) of at least 1 level of severity in the CR-PCSS as assessed live by the clinician of the buttocks;
e. An improvement in severity at Day 22, 43, 71, 90, 180 or 365 from baseline (Day 1) of at least 1 level of severity in the PR-PCSS as assessed by the patient while viewing a digital image of the buttocks;
f. An improvement demonstrated by a 1-level composite response at Day 22, 43, 71, 90, 180 or 365 defined as a patient with an improvement from baseline of at least 1 level of severity in the CR-PCSS and an improvement from baseline of at least 1 level of severity in the PR-PCSS; and
g. In a population of patients who all had CR-PCSS or PR-PCSS ratings of moderate or severe, the improvement in at least one treatment area was statistically significant compared to placebo wherein the improvement is one or more of a. to f. above.

1. A method of reducing the severity of cellulite in buttocks of a human patient, comprising the steps of:
a. providing a collagenase composition having at least two of the following characteristics:
i. Vₘₐₓ (min⁻¹) of about 0.08 to 7.70 (SRC assay), or about 0.3 to 30.5 (GPA assay);
ii. K_{M}, of about 4.1 to 410 nanoMolar (SRC assay), or about 0.03 to 3.1 mM (GPA assay);
iii. K_{cat}(sec⁻¹) of about 1.1 to 107 (SRC assay), or about 93 to 9,179 (GPA assay);
iv. 1/K_{cat}, microseconds of about 376 to 37,222 (SRC assay), or about 4 to 428 (GPA assay);
v. K_{cat}/K_{M}, mM⁻¹sec⁻¹ of about 5,140 to 508,814 (SRC assay), or about 60 to 5,934 (GPA assay);
vi. A molecular mass from about 60 kDa to about 130 kDa, or about 70 kDa to about 130 kDa, or about 80 kDa to about 120 kDa, or about 90 kDa to about 120 kDa, or about 100 kDa to about 110 kDa;
vii. A purity by area of at least 80% as measured by reverse phase HPLC (high pressure liquid chromatography);
viii. A potency of about 5,000 to about 30,000 f-SRC units/mg;
ix. A potency of about 175,000 to about 500,000 f-GPA units/mg;
x. A potency of about 5,000 to about 25,000 ABC units/mg;
xi. Less than or equal to 1% by area of an impurity selected from the group consisting of clostripain, gelatinase, and leupeptin;
xii. Less than or equal to 1 cfu/mL bioburden; and
b. injecting a therapeutically effective amount of the collagenase composition into dimples in the buttocks according to Treatment I,
wherein an improvement in an appearance of the cellulite is established by a scale or other measurement tools selected from the group consisting of Hexsel Cellulite Severity Scale (Hexsel CSS), Hexsel Depression Depth Score, Likert Scale, Dimple Analysis, Clinician Reported Photonumeric Cellulite Severity Scale (CR-PCSS), Patient Reported Photonumeric Cellulite Severity Scale (PR-PCSS), Investigator Global Aesthetic Improvement Scale (I-GAIS), Subject Global Aesthetic Improvement Scale (S-GAIS), Patient Reported Cellulite Impact Scale (PR-CIS), PR-CIS Abbreviated, Subject Self-Rating Scale (SSRS), Subject Satisfaction with Cellulite Treatment (SSCT), Clinician assessment of cellulite severity (photography or other imagery), Body-Q, and a validated photonumeric or other scale used by clinicians and/or patients to assess cellulite severity, improvement, and/or patient satisfaction; or
wherein bruising significantly decreases or resolves in color intensity at between about 3 days and 20 days after a treatment visit.

2. The method of claim 1 wherein the dimples treated are not dependent on their size or distance from each other.

3. The method of claim 1 wherein the dimples treated are devoid of skin laxity, flaccidity or sagging skin.

4. The method of claim 1 wherein the patient has a plurality of treatment visits and different dimples are treated at different treatment visits.

5. The method of claim 1 wherein the injections are made with a 1% inch needle.

6. The method of claim 1 wherein the injections are administered by a clinician who does not rely on a spacer, ruler, paper or other device to limit the location of the injections.

7. The method of claim 1 wherein at least one injection occurs at a nadir of the dimple.

8. The method of claim 1 wherein a plurality of injections occur within 2 cm of each other.

9. The method of claim 1 wherein the dimples treated are less than 1 cm long or are more than 2 cm long.

10. The method of claim 1 wherein the patient experiences a rapid rate of response to therapy.

11. The method of claim 1 wherein when the treatment is administered to a population of patients who all have CR-PCSS baseline ratings of moderate or severe, the treatment results in an outcome selected from the group consisting of:
a. At least 50% of the patients show improvement in severity at Day 22, 43, or 71 from baseline of at least 1 level of severity in the CR-PCSS as assessed live by the clinician of the buttocks;
b. at least 50% of the patients show improvement in severity at Day 22, 43, or 71 from baseline of at least 1 level of severity in the PR-PCSS as assessed by the subject while viewing a digital image of the buttocks;
c. at least 5% of the patients show improvement in severity at Day 22, 43, or 71 from baseline of at least 2 levels of severity in the CR-PCSS as assessed live by the clinician of the buttocks;
d. at least 5% of the patients show improvement in severity at Day 22, 43, or 71 from baseline of at least 2 levels of severity in the PR-PCSS as assessed by the subject while viewing a digital image of the buttocks; and
e. at least 5% of the patients experience a decrease in dimple size.

12. The method of claim 11 wherein the dimple size reduction parameter is selected from the group consisting of volume, length, width, and depth.

13. The method of claim 11 wherein the dimple size reduction is at least a 10% decrease at Day 22, 43, or 71 from baseline.

14. The method of claim 1 wherein the cumulative collagenase dose injected is about 5.04 mg.

15. The method of claim 1 wherein the collagenase composition comprises AUX-I and AUX-II having the following characteristics:
a. AUX-I (SRC assay):
i. Vₘₐₓ, min⁻¹: About 0.08 to 7.70
ii. K_{M}: About 4.1 to 410 nanoMolar
iii. K_{cat}, sec⁻¹: About 1.1 to 107
iv. 1/K_{cat}, microseconds: About 376 to 37,222
v. K_{cat}/K_{M}, mM⁻¹ sec⁻¹: About 5,140 to 508,814
b. AUX-II (GPA assay)
i. Vₘₐₓ, min⁻¹: About 0.3 to 30.5
ii. K_{M}, mM: About 0.03 to 3.1
iii. K_{cat} sec⁻¹: About 93 to 9,179
iv. 1/K_{cat}, microseconds: About 4 to 428
v. K_{cat}/K_{M}, mM⁻¹ sec⁻¹: About 60 to 5,93

16. (canceled)

17. The method of claim 1 wherein the composition comprises at least 3 of the characteristics.

18. The method of claim 1 wherein the composition comprises at least 4 of the characteristics.

19. The method of claim 1 wherein the composition comprises at least 5 of the characteristics.

20. The method of claim 1 wherein the composition comprises about 1 mg to 20 mg of one or more collagenases.

21-22. (canceled)

23. The method of claim 1 wherein the composition has a potency of about 15,000 ABC units/mg to 20,000 ABC units/mg and the therapeutically effective amount is about 1 mg to 20 mg.

24. The method of claim 1 wherein the therapeutically effective amount is about 1 mg to 10 mg and the composition has a potency of about 20,000 to about 30,000 f-SRC units/mg or about 175,000 to about 300,000 f-GPA units/mg.

25. The method of claim 1 wherein when the treatment is administered to a population of patients, the treatment results in at least 5% of patients maintaining their level of improvement versus pretreatment baseline for at least 71 days after the initial dose.

26. The method of claim 25 wherein at least 10% of patients maintain their level of improvement versus pretreatment baseline for at least 71 days after the initial dose.

27. The method of claim 25 wherein at least 20% of patients maintain their level of improvement versus pretreatment baseline for at least 71 days after the initial dose.

28. The method of claim 1 wherein when the treatment is administered to a population of patients, the treatment results in at least 5% of patients demonstrating improvement versus pretreatment baseline and showing an additional increase in improvement over time.

29. The method of claim 1 wherein the treatment results in at least one of the following efficacy endpoints as measured by CR-PCSS and/or PR-PCSS:
a. An improvement in severity at Day 22, 43, 71, 90, 180 or 365 from baseline (pretreatment “Day 1”) of at least 2 levels of severity in the CR-PCSS as assessed live by the clinician of the buttocks;
b. An improvement in severity at Day 22, 43, 71, 90, 180 or 365 from baseline (Day 1) of at least 2 levels of severity in the PR-PCSS as assessed by the patient while viewing a digital image of the buttocks;
c. An improvement demonstrated by a 2-level composite response at Day 22, 43, 71, 90, 180 or 365 defined as a patient with an improvement from baseline of at least 2 levels of severity in the CR-PCSS and an improvement from baseline of at least 2 levels of severity in the PR-PCSS;
d. An improvement in severity at Day 22, 43, 71, 90, 180 or 365 from baseline (Day 1) of at least 1 level of severity in the CR-PCSS as assessed live by the clinician of the buttocks;
e. An improvement in severity at Day 22, 43, 71, 90, 180 or 365 from baseline (Day 1) of at least 1 level of severity in the PR-PCSS as assessed by the patient while viewing a digital image of the buttocks;
f. An improvement demonstrated by a 1-level composite response at Day 22, 43, 71, 90, 180 or 365 defined as a patient with an improvement from baseline of at least 1 level of severity in the CR-PCSS and an improvement from baseline of at least 1 level of severity in the PR-PCSS; and
g. In a population of patients who all had CR-PCSS or PR-PCSS ratings of moderate or severe at baseline, the improvement in at least one treatment area was statistically significant compared to placebo wherein the improvement is one or more of a. to f. above.

30. The method of claim 1 wherein the treatment results in at least one of the following efficacy endpoints as measured by dimple analysis wherein:
a. depth decreases by at least 5%;
b. width decreases by at least 5%;
c. length decreases by at least 5%;
d. overall volume decreases by at least 5%; and
e. surface area decreases by at least 5%.

31-63. (canceled)
